(19) United States
(12) Patent Application Publication
(10) Pub. No.: US 2005/0288340 A1
(43) Pub. Date: Dec. 29, 2005

(54) Substituted heteroaryl- and phenylsulfamoyl compounds

(75) Inventor: Ernest S. Hamanaka, Gales Ferry, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/065,774

(22) Filed: Feb. 24, 2005

Related U.S. Application Data
(60) Provisional application No. 60/583,721, filed on Jun. 29, 2004.

(51) Int. Cl. A61K 31/428; A61K 31/426; A61K 31/421

(52) U.S. Cl. 514/365; 514/367; 514/374; 548/152; 548/203; 548/235

(57) ABSTRACT

The present invention is directed at substituted heteroaryl- and phenylsulfamoyl compounds, pharmaceutical compositions containing such compounds and the use of such compounds as peroxisome proliferator activator receptor (PPAR) agonists. PPAR alpha activators, pharmaceutical compositions containing such compounds and the use of such compounds to elevate certain plasma lipid levels, including high density lipoprotein-cholesterol and to lower certain other plasma lipid levels, such as LDL-cholesterol and triglycerides and accordingly to treat diseases which are exacerbated by low levels of HDL cholesterol and/or high levels of LDL-cholesterol and triglycerides, such as atherosclerosis and cardiovascular diseases, in mammals, including humans. The compounds are also useful for the treatment of negative energy balance (NEB) and associated diseases in ruminants.

### BACKGROUND OF INVENTION

The present invention relates to substituted heteroaryl- and phenylsulfamoyl-compounds, pharmaceutical compositions containing such compounds and the use of such compounds as peroxisome proliferator activator receptor (PPAR) agonists. The subject compounds are particularly useful as PPARα agonists and to treat atherosclerosis, hypercholesterolernia, hypertriglyceridemia, diabetes, obesity, osteoporosis and Syndrome X (also known as metabolic syndrome) in mammals, including humans. The compounds are also useful for the treatment of negative energy balance (NEB) and associated diseases in ruminants.

Atherosclerosis, a disease of the arteries, is recognized to be the leading cause of death in the United States and Western Europe. The pathological sequence leading to atherosclerosis and occlusive heart disease is well known. The earliest stage in this sequence is the formation of “fatty streaks” in the carotid, coronary and cerebral arteries and in the aorta. These lesions are yellow in color due to the presence of lipid deposits found principally within smooth-muscle cells and in macrophages of the intima layer of the arteries and aorta. Further, it is postulated that most of the cholesterol found within the fatty streaks, in turn, gives rise to development of the “fibrous plaque,” which consists of accumulated intimal smooth muscle cells laden with lipid and surrounded by extra-cellular lipid, collagen, elastin and proteoglycans. These cells plus matrix form a fibrous cap that covers a deeper deposit of cell debris and more extracellular lipid. The lipid is primarily free and esterified cholesterol. The fibrous plaque forms slowly, and is likely in time to become calcified and necrotic, advancing to the “complicated lesion,” which accounts for the arterial occlusion and tendency toward mural thrombosis and arterial muscle spasm that characterize advanced atherosclerosis.

Epidemiological evidence has firmly established hyperlipidemia as a primary risk factor in causing cardiovascular disease (CVD) due to atherosclerosis. In recent years, leaders of the medical profession have placed renewed emphasis on lowering plasma cholesterol levels, and low density lipoprotein cholesterol in particular, as an essential step in prevention of CVD. The upper limits of “normal” are now known to be significantly lower than heretofore appreciated. As a result, large segments of Western populations are now realized to be at particularly high risk. Additional independent risk factors include glucose intolerance, left ventricular hypertrophy, hypertension, and being of the male sex. Cardiovascular disease is especially prevalent among diabetic subjects, at least in part because of the existence of multiple independent risk factors in this population. Successful treatment of hyperlipidemia in the general population, and in diabetic subjects in particular, is therefore of exceptional medical importance.

In spite of the early discovery of insulin and its subsequent widespread use in the treatment of diabetes, and the later discovery of and use of sulfonylureas, biguanides and thiazolidenediones, such as troglitazone, rosiglitazone or pioglitazone, as oral hypoglycemic agents, the treatment of diabetes could be improved. The use of insulin typically requires multiple daily doses. Determination of the proper dosage of insulin requires frequent estimations of the sugar in urine or blood. The administration of an excess dose of insulin causes hypoglycemia, with effects ranging from mild abnormalities in blood glucose to coma, or even death. Treatment of non-insulin dependent diabetes mellitus (Type II diabetes, NIDDM) usually consists of a combination of diet, exercise, oral hypoglycemic agents, e.g., thiazolidenediones, and in more severe cases, insulin. However, the clinically available hypoglycemic agents can have side effects that limit their use. In the case of insulin dependent diabetes mellitus (Type I), insulin is usually the primary course of therapy.

Thus, although there are a variety of anti-atherosclerosis and diabetes therapies, there is a continuing need and a continuing search in this field of art for alternative therapies.

Moreover, negative energy balance (NEB) is a problem frequently encountered in ruminants particularly dairy cows. NEB may be experienced at any time during the cows life but it is particularly prevalent during the transition period. The ruminant transition period is defined as the period spanning late gestation to early lactation. This is sometimes defined as from 3 weeks before to three weeks after parturition, but has been expanded to 30 days prepartum to 70 days postpartum (J N Spain and W A Scheer, Tri-State Dairy Nutrition Conference, 2001, 13).

Energy balance is defined as energy intake minus energy output and an animal is descibed as being in negative energy balance if energy intake is insufficient to meet the demands on maintenance and production (eg milk). A cow in NEB has to find the energy to meet the deficit from its body reserves. Thus cows in NEB tend to lose body condition and liveweight, with cows that are more energy deficient tending to lose condition and weight at a faster rate. It is important that the mineral and energy balance and overall health of the cow is managed well in the transition period, since this interval is critically important to the subsequent health, production, and profitability in dairy cows.

Long chain fatty acids (or non esterified fatty acids, NEFAs) are also mobilised from body fat. NEFAs, already elevated from around 7 days prepartum, are a significant source of energy to the cow during the early postpartum period, and the greater the energy deficit the higher the concentration of NEFA in the blood. Some workers suggest that in early lactation (Bell and references therein-see above) mammary uptake of NEFAs accounts for some milk fat synthesis. The circulating NEFAs are taken up by the liver and are oxidised to carbon dioxide or ketone bodies, including 3-hydroxybutyrate, by mitochondria, or reconverted via esterification into triglycerides and stored. In non-ruminant mammals it is thought that entry of NEFAs into the mitochondria is controlled by the enzyme carnitine palmitoyltransferase (CPT-1) however, some studies have shown that in ruminants there is little change in activity of CPT-1 during the transition period (G. N. Douglas, J. K. Drackley, T. R. Overton, H. G. Bateman, J. Dairy Science, 1998, Supp 1, 81, 295). Furthermore, the capacity of the ruminant liver for synthesising very low density lipoproteins to export triglycerides from the liver is limited.

Significantly, if NEFA uptake by the bovine liver becomes excessive, accumulation of ketone bodies can lead to ketosis, and excessive storage of triglycerides may lead to fatty liver. Fatty liver can lead to prolonged recovery for other disorders, increased incidence of health problems, and development of “downer cows” that die.

Thus, fatty liver is a metabolic disease of ruminants, particularly high producing dairy cows; in the transition period that negatively impacts disease resistance (abomasal displacement, lameness), immune function (mastitits, metritis), reproductive performance (oestrus, calving interval, foetal viability, ovarian cysts, metritis, retained placenta), and milk production (peak milk yield, 305 day milk yield). Fatty liver has largely developed by the day after parturition and precedes an induced (secondary) ketosis. It usually results from increased esterification of NEFA absorbed from blood coupled with the low ability of ruminant liver to secrete triglycerides as very low-density lipoproteins.

By improving energy balance, or by treating the negative energy balance, the negative extent of the sequelae will be reduced. This is addressed by the compounds of the present invention.

### SUMMARY OF THE INVENTION

The present invention is directed to compounds of Formula I 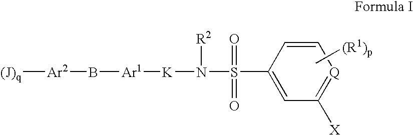
or a prodrug of said compound or a pharmaceutically acceptable salt of said compound or prodrug, wherein
Q is carbon;
each R¹ is independently hydrogen, halo, (C₁-C₅)alkyl optionally substituted with one to eleven halo or with (C₁-C₃)alkoxy, (C₁-C₅)alkoxy optionally substituted with one to eleven halo, (C₁-C₅)alkylthio optionally substituted with one or more halo, or R¹ in conjunction with the two adjacent carbon atoms forms a C₅-C₆ fused fully saturated, partially unsaturated or fully unsaturated five or six membered carbocyclic ring wherein each carbon in the carbon chain may optionally be replaced with one heteroatom selected from oxygen and sulfur;
R² is hydrogen, (C₁-C₅)alkyl optionally substituted with C₁-C₃ alkoxy, Pr benzyl optionally substituted with one to three substituents selected from the group consisting of halo, (C₁-C₄)alkyl optionally substituted with one to nine halo, (C₁-C₄)alkoxy optionally substituted with one to nine halo, and (C₁-C₄)alkylthio optionally substituted with one to nine halo;
K is —O—(CZ₂)ₜ—, —S—(CZ₂)ₜ—, —(CZ₂)ᵤ— or K and R² together form a fully saturated or partially unsaturated four to six membered cyclic carbon chain and wherein each Z is independently hydrogen or (C₁-C₃)alkyl, t is 2, 3 or 4, and u is 1, 2, 3 or 4;
X is —COOR⁴, —O—(CR³₂)—COOR⁴, —S—(CR³₂)—COOR⁴, —CH₂—(CR⁵₂)_{w}—COOR⁴, 1H-tetrazol-5-yl-E- or thiazolidinedione-5-yl-G-; wherein w is 0, 1 or 2; E is (CH₂)ᵣ and r is 0, 1, 2 or 3, and G is (CH₂)ₛ or methylidene and s is 0 or 1;
each R³ is independently hydrogen, (C₁-C₄)alkyl optionally substituted with one to nine halo, or (C₁-C₃)alkoxy optionally substituted with one or more halo, or R³ and the carbon to which it is attached form a 3, 4, 5, or 6 membered carbocyclic ring;
R⁴ is H, (C₁-C₄)alkyl, benzyl or p-nitrobenzyl;
each R⁵ is independently hydrogen, (C₁-C₄)alkyl optionally substituted with one to nine halo or with (C₁-C₃)alkoxy, (C₁-C₄)alkoxy optionally substituted with one to nine halo, (C₁-C₄)alkylthio optionally substituted with one to nine halo or with (C₁-C₃)alkoxy, or R⁵ and the carbon to which it is attached form a 3, 4, 5, or 6 membered carbocyclic ring wherein any carbon of the 5- or 6-membered ring may be replaced by an oxygen atom;
Ar¹ is thiazolyl, oxazolyl, pyridinyl, triazolyl, pyridazyl, or phenyl, wherein phenyl is optionally fused to a member selected from thiazolyl, furanyl, oxazolyl, pyridine, pyrimidine, phenyl, or thienyl wherein Ar¹ is optionally mono-, di- or tri-substituted with Z, wherein each Z is independently: hydrogen, halo, (C₁-C₃)alkyl optionally substituted with one to seven halo, (C₁-C₃)alkoxy optionally substituted with one to seven halo or (C₁-C₃)alkylthio optionally substituted with one to seven halo;
B is a bond, CO, (CY₂)ₙ, CYOH, CY═CY, -L-(CY₂)ₙ—, —(CY₂)ₙ-L-, -L-(CY₂)₂-L-, NY—OC—, —CONY—, —SO₂NY—, —NY—SO₂— wherein each L is independently O, S, SO, or SO₂, each Y is independently hydrogen or (C₁-C₃) alkyl, and n is 0, 1, 2 or 3;
Ar² is a bond, phenyl, phenoxybenzyl, phenoxyphenyl, benzyloxyphenyl, benzyloxybenzyl, pyrimidinyl, pyridinyl, pyrazolyl, imidazolyl, thiazolyl, thiadiazolyl, oxazolyl, oxadiazolyl or phenyl fused to a ring selected from the group consisting of: phenyl, pyrimidinyl, thienyl, furanyl, pyrrolyl, thiazolyl, oxazolyl, pyrazolyl, and imidazolyl;
each J is independently hydrogen, hydroxy, halo, (C₁-C₈)alkyl optionally substituted with one to seventeen halo, (C₁-C₈)alkoxy optionally substituted with one to seventeen halo, (C₁-C₈)alkylthio optionally substituted with one to seventeen halo, (C₃-C₇)cycloalkyl, (C₃-C₇)cycloalkyloxy, (C₃-C₇)cycloalkylthio, or phenyl optionally substituted with one to four substituents from the group consisting of: halo, (C₁-C₃)alkyl optionally substituted with one to seven halo, (C₁-C₃)alkoxy optionally substituted with one to seven halo, and (C₁-C₃)alkylthio optionally substituted with one to seven halo; and
p and q are each independently 0, 1, 2 or 3; and
with the provisos:
a) if Ar¹ is phenyl, B is a bond, Ar² is a bond or phenyl, K is (CH₂)ₜ and X is —COOH then q is other than 0 and J is other than hydrogen; and
b) if Ar¹ is phenyl, B is not a bond, Ar² is phenyl, K is —(CH₂)ₜ— and X is —COOR⁴ then B is attached to Ar¹ para to K.

The present application also is directed to methods for treating dyslipidemia, obesity, overweight condition, hypertriglyceridemia, hyperlipidemia, hypoalphalipoproteinemia, metabolic syndrome, diabetes mellitus (Type I and/or Type II), hyperinsulinemia, impaired glucose tolerance, insulin resistance, diabetic complications, atherosclerosis, hypertension, coronary heart disease, coronary artery disease hypercholesterolemia, inflammation, osteoporosis, thrombosis, peripheral vascular disease, cognitive dysfunction, or congestive heart failure in a mammal by administering to a mammal in need of such treatment a therapeutically effective amount of a compound of any of claims 1-18, or a prodrug of said compound or a pharmaceutically acceptable salt of said compound or prodrug.

The present application also is directed to pharmaceutical compositions which comprises a therapeutically effective amount of a compound of formula I, or a prodrug of said compound or a pharmaceutically acceptable salt of said compound or prodrug and a pharmaceutically acceptable carrier, vehicle or diluent.

In addition, the present application is directed to pharmaceutical combination compositions comprising: a therapeutically effective amount of a composition comprising
a first compound, said first compound being a compound of formula I, or a prodrug of said compound or a pharmaceutically acceptable salt of said compound or prodrug;
a second compound, said second compound being a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, a combination of niacin and lovastatin, an ion-exchange resin, an antioxidant, an ACAT inhibitor, a bile acid sequestrant, or a prodrug of said compound or a pharmaceutically acceptable salt of said compound or prodrug; and
a pharmaceutically acceptable carrier, vehicle or diluent.

Moreover, the present invention is directed to methods for treating atherosclerosis in a mammal comprising administering to a mammal in need of treatment thereof;
a first compound, said first compound being a compound of formula I, or a prodrug of said compound or a pharmaceutically acceptable salt of said compound or prodrug; and
a second compound, said second compound being a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, a combination of niacin and lovastatin, an ion-exchange resin, an antioxidant, an ACAT inhibitor or a bile acid sequestrant
wherein the amounts of first and second compounds result in a therapeutic effect.

Furthermore, the present application also is directed to kits for achieving a therapeutic effect in a mammal comprising packaged in association a first therapeutic agent comprising a therapeutically effective amount of a compound of the formula I, or a prodrug of said compound or a pharmaceutically acceptable salt of said compound or prodrug and a pharmaceutically acceptable carrier, a second therapeutic agent comprising a therapeutically effective amount of an HMG CoA reductase inhibitor, a CETP inhibitor, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant and a pharmaceutically acceptable carrier and directions for administration of said first and second agents to achieve the therapeutic effect.

Another aspect of the present invention is the use of a compound of formula I, in the manufacture of a medicament for the palliative, prophylactic or curative treatment of negative energy balance in ruminants.

Another aspect of the invention is the use of a compound of formula I, in the manufacture of a medicament for the palliative, prophylactic or curative treatment of negative energy balance or a ruminant disease associated with negative energy balance in ruminants, wherein the excessive accumulation of triglycerides in liver tissue is prevented or alleviated, and/or the excessive elevation of non-esterified fatty acid levels in serum is prevented or alleviated.

Another aspect of the invention is where the ruminant disease associated with negative energy balance in ruminants, as mentioned in the aspects of the invention herein, includes one or more diseases selected independently from fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility and lameness, preferably fatty liver syndrome, primary ketosis, downer cow syndrome, (endo-)-metritis and low fertility.

Another aspect of the invention is the use of a compound of formula I, in the improvement of fertility, including decreased return to service rates, normal oestrus cycling, improved conception rates, and improved foetal viability.

Another aspect of the invention is the use of a compound of formula I, in the manufacture of a medicament for the management of effective homeorhesis to accommodate parturition and lactogenesis.

Another aspect of the invention is the use of a compound of formula I, in the manufacture of a medicament for improving or maintaining the functioning of the ruminant liver and homeostatic signals during the transition period.

In one aspect of the invention, the compound of formula I is administered during the period from 30 days prepartum to 70 days postpartum.

In another aspect of the invention, the compound of formula I is administered prepartum and, optionally, also at parturition.

In yet another aspect of the invention, the compound of formula I is administered postpartum.

In yet another aspect of the invention, the compound of formula I is administered at parturition.

More preferably, the compound of formula I is administered during the period from 3 weeks prepartum to 3 weeks postpartum.

In another aspect of the invention, the compound of formula I is administered up to three times during the first seven days postpartum.

Preferably, the compound of formula I is administered once during the first 24 hours postpartum.

In another aspect of the invention, the compound of formula I is administered prepartum and up to four times postpartum.

In another aspect of the invention, the compound of formula I is administered at parturition and then up to four times postpartum.

Another aspect of the invention is the use of the compound of formula I in the manufacture of a medicament for the palliative, prophylactic or curative treatment of negative energy balance in ruminants and to increase ruminant milk quality and/or milk yield. In a preferred aspect of the invention, the milk quality increase is seen in a reduction in the levels of ketone bodies in ruminant milk.

In another aspect of the invention, peak milk yield is increased.

Preferably, the ruminant is a cow or sheep.

In another aspect of the invention, an overall increase in ruminant milk yield is obtained during the 305 days of the bovine lactation period.

In another aspect of the invention, an overall increase in ruminant milk yield is obtained during the first 60 days of the bovine lactation period.

Preferably, the overall increase in ruminant milk yield, or the increase in peak milk yield, or the increase in milk quality, is obtained from a dairy cow.

In another aspect of the invention, the increase in ruminant milk quality and/or milk yield is obtained after administration of a compound of formula I to a healthy ruminant.

In another aspect of the invention, there is provided a compound of formula I, for use in veterinary medicine.

It is to be understood that both the foregoing general description and the following detailed description are exemplary and explanatory only and are not restrictive of the invention, as claimed.

### BRIEF DESCRIPTION OF THE FIGURE

FIG. 1 shows the serum NEFA levels for transition cows administered with compound Z: an exemplary PPARalpha compound not within the scope of the present invention, compared to controls.

### DETAILED DESCRIPTION OF THE INVENTION

The present invention may be understood more readily by reference to the following detailed description of exemplary embodiments of the invention and the examples included therein.

Before the present compounds, compositions and methods are disclosed and described, it is to be understood that this invention is not limited to specific synthetic methods of making that may of course vary. It is also to be understood that the terminology used herein is for the purpose of describing particular embodiments only and is not intended to be limiting.

The present invention also relates to the pharmaceutically acceptable acid addition salts of compounds of the present invention. The acids which are used to prepare the pharmaceutically acceptable acid addition salts of the aforementioned base compounds of this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The invention also relates to base addition salts of the compounds of the present invention. The chemical bases that may be used as reagents to prepare pharmaceutically acceptable base salts of those compounds of the present invention that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

The chemist of ordinary skill will recognize that certain compounds of this invention will contain one or more atoms that may be in a particular stereochemical or geometric configuration, giving rise to stereoisomers and configurational isomers. All such isomers and mixtures thereof are included in this invention. Hydrates and solvates of the compounds of this invention are also included.

Where the compounds of the present invention possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds of the present invention possess one or more stereogenic centers and no stereochemistry is given in the name or structure, it is understood that the name or structure is intended to encompass all forms of the compound, including the racemic form.

The compounds of this invention may contain olefin-like double bonds. When such bonds are present, the compounds of the invention exist as cis and trans configurations and as mixtures thereof. The term “cis” refers to the orientation of two substituents with reference to each other and the plane of the ring (either both “up” or both “down”). Analogously, the term “trans” refers to the orientation of two substituents with reference to each other and the plane of the ring (the substituents being on opposite sides of the ring).

Alpha and Beta refer to the orientation of a substituent with reference to the plane of the ring. Beta is above the plane of the ring and Alpha is below the plane of the ring.

This invention also includes isotopically-labeled compounds, which are identical to those described by Formulas I and II, except for the fact that one or more atoms are replaced by one or more atoms having specific atomic mass or mass numbers. Examples of isotopes that can be incorporated into compounds of the invention include isotopes of hydrogen, carbon, nitrogen, oxygen, sulfur, fluorine, and chlorine such as ²H, ³H, ¹³C, ¹⁴C, ¹⁵N, ¹⁸O, ¹⁷O, ¹⁸F, and ³⁶Cl respectively. Compounds of the present invention, prodrugs thereof, and pharmaceutically acceptable salts of the compounds or of the prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled compounds of this invention and prodrugs thereof can generally be prepared by carrying out the procedures disclosed in the schemes and/or in the Examples below, by substituting a readily available isotopically labeled reagent for a non-isotopically labeled reagent.

In this specification and in the claims that follow, reference will be made to a number of terms that shall be defined to have the following meanings:

The term “treating”, “treat” or “treatment” as used herein includes preventative (e.g., prophylactic) and palliative treatment.

As used herein, “therapeutically effective amount of a compound” means an amount that is effective to exhibit therapeutic or biological activity at the site(s) of activity in a mammalian subject, without undue adverse side effects (such as undue toxicity, irritation or allergic response), commensurate with a reasonable benefit/risk ratio when used in the manner of the present invention.

The term “cerebrovascular disease”, as used herein, is selected, but not limited to, the group consisting of ischemic attacks (e.g., transient), ischemic stroke (transient), acute stroke, cerebral apoplexy, hemorrhagic stroke, neurologic deficits post-stroke, first stroke, recurrent stroke, shortened recovery time after stroke and provision of thrombolytic therapy for stroke. Preferable patient populations include patients with or without pre-existing stroke or coronary heart disease.

The term “coronary artery disease”, as used herein, is selected, but not limited to, the group consisting of atherosclerotic plaque (e.g., prevention, regression, stablilization), vulnerable plaque (e.g., prevention, regression, stabilization), vulnerable plaque area (reduction), arterial calcification (e.g., calcific aortic stenosis), increased coronary artery calcium score, dysfunctional vascular reactivity, vasodilation disorders, coronary artery spasm, first myocardial infarction, myocardia re-infarction, ischemic cardiomyopathy, stent restenosis, PTCA restenosis, arterial restenosis, coronary bypass graft restenosis, vascular bypass restenosis, decreased exercise treadmill time, angina pectoris/chest pain, unstable angina pectoris, exertional dyspnea, decreased exercise capacity, ischemia (reduce time to), silent ischemia (reduce time to), increased severity and frequency of ischemic symptoms, reperfusion after thrombolytic therapy for acute myocardial infarction.

The term “hypertension”, as used herein, is selected, but not limited to, the group consisting of lipid disorders with hypertension, systolic hypertension and diastolic hypertension.

The term “ventricular dysfunction”, as used herein, is selected, but not limited to, the group consisting of systolic dysfunction, diastolic dysfunction, heart failure, congestive heart failure, dilated cardiomyopathy, idiopathic dilated cardiomyopathy, and non-dilated cardiomopathy.

The term “cardiac arrhythmia”, as used herein, is selected, but not limited to, the group consisting of atrial arrhythmias, supraventricular arrhythmias, ventricular arrhythmias and sudden death syndrome.

The term “pulmonary vascular disease”, as used herein, is selected, but not limited to, the group consisting of pulmonary hypertension, peripheral artery block, and pulmonary embolism.

The term “peripheral vascular disease”, as used herein, is selected, but not limited to, the group consisting of peripheral vascular disease and claudication.

The term “vascular hemostatic disease”, as used herein, is selected, but not limited to, the group consisting of deep venous thrombosis, vaso-occlusive complications of sickle cell anemia, varicose veins, pulmonary embolism, transient ischemic attacks, embolic events, including stroke, in patients with mechanical heart valves, embolic events, including stroke, in patients with right or left ventricular assist devices, embolic events, including stroke, in patients with intra-aortic balloon pump support, embolic events, including stroke, in patients with artificial hearts, embolic events, including stroke, in patients with cardiomyopathy, embolic events, including stroke, in patients with atrial fibrillation or atrial flutter.

The term “diabetes”, as used herein, refers to any of a number of diabetogenic states including type I diabetes, type II diabetes, Syndrome X, Metabolic syndrome, lipid disorders associated with insulin resistance, impaired glucose tolerance, non-insulin dependent diabetes, microvascular diabetic complications, reduced nerve conduction velocity, reduced or loss of vision, diabetic retinopathy, increased risk of amputation, decreased kidney function, kidney failure, insulin resistance syndrome, pluri-metabolic syndrome, central adiposity (visceral)(upper body), diabetic dyslipidemia, decreased insulin sensitization, diabetic retinopathy/neuropathy, diabetic nephropathy/micro and macro angiopathy and micro/macro albuminuria, diabetic cardiomyopathy, diabetic gastroparesis, obesity, increased hemoglobin glycoslation (including HbA1C), improved glucose control, impaired renal function (dialysis, endstage) and hepatic function (mild, moderate, severe).

The terms “inflammatory disease, autoimmune disorders and other systemic diseases”, as used herein, are selected, but not limited to, the group consisting of multiple sclerosis, rheumatoid arthritis, osteoarthritis, irritable bowel syndrome, irritable bowel disease, Crohn's disease, colitis, vasculitis, lupus erythematosis, sarcoidosis, amyloidosis, apoptosis, and disorders of the complement systems.

The term “cognitive dysfunction”, as used herein, is selected, but not limited to, the group consisting of dementia secondary to atherosclerosis, transient cerebral ischemic attacks, neurodegeneration (including Parkinson's, Huntington's disease, amyloid deposition and amylotrophic lateral sclerosis), neuronal deficient, and delayed onset or procession of Alzheimer's disease.

“Metabolic syndrome,” also known as “Syndrome X,” refers to a common clinical disorder that is defined as the presence of increased insulin concentrations in association with other disorders including viceral obesity, hyperlipidemia, dyslipidemia, hyperglycemia, hypertension, and potentially hyperuricemis and renal dysfunction.

The “transition period” means from 30 days prepartum to 70 days postpartum.

The term “treating”, “treat”, “treats” or “treatment” as used herein includes prophylactic, palliative and curative treatment.

“Negative energy balance” as used herein means that energy via food does not meet the requirements of maintenance and production (milk).

The term “cow” as used herein includes heifer, primiparous and multiparous cow.

“Healthy ruminant” means where the ruminant does not show signs of the following indications: fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility and/or lameness.

Milk “quality” as used herein refers to the levels in milk of protein, fat, lactose, somatic cells, and ketone bodies. An increase in milk quality is obtained on an increase in fat, protein or lactose content, or a decrease in somatic cell levels or ketone bodies levels.

An increase in milk yield can mean an increase in milk solids or milk fat or milk protein content, as well as, or instead of, an increase in the volume of milk produced.

“Excessive accumulation of triglycerides” as used herein means greater than the physiological triglyceride content of 10% w/w in liver tissue.

“Excessive elevation of non-esterified fatty acid levels in serum” as used herein means non-esterified fatty acid levels of greater than 800 μmol/L in serum.

Unless otherwise specified, “prepartum” means 3 weeks before calving until the day of calving.

Unless otherwise specified, “postpartum” means from when the newborn is “expelled” from the uterus to 6 weeks after the newborn was expelled from the uterus.

“At parturition” means the 24 hours after the newborn was expelled from the uterus.

“Periparturient” means the period from the beginning of the prepartum period, to the end of the postpartum period.

By “pharmaceutically acceptable” is meant the carrier, diluent, excipients, and/or salt must be compatible with the other ingredients of the formulation, and not deleterious to the recipient thereof.

“Compounds” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. By “tautomers” is meant chemical compounds that may exist in two or more forms of different structure (isomers) in equilibrium, the forms differing, usually, in the position of a hydrogen atom. Various types of tautomerism can occur, including keto-enol, ring-chain and ring-ring tautomerism. The expression “prodrug” refers to compounds that are drug precursors which following administration, release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the compounds of the present invention include but are not limited to those having a carboxyl moiety wherein the free hydrogen is replaced by (C₁-C₄)alkyl, (C₂-C₇)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

The following paragraphs describe exemplary ring(s) for the generic ring descriptions contained herein.

Exemplary five to six membered aromatic rings optionally having one or two heteroatoms selected independently from oxygen, nitrogen and sulfur include phenyl, furyl, thienyl, pyrrolyl, oxazolyl, thiazolyl, imidazolyl, pyrazolyl, isoxazolyl, isothiazolyl, pyridinyl, pyridiazinyl, pyrimidinyl and pyrazinyl.

Exemplary partially saturated, fully saturated or fully unsaturated membered carbocyclic rings optionally having one to four heteroatoms selected independently from oxygen, sulfur and nitrogen include cyclopentyl, cyclohexyl, cycloheptyl, cyclooctyl and phenyl.

Further exemplary five membered carbocyclic rings include 2H-pyrrolyl, 3H-pyrrolyl, 2-pyrrolinyl, 3-pyrrolinyl, pyrrolidinyl, 1,3-dioxolanyl, oxazolyl, thiazolyl, imidazolyl, 2H-imidazolyl, 2-imidazolinyl, imidazolidinyl, pyrazolyl, 2-pyrazolinyl, pyrazolidinyl, isoxazolyl, isothiazolyl, 1,2-dithiolyl, 1,3-dithiolyl, 3H-1,2-oxathiolyl, 1,2,3-oxadiazolyl, 1,2,4-oxadiazolyl, 1,2,5-oxadiazolyl, 1,3,4-oxadiazolyl, 1,2,3-triazolyl, 1,2,4-triazolyl, 1,3,4-thiadiazolyl, 1,2,3,4-oxatriazolyl, 1,2,3,5-oxatriazolyl, 3H-1,2,3-dioxazolyl, 1,2,4-dioxazolyl, 1,3,2-dioxazolyl, 1,3,4-dioxazolyl, 5H-1,2,5-oxathiazolyl and 1,3-oxathiolyl.

Further exemplary six membered carbocyclic rings include 2H-pyranyl, 4H-pyranyl, pyridinyl, piperidinyl, 1,2-dioxinyl, 1,3-dioxinyl, 1,4-dioxanyl, morpholinyl, 1,4-dithianyl, thiomorpholinyl, pyridazinyl, pyrimidinyl, pyrazinyl, piperazinyl, 1,3,5-triazinyl, 1,2,4-triazinyl, 1,2,3-triazinyl, 1,3,5-trithianyl, 4H-1,2-oxazinyl, 2H-1,3-oxazinyl, 6H-1,3-oxazinyl, 6H-1,2-oxazinyl, 1,4-oxazinyl, 2H-1,2-oxazinyl, 4H-1,4-oxazinyl, 1,2,5-oxathiazinyl, 1,4-oxazinyl, o-isoxazinyl, p-isoxazinyl, 1,2,5-oxathiazinyl, 1,2,6-oxathiazinyl, 1,4,2-oxadiazinyl and 1,3,5,2-oxadiazinyl.

Further exemplary seven membered rings include azepinyl, oxepinyl, and thiepinyl.

Further exemplary eight membered carbocyclic rings include cyclooctyl, cyclooctenyl and cyclooctadienyl.

Exemplary bicyclic rings consisting of two fused partially saturated, fully saturated or fully unsaturated five or six membered rings, taken independently, optionally having one to four heteroatoms selected independently from nitrogen, sulfur and oxygen include indolizinyl, indolyl, isoindolyl, 3H-indolyl, 1H-isoindolyl, indolinyl, cyclopenta(b)pyridinyl, pyrano(3,4-b)pyrrolyl, benzofuryl, isobenzofuryl, benzo(b)thienyl, benzo(c)thienyl, 1H-indazolyl, indoxazinyl, benzoxazolyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2H-1-benzopyranyl, pyrido(3,4-b)-pyridinyl, pyrido(3,2-b)-pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl.

The carbon atom content of various hydrocarbon-containing moieties is indicated by a prefix designating the minimum and maximum number of carbon atoms in the moiety, i.e., the prefix Cᵢ-Cⱼ indicates a moiety of the integer “i” to the integer “j” carbon atoms, inclusive. Thus, for example, C₁-C₃ alkyl refers to alkyl of one to three carbon atoms, inclusive, or methyl, ethyl, propyl and isopropyl, and all isomeric forms and straight and branched forms thereof.

By “aryl” is meant an optionally substituted six-membered aromatic ring, including polyaromatic rings. Examples of aryl include phenyl, naphthyl and biphenyl.

“Heteroaryl” as used herein means an optionally substituted five- or six-membered aromatic ring, including polyaromatic rings where appropriate carbon atoms are substituted by nitrogen, sulfur or oxygen. Examples of heteroaryl include pyridine, pyrimidine, thiazole, oxazole, quinoline, quinazoline, benzothiazole and benzoxazole.

By “halo” or “halogen” is meant chloro, bromo, iodo, or fluoro.

By “alkyl” is meant straight chain saturated hydrocarbon or branched chain saturated hydrocarbon. Exemplary of such alkyl groups (assuming the designated length encompasses the particular example) are methyl, ethyl, propyl, isopropyl, butyl, sec-butyl, tertiary butyl, pentyl, isopentyl, neopentyl, tertiary pentyl, 1-methylbutyl, 2-methylbutyl, 3-methylbutyl, hexyl, isohexyl, heptyl and octyl. This term also includes a saturated hydrocarbon (straight chain or branched) wherein a hydrogen atom is removed from each of the terminal carbons.

“Alkenyl” referred to herein may be linear or branched, and they may also be cyclic (e.g. cyclobutenyl, cyclopentenyl, cyclohexenyl) or bicyclic or contain cyclic groups. They contain 1-3 carbon-carbon double bonds, which can be cis or trans.

By “alkoxy” is meant straight chain saturated alkyl or branched chain saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3- or 4-pyridyl, the term “thienyl” means 2- or 3-thienyl, and so forth.

The term “HMG CoA reductase inhibitor” is selected, but not limited to, the group consisting of lovastatin, simvastatin, pravastatin, fluindostatin, velostatin, dihydrocompactin, compactin, fluvastatin, atorvastatin, glenvastatin, dalvastatin, carvastatin, crilvastatin, bervastatin, cerivastatin, rosuvastatin, pitavastatin, mevastatin, or rivastatin, or a pharmaceutically acceptable salt thereof.

The term “antihypertensive agent” is selected, but not limited to, a calcium channel blocker (including, but not limited to, verapamil, diltiazem, mibefradil, isradipine, lacidipine, nicardipine, nifedipine, nimodipine, nisoldipine, nitrendipine, avanidpine, amlodipine, amlodipine besylate, manidipine, cilinidipine, lercanidipine and felodipine), an ACE inhibitor (including, but not limited to, benazepril, captopril, enalapril, fosinopril, lisinopril, perindopril, quinapril, trandolapri, ramipril, zestril, zofenopril, cilaapril, temocapril, spirapril, moexipril, delapril, imidapril, ramipril, terazosin, urapidin, indoramin, amolsulalol, and alfuzosin), an A-II antagonist (including, but not limited to, losartan, irbesartan, telmisartan and valsartan), a diuretic (including, but not limited to, amiloride, and bendroflumethiazide), a beta-adrenergic receptor blocker (such as carvedilol) or an alpha-adrenergic receptor blocker (including, but not limited to, doxazosin, prazosin, and trimazosin), or a pharmaceutically acceptable salt of such compounds.

In one embodiment of the present invention, p is 1 or 2 and at least one R¹ is bonded to Q.

In another embodiment of the present invention, Ar¹ is: 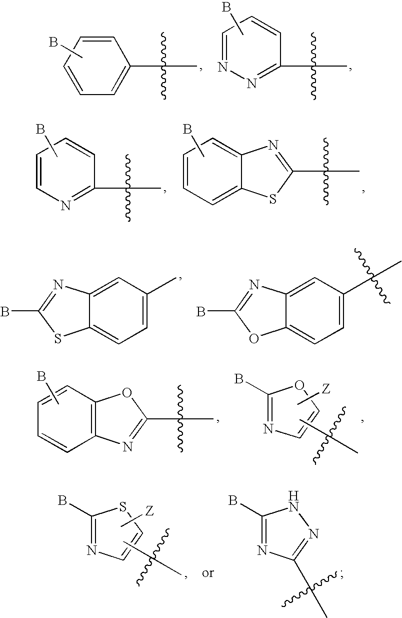
wherein Z is hydrogen or (C₁-C₃)alkyl optionally substituted with one to seven halo.

In another embodiment of the present invention, Ar² is

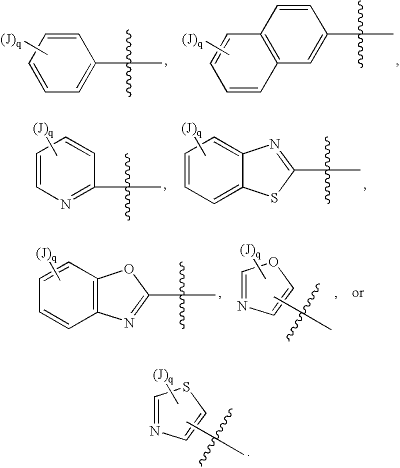

In another embodiment of the present invention,
Ar¹ is phenyl or phenyl fused to oxazolyl or thiazolyl; and
Ar² is phenyl or phenyl fused to a ring selected from the group consisting of: phenyl, pyridinyl, thienyl, thiazolyl, oxazolyl, and imidazolyl.

In another embodiment of the present invention, K is —(CH₂)ᵤ—.

In another embodiment of the present invention, B is a bond or -L-(CY₂)ₙ— or —(CY₂)ₙ-L-, and L is O or S, and n is 0, 1 or 2.

In another embodiment,
B is a bond or -L-(CY₂)ₙ— or —(CY₂)ₙ-L-;
L is O or S;
K is —(CH₂)ᵤ— and u is 1, 2, or 3;
n is 0, 1 or 2;
p is 1, 2, or 3 and at least one R¹ is attached at Q;
Ar¹ is oxazolyl, thiazolyl, phenyl or phenyl fused to oxazolyl or thiazolyl; and
Ar² is phenyl or a bond.

In another embodiment of the present invention,
X is —COOR⁴;
K is —O—(CH₂)ₜ—, —S—(CH₂)ₜ—, —(CH₂)ᵤ—,
B is a bond;
Ar¹ is oxazolyl, thiazolyl, phenyl or phenyl fused to oxazolyl or thiazolyl; and
Ar² is a bond or is phenyl.

In another embodiment of the present invention, Ar¹ is: 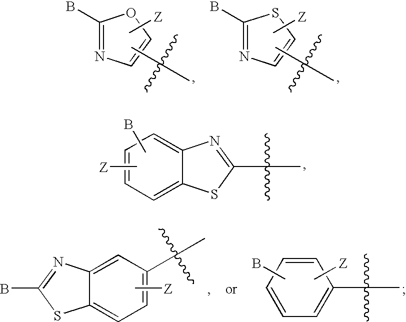
wherein Z is (C₁-C₃)alkyl optionally substituted with one to seven halo.

Ar¹ is: 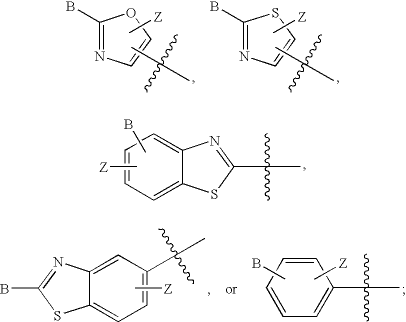
wherein Z is (C₁-C₃)alkyl optionally substituted with one to seven halo.

In another embodiment of the present invention, p is 1 or 2 and R⁴ is H or (C₁-C₃)alkyl.

In another embodiment of the present invention, X is —COOR⁴; K is —O—(CH₂)ₜ—, —S—(CH₂)ₜ—, or —(CH₂)ᵤ— wherein t is 2 or 3 and u is 1, 2 or 3; B is -L-(CY₂)ₙ— or —(CY₂)ₙ-L-, and L is O or S, and n is 0, 1 or 2; Ar¹ is oxazolyl, thiazolyl, phenyl, or phenyl fused to oxazolyl or thiazolyl; and Ar² is a bond or is phenyl.

In another embodiment of the present invention, Ar¹ is phenyl; and Ar² is phenyl.

In another embodiment of the present invention, L is 0 and n is 0 or 1.

In another embodiment, X is —COOR⁴; K is —O—(CH₂)ₜ—, —S—(CH₂)ₜ—, or —(CH₂)ᵤ— wherein t is 2 or 3 and u is 1, 2 or 3; B is a bond; p is 1, 2, or 3 and at least one R¹ is attached at Q; Ar¹ is oxazolyl, thiazolyl, phenyl or phenyl fused to oxazolyl or thiazolyl; and Ar² is a bond or is phenyl.

In another embodiment, K is —(CH₂)ᵤ— and u is 1, 2, or 3; p is 1 or 2; R⁴ is H or (C₁-C₃)alkyl; and Ar¹ is: 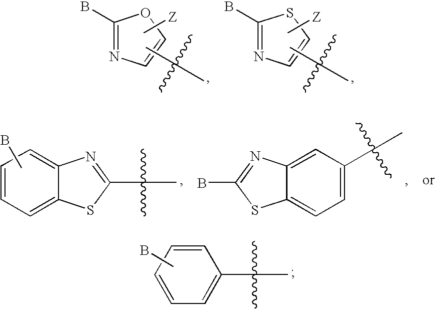
wherein Z is hydrogen or (C₁-C₃)alkyl optionally substituted with one to seven halo.

In one embodiment of the methods of the present invention, atherosclerosis is treated.

In one embodiment of the methods of the present invention, peripheral vascular disease is treated.

In one embodiment of the methods of the present invention, dyslipidemia is treated.

In one embodiment of the methods of the present invention, diabetes is treated.

In one embodiment of the methods of the present invention, hypoalphalipoproteinemia is treated.

In one embodiment of the methods of the present invention, hypercholesterolemia is treated.

In one embodiment of the methods of the present invention, hypertriglyceridemia is treated.

In one embodiment of the methods of the present invention, obesity is treated.

In one embodiment of the methods of the present invention, osteoporosis is treated.

In one embodiment of the methods of the present invention, metabolic syndrome is treated.

In another embodiment of the present invention, the pharmaceutical composition is for the treatment of atherosclerosis in a mammal which comprises an atherosclerosis treating amount of a compound of formula I, or a prodrug of said compound or a pharmaceutically acceptable salt of said compound or prodrug and a pharmaceutically acceptable carrier, vehicle or diluent.

In one embodiment of the pharmaceutical combination compositions, methods and kits of the present invention, the second compound is an HMG-CoA reductase inhibitor or a CETP inhibitor.

In one embodiment of the pharmaceutical combination compositions, methods and kits of the present invention, the second compound is rosuvastatin, rivastatin, pitavastatin, lovastatin, simvastatin, pravastatin, fluvastatin, atorvastatin or cerivastatin or a prodrug of said compound or a pharmaceutically acceptable salt of said compound or prodrug.

In one embodiment of the pharmaceutical combination compositions, methods and kits of the present invention, the second compound is [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester or (2R)-3-{[3-(4-Chloro-3-ethyl-phenoxy)-phenyl]-[[3-(1,1,2,2-tetrafluoro-ethoxy)-phenyl]-methyl]-amino}-1,1,1-trifluoro-2-propanol.

In one embodiment of the pharmaceutical combination compositions, methods and kits of the present invention, the composition further comprises a cholesterol absorption inhibitor.

In one embodiment of the pharmaceutical combination compositions, methods and kits of the present invention, the cholesterol absorption inhibitor is ezetimibe.

In one embodiment of the pharmaceutical combination compositions, methods and kits of the present invention, the composition further comprises an antihypertensive agent.

In one embodiment of the pharmaceutical combination compositions, methods and kits of the present invention, said antihypertensive agent is a calcium channel blocker, an ACE inhibitor, an A-II antagonist, a diuretic, a beta-adrenergic receptor blocker or an alpha-adrenergic receptor blocker.

In one embodiment of the pharmaceutical combination compositions, methods and kits of the present invention, the antihypertensive agent is a calcium channel blocker, said calcium channel blocker being verapamil, diltiazem, mibefradil, isradipine, lacidipine, nicardipine, nifedipine, nimodipine, nisoldipine, nitrendipine, avanidpine, amlodipine, amlodipine besylate, manidipine, cilinidipine, lercanidipine or felodipine or a prodrug of said compound or a pharmaceutically acceptable salt of said compound or prodrug.

In general, the compounds of this invention can be made by processes that include processes analogous to those known in the chemical arts, particularly in light of the description contained herein. Certain processes for the manufacture of the compounds of this invention are provided as further features of the invention and are illustrated by the following reaction schemes. Other processes may be described in the experimental section.

The Reaction Schemes herein described are intended to provide a general description of the methodology employed in the preparation of many of the Examples given. However, it will be evident from the detailed descriptions given in the Experimental section that the modes of preparation employed extend further than the general procedures described herein. In particular, it is noted that the compounds prepared according to these Schemes may be modified further to provide new Examples within the scope of this invention. For example, an ester functionality may be reacted further using procedures well known to those skilled in the art to give another ester, an amide, an acid, a carbinol or a ketone.

As an initial note, in the preparation of compounds of the present invention, it is noted that some of the preparation methods useful for the preparation of the compounds described herein may require protection of remote functionality (e.g., primary amine, secondary amine, carboxyl in intermediates). The need for such protection will vary depending on the nature of the remote functionality and the conditions of the preparative methods and can be readily determined by one of ordinary skill in the art. The use of such protection/deprotection methods is also within the ordinary skill in the art. For a general description of protecting groups and their use, see T. W. Greene, Protective Groups in Organic Synthesis, John Wiley & Sons, New York, 1991.

For example, in the reaction schemes below, certain compounds contain primary amines or carboxylic acid functionalities, which may interfere with reactions at other sites of the molecule if left unprotected. Accordingly, such functionalities may be protected by an appropriate protecting group, which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the compound.

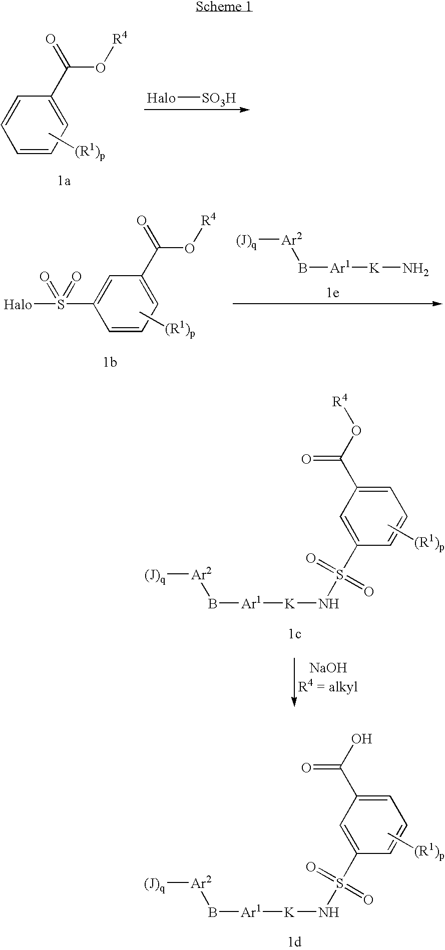

According to Scheme 1, the compounds of formula 1d, which are compounds of Formula 1 wherein X is —COOR⁴, R² is H and K, R¹, B, Ar¹. Ar², J, p, and q are as described above, are prepared by procedures well known in the art. For example, treatment of the benzoic acid or ester 1a (which are commercially available or are known in the literature or may be prepared according to methods familiar to those skilled in the art) with chlorosulfonic acid (halo is chloro) at temperatures between about 90 and 110° C., preferably 100° C., for a period of about 15 min to 3 hours, preferably 2.5 hours for the acid and 15 min for the ester, leads to the sulfonyl halide 1b.

The reaction of the sulfonyl halide 1b with appropriately substituted amines 1e (preparations of amines 1e are described in Schemes 6-12 to form the sulfonamides 1c may be performed under reaction conditions well known to those skilled in the art. For example, the reaction of the sulfonyl halide 1b and an amine 1e may be performed in a solvent such as tetrahydrofuran, dimethylformamide or a mixture of acetone and water, in the presence of a base such as pyridine, potassium carbonate or sodium carbonate, at temperatures between about 20° C. and 65° C., preferably at room temperature for a period of about 10 to 36 hours, preferably about 20 hours. If 1b is a chlorosulfonyl benzoic ester (R⁴=CH₃ and halo is chloro), it may be preferable to perform the reaction in an organic solvent such as tetrahydrofuran in the presence of an amine base such pyridine and triethylamine.

The ester product 1c may be converted to the benzoic acid 1d by hydrolysis with an alkali metal hydroxide, preferably sodium hydroxide, in a mixture of an alcohol, preferably methanol, and water at a temperature of about 50° C. to 100° C. for a period of about 2 to 30 hours, preferably at reflux temperature overnight.

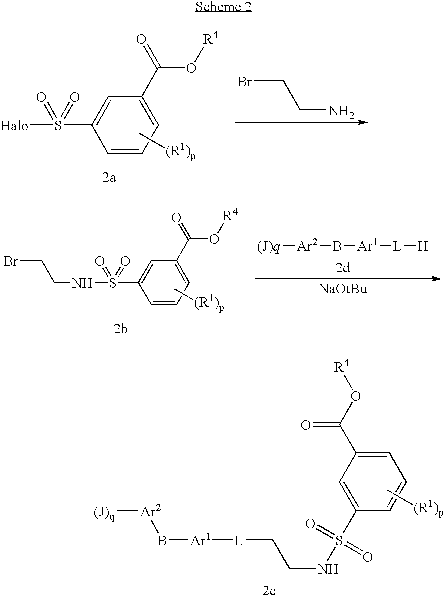

According to reaction Scheme 2, the desired Formula 1 compounds wherein X is —COOR⁴, R² is H, K is -L-(CH₂)₂— where L is O or S, and R¹, Ar¹, B, Ar², J, p and q are as described above, are prepared by procedures well known in the art. For example, treatment of sulfonyl chloride 2a (Halo is chloro and R⁴=methyl) with bromoethylamine using reaction conditions previously exemplified in Scheme 1 leads to bromoethylsulfonamide 2b.

The desired compounds of Formula 2c are formed by the reaction of bromoethylsulfonamide 2b with phenol (L=O) or thiophenol (L=S) 2d (which are commercially available or are known in the literature or may be prepared according to methods familiar to those skilled in the art) in the presence of a base such as sodium tert-butoxide or sodium hydride in an inert solvent such as tetrahydrofuran, dimethoxyethane or dimethylformide, at temperataures between about 20° C. and 85° C., for a period of about 4 to 36 hours, preferably sodium tert-butoxide in dimethylormamide at 80° C. overnight for phenol 2d and sodium tert-butoxide in tetrahydrofuran at room temperature overnight for thiophenol 2d. Ester 2c may be converted to the corresponding acid by basic hydrolysis such as the reaction conditions previously described in Scheme 1.

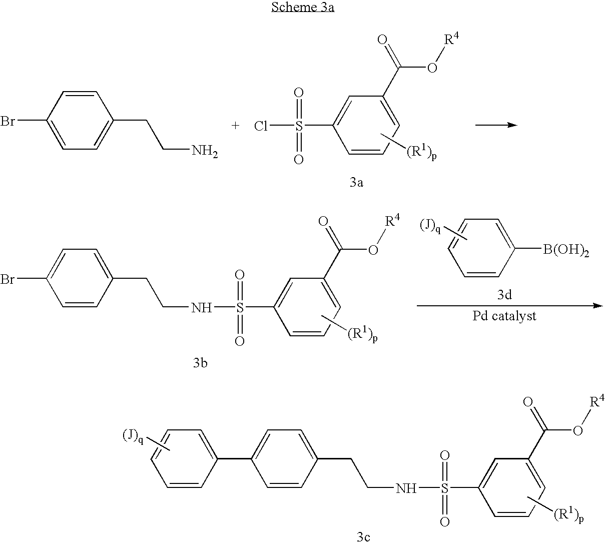

According to reaction Scheme 3a, the desired Formula I compounds wherein X is —COOR⁴, R² is H, K is (CH₂)₂, Ar¹ and Ar² are phenyl, B is a bond and R¹, J, p and q are as described above, are prepared by procedures well known in the art. For example, treatment of sulfonyl chloride 3a (R⁴=methyl and halo is chloro) with 4-bromophenylethylamine using reaction conditions previously described in Scheme 1 leads to bromophenethylsulfonamide 3b.

Reaction of 3b with an appropriately substituted benzeneboronic acid in a solvent such as tetrahydrofuran, dioxane, dimethoxyethane or dioxane/water, preferably dioxane/water, under palladium catalysis in the presence of a base such as potassium carbonate, cesium carbonate or sodium carbonate, preferably potassium carbonate, at temperatures between about 80° C. and 110° C., for about 6 to 30 hours, preferably at reflux temperature overnight, using procedures known to those skilled in the art, leads to the biphenethylsulfonamide 3c. The palladium catalysts, phosphine ligands, solvents, bases and reaction temperatures that can be used are exemplified in Chemical Reviews 102, 1359 (2002). For example, reaction of bromophenethylsulonamide 3b with an arylboronic acid 3d in the presence of a catalytic amount of dichloro[1,1′-bis(diphenylphosphino)ferrocene]palladium(II) dichloromethane adduct and 1,1′-bis(diphenylphosphino)ferrocene, with potassium carbonate as base and aqueous dioxane as solvent, yields biphenethylsulfonamede 3c. As shown in Scheme 1, the ester group of compound 3c (R⁴=methyl) may be converted to an acid group by basic hydrolysis.

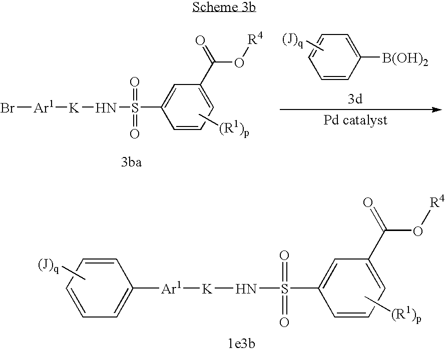

According to reaction Scheme 3b, the desired Formula I compounds wherein X is —COOR⁴, R² is H, B is a bond and Ar¹ and Ar², R¹, J, p and q are as described above, are prepared by procedures exemplified in Scheme 3a. Reaction of bromoarylsulfonamide 3ba, prepared by methods analogous to those used for the preparation of sulfonamide 3b (Scheme 3a), with an appropriately substituted benzeneboronic acid 3d mediated by palladium catalysis, as described in Scheme 3a leads to the Formula 1 compound 1e3b.

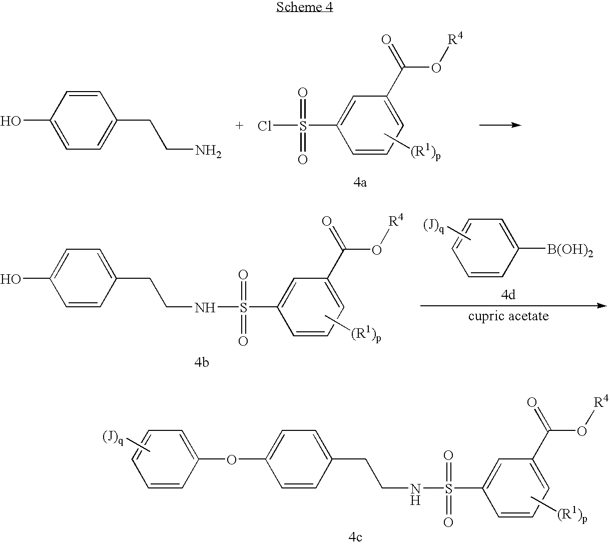

According to reaction Scheme 4, the desired Formula I compounds wherein X is —COOR⁴, R² is H, K is (CH₂)₂, Ar¹ and Ar² are phenyl, B is 0 and R¹, J, p and q are as described above, are prepared by procedures well known in the art, such as those taught in Tetrahedron Lett. 39, 2933-2936, 2937-2940 (1998). For example, treatment of sulfonyl chloride 4a (R⁴=methyl and halo is chloro) with tyramine using reaction conditions previously described in Scheme 1 leads to hydroxyphenethylsulfonamide 4b. Reaction of 4b with an appropriately substituted benzeneboronic acid in a solvent such as methylene chloride, acetonitrile or toluene, preferably methylene chloride, in the presence of cupric acetate and a tertiary amine base, preferably triethylamine or pyridine, leads to biphenyl ether 4c (R⁴=methyl). As shown in Scheme 1, the ester group of compound 4c (R⁴=methyl) may be converted to an acid group by basic hydrolysis.

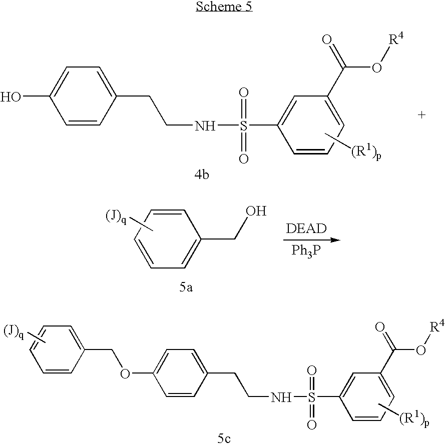

According to reaction Scheme 5, the desired Formula I compounds wherein X is —COOR⁴, R² is H, K is (CH₂)₂, Ar¹ and Ar² are phenyl, B is —CH₂O— and R¹, J, p and q are as described above, are prepared by procedures well known in the art. For example, the Mitsunobu reaction of hydroxyphenethylsulfonamide 4b (R⁴=methyl) (described in Scheme 4) with appropriately substituted benzyl alcohols, which are commercially available or readily prepared by those skilled in the art, in the presence of diethyl azodicarboxylate (DEAD) and triphenylphosphine (Ph₃P), in a solvent such as tetrahydrofuran, dimethylformamide, methylene chloride or dioxane, at about 15° C. to 35° C. for about 10 to 30 hours, preferably in tetrahydrofuran at room temperature overnight (Scheme 5) leads to benzyloxyphenethylsulfonamide 5c. The reaction conditions, reagents, solvents, temperature and reaction time for the Mitsunobu reaction are reviewed in Organic Reactions, Vol 42, 1992, 335, John Wiley, 2002. As shown in Scheme 1, the ester group of compound 5c (R⁴=methyl) may be converted to an acid group by basic hydrolysis.

Schemes 6-11 describe the preparation of amines 1e, used in the synthetic route shown in Scheme 1. Alternatively, the amines 1e in Scheme 1 are commercially available or are known in the literature or may be prepared according to procedures well known in the art.

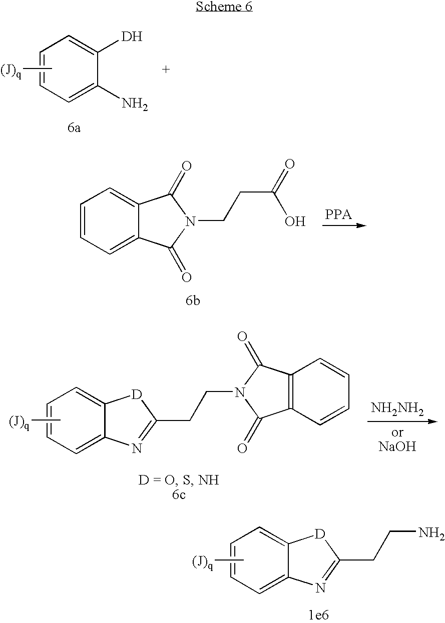

The desired Formula 1e compounds wherein R² is hydrogen, K is —(CH₂)₂—, Ar² and B are bonds, Ar¹ is a phenyl ring fused to an imidazole, oxazole, or thiazole ring (D is N, O or S) and J and q are as described above, may be prepared by reaction of an appropriately substituted 2-aminoaniline, 2-aminophenol or 2-aminothiophenol 6a and N-phthaloyl-β-alanine 6b (Scheme 6), followed by deprotection of the product 6c, or by similar synthetic routes familiar to those skilled in the art. In Scheme 6, a 2-aminophenol, 2-aminothiophenol or 2-aminoaniline derivative 6a is heated with N-phthaloyl-β-alanine 6b in polyphosphoric acid at about 170° C. to 200° C. for about 4 to 10 hours, preferably 190° C. for 6 hours, to yield the corresponding benzoxazole, benzothiazole or benzimidazole derivative 6c.

Reaction of phthalimide 6c with hydrazine hydrate in an alcoholic solvent at a temperature between about 25° C. to 85° C. for a period of about 3 to 30 hours, preferably ethanol at reflux temperature for 3 hoursleads to the amine 1e6. Alternatively, amine 1e6 can be obtained by irradiating phthalimide 6c in a microwave oven at high power with hydrazine hydrate or an alkali metal hydroxide such as sodium hydroxide in an alcoholic solvent at a temperature between about 150 to 200° C. for 6 to 20 min, preferably with hydrazine hydrate in ethanol at 160° C. for 20 min or with sodium hydroxide in ethanol at 200° C. for 6 min. References to other reagents, solvents and reaction conditions and temperatures for converting phtalimides to amines can be found in T. W. Greene and P. G. M. Wuts, Protective Groups in Organic Synthesis, John Wiley & Sons, New York, 1999.

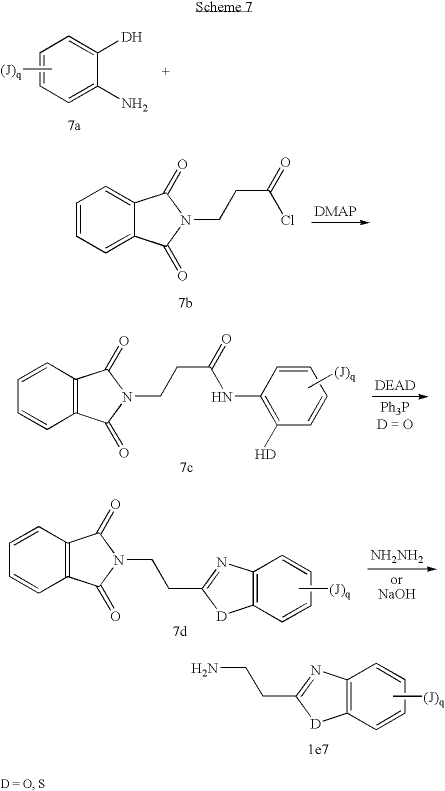

Alternatively, as outlined in Scheme 7, acylation of a 2-aminophenol or 2-aminothiophenol derivative 7a with N-phthaloyl-β-alanine acid chloride 7b, in an inert solvent such as methylene chloride, in the presence of an amine base such as 4-dimethylaminopyridine, at a temperature of about 20° C. to 50° C. for about 10 to 30 hours, preferably at room temperature for 20 hours, yields the corresponding amide 7c.

Under the acylation reaction conditions, the thiophenol derivative 7c (D=S) spontaneously cyclizes to the benzothiazole derivative 7d (D=S). The phenol derivative 7c (D=O) may be cyclized to the benzoxazole derivative 7d (D=O) by treatment with diethyl azodicarboxylate (DEAD) and triphenylphosphine (Ph₃P) (Mitsunobu reaction), in a solvent such as tetrahydrofuran, dimethylformamide, methylene chloride or dioxane, preferably tetrahydrofuran at about 15° C. to 35° C. for about 10 to 30 hours, preferably at room temperature overnight. The reaction conditions, reagents, solvents, temperature and reaction time for the Mitsunobu reaction are reviewed in Organic Reactions, Vol 42, 1992, 335, John Wiley, 2002. The desired amine 1e7 may be prepared from phthalimide 7d by methods known to those skilled in the art, including those described in Scheme 6.

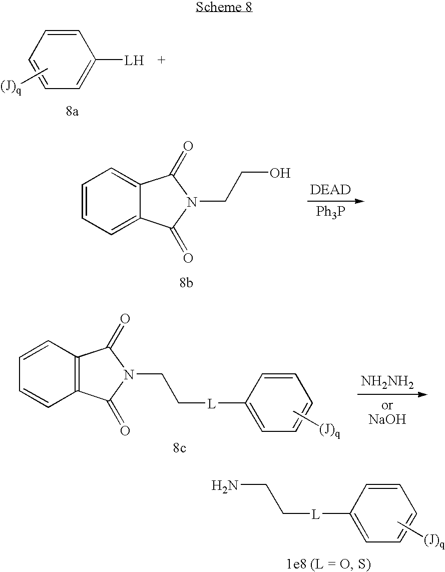

The desired Formula 1e compounds wherein R² is hydrogen, K is —CH₂CH₂L-, Ar² and B are bonds, Ar¹ is a phenyl ring and J and q are as described above, may be prepared by the Mitsunobu reaction of an appropriately substituted phenol (L=O) or thiophenol (L=S) 8a with hydroxyethylphthalimide 8b in the presence of diethyl azodicarboxylate and triphenylphosphine in an inert solvent such as tetrahydrofuran, dimethoxyethane or dimethylformamide at temperature between about 15° C. to 35° C. for about 10 to 30 hours, preferably in tetrahydrofuran at room temperature overnight (Scheme 8) The desired amine 1e8 may be prepared from phthalimide 8c by methods known to those skilled in the art, including those described in Scheme 6.

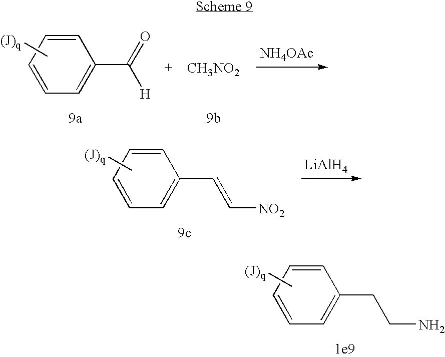

The desired Formula 1e compounds wherein R² is hydrogen, K is —CH₂CH₂—, Ar² and B are bonds, Ar¹ is a phenyl ring and J and q are as described above, may be prepared by the reaction sequence shown in Scheme 9. Condensation of nitromethane 9b with an appropriately substituted benzaldehyde in the presence of a base such as ammonium acetate or butylamine in a solvent such as nitromethane, acetic acid or toluene at a temperature of about 95° C. to 129° C. for about 15 min to 2 hours leads to nitroolefin 9c.

Reduction of nitroolefin 9c to amine 1e9 may be carried out by methods known to those skilled in the art, including the use of reducing agents such as lithium aluminum hydride, Red-AI or sodium aluminum hydride in an inert solvent such as tetrahydrofuran or dimethoxyethane at a temperature between about 20° C. to 40° C. for about 8 to 30 hours, preferably lithium aluminum hydride in tetrahydrofuran at room temperature overnight. Alternatively, nitroolefin 9c may be converted to amine 1e9 by catalytic hydrogenation in the presence of a catalyst such as palladium on carbon, in an alcoholic solvent such a ethanol at a hydrogen pressure of about 10 to 50 psi at about 20° C. to 30° C. for about 3 to 24 hours, preferably at room temperature at 45 psi overnight.

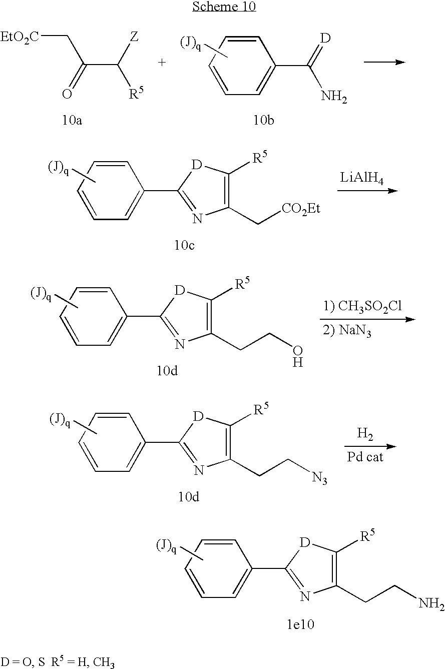

The desired Formula 1e compounds wherein R² is hydrogen, K is —CH₂CH₂—, Ar¹ is thiazolyl or oxazolyl, B is a bond, Ar² is phenyl and J and q are as described above, may be prepared by the reaction sequence shown in Scheme 10. Reaction of an appropriately substituted thiobenzamide 10b (D=S), which are commercially available, known in the literature or readily prepared by those skilled in the art, with an appropriately substituted 4-halo-3-oxoester 10a (Z=Cl, Br), which are commercially available, known in the literature or readily prepared by those skilled in the art, in an inert solvent such as ethanol or dimethylformamide, at a temperature of about 60° C. to 100° C. for about 2 to 24 hours, preferably in ethanol at reflux for 2 hours, leads to thiazolyl ester 10c (D=S).

Irradiation of a mixture of an appropriately substituted benzamide 10b (D=O), which are commercially available, known in the literature or readily prepared by those skilled in the art, an appropriately substituted 4-halo-3-oxoester 10a (Z=Cl, Br) and a catalytic amount of an acid such as p-toluenesulfonic acid in an inert solvent solvent such as ethanol or N-methylpyrollidone, in a microwave oven (high power) at a temperature of about 160° C. to 200° C. for about 15 to 40 min, preferably in ethanol at 170° C. for 20 min, yields oxazolyl ester 10c (D=O).

Reduction of ester 10c with a reducing agent such as lithium aluminum hydride or lithium borohydride, in an inert solvent such tetrahydrofuran or diethyl ether, at a temperature of about 0° C. to 20° C. for about 1 to 12 hours, preferably lithium aluminum hydride in tetrahydrofuran at 0° C. for 2 hours, leads to alcohol 10c. Alcohol 10c may be converted to azide 10d by reaction with methanesulfonyl chloride in an inert solvent such as methylene chloride or tetrahydrofuran, in the presence of an amine base such as 4-dimethylaminopyridine or triethylamine at a temperature of about 15° C. to 35° C. for about 15 to 30 hours, preferably in methylene chloride at room temperature overnight, followed by treatment of the resulting methanesulfonate with sodium azide in a solvent such as dimethylformamide or N-methylpyrrolidone at a temperature of about 60° C. to 90° C. for about 15 to 30 hours, preferably in dimethylformamide at 80° C. overnight.

The amine 1e10 is obtained by reducing azide 10d with hydrogen at a pressure of about 15 to 55 psi, preferably 50 psi, in an alcoholic solvent, preferably methanol, in the presence of a catalyst such as palladium on celite or palladium on carbon, preferably palladium on celite at a temperature of about 18° C. to 30° C. for about 5 to 30 hours, preferably at room temperature overnight.

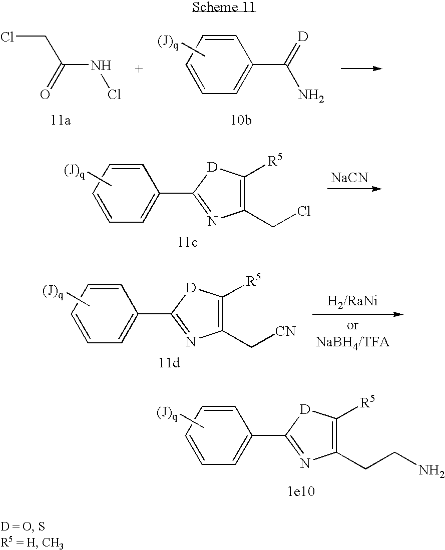

Alternatively, the desired Formula 1e compounds wherein R² is hydrogen, K is —CH₂CH₂—, Ar¹ is thiazolyl or oxazolyl, B is a bond, Ar² is phenyl and J and q are as described above, may be prepared by the reaction sequence shown in Scheme 11. Reaction of an appropriately substituted thiobenzamide 10b (D=S) with dichloroacetone 11a, which is commercially available, in a solvent such as ethanol or dimethylformamide, preferably ethanol, at a temperature of about 70° C. to 100° C. for about 2 to 24 hours, preferably 80° C. for 2 hours, leads to chloromethylthiazole 11c (D=S).

Chloromethyloxazole 11c (D=O) may be obtained by heating an appropriately substituted benzamide 10b (D=O) with dichloroacetone 11a at a temperature of about 110° C. to 150° C. for about 2 to 8 hours, preferably at 120° C. for 2 hours. Reaction of chloromethylazole 11c with sodium cyanide in a solvent such as dimethylformamide or N-methylpyrrolidone, preferably dimethylformamide, at a temperature of about 20° C. to 35° C. for about 12 to 30 hours, preferably at room temperature overnight, leads to nitrile 11d.

Amine 1e10 may be obtained by reducing nitrile 11d with hydrogen at a pressure of about 45 to 60 psi, preferably 50 psi, in the presence of Raney nickel in an alcoholic solvent containing ammonia, preferably ammonia in methanol, at a temperature of about 20° C. to 30° C. for about 15 to 30 hours, preferably at room temperature overnight. Alternatively reduction of nitrile 1e10 with sodium borohydride/trifluoroacetic acid in a solvent such as tetrahydrofuran leads to amine 1e10.

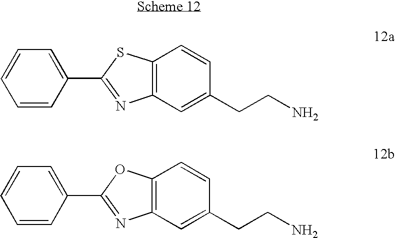

The desired Formula 1e compounds (depicted as 12a and 12b) wherein R² is hydrogen, K is —CH₂CH₂—, Ar¹ is benzothiazolyl or benzoxazolyl, B is a bond, Ar² is phenyl and J and q are as described above, may be prepared by methods known in the literature.

Synthetic procedures for 2-phenyl-5-aminoethylbenzothiazole (12a) amd 2-phenyl-5-aminoethylbenzoxazole (12b) derivatives (Scheme 12) are reported in J. Med. Chem., 16, 930 (1973) and J. Med. Chem., 18, 53 (1975), respectively.

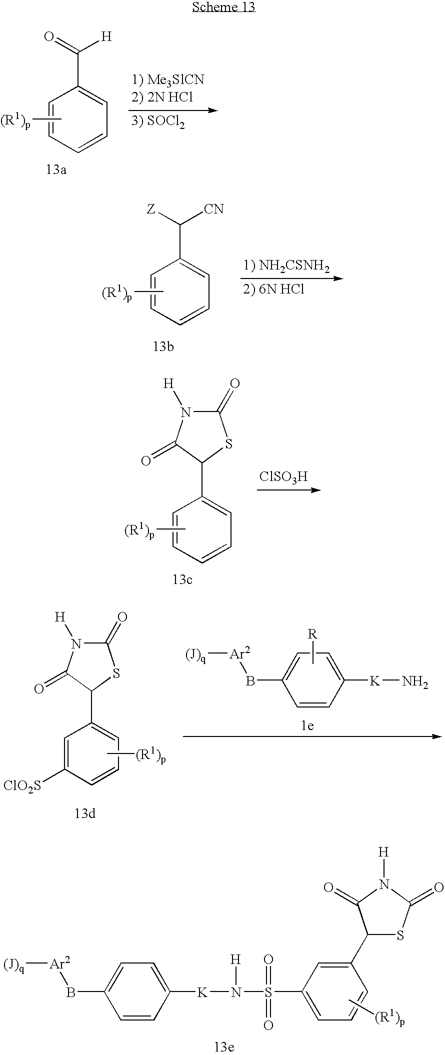

Compounds of Formula I wherein X is thiazolidinedione-5-yl-G-, G is (CH₂)ₛ, s is 0, R² is H, R (optionally present) is halo, alkyl, alkoxy or alkylthio and R¹, K, B, Ar², J, p and q are as described above, may be prepared by the synthetic sequence outlined in Scheme 13, as taught by J. Med. Chem., 29, 773 (1986) and Chem. Pharm. Bull., 30, 3601 (1982). An appropriately substituted benzaldehyde 13a is treated with trimethylsilyl cyanide and a catalytic amount of zinc iodide in anhydrous methylene chloride or chloroform at about 20° C. to 30° C. for about 15 to 30 hours, preferably in methylene chloride at room temperature overnight to yield the cyanohydrin 13b (Z=OH).

The cyanohydrin 13b (Z=OH) is converted to the chlorocyanide 13b (Z=Cl) with thionyl chloride in chloroform or methylene chloride at about 30° C. to 65° C. for about 30 to 60 min, preferably in chloroform at reflux temperature for 45 min. Reaction of chlorocyanide 13b (Z=Cl) with thiourea in an alcoholic solvent such as ethanol at about 60° C. to 80° C. for about 4 to 10 hours, preferably in ethanol at reflux temperature for 5 hours followed, by hydrolysis of the intermediate iminothiazolidinone with aqueous acid at about 95° C. to 120° C. for about 4 to 10 hours, preferably 6N aqueous hydrochloric acid at reflux temperature for 5 hours leads to the thiazolidinedione 13c.

Alternatively, appropriate benzaldehyde 13a is treated with sodium cyanide in a mixture of water, acetic acid and ethylene glycol monomethyl ether at room temperature for about 1.5 hours followed by the addition of thiourea and concentrated hydrochloric acid and heating at about 100° C. for about 18 hours to yield thiazolidinedione 13c (Chem. Pharm. Bull., 45, 1984 (1997).

Heating thiazolidinedione 13c in neat chlorosulfonic acid at about 90° C. to 110° C. for about 15 to 30 min, preferably at 100° C. for 15 min yields sulfonyl chloride 13d. Reaction of sulfonyl chloride 13d with appropriately substituted amines 1e using procedures known to those skilled in the art, such as the reaction described in Scheme 1, leads to the desired thiazolidinedione derivatives 13e.

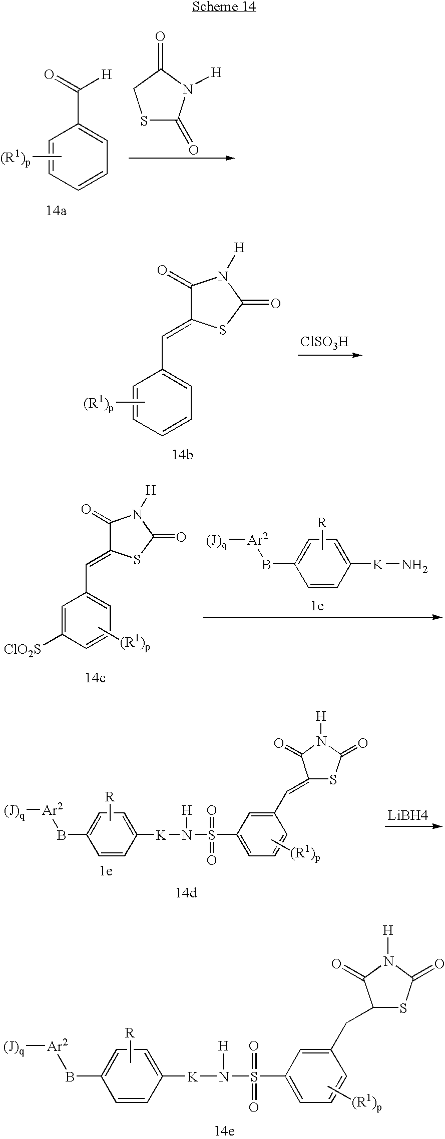

Compounds of Formula I wherein X is thiazolidinedione-5-yl-G-, G is methylidine or (CH₂)ₛ and s is 1, R² is H, R (optionally present) is halo, alkyl, alkoxy or alkylthio and R¹, K, B, Ar², J, p and q are as described above, may be synthesized by the reaction sequence outlined in Scheme 14, as taught by Chem. Pharm. Bull., 45, 1984 (1997). Condensation of an appropriately substituted benzaldehyde 14a and thiazolidinedione mediated by piperidine in acetic acid or ethanol or ammonium acetate in acetic acid at about 110° C. to 120° C. for about 8 to 30 hours, preferably piperidine in acetic acid at reflux for about 20 hours, or by piperidine and benzoic acid in toluene at reflux for about 3 to 10 hours leads to benzylidene thiazolidinedione 14b. Heating thiazolidinedione 14b in neat chlorosulfonic acid at about 90° C. to 110° C. for about 15 to 25 min, preferably about 100° C. for 15 min yields sulfonyl chloride 14c.

Reaction of sulfonyl chloride 14c with appropriately substituted amines 1e using procedures known to those skilled in the art, such as the process described in Scheme 1, leads to benzylidene thiazolidinedione derivatives 14d.

Reduction of the olefinic bond of 14d using methods familiar to those skilled in the art, such as lithium borohydride in pyridine/tetrahydrofuran at about 65° C. to 90° C. for about 2 to 6 hours or sodium borohydride/lithium chloride in pyridine/tetrahydrofuran at about 65° C. to 90° C. for about 3 to 6 hours, or catalytic hydrogenation with 10% Pd—C in 1,4-dioxane or methanol at about 50 to 60 psi for about 36 to 60 hours, preferably lithium borohydride in pyridine/tetrahydrofuran at reflux for 3 hours, yields the desired thiazolidinedione derivative 14e.

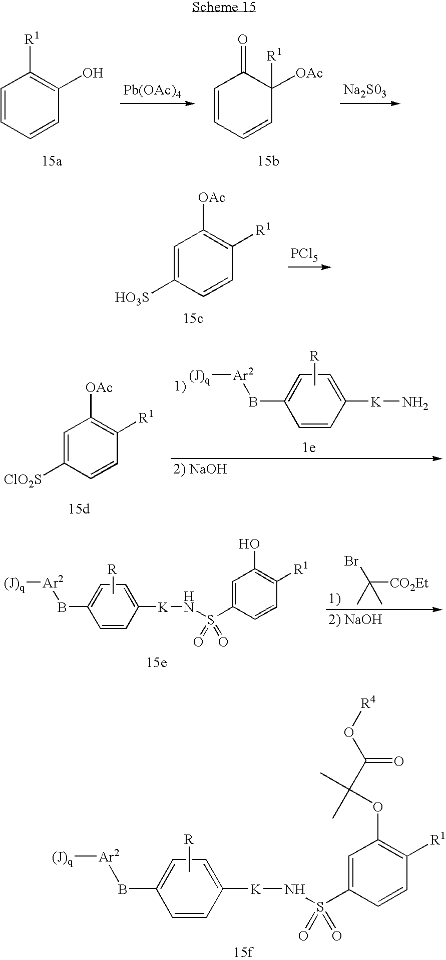

Compounds of Formula I, wherein X is —O—(CR³₂)—COOR⁴, R³ is CH₃, R¹ is alkyl, R² is H, R (optionally present) is halo, alkyl, alkoxy or alkylthio and, B, Ar², R⁴, J and q are as described above, may be prepared by the synthetic route outlined in Scheme 15 as taught by Monat. Chem. 99, 2048 (1968). The reaction of substituted phenol 15a with lead tetraacetate in acetic acid at about 20° C. to 30° C. for about 3 to 6 hours, preferably at room temperature for about 3 hours yields quinol acetate 15b.

Upon treatment with sodium sulfite in water at about 20° C. to 30° C. for about 3 to 6 hours, preferably room temperature for 3 hours, quinol acetate 15b is converted to sulfonic acid 15c.

Sulfonyl chloride 15d is prepared by heating sulfonic acid 15c with phosphorus pentachloride at about 110° C. to 130° C. for about 25 to 55 min, preferably about 120° C. for about 30 min.

Reaction of sulfonyl chloride 15d with appropriately substituted amines 1e using procedures known to those skilled in the art, such as the process described in Scheme 1, followed by alkaline hydrolysis of the acetate yields sulfonamide 15e.

Alkylation of sulfonamide 15e with ethyl 2-bromoisobutyrate and potassium carbonate in dimethylformamide or ethanol at about 80° C. to 100° C. for about 12 to 24 hours, preferably dimethylformamide at about 95° C. for about 18 hours, followed by basic hydrolysis of the product, leads to the desired acid 15f, wherein R⁴ is H. 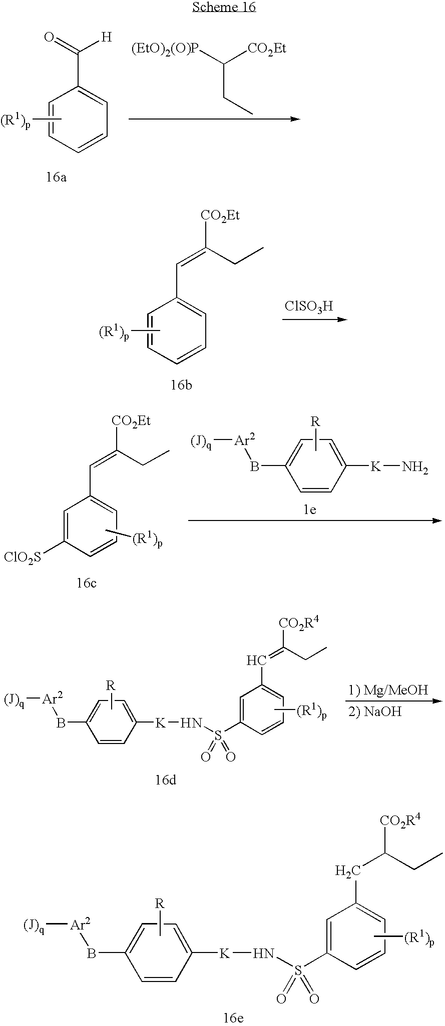
Compounds of Formula I wherein X is —CH₂(CR⁵_{w})—COOR⁴ and R⁵ is CH₃CH₂, w is 1, R² is H, R (optionally present) is halo, alkyl, alkoxy or alkylthio and R¹, R⁴, K, B, Ar², J, p and q are as described above, may be synthesized by the reaction sequence outlined in Scheme 16. Reaction of an appropriately substituted benzaldehyde 16a with the carbanion formed from triethyl-2-phosphonobutyrate and potassium t-butoxide or sodium hydride in tetrahydrofuran or dimethoxyethane at about 20° C. to 30° C. for about 2 to 5 hours, preferably at room temperature for 3 hours, yields olefinic ester 16b.

Ester 16b is converted to sulfonyl chloride 16c by heating in chlorosulfonic acid at about 55° C. to 70° C. for about 15 to 25 min, preferably at about 60° C. for about 15 min.

Reaction of sulfonyl chloride 16c with appropriately substituted amines 1e using methods know to those skilled in the art, such as the process described in Scheme 1, yields sulfonamide 16d.

Reduction of the olefinic bond of 16c using procedures known to those skilled in the art, such as magnesium in methanol or ethanol at about 60° C. to 85° C. until the magnesium is consumed, or catalytic hydrogenation with 10% Pd—C in 1,4-dioxane or methanol at about 50 to 60 psi for about 36 to 60 hours, preferably magnesium in methanol at about 65° C., followed by alkaline hydrolysis of the product, yields the desired acid 16e.

The compounds of this invention may also be used in conjunction with other pharmaceutical agents (e.g., LDL-cholesterol lowering agents, triglyceride lowering agents) for the treatment of the disease/conditions described herein. For example, they may be used in combination with a HMG-CoA reductase inhibitor, a cholesterol synthesis inhibitor, a cholesterol absorption inhibitor, a CETP inhibitor, a MTP/Apo B secretion inhibitor, another PPAR modulator and other cholesterol lowering agents such as a fibrate, niacin, an ion-exchange resin, an antioxidant, an ACAT inhibitor, and a bile acid sequestrant. Other pharmaceutical agents would also include the following: a bile acid reuptake inhibitor, an ileal bile acid transporter inhibitor, an ACC inhibitor, an antihypertensive (such as NORVASC®), a selective estrogen receptor modulator, a selective androgen receptor modulator, an antibiotic, an antidiabetic (such as mefformin, a PPARγ activator, a sulfonylurea, insulin, an aldose reductase inhibitor (ARI) and a sorbitol dehydrogenase inhibitor (SDI)), and aspirin (acetylsalicylic acid or a nitric oxide releasing asprin). A slow-release form of niacin is available and is known as Niaspan. Niacin may also be combined with other therapeutic agents such as statins, i.e. lovastatin, which is an HMG-CoA reductase inhibitor and described further below. This combination therapy is known as ADVICOR® (Kos Pharmaceuticals Inc.) In combination therapy treatment, both the compounds of this invention and the other drug therapies are administered to mammals (e.g., humans, male or female) by conventional methods.

Any HMG-CoA reductase inhibitor may be used in the combination aspect of this invention. The conversion of 3-hydroxy-3-methylglutaryl-coenzyme A (HMG-CoA) to mevalonate is an early and rate-limiting step in the cholesterol biosynthetic pathway. This step is catalyzed by the enzyme HMG-CoA reductase. Statins inhibit HMG-CoA reductase from catalyzing this conversion. The following paragraphs describe exemplary statins.

The term HMG-CoA reductase inhibitor refers to compounds which inhibit the bioconversion of hydroxymethylglutaryl-coenzyme A to mevalonic acid catalyzed by the enzyme HMG-CoA reductase. Such inhibition is readily determined by those skilled in the art according to standard assays (e.g., Meth. Enzymol. 1981; 71:455-509 and references cited therein). A variety of these compounds are described and referenced below however other HMG-CoA reductase inhibitors will be known to those skilled in the art. U.S. Pat. No. 4,231,938 (the disclosure of which is hereby incorporated by reference) discloses certain compounds isolated after cultivation of a microorganism belonging to the genus Aspergillus, such as lovastatin. Also, U.S. Pat. No. 4,444,784 (the disclosure of which is hereby incorporated by reference) discloses synthetic derivatives of the aforementioned compounds, such as simvastatin. Also, U.S. Pat. No. 4,739,073 (the disclosure of which is incorporated by reference) discloses certain substituted indoles, such as fluvastatin. Also, U.S. Pat. No. 4,346,227 (the disclosure of which is incorporated by reference) discloses ML-236B derivatives, such as pravastatin. Also, EP-491226A (the disclosure of which is incorporated by reference) discloses certain pyridyldihydroxyheptenoic acids, such as cerivastatin. In addition, U.S. Pat. No. 5,273,995 (the disclosure of which is incorporated by reference) discloses certain 6-[2-(substituted-pyrrol-1-yl)alkyl]pyran-2-ones such as atorvastatin and any pharmaceutically acceptable form thereof (i.e. LIPITOR®). Additional HMG-CoA reductase inhibitors include rosuvastatin and pitavastatin.

Atorvastatin calcium (i.e., atorvastatin hemicalcium), disclosed in U.S. Pat. No. 5,273,995, which is incorporated herein by reference, is currently sold as Lipitor® and has the formula 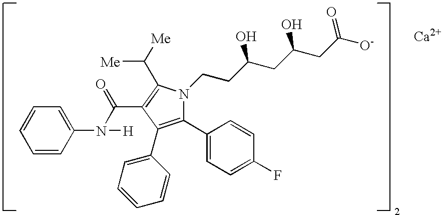
Atorvastatin calcium is a selective, competitive inhibitor of HMG-CoA. As such, atorvastatin calcium is a potent lipid lowering compound. The free carboxylic acid form of atorvastatin may exist predominantly as the lactone of the formula 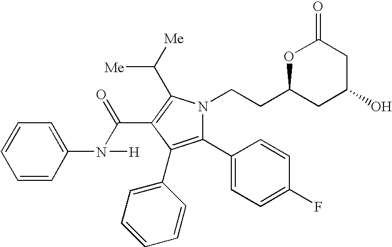
and is disclosed in U.S. Pat. No. 4,681,893, which is incorporated herein by reference.

Statins also include such compounds as rosuvastatin disclosed in U.S. RE37,314 E, pitivastatin disclosed in EP 304063 B1 and U.S. Pat. No. 5,011,930, simvastatin, disclosed in U.S. Pat. No. 4,444,784, which is incorporated herein by reference; pravastatin, disclosed in U.S. Pat. No. 4,346,227 which is incorporated herein by reference; cerivastatin, disclosed in U.S. Pat. No. 5,502,199, which is incorporated herein by reference; mevastatin, disclosed in U.S. Pat. No. 3,983,140, which is incorporated herein by reference; velostatin, disclosed in U.S. Pat. No. 4,448,784 and U.S. Pat. No. 4,450,171, both of which are incorporated herein by reference; fluvastatin, disclosed in U.S. Pat. No. 4,739,073, which is incorporated herein by reference; compactin, disclosed in U.S. Pat. No. 4,804,770, which is incorporated herein by reference; lovastatin, disclosed in U.S. Pat. No. 4,231,938, which is incorporated herein by reference; dalvastatin, disclosed in European Patent Application Publication No. 738510 A2; fluindostatin, disclosed in European Patent Application Publication No. 363934 A1; and dihydrocompactin, disclosed in U.S. Pat. No. 4,450,171, which is incorporated herein by reference.

Any HMG-CoA synthase inhibitor may be used in the combination aspect of this invention. The term HMG-CoA synthase inhibitor refers to compounds which inhibit the biosynthesis of hydroxymethylglutaryl-coenzyme A from acetyl-coenzyme A and acetoacetyl-coenzyme A, catalyzed by the enzyme HMG-CoA synthase. Such inhibition is readily determined by those skilled in the art according to standard assays (Meth Enzymol. 1975; 35:155-160: Meth. Enzymol. 1985; 110:19-26 and references cited therein). A variety of these compounds are described and referenced below, however other HMG-CoA synthase inhibitors will be known to those skilled in the art. U.S. Pat. No. 5,120,729 (the disclosure of which is hereby incorporated by reference) discloses certain beta-lactam derivatives. U.S. Pat. No. 5,064,856 (the disclosure of which is hereby incorporated by reference) discloses certain spiro-lactone derivatives prepared by culturing a microorganism (MF5253). U.S. Pat. No. 4,847,271 (the disclosure of which is hereby incorporated by reference) discloses certain oxetane compounds such as 11-(3-hydroxymethyl-4-oxo-2-oxetayl)-3,5,7-trimethyl-2,4-undeca-dienoic acid derivatives.

Any compound that decreases HMG-CoA reductase gene expression may be used in the combination aspect of this invention. These agents may be HMG-CoA reductase transcription inhibitors that block the transcription of DNA or translation inhibitors that prevent or decrease translation of mRNA coding for HMG-CoA reductase into protein. Such compounds may either affect transcription or translation directly, or may be biotransformed to compounds that have the aforementioned activities by one or more enzymes in the cholesterol biosynthetic cascade or may lead to the accumulation of an isoprene metabolite that has the aforementioned activities. Such compounds may cause this effect by decreasing levels of SREBP (sterol regulatory element binding protein) by inhibiting the activity of site-1 protease (S1P) or agonizing the oxysterol receptor or antagonizing SCAP. Such regulation is readily determined by those skilled in the art according to standard assays (Meth. Enzymol. 1985; 110:9-19). Several compounds are described and referenced below, however other inhibitors of HMG-CoA reductase gene expression will be known to those skilled in the art. U.S. Pat. No. 5,041,432 (the disclosure of which is incorporated by reference) discloses certain 15-substituted lanosterol derivatives.

Other oxygenated sterols that suppress synthesis of HMG-CoA reductase are discussed by E. I. Mercer (Prog. Lip. Res. 1993; 32:357-416).

Any compound having activity as a CETP inhibitor can serve as the second compound in the combination therapy aspect of the present invention. The term CETP inhibitor refers to compounds that inhibit the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). A variety of CETP inhibitors will be known to those skilled in the art, for example, those disclosed in commonly assigned U.S. Pat. No. 6,140,343 and commonly assigned U.S. Pat. No. 6,197,786. CETP inhibitors disclosed in these patents include compounds, such as [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, which is also known as torcetrapib. CETP inhibitors are also described in U.S. Pat. No. 6,723,752, which includes a number of CETP inhibitors including (2R)-3-{[3-(4-Chloro-3-ethyl-phenoxy)-phenyl]-[[3-(1,1,2,2-tetrafluoro-ethoxy)-phenyl]-methyl]-amino}-1,1,1-trifluoro-2-propanol. Moreover, CETP inhibitors included herein are also described in U.S. patent application Ser. No. 10/807,838 filed Mar. 23, 2004. U.S. Pat. No. 5,512,548 discloses certain polypeptide derivatives having activity as CETP inhibitors, while certain CETP-inhibitory rosenonolactone derivatives and phosphate-containing analogs of cholesteryl ester are disclosed in J. Antibiot., 49(8): 815-816 (1996), and Bioorg. Med. Chem. Lett.; 6:1951-1954 (1996), respectively.

Any PPAR modulator may be used in the combination aspect of this invention. The term PPAR modulator refers to compounds which modulate peroxisome proliferator activator receptor (PPAR) activity in mammals, particularly humans. Such modulation is readily determined by those skilled in the art according to standard assays known in the literature. It is believed that such compounds, by modulating the PPAR receptor, regulate transcription of key genes involved in lipid and glucose metabolism such as those in fatty acid oxidation and also those involved in high density lipoprotein (HDL) assembly (for example, apolipoprotein AI gene transcription), accordingly reducing whole body fat and increasing HDL cholesterol. By virtue of their activity, these compounds also reduce plasma levels of triglycerides, VLDL cholesterol, LDL cholesterol and their associated components such as apolipoprotein B in mammals, particularly humans, as well as increasing HDL cholesterol and apolipoprotein AI. Hence, these compounds are useful for the treatment and correction of the various dyslipidemias observed to be associated with the development and incidence of atherosclerosis and cardiovascular disease, including hypoalphalipoproteinemia and hypertriglyceridemia. A variety of these compounds are described and referenced below, however, others will be known to those skilled in the art. International Publication Nos. WO 02/064549 and 02/064130 and U.S. patent application Ser. No. 10/720,942, filed Nov. 24, 2003 and U.S. patent application 60/552,114 filed Mar. 10, 2004 (the disclosures of which are hereby incorporated by reference) disclose certain compounds which are PPARα activators.

Any other PPAR modulator may be used in the combination aspect of this invention. In particular, modulators of PPARβ and/or PPARγ may be useful incombination with compounds of the present invention. An example PPAR inhibitor is described in U.S. 2003/0225158 as {5-Methoxy-2-methyl-4-[4-(4-trifluoromethyl-benzyloxy)-benzylsulfany]-phenoxy}-acetic acid.

Any MTP/Apo B (microsomal triglyceride transfer protein and or apolipoprotein B) secretion inhibitor may be used in the combination aspect of this invention. The term MTP/Apo B secretion inhibitor refers to compounds which inhibit the secretion of triglycerides, cholesteryl ester, and phospholipids. Such inhibition is readily determined by those skilled in the art according to standard assays (e.g., Wefterau, J. R. 1992; Science 258:999). A variety of these compounds are described and referenced below however other MTP/Apo B secretion inhibitors will be known to those skilled in the art, including imputapride (Bayer) and additional compounds such as those disclosed in WO 96/40640 and WO 98/23593, (two exemplary publications).

For example, the following MTP/Apo B secretion inhibitors are particularly useful:
4′-trifluoromethyl-biphenyl-2-carboxylic acid [2-(1H-[1,2,4,]triazol-3-ylmethyl)-1,2,3,4-tetrahydro-isoquinolin-6-yl]-amide;
4′-trifluoromethyl-biphenyl-2-carboxylic acid [2-(2-acetylamino-ethyl)-1,2,3,4-tetrahydro-isoquinolin-6-yl]-amide;
(2-{6-[(4′-trifluoromethyl-biphenyl-2-carbonyl)-amino]-3,4-dihydro-1H-isoquinolin-2-yl}-ethyl)-carbamic acid methyl ester;
4′-trifluoromethyl-biphenyl-2-carboxylic acid [2-(1H-imidazol-2-ylmethyl)-1,2,3,4-tetrahydro-isoquinolin-6-yl]-amide;
4′-trifluoromethyl-biphenyl-2-carboxylic acid [2-(2,2-diphenyl-ethyl)-1,2,3,4-tetrahydro-isoquinolin-6-yl]-amide; and
4′-trifluoromethyl-biphenyl-2-carboxylic acid [2-(2-ethoxy-ethyl)-1,2,3,4-tetrahydro-isoquinolin-6-yl]-amide.
(S)-N-{2-[benzyl(methyl)amino]-2-oxo-1-phenylethyl}-1-methyl-5-[4′-(trifluoromethyl)[1,1′-biphenyl]-2-carboxamido]-1H-indole-2-carboxamide;
(S)-2-[(4′-Trifluoromethyl-biphenyl-2-carbonyl)-amino]-quinoline-6-carboxylic acid (pentylcarbamoyl-phenyl-methyl)-amide;
1H-indole-2-carboxamide,1-methyl-N-[(1S)-2-[methyl(phenylmethyl)amino]-2-oxo-1-phenylethyl]-5-[[[4′-(trifluoromethyl)[1,1′-biphenyl]-2-yl]carbonyl]amino]; and
N-[(1S)-2-(benzylmethylamino)-2-oxo-1-phenylethyl]-1-methyl-5-[[[4′-(trifluoromethyl)biphenyl-2-yl]carbonyl]amino]-1H-indole-2-carboxamide.

Any squalene synthetase inhibitor may be used in the combination aspect of this invention. The term squalene synthetase inhibitor refers to compounds which inhibit the condensation of 2 molecules of farnesylpyrophosphate to form squalene, catalyzed by the enzyme squalene synthetase. Such inhibition is readily determined by those skilled in the art according to standard assays (Meth. Enzymol. 1969; 15: 393-454 and Meth. Enzymol. 1985; 110:359-373 and references contained therein). A variety of these compounds are described in and referenced below however other squalene synthetase inhibitors will be known to those skilled in the art. U.S. Pat. No. 5,026,554 (the disclosure of which is incorporated by reference) discloses fermentation products of the microorganism MF5465 (ATCC 74011) including zaragozic acid. A summary of other patented squalene synthetase inhibitors has been compiled (Curr. Op. Ther. Patents (1993) 861-4).

Any squalene epoxidase inhibitor may be used in the combination aspect of this invention. The term squalene epoxidase inhibitor refers to compounds which inhibit the bioconversion of squalene and molecular oxygen into squalene-2,3-epoxide, catalyzed by the enzyme squalene epoxidase. Such inhibition is readily determined by those skilled in the art according to standard assays (Biochim. Biophys. Acta 1984; 794:466-471). A variety of these compounds are described and referenced below, however other squalene epoxidase inhibitors will be known to those skilled in the art. U.S. Pat. Nos. 5,011,859 and 5,064,864 (the disclosures of which are incorporated by reference) disclose certain fluoro analogs of squalene. EP publication 395,768 A (the disclosure of which is incorporated by reference) discloses certain substituted allylamine derivatives. PCT publication WO 9312069 A (the disclosure of which is hereby incorporated by reference) discloses certain amino alcohol derivatives. U.S. Pat. No. 5,051,534 (the disclosure of which is hereby incorporated by reference) discloses certain cyclopropyloxy-squalene derivatives.

Any squalene cyclase inhibitor may be used as the second component in the combination aspect of this invention. The term squalene cyclase inhibitor refers to compounds which inhibit the bioconversion of squalene-2,3-epoxide to lanosterol, catalyzed by the enzyme squalene cyclase. Such inhibition is readily determined by those skilled in the art according to standard assays (FEBS Lett. 1989; 244:347-350.). In addition, the compounds described and referenced below are squalene cyclase inhibitors, however other squalene cyclase inhibitors will also be known to those skilled in the art. PCT publication WO9410150 (the disclosure of which is hereby incorporated by reference) discloses certain 1,2,3,5,6,7,8,8a-octahydro-5,5,8(beta)-trimethyl-6-isoquinolineamine derivatives, such as N-trifluoroacetyl-1,2,3,5,6,7,8,8a-octahydro-2-allyl-5,5,8(beta)-trimethyl-6(beta)-isoquinolineamine. French patent publication 2697250 (the disclosure of which is hereby incorporated by reference) discloses certain beta, beta-dimethyl-4-piperidine ethanol derivatives such as 1-(1,5,9-trimethyldecyl)-beta,beta-dimethyl-4-piperidineethanol

Any combined squalene epoxidase/squalene cyclase inhibitor may be used as the second component in the combination aspect of this invention. The term combined squalene epoxidase/squalene cyclase inhibitor refers to compounds that inhibit the bioconversion of squalene to lanosterol via a squalene-2,3-epoxide intermediate. In some assays it is not possible to distinguish between squalene epoxidase inhibitors and squalene cyclase inhibitors, however, these assays are recognized by those skilled in the art. Thus, inhibition by combined squalene epoxidase/squalene cyclase inhibitors is readily determined by those skilled in art according to the aforementioned standard assays for squalene cyclase or squalene epoxidase inhibitors. A variety of these compounds are described and referenced below, however other squalene epoxidase/squalene cyclase inhibitors will be known to those skilled in the art. U.S. Pat. Nos. 5,084,461 and 5,278,171 (the disclosures of which are incorporated by reference) disclose certain azadecalin derivatives. EP publication 468,434 (the disclosure of which is incorporated by reference) discloses certain piperidyl ether and thio-ether derivatives such as 2-(1-piperidyl)pentyl isopentyl sulfoxide and 2-(1-piperidyl)ethyl ethyl sulfide. PCT publication WO 9401404 (the disclosure of which is hereby incorporated by reference) discloses certain acyl-piperidines such as 1-(1-oxopentyl-5-phenylthio)-4-(2-hydroxy-1-methyl)-ethyl)piperidine. U.S. Pat. No. 5,102,915 (the disclosure of which is hereby incorporated by reference) discloses certain cyclopropyloxy-squalene derivatives.

The compounds of the present invention can also be administered in combination with naturally occurring compounds that act to lower plasma cholesterol levels. These naturally occurring compounds are commonly called nutraceuticals and include, for example, garlic extract and niacin. A slow-release form of niacin is available and is known as Niaspan. Niacin may also be combined with other therapeutic agents such as lovastatin, or another HMG-CoA reductase inhibitor. This combination therapy with lovastatin is known as ADVICOR™ (Kos Pharmaceuticals Inc.).

Any cholesterol absorption inhibitor can be used as an additional in the combination aspect of the present invention. The term cholesterol absorption inhibition refers to the ability of a compound to prevent cholesterol contained within the lumen of the intestine from entering into the intestinal cells and/or passing from within the intestinal cells into the lymph system and/or into the blood stream. Such cholesterol absorption inhibition activity is readily determined by those skilled in the art according to standard assays (e.g., J. Lipid Res. (1993) 34: 377-395). Cholesterol absorption inhibitors are known to those skilled in the art and are described, for example, in PCT WO 94/00480. An example of a cholesterol absorption inhibitor is ZETIA™ (ezetimibe) (Schering-Plough/Merck).

Any ACAT inhibitor may be used in the combination therapy aspect of the present invention. The term ACAT inhibitor refers to compounds that inhibit the intracellular esterification of dietary cholesterol by the enzyme acyl CoA: cholesterol acyltransferase. Such inhibition may be determined readily by one of skill in the art according to standard assays, such as the method of Heider et al. described in Journal of Lipid Research., 24:1127 (1983). A variety of these compounds are known to those skilled in the art, for example, U.S. Pat. No. 5,510,379 discloses certain carboxysulfonates, while WO 96/26948 and WO 96/10559 both disclose urea derivatives having ACAT inhibitory activity. Examples of ACAT inhibitors include compounds such as Avasimibe (Pfizer), CS-505 (Sankyo) and Eflucimibe (Eli Lilly and Pierre Fabre).

A lipase inhibitor may be used in the combination therapy aspect of the present invention. A lipase inhibitor is a compound that inhibits the metabolic cleavage of dietary triglycerides or plasma phospholipids into free fatty acids and the corresponding glycerides (e.g. EL, HL, etc.). Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a glyceride and fatty acid. In the intestine, the resultant free fatty acids and monoglycerides are incorporated into bile acid-phospholipid micelles, which are subsequently absorbed at the level of the brush border of the small intestine. The micelles eventually enter the peripheral circulation as chylomicrons. Such lipase inhibition activity is readily determined by those skilled in the art according to standard assays (e.g., Methods Enzymol. 286: 190-231).

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology, 92,125 (1987). Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art. Preferred lipase inhibitors are those inhibitors that are selected from the group consisting of lipstatin, tetrahydrolipstatin (orlistat), valilactone, esterastin, ebelactone A, and ebelactone B. The compound tetrahydrolipstatin is especially preferred. The lipase inhibitor, N-3-trifluoromethylphenyl-N′-3-chloro-4′-trifluoromethylphenylurea, and the various urea derivatives related thereto, are disclosed in U.S. Pat. No. 4,405,644. The lipase inhibitor, esteracin, is disclosed in U.S. Pat. Nos. 4,189,438 and 4,242,453. The lipase inhibitor, cyclo-O,O′-[(1,6-hexanediyl)-bis-(iminocarbonyl)]dioxime, and the various bis(iminocarbonyl)dioximes related thereto may be prepared as described in Petersen et al., Liebig's Annalen, 562, 205-229 (1949).

A variety of pancreatic lipase inhibitors are described herein below. The pancreatic lipase inhibitors lipstatin, (2S,3S,5S,7Z,10Z)-5-[(S)-2-formamido-4-methyl-valeryloxy]-2-hexyl-3-hydroxy-7,10-hexadecanoic acid lactone, and tetrahydrolipstatin (orlistat), (2S,3S,5S)-5-[(S)-2-formamido-4-methyl-valeryloxy]-2-hexyl-3-hydroxy-hexadecanoic 1,3 acid lactone, and the variously substituted N-formylleucine derivatives and stereoisomers thereof, are disclosed in U.S. Pat. No. 4,598,089. For example, tetrahydrolipstatin is prepared as described in, e.g., U.S. Pat. Nos. 5,274,143; 5,420,305; 5,540,917; and 5,643,874. The pancreatic lipase inhibitor, FL-386, 1-[4-(2-methylpropyl)cyclohexyl]-2-[(phenylsulfonyl)oxy]-ethanone, and the variously substituted sulfonate derivatives related thereto, are disclosed in U.S. Pat. No. 4,452,813. The pancreatic lipase inhibitor, WAY-121898, 4-phenoxyphenyl-4-methylpiperidin-1-yl-carboxylate, and the various carbamate esters and pharmaceutically acceptable salts related thereto, are disclosed in U.S. Pat. Nos. 5,512,565; 5,391,571 and 5,602,151. The pancreatic lipase inhibitor, valilactone, and a process for the preparation thereof by the microbial cultivation of Actinomycetes strain MG147-CF2, are disclosed in Kitahara, et al., J. Antibiotics, 40 (11), 1647-1650 (1987). The pancreatic lipase inhibitors, ebelactone A and ebelactone B, and a process for the preparation thereof by the microbial cultivation of Actinomycetes strain MG7-G1, are disclosed in Umezawa, et al., J. Antibiotics, 33, 1594-1596 (1980). The use of ebelactones A and B in the suppression of monoglyceride formation is disclosed in Japanese Kokai 08-143457, published Jun. 4, 1996.

Other compounds that are marketed for hyperlipidemia, including hypercholesterolemia and which are intended to help prevent or treat atherosclerosis include bile acid sequestrants, such as Welchol®, Colestid®, LoCholest® and Questran®; and fibric acid derivatives, such as Atromid®, Lopid® and Tricor®.

Diabetes can be treated by administering to a patient having diabetes (especially Type II), insulin resistance, impaired glucose tolerance, metabolic syndrome, or the like, or any of the diabetic complications such as neuropathy, nephropathy, retinopathy or cataracts, a therapeutically effective amount of a compound of the present invention in combination with other agents (e.g., insulin) that can be used to treat diabetes. This includes the classes of anti-diabetic agents (and specific agents) described herein.

Any glycogen phosphorylase inhibitor can be used as the second agent in combination with a compound of the present invention. The term glycogen phosphorylase inhibitor refers to compounds that inhibit the bioconversion of glycogen to glucose-1-phosphate which is catalyzed by the enzyme glycogen phosphorylase. Such glycogen phosphorylase inhibition activity is readily determined by those skilled in the art according to standard assays (e.g., J. Med. Chem. 41 (1998) 2934-2938). A variety of glycogen phosphorylase inhibitors are known to those skilled in the art including those described in WO 96/39384 and WO 96/39385.

Any aldose reductase inhibitor can be used in combination with a compound of the present invention. The term aldose reductase inhibitor refers to compounds that inhibit the bioconversion of glucose to sorbitol, which is catalyzed by the enzyme aldose reductase. Aldose reductase inhibition is readily determined by those skilled in the art according to standard assays (e.g., J. Malone, Diabetes, 29:861-864 (1980). “Red Cell Sorbitol, an Indicator of Diabetic Control”). A variety of aldose reductase inhibitors are known to those skilled in the art, such as those described in U.S. Pat. No. 6,579,879, which includes 6-(5-chloro-3-methyl-benzofuran-2-sulfonyl)-2H-pyridazin-3-one.

Any sorbitol dehydrogenase inhibitor can be used in combination with a compound of the present invention. The term sorbitol dehydrogenase inhibitor refers to compounds that inhibit the bioconversion of sorbitol to fructose which is catalyzed by the enzyme sorbitol dehydrogenase. Such sorbitol dehydrogenase inhibitor activity is readily determined by those skilled in the art according to standard assays (e.g., Analyt. Biochem (2000) 280: 329-331). A variety of sorbitol dehydrogenase inhibitors are known, for example, U.S. Pat. Nos. 5,728,704 and 5,866,578 disclose compounds and a method for treating or preventing diabetic complications by inhibiting the enzyme sorbitol dehydrogenase.

Any glucosidase inhibitor can be used in combination with a compound of the present invention. A glucosidase inhibitor inhibits the enzymatic hydrolysis of complex carbohydrates by glycoside hydrolases, for example amylase or maltase, into bioavailable simple sugars, for example, glucose. The rapid metabolic action of glucosidases, particularly following the intake of high levels of carbohydrates, results in a state of alimentary hyperglycemia which, in adipose or diabetic subjects, leads to enhanced secretion of insulin, increased fat synthesis and a reduction in fat degradation. Following such hyperglycemias, hypoglycemia frequently occurs, due to the augmented levels of insulin present. Additionally, it is known chyme remaining in the stomach promotes the production of gastric juice, which initiates or favors the development of gastritis or duodenal ulcers. Accordingly, glucosidase inhibitors are known to have utility in accelerating the passage of carbohydrates through the stomach and inhibiting the absorption of glucose from the intestine. Furthermore, the conversion of carbohydrates into lipids of the fatty tissue and the subsequent incorporation of alimentary fat into fatty tissue deposits is accordingly reduced or delayed, with the concomitant benefit of reducing or preventing the deleterious abnormalities resulting therefrom. Such glucosidase inhibition activity is readily determined by those skilled in the art according to standard assays (e.g., Biochemistry (1969) 8: 4214).

A generally preferred glucosidase inhibitor includes an amylase inhibitor. An amylase inhibitor is a glucosidase inhibitor that inhibits the enzymatic degradation of starch or glycogen into maltose. Such amylase inhibition activity is readily determined by those skilled in the art according to standard assays (e.g., Methods Enzymol. (1955) 1: 149). The inhibition of such enzymatic degradation is beneficial in reducing amounts of bioavailable sugars, including glucose and maltose, and the concomitant deleterious conditions resulting therefrom.

A variety of glucosidase inhibitors are known to one of ordinary skill in the art and examples are provided below. Preferred glucosidase inhibitors are those inhibitors that are selected from the group consisting of acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are disclosed in U.S. Pat. Nos. 4,062,950 and 4,174,439 respectively. The glucosidase inhibitor, adiposine, is disclosed in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethyl)-D-epi-inositol, and the various N-substituted pseudo-aminosugars related thereto, are disclosed in U.S. Pat. No. 4,701,559. The glucosidase inhibitor, miglitol, (2R,3R,4R,5S)-1-(2-hydroxyethyl)-2-(hydroxymethyl)-3,4,5-piperidinetriol, and the various 3,4,5-trihydroxypiperidines related thereto, are disclosed in U.S. Pat. No. 4,639,436. The glucosidase inhibitor, emiglitate, ethyl p-[2-[(2R,3R,4R,5S)-3,4,5-trihydroxy-2-(hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor, MDL-25637, 2,6-dideoxy-7-O-β-D-glucopyrano-syl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor, camiglibose, methyl 6-deoxy-6-[(2R,3R,4R,5S)-3,4,5-trihydroxy-2-(hydroxymethyl)piperidino]-α-D-glucopyranoside sesquihydrate, the deoxy-nojirimycin derivatives related thereto, the various pharmaceutically acceptable salts thereof and synthetic methods for the preparation thereof, are disclosed in U.S. Pat. Nos. 5,157,116 and 5,504,078. The glycosidase inhibitor, salbostatin and the various pseudosaccharides related thereto, are disclosed in U.S. Pat. No. 5,091,524.

A variety of amylase inhibitors are known to one of ordinary skill in the art. The amylase inhibitor, tendamistat and the various cyclic peptides related thereto, are disclosed in U.S. Pat. No. 4,451,455. The amylase inhibitor AI-3688 and the various cyclic polypeptides related thereto are disclosed in U.S. Pat. No. 4,623,714. The amylase inhibitor, trestatin, consisting of a mixture of trestatin A, trestatin B and trestatin C and the various trehalose-containing aminosugars related thereto are disclosed in U.S. Pat. No. 4,273,765.

Additional anti-diabetic compounds, which can be used as the second agent in combination with a compound of the present invention, includes, for example, the following: biguanides (e.g., mefformin), insulin secretagogues (e.g., sulfonylureas and glinides), glitazones, non-glitazone PPARγ agonists, PPARβ agonists, inhibitors of DPP-IV, inhibitors of PDE5, inhibitors of GSK-3, glucagon antagonists, inhibitors of f-1,6-BPase(Metabasis/Sankyo), GLP-1/analogs (AC 2993, also known as exendin-4), insulin and insulin mimetics (Merck natural products). Other examples would include PKC-β inhibitors and AGE breakers.

The compounds of the present invention can be used in combination with other anti-obesity agents. Any anti-obesity agent can be used as the second agent in such combinations and examples are provided herein. Such anti-obesity activity is readily determined by those skilled in the art according to standard assays known in the art.

Suitable anti-obesity agents include phenylpropanolamine, ephedrine, pseudoephedrine, phentermine, β₃ adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine), sympathomimetic agents, serotoninergic agents, cannabinoid-1 receptor (CB-1) antagonists (e.g., rimonabant described in U.S. Pat. No. 5,624,941 (SR-141,716A), purine compounds, such as those described in U.S. Patent Publication No. 2004/0092520; pyrazolo[1,5-a][1,3,5]triazine compounds, such as those described in U.S. Non-Provisional patent application Ser. No. 10/763,105 filed on Jan. 21, 2004; and bicyclic pyrazolyl and imidazolyl compounds, such as those described in U.S. Provisional Application No. 60/518,280 filed on Nov. 7, 2003), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-Y antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine™), human agouti-related proteins (AGRP), ghrelin receptor antagonists, histamine 3 receptor antagonists or inverse agonists, neuromedin U receptor agonists, and the like.

Rimonabant (SR141716A also known under the tradename Acomplia™ available from Sanofi-Synthelabo) can be prepared as described in U.S. Pat. No. 5,624,941. Other suitable CB-1 antagonists include those described in U.S. Pat. Nos. 5,747,524, 6,432,984 and 6,518,264; U.S. Patent Publication Nos. U.S. 2004/0092520, U.S. 2004/0157839, U.S. 2004/0214855, and U.S. 2004/0214838; U.S. patent application Ser. No. 10/971,599 filed on Oct. 22, 2004; and PCT Patent Publication Nos. WO 02/076949, WO 03/075660, WO04/048317, WO04/013120, and WO 04/012671.

Preferred apolipoprotein-B secretion/microsomal triglyceride transfer protein (apo-B/MTP) inhibitors for use as anti-obesity agents are gut-selective MTP inhibitors, such as dirlotapide described in U.S. Pat. No. 6,720,351; 4-(4-(4-(4-((2-((4-methyl-4H-1,2,4-triazol-3-ylthio)methyl)-2-(4-chlorophenyl)-1,3-dioxolan-4-yl)methoxy)phenyl)piperazin-1-yl)phenyl)-2-sec-butyl-2H-1,2,4-triazol-3 (4H)-one (R103757) described in U.S. Pat. Nos. 5,521,186 and 5,929,075; and implitapide (BAY 13-9952) described in U.S. Pat. No. 6,265,431. As used herein, the term “gut-selective” means that the MTP inhibitor has a higher exposure to the gastro-intestinal tissues versus systemic exposure.

Any thyromimetic can be used as the second agent in combination with a compound of the present invention. Such thyromimetic activity is readily determined by those skilled in the art according to standard assays (e.g., Atherosclerosis (1996) 126: 53-63). A variety of thyromimetic agents are known to those skilled in the art, for example those disclosed in U.S. Pat. Nos. 4,766,121; 4,826,876; 4,910,305; 5,061,798; 5,284,971; 5,401,772; 5,654,468; and 5,569,674. Other antiobesity agents include sibutramine which can be prepared as described in U.S. Pat. No. 4,929,629 and bromocriptine which can be prepared as described in U.S. Pat. Nos. 3,752,814 and 3,752,888.

The compounds of the present invention can also be used in combination with other antihypertensive agents. Any anti-hypertensive agent can be used as the second agent in such combinations and examples are provided herein. Such antihypertensive activity is readily determined by those skilled in the art according to standard assays (e.g., blood pressure measurements).

Amlodipine and related dihydropyridine compounds are disclosed in U.S. Pat. No. 4,572,909, which is incorporated herein by reference, as potent anti-ischemic and antihypertensive agents. U.S. Pat. No. 4,879,303, which is incorporated herein by reference, discloses amlodipine benzenesulfonate salt (also termed amlodipine besylate). Amlodipine and amlodipine besylate are potent and long lasting calcium channel blockers. As such, amlodipine, amlodipine besylate, amlodipine maleate and other pharmaceutically acceptable acid addition salts of amlodipine have utility as antihypertensive agents and as antiischemic agents. Amlodipine besylate is currently sold as Norvasc®. Amlodipine has the formula

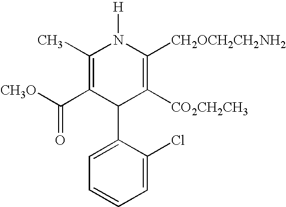

Calcium channel blockers which are within the scope of this invention include, but are not limited to: bepridil, which may be prepared as disclosed in U.S. Pat. No. 3,962,238 or U.S. Reissue No. 30,577; clentiazem, which may be prepared as disclosed in U.S. Pat. No. 4,567,175; diltiazem, which may be prepared as disclosed in U.S. Pat. No. 3,562, fendiline, which may be prepared as disclosed in U.S. Pat. No. 3,262,977; gallopamil, which may be prepared as disclosed in U.S. Pat. No. 3,261,859; mibefradil, which may be prepared as disclosed in U.S. Pat. No. 4,808,605; prenylamine, which may be prepared as disclosed in U.S. Pat. No. 3,152,173; semotiadil, which may be prepared as disclosed in U.S. Pat. No. 4,786,635; terodiline, which may be prepared as disclosed in U.S. Pat. No. 3,371,014; verapamil, which may be prepared as disclosed in U.S. Pat. No. 3,261,859; aranipine, which may be prepared as disclosed in U.S. Pat. No. 4,572,909; barnidipine, which may be prepared as disclosed in U.S. Pat. No. 4,220,649; benidipine, which may be prepared as disclosed in European Patent Application Publication No. 106,275; cilnidipine, which may be prepared as disclosed in U.S. Pat. No. 4,672,068; efonidipine, which may be prepared as disclosed in U.S. Pat. No. 4,885,284; elgodipine, which may be prepared as disclosed in U.S. Pat. No. 4,952,592; felodipine, which may be prepared as disclosed in U.S. Pat. No. 4,264,611; isradipine, which may be prepared as disclosed in U.S. Pat. No. 4,466,972; lacidipine, which may be prepared as disclosed in U.S. Pat. No. 4,801,599; lercanidipine, which may be prepared as disclosed in U.S. Pat. No. 4,705,797; manidipine, which may be prepared as disclosed in U.S. Pat. No. 4,892,875; nicardipine, which may be prepared as disclosed in U.S. Pat. No. 3,985,758; nifedipine, which may be prepared as disclosed in U.S. Pat. No. 3,485,847; nilvadipine, which may be prepared as disclosed in U.S. Pat. No. 4,338,322; nimodipine, which may be prepared as disclosed in U.S. Pat. No. 3,799,934; nisoldipine, which may be prepared as disclosed in U.S. Pat. No. 4,154,839; nitrendipine, which may be prepared as disclosed in U.S. Pat. No. 3,799,934; cinnarizine, which may be prepared as disclosed in U.S. Pat. No. 2,882,271; flunarizine, which may be prepared as disclosed in U.S. Pat. No. 3,773,939; lidoflazine, which may be prepared as disclosed in U.S. Pat. No. 3,267,104; lomerizine, which may be prepared as disclosed in U.S. Pat. No. 4,663,325; bencyclane, which may be prepared as disclosed in Hungarian Patent No. 151,865; etafenone, which may be prepared as disclosed in German Patent No. 1,265,758; and perhexiline, which may be prepared as disclosed in British Patent No. 1,025,578. The disclosures of all such U.S. patents are incorporated herein by reference. Examples of presently marketed products containing antihypertensive agents include calcium channel blockers, such as Cardizem®, Adalat®, Calan®, Cardene®, Covera®, Dilacor®, DynaCirc® Procardia XL®, Sular®, Tiazac®, Vascor®, Verelan®, Isoptin®, Nimotop® Norvasc®, and Plendil®; angiotensin converting enzyme (ACE) inhibitors, such as Accupril®, Altace®, Captopril®, Lotensin®, Mavik®, Monopril®, Prinivil®, Univasc®, Vasotec® and Zestril®.

Angiotensin Converting Enzyme Inhibitors (ACE-Inhibitors) which are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, which may be prepared as disclosed in U.S. Pat. Nos. 4,046,889 and 4,105,776; ceronapril, which may be prepared as disclosed in U.S. Pat. No. 4,452,790; delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727; lisinopril, which may be prepared as disclosed in U.S. Pat. No. 4,555,502; moveltopril, which may be prepared as disclosed in Belgian Patent No. 893,553; perindopril, which may be prepared as disclosed in U.S. Pat. No. 4,508,729; quinapril, which may be prepared as disclosed in U.S. Pat. No. 4,344,949; ramipril, which may be prepared as disclosed in U.S. Pat. No. 4,587,258; spirapril, which may be prepared as disclosed in U.S. Pat. No. 4,470,972; temocapril, which may be prepared as disclosed in U.S. Pat. No. 4,699,905; and trandolapril, which may be prepared as disclosed in U.S. Pat. No. 4,933,361. The disclosures of all such U.S. patents are incorporated herein by reference.

Angiotensin-II receptor antagonists (A-II antagonists) which are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317; losartan, which may be prepared as disclosed in U.S. Pat. No. 5,138,069; and valsartan, which may be prepared as disclosed in U.S. Pat. No. 5,399,578. The disclosures of all such U.S. patents are incorporated herein by reference.

Beta-adrenergic receptor blockers (beta- or β-blockers) which are within the scope of this invention include, but are not limited to: acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arotinolol, which may be prepared as disclosed in U.S. Pat. No. 3,932,400; atenolol, which may be prepared as disclosed in U.S. Pat. No. 3,663,607 or 3,836,671; befunolol, which may be prepared as disclosed in U.S. Pat. No. 3,853,923; betaxolol, which may be prepared as disclosed in U.S. Pat. No. 4,252,984; bevantolol, which may be prepared as disclosed in U.S. Pat. No. 3,857,981; bisoprolol, which may be prepared as disclosed in U.S. Pat. No. 4,171,370; bopindolol, which may be prepared as disclosed in U.S. Pat. No. 4,340,541; bucumolol, which may be prepared as disclosed in U.S. Pat. No. 3,663,570; bufetolol, which may be prepared as disclosed in U.S. Pat. No. 3,723,476; bufuralol, which may be prepared as disclosed in U.S. Pat. No. 3,929,836; bunitrolol, which may be prepared as disclosed in U.S. Pat. Nos. 3,940,489 and 3,961,071; buprandolol, which may be prepared as disclosed in U.S. Pat. No. 3,309,406; butiridine hydrochloride, which may be prepared as disclosed in French Patent No. 1,390,056; butofilolol, which may be prepared as disclosed in U.S. Pat. No. 4,252,825; carazolol, which may be prepared as disclosed in German Patent No. 2,240,599; carteolol, which may be prepared as disclosed in U.S. Pat. No. 3,910,924; carvedilol, which may be prepared as disclosed in U.S. Pat. No. 4,503,067; celiprolol, which may be prepared as disclosed in U.S. Pat. No. 4,034,009; cetamolol, which may be prepared as disclosed in U.S. Pat. No. 4,059,622; cloranolol, which may be prepared as disclosed in German Patent No. 2,213,044; dilevalol, which may be prepared as disclosed in Clifton et al., Journal of Medicinal Chemistry, 1982, 25, 670; epanolol, which may be prepared as disclosed in European Patent Publication Application No. 41,491; indenolol, which may be prepared as disclosed in U.S. Pat. No. 4,045,482; labetalol, which may be prepared as disclosed in U.S. Pat. No. 4,012,444; levobunolol, which may be prepared as disclosed in U.S. Pat. No. 4,463,176; mepindolol, which may be prepared as disclosed in Seeman et al., Helv. Chim. Acta, 1971, 54, 241; metipranolol, which may be prepared as disclosed in Czechoslovakian Patent Application No. 128,471; metoprolol, which may be prepared as disclosed in U.S. Pat. No. 3,873,600; moprolol, which may be prepared as disclosed in U.S. Pat. No. 3,501,7691; nadolol, which may be prepared as disclosed in U.S. Pat. No. 3,935,267; nadoxolol, which may be prepared as disclosed in U.S. Pat. No. 3,819,702; nebivalol, which may be prepared as disclosed in U.S. Pat. No. 4,654,362; nipradilol, which may be prepared as disclosed in U.S. Pat. No. 4,394,382; oxprenolol, which may be prepared as disclosed in British Patent No. 1,077,603; perbutolol, which may be prepared as disclosed in U.S. Pat. No. 3,551,493; pindolol, which may be prepared as disclosed in Swiss Patent Nos. 469,002 and 472,404; practolol, which may be prepared as disclosed in U.S. Pat. No. 3,408,387; pronethalol, which may be prepared as disclosed in British Patent No. 909,357; propranolol, which may be prepared as disclosed in U.S. Pat. Nos. 3,337,628 and 3,520,919; sotalol, which may be prepared as disclosed in Uloth et al., Journal of Medicinal Chemistry, 1966, 9, 88; sufinalol, which may be prepared as disclosed in German Patent No. 2,728,641; talindol, which may be prepared as disclosed in U.S. Pat. Nos. 3,935,259 and 4,038,313; tertatolol, which may be prepared as disclosed in U.S. Pat. No. 3,960,891; tilisolol, which may be prepared as disclosed in U.S. Pat. No. 4,129,565; timolol, which may be prepared as disclosed in U.S. Pat. No. 3,655,663; toliprolol, which may be prepared as disclosed in U.S. Pat. No. 3,432,545; and xibenolol, which may be prepared as disclosed in U.S. Pat. No. 4,018,824. The disclosures of all such U.S. patents are incorporated herein by reference.

Alpha-adrenergic receptor blockers (alpha- or α-blockers) which are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol, which may be prepared as disclosed above; naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,511,836; tamsulosin, which may be prepared as disclosed in U.S. Pat. No. 4,703,063; tolazoline, which may be prepared as disclosed in U.S. Pat. No. 2,161,938; trimazosin, which may be prepared as disclosed in U.S. Pat. No. 3,669,968; and yohimbine, which may be isolated from natural sources according to methods well known to those skilled in the art. The disclosures of all such U.S. patents are incorporated herein by reference.

The term “vasodilator,” where used herein, is meant to include cerebral vasodilators, coronary vasodilators and peripheral vasodilators. Cerebral vasodilators within the scope of this invention include, but are not limited to: bencyclane, which may be prepared as disclosed above; cinnarizine, which may be prepared as disclosed above; citicoline, which may be isolated from natural sources as disclosed in Kennedy et al., Journal of the American Chemical Society, 1955, 77, 250 or synthesized as disclosed in Kennedy, Journal of Biological Chemistry, 1956, 222, 185; cyclandelate, which may be prepared as disclosed in U.S. Pat. No. 3,663,597; ciclonicate, which may be prepared as disclosed in German Patent No. 1,910,481; diisopropylamine dichloroacetate, which may be prepared as disclosed in British Patent No. 862,248; eburnamonine, which may be prepared as disclosed in Hermann et al., Journal of the American Chemical Society, 1979, 101, 1540; fasudil, which may be prepared as disclosed in U.S. Pat. No. 4,678,783; fenoxedil, which may be prepared as disclosed in U.S. Pat. No. 3,818,021; flunarizine, which may be prepared as disclosed in U.S. Pat. No. 3,773,939; ibudilast, which may be prepared as disclosed in U.S. Pat. No. 3,850,941; ifenprodil, which may be prepared as disclosed in U.S. Pat. No. 3,509,164; lomerizine, which may be prepared as disclosed in U.S. Pat. No. 4,663,325; nafronyl, which may be prepared as disclosed in U.S. Pat. No. 3,334,096; nicametate, which may be prepared as disclosed in Blicke et al., Journal of the American Chemical Society, 1942, 64, 1722; nicergoline, which may be prepared as disclosed above; nimodipine, which may be prepared as disclosed in U.S. Pat. No. 3,799,934; papaverine, which may be prepared as reviewed in Goldberg, Chem. Prod. Chem. News, 1954, 17, 371; pentifylline, which may be prepared as disclosed in German Patent No. 860,217; tinofedrine, which may be prepared as disclosed in U.S. Pat. No. 3,563,997; vincamine, which may be prepared as disclosed in U.S. Pat. No. 3,770,724; vinpocetine, which may be prepared as disclosed in U.S. Pat. No. 4,035,750; and viquidil, which may be prepared as disclosed in U.S. Pat. No. 2,500,444. The disclosures of all such U.S. patents are incorporated herein by reference.

Coronary vasodilators within the scope of this invention include, but are not limited to: amotriphene, which may be prepared as disclosed in U.S. Pat. No. 3,010,965; bendazol, which may be prepared as disclosed in J. Chem. Soc. 1958, 2426; benfurodil hemisuccinate, which may be prepared as disclosed in U.S. Pat. No. 3,355,463; benziodarone, which may be prepared as disclosed in U.S. Pat. No. 3,012,042; chloracizine, which may be prepared as disclosed in British Patent No. 740,932; chromonar, which may be prepared as disclosed in U.S. Pat. No. 3,282,938; clobenfural, which may be prepared as disclosed in British Patent No. 1,160,925; clonitrate, which may be prepared from propanediol according to methods well known to those skilled in the art, e.g., see Annalen, 1870, 155, 165; cloricromen, which may be prepared as disclosed in U.S. Pat. No. 4,452,811; dilazep, which may be prepared as disclosed in U.S. Pat. No. 3,532,685; dipyridamole, which may be prepared as disclosed in British Patent No. 807,826; droprenilamine, which may be prepared as disclosed in German Patent No. 2,521,113; efloxate, which may be prepared as disclosed in British Patent Nos. 803,372 and 824,547; erythrityl tetranitrate, which may be prepared by nitration of erythritol according to methods well-known to those skilled in the art; etafenone, which may be prepared as disclosed in German Patent No. 1,265,758; fendiline, which may be prepared as disclosed in U.S. Pat. No. 3,262,977; floredil, which may be prepared as disclosed in German Patent No. 2,020,464; ganglefene, which may be prepared as disclosed in U.S. R. Pat. No. 115,905; hexestrol, which may be prepared as disclosed in U.S. Pat. No. 2,357,985; hexobendine, which may be prepared as disclosed in U.S. Pat. No. 3,267,103; itramin tosylate, which may be prepared as disclosed in Swedish Patent No. 168,308; khellin, which may be prepared as disclosed in Baxter et al., Journal of the Chemical Society, 1949, S 30; lidoflazine, which may be prepared as disclosed in U.S. Pat. No. 3,267,104; mannitol hexanitrate, which may be prepared by the nitration of mannitol according to methods well-known to those skilled in the art; medibazine, which may be prepared as disclosed in U.S. Pat. No. 3,119,826; nitroglycerin; pentaerythritol tetranitrate, which may be prepared by the nitration of pentaerythritol according to methods well-known to those skilled in the art; pentrinitrol, which may be prepared as disclosed in German Patent No. 638,422-3; perhexilline, which may be prepared as disclosed above; pimethylline, which may be prepared as disclosed in U.S. Pat. No. 3,350,400; prenylamine, which may be prepared as disclosed in U.S. Pat. No. 3,152,173; propatyl nitrate, which may be prepared as disclosed in French Patent No. 1,103,113; trapidil, which may be prepared as disclosed in East German Patent No. 55,956; tricromyl, which may be prepared as disclosed in U.S. Pat. No. 2,769,015; trimetazidine, which may be prepared as disclosed in U.S. Pat. No. 3,262,852; troInitrate phosphate, which may be prepared by nitration of triethanolamine followed by precipitation with phosphoric acid according to methods well-known to those skilled in the art; visnadine, which may be prepared as disclosed in U.S. Pat. Nos. 2,816,118 and 2,980,699. The disclosures of all such U.S. patents are incorporated herein by reference.

Peripheral vasodilators within the scope of this invention include, but are not limited to: aluminum nicotinate, which may be prepared as disclosed in U.S. Pat. No. 2,970,082; bamethan, which may be prepared as disclosed in Corrigan et al., Journal of the American Chemical Society, 1945, 67, 1894; bencyclane, which may be prepared as disclosed above; betahistine, which may be prepared as disclosed in Walter et al.; Journal of the American Chemical Society, 1941, 63, 2771; bradykinin, which may be prepared as disclosed in Hamburg et al., Arch. Biochem. Biophys., 1958, 76, 252; brovincamine, which may be prepared as disclosed in U.S. Pat. No. 4,146,643; bufeniode, which may be prepared as disclosed in U.S. Pat. No. 3,542,870; buflomedil, which may be prepared as disclosed in U.S. Pat. No. 3,895,030; butalamine, which may be prepared as disclosed in U.S. Pat. No. 3,338,899; cetiedil, which may be prepared as disclosed in French Patent Nos. 1,460,571; ciclonicate, which may be prepared as disclosed in German Patent No. 1,910,481; cinepazide, which may be prepared as disclosed in Belgian Patent No. 730,345; cinnarizine, which may be prepared as disclosed above; cyclandelate, which may be prepared as disclosed above; diisopropylamine dichloroacetate, which may be prepared as disclosed above; eledoisin, which may be prepared as disclosed in British Patent No. 984,810; fenoxedil, which may be prepared as disclosed above; flunarizine, which may be prepared as disclosed above; hepronicate, which may be prepared as disclosed in U.S. Pat. No. 3,384,642; ifenprodil, which may be prepared as disclosed above; iloprost, which may be prepared as disclosed in U.S. Pat. No. 4,692,464; inositol niacinate, which may be prepared as disclosed in Badgett et al., Journal of the American Chemical Society, 1947, 69, 2907; isoxsuprine, which may be prepared as disclosed in U.S. Pat. No. 3,056,836; kallidin, which may be prepared as disclosed in Biochem. Biophys. Res. Commun., 1961, 6, 210; kallikrein, which may be prepared as disclosed in German Patent No. 1,102,973; moxisylyte, which may be prepared as disclosed in German Patent No. 905,738; nafronyl, which may be prepared as disclosed above; nicametate, which may be prepared as disclosed above; nicergoline, which may be prepared as disclosed above; nicofuranose, which may be prepared as disclosed in Swiss Patent No. 366,523; nylidrin, which may be prepared as disclosed in U.S. Pat. Nos. 2,661,372 and 2,661,373; pentifylline, which may be prepared as disclosed above; pentoxifylline, which may be prepared as disclosed in U.S. Pat. No. 3,422,107; piribedil, which may be prepared as disclosed in U.S. Pat. No. 3,299,067; prostaglandin E1, which may be prepared by any of the methods referenced in the Merck Index, Twelfth Edition, Budaveri, Ed., New Jersey, 1996, p. 1353; suloctidil, which may be prepared as disclosed in German Patent No. 2,334,404; tolazoline, which may be prepared as disclosed in U.S. Pat. No. 2,161,938; and xanthinol niacinate, which may be prepared as disclosed in German Patent No. 1,102,750 or Korbonits et al., Acta. Pharm. Hung., 1968, 38, 98. The disclosures of all such U.S. patents are incorporated herein by reference.

The term “diuretic,” within the scope of this invention, is meant to include diuretic benzothiadiazine derivatives, diuretic organomercurials, diuretic purines, diuretic steroids, diuretic sulfonamide derivatives, diuretic uracils and other diuretics such as amanozine, which may be prepared as disclosed in Austrian Patent No. 168,063; amiloride, which may be prepared as disclosed in Belgian Patent No. 639,386; arbutin, which may be prepared as disclosed in Tschitschibabin, Annalen, 1930, 479, 303; chlorazanil, which may be prepared as disclosed in Austrian Patent No. 168,063; ethacrynic acid, which may be prepared as disclosed in U.S. Pat. No. 3,255,241; etozolin, which may be prepared as disclosed in U.S. Pat. No. 3,072,653; hydracarbazine, which may be prepared as disclosed in British Patent No. 856,409; isosorbide, which may be prepared as disclosed in U.S. Pat. No. 3,160,641; mannitol; metochalcone, which may be prepared as disclosed in Freudenberg et al., Ber., 1957, 90, 957; muzolimine, which may be prepared as disclosed in U.S. Pat. No. 4,018,890; perhexiline, which may be prepared as disclosed above; ticrynafen, which may be prepared as disclosed in U.S. Pat. No. 3,758,506; triamterene which may be prepared as disclosed in U.S. Pat. No. 3,081,230; and urea. The disclosures of all such U.S. patents are incorporated herein by reference.

Diuretic benzothiadiazine derivatives within the scope of this invention include, but are not limited to: althiazide, which may be prepared as disclosed in British Patent No. 902,658; bendroflumethiazide, which may be prepared as disclosed in U.S. Pat. No. 3,265,573; benzthiazide, McManus et al., 136th Am. Soc. Meeting (Atlantic City, September 1959), Abstract of papers, pp 13-O; benzylhydrochlorothiazide, which may be prepared as disclosed in U.S. Pat. No. 3,108,097; buthiazide, which may be prepared as disclosed in British Patent Nos. 861,367 and 885,078; chlorothiazide, which may be prepared as disclosed in U.S. Pat. Nos. 2,809,194 and 2,937,169; chlorthalidone, which may be prepared as disclosed in U.S. Pat. No. 3,055,904; cyclopenthiazide, which may be prepared as disclosed in Belgian Patent No. 587,225; cyclothiazide, which may be prepared as disclosed in Whitehead et al., Journal of Organic Chemistry, 1961, 26, 2814; epithiazide, which may be prepared as disclosed in U.S. Pat. No. 3,009,911; ethiazide, which may be prepared as disclosed in British Patent No. 861,367; fenquizone, which may be prepared as disclosed in U.S. Pat. No. 3,870,720; indapamide, which may be prepared as disclosed in U.S. Pat. No. 3,565,911; hydrochlorothiazide, which may be prepared as disclosed in U.S. Pat. No. 3,164,588; hydroflumethiazide, which may be prepared as disclosed in U.S. Pat. No. 3,254,076; methyclothiazide, which may be prepared as disclosed in Close et al., Journal of the American Chemical Society, 1960, 82, 1132; meticrane, which may be prepared as disclosed in French Patent Nos. M2790 and 1,365,504; metolazone, which may be prepared as disclosed in U.S. Pat. No. 3,360,518; paraflutizide, which may be prepared as disclosed in Belgian Patent No. 620,829; polythiazide, which may be prepared as disclosed in U.S. Pat. No. 3,009,911; quinethazone, which may be prepared as disclosed in U.S. Pat. No. 2,976,289; teclothiazide, which may be prepared as disclosed in Close et al., Journal of the American Chemical Society, 1960, 82, 1132; and trichlormethiazide, which may be prepared as dislcosed in deStevens et al., Experientia, 1960, 16, 113. The disclosures of all such U.S. patents are incorporated herein by reference.

Diuretic sulfonamide derivatives within the scope of this invention include, but are not limited to: acetazolamide, which may be prepared as disclosed in U.S. Pat. No. 2,980,679; ambuside, which may be prepared as disclosed in U.S. Pat. No. 3,188,329; azosemide, which may be prepared as disclosed in U.S. Pat. No. 3,665,002; bumetanide, which may be prepared as disclosed in U.S. Pat. No. 3,634,583; butazolamide, which may be prepared as disclosed in British Patent No. 769,757; chloraminophenamide, which may be prepared as disclosed in U.S. Pat. Nos. 2,809,194, 2,965,655 and 2,965,656; clofenamide, which may be prepared as disclosed in Olivier, Rec. Trav. Chim., 1918, 37, 307; clopamide, which may be prepared as disclosed in U.S. Pat. No. 3,459,756; clorexolone, which may be prepared as disclosed in U.S. Pat. No. 3,183,243; disulfamide, which may be prepared as disclosed in British Patent No. 851,287; ethoxolamide, which may be prepared as disclosed in British Patent No. 795,174; furosemide, which may be prepared as disclosed in U.S. Pat. No. 3,058,882; mefruside, which may be prepared as disclosed in U.S. Pat. No. 3,356,692; methazolamide, which may be prepared as disclosed in U.S. Pat. No. 2,783,241; piretanide, which may be prepared as disclosed in U.S. Pat. No. 4,010,273; torasemide, which may be prepared as disclosed in U.S. Pat. No. 4,018,929; tripamide, which may be prepared as disclosed in Japanese Patent No. 73 05,585; and xipamide, which may be prepared as disclosed in U.S. Pat. No. 3,567,777. The disclosures of all such U.S. patents are incorporated herein by reference.

Osteoporosis is a systemic skeletal disease, characterized by low bone mass and deterioration of bone tissue, with a consequent increase in bone fragility and susceptibility to fracture. In the U.S., the condition affects more than 25 million people and causes more than 1.3 million fractures each year, including 500,000 spine, 250,000 hip and 240,000 wrist fractures annually. Hip fractures are the most serious consequence of osteoporosis, with 5-20% of patients dying within one year, and over 50% of survivors being incapacitated.

The elderly are at greatest risk of osteoporosis, and the problem is therefore predicted to increase significantly with the aging of the population. Worldwide fracture incidence is forecasted to increase three-fold over the next 60 years, and one study has estimated that there will be 4.5 million hip fractures worldwide in 2050.

Women are at greater risk of osteoporosis than men. Women experience a sharp acceleration of bone loss during the five years following menopause. Other factors that increase the risk include smoking, alcohol abuse, a sedentary lifestyle and low calcium intake.

Those skilled in the art will recognize that anti-resorptive agents (for example progestins, polyphosphonates, bisphosphonate(s), estrogen agonists/antagonists, estrogen, estrogen/progestin combinations, Premarin®, estrone, estriol or 17α- or 17β-ethynyl estradiol) may be used in conjunction with the compounds of the present invention.

Exemplary progestins are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, flurogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol.

Preferred progestins are medroxyprogestrone, norethindrone and norethynodrel.

Exemplary bone resorption inhibiting polyphosphonates include polyphosphonates of the type disclosed in U.S. Pat. No. 3,683,080, the disclosure of which is incorporated herein by reference. Preferred polyphosphonates are geminal diphosphonates (also referred to as bis-phosphonates). Tiludronate disodium is an especially preferred polyphosphonate. Ibandronic acid is an especially preferred polyphosphonate. Alendronate and resindronate are especially preferred polyphosphonates. Zoledronic acid is an especially preferred polyphosphonate. Other preferred polyphosphonates are 6-amino-1-hydroxy-hexylidene-bisphosphonic acid and 1-hydroxy-3(methylpentylamino)-propylidene-bisphosphonic acid. The polyphosphonates may be administered in the form of the acid, or of a soluble alkali metal salt or alkaline earth metal salt. Hydrolyzable esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid, N,N-dimethylamino methane diphosphonic acid, N(2-hydroxyethyl) amino methane diphosphonic acid, butane-4-amino-1-hydroxy-1,1-diphosphonic acid, pentane-5-amino-1-hydroxy-1,1-diphosphonic acid, hexane-6-amino-1-hydroxy-1,1-diphosphonic acid and pharmaceutically acceptable esters and salts thereof.

In particular, the compounds of this invention may be combined with a mammalian estrogen agonist/antagonist. Any estrogen agonist/antagonist may be used in the combination aspect of this invention. The term estrogen agonist/antagonist refers to compounds which bind with the estrogen receptor, inhibit bone turnover and/or prevent bone loss. In particular, estrogen agonists are herein defined as chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue, and blocking the actions of estrogen in one or more tissues. Such activities are readily determined by those skilled in the art of standard assays including estrogen receptor binding assays, standard bone histomorphometric and densitometer methods, and Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below.

Another preferred estrogen agonist/antagonist is 3-(4-(1,2-diphenyl-but-1-enyl)-phenyl)-acrylic acid, which is disclosed in Willson et al., Endocrinology, 1997, 138, 3901-3911.

Another preferred estrogen agonist/antagonist is tamoxifen: (ethanamine, 2-(−4-(1,2-diphenyl-1-butenyl)phenoxy)-N,N-dimethyl, (Z)-2-, 2-hydroxy-1,2,3-propanetricarboxylate (1:1)) and related compounds which are disclosed in U.S. Pat. No. 4,536,516, the disclosure of which is incorporated herein by reference.

Another related compound is 4-hydroxy tamoxifen, which is disclosed in U.S. Pat. No. 4,623,660, the disclosure of which is incorporated herein by reference.

A preferred estrogen agonist/antagonist is raloxifene: (methanone, (6-hydroxy-2-(4-hydroxyphenyl)benzo[b]thien-3-yl)(4-(2-(1-piperidinyl)ethoxy)phenyl)-hydrochloride) which is disclosed in U.S. Pat. No. 4,418,068, the disclosure of which is incorporated herein by reference.

Another preferred estrogen agonist/antagonist is toremifene: (ethanamine, 2-(4-(4-chloro-1,2-diphenyl-1-butenyl)phenoxy)-N,N-dimethyl-, (Z)-, 2-hydroxy-1,2,3-propanetricarboxylate (1:1) which is disclosed in U.S. Pat. No. 4,996,225, the disclosure of which is incorporated herein by reference.

Another preferred estrogen agonist/antagonist is centchroman: 1-(2-((4-(-methoxy-2,2, dimethyl-3-phenyl-chroman-4-yl)-phenoxy)-ethyl)-pyrrolidine, which is disclosed in U.S. Pat. No. 3,822,287, the disclosure of which is incorporated herein by reference. Also preferred is levormeloxifene.

Another preferred estrogen agonist/antagonist is idoxifene: (E)-1-(2-(4-(1-(4-iodo-phenyl)-2-phenyl-but-1-enyl)-phenoxy)-ethyl)-pyrrolidinone, which is disclosed in U.S. Pat. No. 4,839,155, the disclosure of which is incorporated herein by reference.

Another preferred estrogen agonist/antagonist is 2-(4-methoxy-phenyl)-3-[4-(2-piperidin-1-yl-ethoxy)-phenoxy]-benzo[b]thiophen-6-ol which is disclosed in U.S. Pat. No. 5,488,058, the disclosure of which is incorporated herein by reference.

Another preferred estrogen agonist/antagonist is 6-(4-hydroxy-phenyl)-5-(4-(2-piperidin-1-yl-ethoxy)-benzyl)-naphthalen-2-ol, which is disclosed in U.S. Pat. No. 5,484,795, the disclosure of which is incorporated herein by reference.

Another preferred estrogen agonist/antagonist is (4-(2-(2-aza-bicyclo[2.2.1]hept-2-yl)-ethoxy)-phenyl)-(6-hydroxy-2-(4-hydroxy-phenyl)-benzo[b]thiophen-3-yl)-methanone which is disclosed, along with methods of preparation, in PCT publication no. WO 95/10513 assigned to Pfizer Inc.

Other preferred estrogen agonist/antagonists include the compounds, TSE-424 (Wyeth-Ayerst Laboratories) and arazoxifene.

Other preferred estrogen agonist/antagonists include compounds as described in commonly assigned U.S. Pat. No. 5,552,412, the disclosure of which is incorporated herein by reference. Especially preferred compounds described therein are:
cis-6-(4-fluoro-phenyl)-5-(4-(2-piperidin-1-yl-ethoxy)-phenyl)-5,6,7,8-tetrahydro-naphthalene-2-ol;
(−)-cis-6-phenyl-5-(4-(2-pyrrolidin-1-yl-ethoxy)-phenyl)-5,6,7,8-tetrahydro-naphthalene-2-ol (also known as lasofoxifene);
cis-6-phenyl-5-(4-(2-pyrrolidin-1-yl-ethoxy)-phenyl)-5,6,7,8-tetrahydro-naphthalene-2-ol;
cis-1-(6′-pyrrolodinoethoxy-3′-pyridyl)-2-phenyl-6-hydroxy-1,2,3,4-tetrahydronaphthalene;
1-(4′-pyrrolidinoethoxyphenyl)-2-(4″-fluorophenyl)-6-hydroxy-1,2,3,4-tetrahydroisoquinoline;
cis-6-(4-hydroxyphenyl)-5-(4-(2-piperidin-1-yl-ethoxy)-phenyl)-5,6,7,8-tetrahydro-naphthalene-2-ol; and
1-(4′-pyrrolidinolethoxyphenyl)-2-phenyl-6-hydroxy-1,2,3,4-tetrahydroisoquinoline.

Other estrogen agonist/antagonists are described in U.S. Pat. No. 4,133,814 (the disclosure of which is incorporated herein by reference). U.S. Pat. No. 4,133,814 discloses derivatives of 2-phenyl-3-aroyl-benzothiophene and 2-phenyl-3-aroylbenzothiophene-1-oxide.

Other anti-osteoporosis agents, which can be used as the second agent in combination with a compound of the present invention, include, for example, the following: parathyroid hormone (PTH) (a bone anabolic agent); parathyroid hormone (PTH) secretagogues (see, e.g., U.S. Pat. No. 6,132,774), particularly calcium receptor antagonists; calcitonin; and vitamin D and vitamin D analogs.

Any selective androgen receptor modulator (SARM) can be used in combination with a compound of the present invention. A selective androgen receptor modulator (SARM) is a compound that possesses androgenic activity and which exerts tissue-selective effects. SARM compounds can function as androgen receptor agonists, partial agonists, partial antagonists or antagonists. Examples of suitable SARMs include compounds such as cyproterone acetate, chlormadinone, flutamide, hydroxyflutamide, bicalutamide, nilutamide, spironolactone, 4-(trifluoromethyl)-2 (1H)-pyrrolidino[3,2-g]quinoline derivatives, 1,2-dihydropyridino[5,6-g]quinoline derivatives and piperidino[3,2-g]quinolinone derivatives.

Cypterone, also known as (1b,2b)-6-chloro-1,2-dihydro-17-hydroxy-3′H-cyclopropa[1,2]pregna-1,4,6-triene-3,20-dione is disclosed in U.S. Pat. No. 3,234,093. Chlormadinone, also known as 17-(acetyloxy)-6-chloropregna-4,6-diene-3,20-dione, in its acetate form, acts as an anti-androgen and is disclosed in U.S. Pat. No. 3,485,852. Nilutamide, also known as 5,5-dimethyl-3-[4-nito-3-(trifluoromethyl)phenyl]-2,4-imidazolidinedione and by the trade name Nilandron® is disclosed in U.S. Pat. No. 4,097,578. Flutamide, also known as 2-methyl-N-[4-nitro-3-(trifluoromethyl)phenyl]propanamide and the trade name Eulexin® is disclosed in U.S. Pat. No. 3,847,988. Bicalutamide, also known as 4′-cyano-a′,a′,a′-trifluoro-3-(4-fluorophenylsulfonyl)-2-hydroxy-2-methylpropiono-m-toluidide and the trade name Casodex® is disclosed in EP-100172. The enantiomers of biclutamide are discussed by Tucker and Chesterton, J. Med. Chem. 1988, 31, 885-887. Hydroxyflutamide, a known androgen receptor antagonist in most tissues, has been suggested to function as a SARM for effects on IL-6 production by osteoblasts as disclosed in Hofbauer et al. J. Bone Miner. Res. 1999, 14, 1330-1337. Additional SARMs have been disclosed in U.S. Pat. No. 6,017,924; WO 01/16108, WO 01/16133, WO 01/16139, WO 02/00617, WO 02/16310, U.S. Patent Application Publication No. U.S. 2002/0099096, U.S. Patent Application Publication No. U.S. 2003/0022868, WO 03/011302 and WO 03/011824. All of the above refences are hereby incorporated by reference herein.

The starting materials and reagents for the above-described compounds of the present invention and combination agents, are also readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

Some of the compounds of the present invention or intermediates in their synthesis have asymmetric carbon atoms and therefore are enantiomers or diastereomers. Diasteromeric mixtures can be separated into their individual diastereomers on the basis of their physical chemical differences by methods known per se., for example, by chromatography and/or fractional crystallization. Enantiomers can be separated by, for example, chiral HPLC methods or converting the enantiomeric mixture into a diasteromeric mixture by reaction with an appropriate optically active compound (e.g., alcohol), separating the diastereomers and converting (e.g., hydrolyzing) the individual diastereomers to the corresponding pure enantiomers. Also, an enantiomeric mixture of the compounds or an intermediate in their synthesis which contain an acidic or basic moiety may be separated into their compounding pure enantiomers by forming a diastereomeric salt with an optically pure chiral base or acid (e.g., 1-phenyl-ethyl amine or tartaric acid) and separating the diasteromers by fractional crystallization followed by neutralization to break the salt, thus providing the corresponding pure enantiomers. All such isomers, including diastereomers, enantiomers and mixtures thereof are considered as part of the present invention. Also, some of the compounds of the present invention are atropisomers (e.g., substituted biaryls) and are considered as part of the present invention.

More specifically, the compounds of the present invention can be obtained by fractional crystallization of the basic intermediate with an optically pure chiral acid to form a diastereomeric salt. Neutralization techniques are used to remove the salt and provide the enantiomerically pure compounds. Alternatively, the compounds of the present invention may be obtained in enantiomerically enriched form by resolving the racemate of the final compound or an intermediate in its synthesis (preferably the final compound) employing chromatography (preferably high pressure liquid chromatography [HPLC]) on an asymmetric resin (preferably Chiralcel™ AD or OD (obtained from Chiral Technologies, Exton, Pa.)) with a mobile phase consisting of a hydrocarbon (preferably heptane or hexane) containing between 0 and 50% isopropanol (preferably between 2 and 20%) and between 0 and 5% of an alkyl amine (preferably 0.1% of diethylamine). Concentration of the product containing fractions affords the desired materials.

Some of the compounds of the present invention are acidic and they form a salt with a pharmaceutically acceptable cation. Some of the compounds of the present invention are basic and they form a salt with a pharmaceutically acceptable anion. All such salts are within the scope of the present invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds can be obtained in crystalline form by dissolution in an appropriate solvent(s) such as ethanol, hexanes or water/ethanol mixtures.

The compounds of the present invention, their prodrugs and the salts of such compounds and prodrugs are all adapted to therapeutic use as agents that activate peroxisome proliferator activator receptor (PPAR) activity in mammals, particularly humans. Thus, it is believed the compounds of the present invention, by activating the PPAR receptor, stimulate transcription of key genes involved in fatty acid oxidation and also those involved in high density lipoprotein (HDL) assembly (for example apolipoprotein AI gene transcription), accordingly reducing whole body fat and increasing HDL cholesterol. By virtue of their activity, these agents also reduce plasma levels of triglycerides, VLDL cholesterol, LDL cholesterol and their associated components in mammals, particularly humans, as well as increasing HDL cholesterol and apolipoprotein AI. Hence, these compounds are useful for the treatment and correction of the various dyslipidemias observed to be associated with the development and incidence of atherosclerosis and cardiovascular disease, including hypoalphalipoproteinemia and hypertriglyceridemia.

The present compounds are also useful for modulation of plasma and or serum or tissue lipids or lipoproteins, such as HDL subtypes (e.g., increase, including pre-beta HDL, HDL-1, -2 and 3 particles) as measured by precipitation or by apo-protein content, size, density, NMR profile, FPLC and charge and particle number and its constituents; and LDL subtypes (including LDL subtypes e.g., decreasing small dense LDL, oxidized LDL, VLDL, apo(a) and Lp(a)) as measured by precipitation, or by apo-protein content, size density, NMR profile, FPLC and charge; IDL and remnants (decrease); phospholipids (e.g., increase HDL phospholipids); apo-lipoproteins (increase A-I, A-II, A-IV, decrease total and LDL B-100, decrease B-48, modulate C-II, C-III, E, J); paraoxonase (increase, anti-oxidant effects, anti-inflammatory effects); decrease post-prandial (hyper)lipemia; decrease triglycerides, decrease non-HDL; elevate HDL in subjects with low HDL and optimize and increase ratios of HDL to LDL (e.g., greater than 0.25).

Given the positive correlation between triglycerides, LDL cholesterol, and their associated apolipoproteins in blood with the development of cardiovascular, cerebral vascular and peripheral vascular diseases, the compounds of the present invention, their prodrugs and the salts of such compounds and prodrugs, by virtue of their pharmacologic action, are useful for the prevention, arrestment and/or regression of atherosclerosis and its associated disease states. These include cardiovascular disorders (e.g., cerebrovascular disease, coronary artery disease, ventricular dysfunction, cardiac arrhythmia, pulmonary vascular disease, vascular hemostatic disease, cardiac ischemia and myocardial infarction), complications due to cardiovascular disease, and cognitive dysfunction (including, but not limited to, dementia secondary to atherosclerosis, transient cerebral ischemic attacks, neurodegeneration, neuronal deficient, and delayed onset or procession of Alzheimer's disease).

Thus, given the ability of the compounds of the present invention, their prodrugs and the salts of such compounds and prodrugs to reduce plasma triglycerides and total plasma cholesterol, and increase plasma HDL cholesterol, they are of use in the treatment of diabetes, including impaired glucose tolerance, diabetic complications, insulin resistance and metabolic syndrome, as described previously. In addition, the compounds are useful for the treatment of polycystic ovary syndrome. Also, the compounds are useful in the treatment of obesity given the ability of the compounds of this invention, their prodrugs and the salts of such compounds and prodrugs to increase hepatic fatty acid oxidation.

The utility of the compounds of the present invention, their prodrugs and the salts of such compounds and prodrugs as medical agents in the treatment of the above described disease/conditions in mammals (e.g. humans, male or female) is demonstrated by the activity of the compounds of the present invention in one or more of the conventional assays and in vivo assays described below. The in vivo assays (with appropriate modifications within the skill in the art) can be used to determine the activity of other lipid or triglyceride controlling agents as well as the compounds of the present invention. Thus, the protocols described below can also be used to demonstrate the utility of the combinations of the agents (i.e., the compounds of the present invention) described herein. In addition, such assays provide a means whereby the activities of the compounds of the present invention, their prodrugs and the salts of such compounds and prodrugs (or the other agents described herein) can be compared to each other and with the activities of other known compounds. The results of these comparisons are useful for determining dosage levels in mammals, including humans, for the treatment of such diseases. The following protocols can of course be varied by those skilled in the art.

### PPAR FRET Assay

Measurement of coactivator recruitment by a nuclear receptor-ligand association is a method for evaluating the ability of a ligand to produce a functional response through a nuclear receptor. The PPAR FRET (Fluorescence Resonance Energy Transfer) assay measures the ligand-dependent interaction between nuclear receptor and coactivator. GST/PPAR (α, β, and γ) ligand binding domain (LBD) is labeled with a europium-tagged anti-GST antibody, while an SRC-1 (Sterol Receptor Coactivator-1) synthetic peptide containing an amino terminus long chain biotin molecule is labeled with streptavidin-linked allophycocyanin (APC). Binding of ligand to the PPAR LBD causes a conformational change that allows SRC-1 to bind. Upon SRC-1 binding, the donor FRET molecule (europium) comes in close proximity to the acceptor molecule (APC), resulting in fluorescence energy transfer between donor (337 nm excitation and 620 nm emission) and acceptor (620 nm excitation and 665 nm emission). Increases in the ratio of 665 nm emission to 620 nm emission is a measure of the ability of the ligand-PPAR LBD to recruit SRC-1 synthetic peptide and therefore a measure of the ability of a ligand to produce a functional response through the PPAR receptor.

[1] GST/PPAR LBD Expression. The human PPARα LBD (amino acids 235-507) is fused to the carboxy terminus of glutathione S-transferase (GST) in pGEX-6P-1 (Pfizer, Inc.). The GST/PPARα LBD fusion protein is expressed in BL21 [DE3]pLysS cells using a 50 uM IPTG induction at room temperature for about 16 hours (cells induced at an A₆₀₀ of ˜0.6). Fusion protein is purified on glutathione sepharose 4B beads, eluted in 10 mM reduced glutathione, and dialyzed against 1×PBS at 4° C. Fusion protein is quantitated by Bradford assay (M. M. Bradford, Analst. Biochem. 72:248-254; 1976), and stored at −20° C. in 1×PBS containing 40% glycerol and 5 mM dithiothreitol.

[2] FRET Assay. The FRET assay reaction mix consists of 1×FRET buffer (50 mM Tris-Cl pH 8.0, 50 mM KCl, 0.1 mg/ml BSA, 1 mM EDTA, and 2 mM dithiothreitol) containing 20 nM GST/PPARα LBD, 40 nM of SRC-1 peptide (amino acids 676-700, 5′-long chain biotin-CPSSHSSLTERHKILHRLLQEGSPS-NH₂, purchased from American Peptide Co., Sunnyvale, Calif.), 2 nM of europium-conjugated anti-GST antibody (Wallac, Gaithersburg, Md.), 40 nM of streptavidin-conjugated APC (Wallac), and control and test compounds. The final volume is brought to 100 ul with water and transferred to a black 96-well plate (Microfuor B, Dynex (Chantilly, Va.)). The reaction mixes are incubated for 1 hr at 4° C. and fluorescence is read in Victor 2 plate reader (Wallac). Data is presented as a ratio of the emission at 665 nm to the emission at 615 nm.

### Assessment of lipid-Modulating Activity in Mice

[1] Triglyceride lowering. The hypolipidemic treating activity of the compounds of the present invention can be demonstrated by methods based on standard procedures. For example, the in vivo activity of these compounds in decreasing plasma triglyceride levels may be determined in hybrid B6CBAF1/J mice.

Male B6CVAF1/J mice (8-11 week old) are obtained from The Jackson Laboratory and housed 4-5/cage and maintained in a 12 hr light/12 hr dark cycle. Animals have ad lib. access to Purina rodent chow and water. The animals are dosed daily (9 AM) by oral gavage with vehicle (water or 0.5% methyl cellulose 0.05% Tween 80) or with vehicle containing test compound at the desired concentration. Plasma triglycerides levels are determined 24 hours after the administration of the last dose (day 3) from blood collected retro-orbitally with heparinized hematocrit tubes. Triglyceride determinations are performed using a commercially available Triglyceride E kit from Wako (Osaka, Japan).

[2] HDL cholesterol elevation. The activity of the compounds of the present invention for raising the plasma level of high density lipoprotein (HDL) in a mammal can be demonstrated in transgenic mice expressing the human apoAI and CETP transgenes (HuAICETPTg). The transgenic mice for use in this study are described previously in Walsh et al., J. Lipid Res. 1993, 34: 617-623, Agellon et al., J. Biol. Chem. 1991, 266: 10796-10801. Mice expressing the human apoAI and CETP transgenes are obtained by mating transgenic mice expressing the human apoAI transgene (HuAITg) with CETP mice (HuCETPTg).

Male HuAICETPTg mice (8-11 week old) are grouped according to their human apo AI levels and have free access to Purina rodent chow and water. Animals are dosed daily by oral gavage with vehicle (water or 0.5% methylcellulose 0.05% Tween 80) or with vehicle containing test compound at the desired dose for 5 days. HDL-cholesterol and human apoAI are determined initially (day 0) and 90 minutes post dose (day 5) using methods based on standard procedures. Mouse HDL is separated from apoB-containing lipoproteins by dextran sulfate precipitation as described elsewhere (Francone et al., J. Lipid. Res. 1996, 37:1268-1277). Cholesterol is measured enzymatically using a commercially available cholesterol/HP Reagent kit (Boehringer MannHeim, Indianapolis, Ind.) and spectrophotometrically quantitated on a microplate reader. Human apoAI is measured by a sandwich enzyme-linked immunosorbent assay as previously described (Francone et al., J. Lipid. Res. 1996, 37:1268-1277).

### Measurement of Glucose Lowering in the ob/ob Mouse

The hypoglycemic activity of the compounds of the present invention can be determined by the amount of test compound that reduces glucose levels relative to a vehicle without test compound in male ob/ob mice. The test also allows the determination of an approximate minimal effective dose (MED) value for the in vivo reduction of plasma glucose concentration in such mice for such test compounds.

Five to eight week old male C57BL/6J-ob/ob mice (obtained from Jackson Laboratory, Bar Harbor, Me.) are housed five per cage under standard animal care practices. After a one-week acclimation period, the animals are weighed and 25 microliters of blood are collected from the retro-orbital sinus prior to any treatment. The blood sample is immediately diluted 1:5 with saline containing 0.025% sodium heparin, and held on ice for metabolite analysis. Animals are assigned to treatment groups so that each group has a similar mean for plasma glucose concentration. After group assignment, animals are dosed orally each day for four days with the vehicle consisting of either: (1) 0.25% w/v methyl cellulose in water without pH adjustment; or (2) 0.1% Pluronic® P105 Block Copolymer Surfactant (BASF Corporation, Parsippany, N.J.) in 0.1% saline without pH adjustment. On day 5, the animals are weighed again and then dosed orally with a test compound or the vehicle alone. All compounds are administered in vehicle consisting of either: (1) 0.25% w/v methyl cellulose in water; (2) 10% DMSO/0.1% Pluronic® in 0.1% saline without pH adjustment; or 3) neat PEG 400 without pH adjustment. The animals are then bled from the retro-orbital sinus three hours later for determination of blood metabolite levels. The freshly collected samples are centrifuged for two minutes at 10,000×g at room temperature. The supernatant is analyzed for glucose, for example, by the Abbott VP™ (Abbott Laboratories, Diagnostics Division, Irving, Tex.) and VP Super System® Autoanalyzer (Abbott Laboratories, Irving, Tex.), or by the Abbott Spectrum CCX™ (Abbott Laboratories, Irving, Tex.) using the A-Gent™Glucose-UV Test reagent system (Abbott Laboratories, Irving, Tex.) (a modification of the method of Richterich and Dauwalder, Schweizerische Medizinische Wochenschrift, 101: 860 (1971)) (hexokinase method) using a 100 mg/dl standard. Plasma glucose is then calculated by the equation: Plasma glucose (mg/dl)=Sample value×8.14 where 8.14 is the dilution factor, adjusted for plasma hematocrit (assuming the hematocrit is 44%).

The animals dosed with vehicle maintain substantially unchanged hyperglycemic glucose levels (e.g., greater than or equal to 250 mg/dl), animals treated with compounds having hypoglycemic activity at suitable doses have significantly depressed glucose levels. Hypoglycemic activity of the test compounds is determined by statistical analysis (unpaired t-test) of the mean plasma glucose concentration between the test compound group and vehicle-treated group on day 5. The above assay carried out with a range of doses of a test compound allows the determination of an approximate minimal effective dose (MED) value for the in vivo reduction of plasma glucose concentration.

### Measurement of Insulin, Triglyceride, and Cholesterol Levels in the ob/ob Mouse

The compounds of the present invention are readily adapted to clinical use as hyperinsulinemia reversing agents, triglyceride lowering agents and hypocholesterolemic agents. Such activity can be determined by the amount of test compound that reduces insulin, triglycerides or cholesterol levels relative to a control vehicle without test compound in male ob/ob mice.

Since the concentration of cholesterol in blood is closely related to the development of cardiovascular, cerebral vascular or peripheral vascular disorders, the compounds of the present invention, by virtue of their hypocholesterolemic action, prevent, arrest and/or regress atherosclerosis.

Since the concentration of insulin in blood is related to the promotion of vascular cell growth and increased renal sodium retention, (in addition to the other actions, e.g., promotion of glucose utilization) and these functions are known causes of hypertension, the compounds of the present invention, by virtue of their hypoinsulinemic action, prevent, arrest and/or regress hypertension.

Since the concentration of triglycerides in blood contributes to the overall levels of blood lipids, the compounds of the present invention, by virtue of their triglyceride lowering and/or free fatty acid lowering activity prevent, arrest and/or regress hyperlipidemia.

Free fatty acids contribute to the overall level of blood lipids and independently have been negatively correlated with insulin sensitivity in a variety of physiologic and pathologic states.

Five to eight week old male C57BU6J-ob/ob mice (obtained from Jackson Laboratory, Bar Harbor, Me.) are housed five per cage under standard animal care practices and fed standard rodent diet ad libitum. After a one-week acclimation period, the animals are weighed and 25 microliters of blood are collected from the retro-orbital sinus prior to any treatment. The blood sample is immediately diluted 1:5 with saline containing 0.025% sodium heparin, and held on ice for plasma glucose analysis. Animals are assigned to treatment groups so that each group has a similar mean for plasma glucose concentration. The compound to be tested is administered by oral gavage as an about 0.02% to 2.0% solution (weight/volume (w/v)) in either (1) 10% DMSO/0.1% Pluronic® P105 Block Copolymer Surfactant (BASF Corporation, Parsippany, N.J.) in 0.1% saline without pH adjustment or (2) 0.25% w/v methylcellulose in water without pH adjustment. Alternatively, the compound to be tested can be administered by oral gavage dissolved in or in suspension in neat PEG 400. Single daily dosing (s.i.d.) or twice daily dosing (b.i.d.) is maintained for 1 to, for example, 15 days. Control mice receive the 10% DMSO/0.1% Pluronic® P105 in 0.1% saline without pH adjustment or the 0.25% w/v methylcellulose in water without pH adjustment, or the neat PEG 400 without pH adjustment.

Three hours after the last dose is administered, the animals are sacrificed and blood is collected into 0.5 ml serum separator tubes containing 3.6 mg of a 1:1 weight/weight sodium fluoride: potassium oxalate mixture. The freshly collected samples are centrifuged for two minutes at 10,000×g at room temperature, and the serum supernatant is transferred and diluted 1:1 volume/volume with a 1TIU/ml aprotinin solution in 0.1% saline without pH adjustment.

The diluted serum samples are then stored at −80° C. until analysis. The thawed, diluted serum samples are analyzed for insulin, triglycerides, free fatty acids and cholesterol levels. Serum insulin concentration is determined using Equate® RIA INSULIN kits (double antibody method; as specified by the manufacturer) available from Binax, South Portland, Me. The interassay coefficient of variation is ≦10%. Serum triglycerides are determined using the Abbott VP™ and VP Super System® Autoanalyzer (Abbott Laboratories, Irving, Tex.), or the Abbott Spectrum CCX™ (Abbott Laboratories, Irving, Tex.) using the A-Gent™ Triglycerides Test reagent system (Abbott Laboratories, Diagnostics Division, Irving, Tex.) (lipase-coupled enzyme method; a modification of the method of Sampson, et al., Clinical Chemistry 21: 1983 (1975)). Serum total cholesterol levels are determined using the Abbott VP™ and VP Super System® Autoanalyzer (Abbott Laboratories, Irving, Tex.), and A-Gent™ Cholesterol Test reagent system (cholesterol esterase-coupled enzyme method; a modification of the method of Allain, et al. Clinical Chemistry 20: 470 (1974)) using 100 and 300 mg/dl standards. Serum free fatty acid concentration is determined utilizing a kit from WAKO (Osaka, Japan), as adapted for use with the Abbott VP™ and VP Super System® Autoanalyzer (Abbott Laboratories, Irving, Tex.), or the Abbott Spectrum CCX™ (Abbott Laboratories, Irving, Tex.). Serum insulin, triglycerides, free fatty acids and total cholesterol levels are then calculated by the equations: Serum insulin (μU/ml)=Sample value×2; Serum triglycerides (mg/dl)=Sample value×2; Serum total cholesterol (mg/dl)=Sample value×2; Serum free fatty acid (μEq/l)=Sample value×2; where 2 is the dilution factor.

The animals dosed with vehicle maintain substantially unchanged, elevated serum insulin (e.g., 275 μU/ml), serum triglycerides (e.g., 235 mg/dl), serum free fatty acid (1500 mEq/ml) and serum total cholesterol (e.g., 190 mg/dl) levels. The serum insulin, triglycerides, free fatty acid and total cholesterol lowering activity of the test compounds are determined by statistical analysis (unpaired t-test) of the mean serum insulin, triglycerides, or total cholesterol concentration between the test compound group and the vehicle-treated control group.

### Measurement of Energy Expenditure in Rats

As would be appreciated by those skilled in the relevant art, during increased energy expenditure, animals generally consume more oxygen. In addition, metabolic fuels such as, for example, glucose and fatty acids, are oxidized to CO₂ and H₂O with the concomitant evolution of heat, commonly referred to in the art as thermogenesis. Thus, the measurement of oxygen consumption in animals, including humans and companion animals, is an indirect measure of thermogenesis. Indirect calorimetry is commonly used in animals, e.g., humans, by those skilled in the relevant art to measure such energy expenditures.

Those skilled in the art understand that increased energy expenditure and the concomitant burning of metabolic fuels resulting in the production of heat may be efficacious with respect to the treatment of, e.g., obesity.

The ability of the compounds of the present invention to generate a thermogenic response can be demonstrated according to the following protocol: This in vivo screen is designed to evaluate the efficacy of compounds that are PPAR agonists, using as an efficacy endpoint measurement of whole body oxygen consumption. The protocol involves: (a) dosing fatty Zucker rats for about 6 days, and (b) measuring oxygen consumption. Male fatty Zucker rats having a body weight range of from about 400 g to about 500 g are housed for from about 3 to about 7 days in individual cages under standard laboratory conditions prior to the initiation of the study. A compound of the present invention and a vehicle is administered by oral gavage as a single daily dose given between about 3 p.m. to about 6 p.m. for about 6 days. A compound of the present invention is dissolved in vehicle containing about 0.25% of methyl cellulose. The dosing volume is about 1 ml.

About 1 day after the last dose of the compound is administered, oxygen consumption is measured using an open circuit, indirect calorimeter (Oxymax, Columbus Instruments, Columbus, Ohio 43204). The Oxymax gas sensors are calibrated with N₂ gas and a gas mixture (about 0.5% of CO₂, about 20.5% of O₂, about 79% of N₂) before each experiment. The subject rats are removed from their home cages and their body weights recorded. The rats are placed into the sealed chambers (43×43×10 cm) of the Oxymax, the chambers are placed in the activity monitors, and the air flow rate through the chambers is then set at from about 1.6 L/min to about 1.7 L/min. The Oxymax software then calculates the oxygen consumption (mL/kg/h) by the rats based on the flow rate of air through the chambers and the difference in oxygen content at the inlet and output ports. The activity monitors have 15 infrared light beams spaced about one inch apart on each axis, and ambulatory activity is recorded when two consecutive beams are broken, and the results are recorded as counts.

Oxygen consumption and ambulatory activity are measured about every 10 min for from about 5 h to about 6.5 h. Resting oxygen consumption is calculated on individual rats by averaging the values excluding the first 5 values and the values obtained during time periods where ambulatory activity exceeds about 100 counts.

### In Vivo Atherosclerosis Assay

Anti-atherosclerotic effects of the compounds of the present invention can be determined by the amount of compound required to reduce the lipid deposition in rabbit aorta. Male New Zealand White rabbits are fed a diet containing 0.2% cholesterol and 10% coconut oil for 4 days (meal-fed once per day). Rabbits are bled from the marginal ear vein and total plasma cholesterol values are determined from these samples. The rabbits are then assigned to treatment groups so that each group has a similar mean±SD for total plasma cholesterol concentration, HDL cholesterol concentration and triglyceride concentration. After group assignment, rabbits are dosed daily with compound given as a dietary admix or on a small piece of gelatin based confection. Control rabbits receive only the dosing vehicle, be it the food or the gelatin confection. The cholesterol/coconut oil diet is continued along with the compound administration throughout the study. Plasma cholesterol, HDL-cholesterol, LDL cholesterol and triglyceride values can be determined at any point during the study by obtaining blood from the marginal ear vein. After 3-5 months, the rabbits are sacrificed and the aortae are removed from the thoracic arch to the branch of the iliac arteries. The aortae are cleaned of adventitia, opened longitudinally and then stained with Sudan IV as described by Holman et. al. (Lab. Invest. 1958, 7, 42-47). The percent of the surface area stained is quantitated by densitometry using an Optimas Image Analyzing System (Image Processing Solutions; North Reading Mass.). Reduced lipid deposition is indicated by a reduction in the percent surface area stained in the compound-receiving group in comparison with the control rabbits.

The utility of the formula I compounds useful in the present invention, their prodrugs and the salts of such compounds and prodrugs as agents in the treatment of the above described disease/conditions in ruminants is additionally demonstrated by the activity of the compounds of the present invention in the assays described below.

Negative Energy Balance

To determine negative energy balance, serum concentrations of NEFAs or ketone bodies, or levels of triglycerides in liver tissues, are measured. Higher than ‘normal’ levels of NEFA's and/or triglycerides and/or ketone bodies are indicators of negative energy balance. Levels considered ‘higher than normal’ or ‘excessive’ are:
NEFA's>800 μmol/L in serum.
Triglycerides>10% w/w in liver tissue.
Ketone bodies>1.2 □mol/L in serum.

Determination of Changes in Blood Non-Esterified Fatty Acid (NEFA) Concentrations and Liver Triglycerides Levels:

Compounds are administered once or several times in the transition period at dose levels predicted to be effective by comparing results of in-vitro receptor affinity tests in laboratory species and pharmacokinetic evaluations in cattle. NEFA levels are determined via standard laboratory methods, for example, using the commercial WAKO NEFA kit (Wako Chemical Co., USA, Dallas, Tex., 994-75409), and liver triglyceride content is determined using the method as described in the literature (J. K. Drackley, J. J. Veenhuizen, M. J. Richard and J. W. Young, J Dairy Sci, 1991, 74, 4254)).

All animals may be obtained from a commercial dairy farm approximately thirty days prior to anticipated calving date. The cows are moved into separate building, approximately 10-14 days prior to their anticipated calving dates and switched to the TMR-Close-Up dry diet. Enrolment of animals in the study begins approximately 7 days prior to their anticipated calving dates. The animals may be moved to the “on-test” pen, weighed and are locked each AM into feed stanchions. At that time, appropriate doses are administered and appropriate blood samples obtained (see table below for sample data for a PPAR alpha agonist not within the scope of the present invention, compound Z).

Animals enrolled in T01 were treated with vehicle control every other day (eod) beginning at the estimated Day −7 prior to calving, and once again at calving. Animals enrolled in T02 were treated with compound Z every other day beginning at the estimated Day −7 prior to calving, and once again at calving.

| | | | Pre Partum Dosing | |
|---|---|---|---|---|
| | | | (every other day = | |
| | | Animals per | eod-beginning | Treatment |
| Treatment | Dosage | Treatment | targeted day-7) | at Calving |
| T0l | — | 11 | X | X |
| Vehicle | | | | |
| Control | | | | |
| T02 | 0.5 mg/kg | 9 | X | X |
| Compound Z | | | | |

As soon as possible post-calving (−30 minutes) the cow is transferred to the freestall barn for the next scheduled milking (6:00 hrs and 19:00 hrs). Treatments on postpartum animals are administered every other day through day 8. Pre and post-calving NEFA samples are analyzed using the WAKO NEFA-C test kit (#994-75409). Post-calving liver biopsies are performed on all cows on days 5, 10 and 14 post-calving. Tissues are transported on ice and stored frozen at −70° F. At the conclusion of the study, samples are analysed of liver triglyceride levels using the method described by Drackley, J. K. et al. (1991, J Dairy Sci (74):4254-4264).

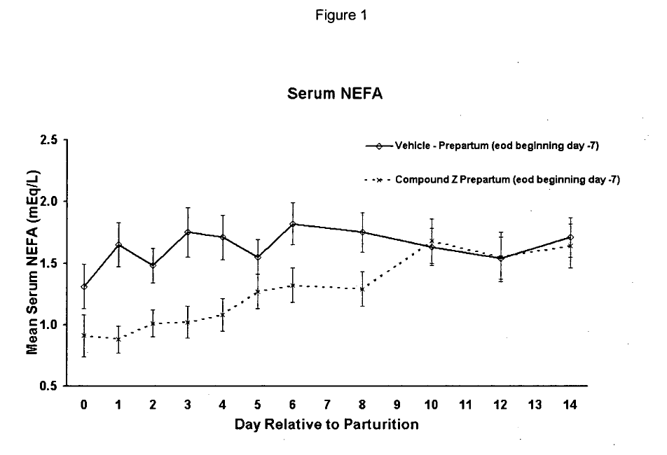

All animals treated with test article (T02) exhibited significantly lower (p<0.10) serum NEFA levels as compared to control on days 1-8, with the exceptions of T02 on day 5 (p=0.17). All treatment regimens significantly lowered liver triglyceride levels compared to placebo at all time points measured (Days 5, 10 and 14 postcalving) Results are depicted in FIG. 1.

Ketone Bodies

Levels of ketone bodies in serum can be measured by standard methods well known to the person skilled in the art, for example, by using the commercially available kits for this purpose, including Sigma BHBA kit of order number 310-A.

Milk Content:

Machines to assay for milk protein, fat, or lactose content are commercially available (MilkoScan™ 50, MilkoScan™ 4000, MilkoScan™ FT 6000 available from Foss Group). Machines to assay for somatic cell content are also commercially available (Fossomatic™ FC, Fossomatic™ Minor available from Foss Group).

Compounds used in this invention may be administered alone or in combination with one or more other compounds of the invention or in combination with one or more other drugs (or as any combination thereof).

For example, compounds of this invention can also be mixed with one or more biologically active compounds or agents selected from sedatives, analgesics, antiinflammatories, analeptics, antibacterials, antidiarrhoeals, anti-endotoxin, antifungals, respiratory stimulants, corticosteroids, diuretics, parasiticides, electrolyte preparations and nutritional supplements, growth promoters, hormones, and metabolic disease treatments, giving an even broader spectrum of veterinary or agricultural utility.

Examples of suitable active compounds or agents are found below:
Amylase inhibitors: Acarbose;
Glucosidase Inhibitors: Acarbose;
Sedatives: xylazine;
Analgesics and antiinflammatories: Lignocaine, Procaine, flunixin, oxytetracycline, ketoprofen, meloxicam and carprofen;
Analeptics: Etamiphylline, Doxapram, Diprenorphine, Hyoscine, Ketoprofen, Meloxicam, Pethidine, Xylazine and Butorphanol;
Antibacterials: Chlortetracycline, Tylosin, Amoxycillin, Ampicillin, Aproamycin, Cefquinome, Cephalexin, Clavulanic acid, Florfenicol, Danofloxacin, Enrofloxacin, Marbofloxacin, Framycetin, Procaine penicillin, procaine benzylpenicillin, Benzathine penicillin, sulfadoxine, Trimethoprim, sulphadimidine, baquiloprim, streptomycin, dihydrostreptomycin, sulphamethoxypyridazine, sulphamethoxypuridazine, oxytetracycline, flunixin, tilmicosin, cloxacillin, ethyromycin, neomycin, nafcillin, Aureomycin, lineomycin, cefoperazone, cephalonium, oxytetracycline, formosulphathiazole, sulphadiazine and zinc;
Antidiarrhoeals: Hyoscine, Dipyrone, charcoal, attapulgite, kaolin, Isphaghula husk;
Anti-endotoxins: Flunixin, ketoprofen;
Antifungals: Enilconazole, Natamycin; Respiratory stimulants: florfenicol;
Corticosteroids: dexamethasone, betamethasone;
Diuretics: frusemide;
Parasiticides—amitraz, deltamethrin, moxidectin, doramectin, alpha cypermethrin, fenvalerate, eprinomectin, permethrin, ivermectin, abamectin, ricobendazole, levamisole, febantel, triclabendazole, fenbendazole, albendazole, netobimin, oxfenazole, oxyclozanide, nitroxynil, morantel;
Electrolyte preparations and nutritional supplements: dextrose, lactose, propylene glycol, whey, glucose, glycine, calcium, cobalt, copper, iodine, iron, magnesium, manganese, phosphorous, selenium, zinc, Biotin, vitamin B₁₂, Vitamin E, and other vitamins;
Growth Promoters: monensin, flavophospholipol, bambermycin, salinomycin, tylosin;
Hormones: chorionic gonadotrophin, serum gonadotrophin, atropine, melatonin, oxytocin, dinoprost, cloprostenol, etiproston, luprostiol, buserelin, oestradiol, progesterone, and bovine somatotropin; and
Metabolic Disease Treatments: calcium gluconate, calcium borogluconate, propylene glycol, magnesium sulphate.

Compounds of this invention can also be mixed with one or more biologically active compounds or agents selected from antiprotozoals such as imidocarb, bloat remedies such as dimethicone and poloxalene, and probiotics such as Lactobacilli and streptococcus.

Administration of the compounds of the present invention can be via any method which delivers a compound of this invention systemically and/or locally. These methods include oral routes, parenteral, intraduodenal routes, etc. Generally, the compounds of this invention are administered orally, but parenteral administration (e.g., intravenous, intramuscular, subcutaneous or intramedullary) may be utilized, for example, where oral administration is inappropriate or where the patient is unable to ingest the drug.

In general an amount of a compound of the present invention is used that is sufficient to achieve the therapeutic effect desired (e.g., lipid lowering).

In general an effective dosage for the compounds of the present invention, their prodrugs and the salts of such compounds and prodrugs is in the range of about 0.001 to about 100 mg/kg/day, preferably about 0.005 to about 5 mg/kg/day.

A dosage of the combination pharmaceutical agents to be used in conjuction with the PPAR agonists is used that is effective for the indication being treated. Such dosages can be determined by standard assays such as those referenced above and provided herein. The combination agents may be administered simultaneously or sequentially in any order.

For example, typically an effective dosage for HMG-CoA reductase inhibitors is in the range of about 0.01 to about 100 mg/kg/day.

The compounds of the present invention are generally administered in the form of a pharmaceutical composition comprising at least one of the compounds of this invention together with a pharmaceutically acceptable vehicle, diluent or carrier. Thus, the compounds of the present invention can be administered individually or together in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. A preferred formulation is a solution or suspension in an oil, for example olive oil, Miglyol™ or Capmul™, in a soft gelatin capsule. Antioxidants may be added to prevent long term degradation as appropriate. When aqueous suspensions and/or elixirs are desired for oral administration, the compounds of the present invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 19th Edition (1995).

Pharmaceutical compositions according to the present invention may contain 0.1%-95% of the compound(s) of the present invention, preferably 1%-70%. In any event, the composition or formulation to be administered will contain a quantity of a compound(s) according to the present invention in an amount effective to treat the disease/condition of the subject being treated, e.g., atherosclerosis.

Since the present invention has an aspect that relates to the treatment of the disease/conditions described herein with a combination of active ingredients, which may be administered separately, the invention also relates to combining separate pharmaceutical compositions in kit form. The kit comprises two separate pharmaceutical compositions: a compound of the present invention, a prodrug thereof or a salt of such compound or prodrugs and a second compound as described above. The kit for example comprises means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a compound of the present invention can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compounds of the present invention either alone or in combination with each other or other compounds generally will be administered in a convenient formulation. The following formulation examples only are illustrative and are not intended to limit the scope of the present invention.

In the formulations which follow, “active ingredient” means a compound of the present invention.

Formulation 1: Gelatin Capsules

Hard gelatin capsules are prepared using the following:

| Ingredient | Quantity (mg/capsule) |
|---|---|
| Active ingredient | 0.25-100 |
| Starch, NF | 0-650 |
| Starch flowable powder | 0-50 |
| Silicone fluid 350 centistokes | 0-15 |

A tablet formulation is prepared using the ingredients below:

Formulation 2: Tablets

| Ingredient | Quantity (mg/tablet) |
|---|---|
| Active ingredient | 0.25-100 |
| Cellulose, microcrystalline | 200-650 |
| Silicon dioxide, fumed | 10-650 |
| Stearate acid | 5-15 |

The components are blended and compressed to form tablets.

Alternatively, tablets each containing 0.25-100 mg of active ingredients are made up as follows:

Formulation 3: Tablets

| Ingredient | Quantity (mg/tablet) |
|---|---|
| Active ingredient | 0.25-100 |
| Starch | 45 |
| Cellulose, microcrystalline | 35 |
| Polyvinylpyrrolidone (as 10% solution in water) | 4 |
| Sodium carboxymethyl cellulose | 4.5 |
| Magnesium stearate | 0.5 |
| Talc | 1 |

The active ingredients, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 500-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 0.25-100 mg of active ingredient per 5 ml dose are made as follows:

Formulation 4: Suspensions

| Ingredient | Quantity (mg/5 ml) | |
|---|---|---|
| Active ingredient | 0.25-100 | mg |
| Sodium carboxymethyl cellulose | 50 | mg |
| Syrup | 1.25 | mg |
| Benzoic acid solution | 0.10 | mL |
| Flavor | q.v. | |
| Color | q.v. | |
| Purified Water to | 5 | mL |

The active ingredient is passed through a No. 45 mesh U.S. sieve and mixed with the sodium carboxymethyl cellulose and syrup to form a smooth paste. The benzoic acid solution, flavor, and color are diluted with some of the water and added, with stirring. Sufficient water is then added to produce the required volume.

An aerosol solution is prepared containing the following ingredients:

Formulation 5: Aerosol

| Ingredient | Quantity (% by weight) |
|---|---|
| Active ingredient | 0.25 |
| Ethanol | 25.75 |
| Propellant 22 (Chlorodifluoromethane) | 70.00 |

The active ingredient is mixed with ethanol and the mixture added to a portion of the propellant 22, cooled to 30° C., and transferred to a filling device. The required amount is then fed to a stainless steel container and diluted with the remaining propellant. The valve units are then fitted to the container.

Suppositories are prepared as follows:

Formulation 6: Suppositories

| Ingredient | Quantity (mg/suppository) |
|---|---|
| Active ingredient | 250 |
| Saturated fatty acid glycerides | 2,000 |

The active ingredient is passed through a No. 60 mesh U.S. sieve and suspended in the saturated fatty acid glycerides previously melted using the minimal necessary heat. The mixture is then poured into a suppository mold of nominal 2 g capacity and allowed to cool.

An intravenous formulation is prepared as follows:

Formulation 7: Intravenous Solution

| Ingredient | Quantity | |
|---|---|---|
| Active ingredient dissolved in ethanol 1% | 20 | mg |
| Intralipid ™ emulsion | 1,000 | mL |

The solution of the above ingredients is intravenously administered to a patient at a rate of about 1 mL per minute.

Soft gelatin capsules are prepared using the following:

Formulation 8: Soft Gelatin Capsule with Oil Formulation

| Ingredient | Quantity (mg/capsule) |
|---|---|
| Active ingredient | 10-500 |
| Olive Oil or Miglyol ™ Oil | 500-1000 |

The active ingredient above may also be a combination of therapeutic agents.

### GENERAL EXPERIMENTAL PROCEDURES

The following examples are put forth so as to provide those of ordinary skill in the art with a disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were utilized without further purification. Room or ambient temperature refers to 20-25° C. All non-aqueous reactions were run under a nitrogen atmosphere for convenience and to maximize yields. Concentration in vacuo means that a rotary evaporator was used. The names for the compounds of the invention were-created by the Autonom 2.0 PC-batch version from Beilstein Informationssysteme GmbH (ISBN 3-89536-976-4). “DMSO” means dimethyl sulfoxide.

NMR spectra were recorded on a Varian Unity 400 (Varian Co., Palo Alto, Calif.) NMR spectrometer at ambient temperature. Chemical shifts are expressed in parts per million (δ) relative to an external standard (tetramethylsilane). The peak shapes are denoted as follows: s, singlet; d, doublet, t, triplet, q, quartet, m, multiplet with the prefix br indicating a broadened signal. The coupling constant (J) data given have a maximum error of ±0.41 Hz due to the digitization of the spectra that are acquired. Mass spectra were obtained by (1) atmospheric pressure chemical ionization (APCI) in alternating positive and negative ion mode using a Fisons Platform II Spectrometer or a Micromass MZD Spectrometer (Micromass, Manchester, UK) or (2) electrospray ionization in alternating positive and negative ion mode using a Micromass MZD Spectrometer (Micromass, Manchester, UK) with a Gilson LC-MS interface (Gilson Instruments, Middleton, Wis.) or (3) a QP-8000 mass spectrometer (Shimadzu Corporation, Kyoto, Japan) operating in positive or negative single ion monitoring mode, utilizing electrospray ionization or atmospheric pressure chemical ionization. Where the intensity of chlorine- or bromine-containing ions are described, the expected intensity ratio was observed (approximately 3:1 for ³⁵Cl/³⁷Cl-containing ions and 1:1 for ⁷⁹Br/⁸¹Br-containing ions) and the position of only the lower mass ion is given.

Column chromatography was performed with either Baker Silica Gel (40 μm) (J. T. Baker, Phillipsburg, N.J.) or Silica Gel 60 (40-63 μm)(EM Sciences, Gibbstown, N.J.). Flash chromatography was performed using a Flash 12 or Flash 40 column (Biotage, Dyar Corp., Charlottesville, Va.). Preparative HPLC purification was performed on a Shimadzu 10A preparative HPLC system (Shimadzu Corporation, Kyoto, Japan) using a model SIL-10A autosampler and model 8A HPLC pumps. Preparative HPLC-MS was performed on an identical system, modified with a QP-8000 mass spectrometer operating in positive or negative single ion monitoring mode, utilizing electrospray ionization or atmospheric pressure chemical ionization. Elution was carried out using water/acetonitrile gradients containing either 0.1% formic acid or ammonium hydroxide as a modifier. In acidic mode, typical columns used include Waters Symmetry C8, 5 μm, 19×50 mm or 30×50 mm, Waters XTerra C18, 5 μm, 50×50 (Waters Corp, Milford, Mass.) or Phenomenex Synergi Max-RP 4 μm, 50×50 mm (Phenomenex Inc., Torrance, Calif.). In basic mode, the Phenomenex Synergi Max-RP 4 μm, 21.2×50 mm or 30×50 mm columns (Phenomenex Inc., Torrance, Calif.) were used. Optical rotations were determined using a Jasco P-1020 Polarimeter Jasco Inc., Easton, Md.). Dimethylformamide, tetrahydrofuran, toluene and dichloromethane were the anhydrous grade supplied by Aldrich Chemical Company (Milwaukee, Wis.). Unless otherwise specified, reagents were used as obtained from commercial sources. The terms “concentrated” and “evaporated” refer to removal of solvent at 1-200 mm of mercury pressure on a rotary evaporator with a bath temperature of less than 45° C. The abbreviation “min” stand for “minutes” and “h” or “hr” stand for “hours.” The abbreviation “gm” or “g” stand for grams. The abbreviation “μl” or “μL” stand for microliters.

### Example 1

### 2-Isopropyl-5-{2-[5-methyl-2-(4-trifluoromethyl-phenyl)-thiazol-4-yl]-ethylsulfamoyl}-benzoic acid methyl ester

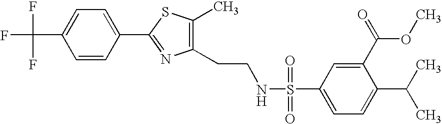

To a mixture of 2-[5-methyl-2-(4-trifluoromethyl-phenyl)-thiazol-4-yl]-ethylamine (0.097 g, 0.34 mmol) and 5-chlorosulfonyl-2-isopropylbenzoic acid methyl ester (0.103 g, 0.37 mmol) in 3 ml acetone was added sufficient dimethylformamide (−1 ml) to effect solution. A solution of sodium bicarbonate (0.085 g, 1.01 mmol) in 1 ml water was added and the reaction mixture was stirred overnight at room temperature. The acetone was then removed under reduced pressure and the residue was partitioned between 50 ml ethyl acetate and 30 ml 1N aqueous hydrochloric acid solution. The ethyl acetate fraction was washed sequentially with 30 ml water and 30 ml brine, dried (anhydrous sodium sulfate) and concentrated under reduced pressure. The residual brown oil (0.18 g) was purified by flash column chromatography (15 g silica gel), eluting with 4:1 hexane/ethyl acetate to yield the title compound as a yellowish solid (0.11 g, 61% yield).

MS: 527.0 (M+1)

The title compounds of EXAMPLES 2-65 were prepared using procedures analogous to that of EXAMPLE 1 from appropriate starting materials.

| Ex. | Compound | Compound Name | Data |
|---|---|---|---|
| 2 | 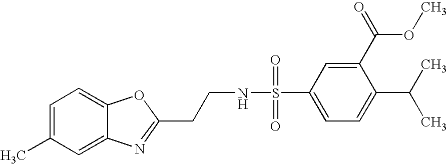 | 2-Isopropyl-5-[2-(5-meth- yl-benzooxazol-2-yl)-eth- ylsulfamoyl]-benzoic acid methyl ester | 18% yield. MS: 417.1 (M + 1) |
| 3 | 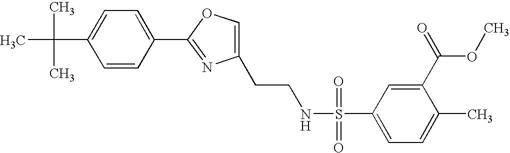 | 5-{2-[2-(4-tert-Butyl-phe- nyl)-oxazol-4-yl]-eth- ylsulfamoyl}-2-meth- yl-benzoic acid methyl ester | 56% yield. MS: 457.1 (M + 1) |
| 4 | 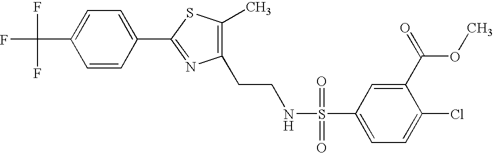 | 2-Chloro-5-{2-[5-meth- yl-2-(4-tri- fluoromethyl-phe- nyl)-thiazol-4-yl]-eth- ylsulfamoyl}-benzoic acid methyl ester | 30% yield. MS: 519.0 (M + 1) |
| 5 | 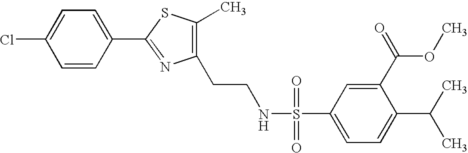 | 5-{2-[2-(4-Chloro-phe- nyl)-5-methyl-thia- zol-4-yl]-eth- ylsulfamoyl}-2-iso- propyl-benzoic acid methyl ester | 68% yield. MS: (493.0 (M + 1) |
| 6 | 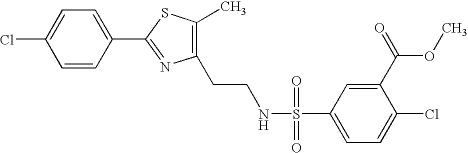 | 2-Chloro-5-{2-[2-(4-chlor- o-phenyl)-5-meth- yl-thiazol-4-yl]-eth- ylsulfamoyl}-benzoic acid methyl ester | 42% yield. MS: 484.9 (M + 1) |
| 7 | 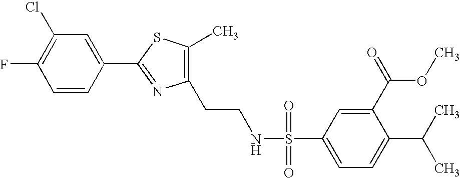 | 5-{2-[2-(3-Chloro-4-fluor- o-phenyl)-5-meth- yl-thiazol-4-yl]-eth- ylsulfamoyl}-2-iso- propyl-benzoic acid methyl ester | 53% yield. MS: 508.9 (M − 1) |
| 8 | 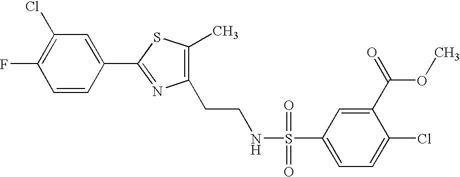 | 2-Chloro-5-{2-[2-(3-chlor- o-4-fluoro-phe- nyl)-5-methyl-thia- zol-4-yl]-eth- ylsulfamoyl}-benzoic acid methyl ester | 23% yield. MS: 502.9 (M + 1) |
| 9 | 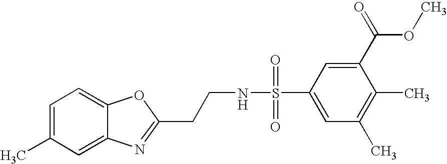 | 2,3-Dimethyl-5-[2-(5-meth- yl-benzooxazol-2-yl)-eth- ylsulfamoyl]-benzoic acid methyl ester | 24% yield. MS: 403.0 (M + 1) |
| 10 | 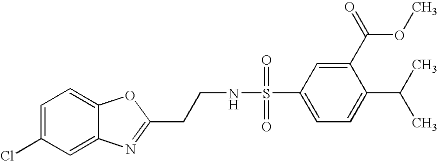 | 5-[2-(5-Chloro-benzo- oxazol-2-yl)-eth- ylsulfamoyl]-2-iso- propyl-benzoic acid methyl ester | 13% yield. MS: 437.0 (M + 1) |
| 11 | 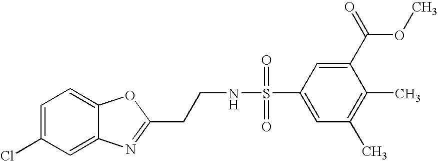 | 5-[2-(5-Chloro-benzo- oxazol-2-yl)-eth- ylsulfamoyl]-2,3-di- methyl-benzoic acid methyl ester | 11% yield. MS: 423.0 (M + 1) |
| 12 | 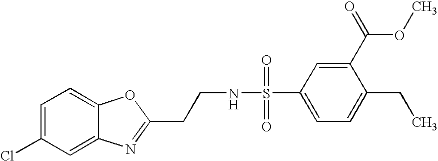 | 5-[2-(5-Chloro-benzo- oxazol-2-yl)-eth- ylsulfamoyl]-2-eth- yl-benzoic acid methyl ester | 6% yield. MS: 423.0 (M + 1) |
| 13 | 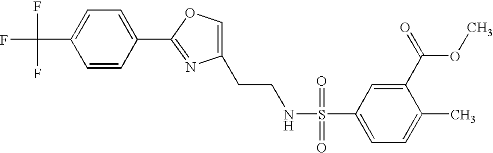 | 2-Methyl-5-{2-[2-(4-tri- fluoromethyl-phe- nyl)-oxazol-4-yl]-eth- ylsulfamoyl}-benzoic acid methyl ester | 25% yield. MS: 469.0 (M + 1) |
| 14 | 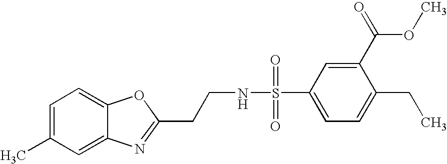 | 2-Ethyl-5-[2-(5-meth- yl-benzooxazol-2-yl)-eth- ylsulfamoyl]-benzoic acid methyl ester | 8% yield. MS: 403.1 (M + 1) |
| 15 | 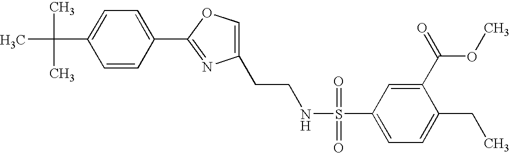 | 5-{2-[2-(4-tert-Butyl-phe- nyl)-oxazol-4-yl]-eth- ylsulfamoyl}-2-eth- yl-benzoic acid methyl ester | 36% yield. MS: 471.1 (M + 1) |
| 16 | 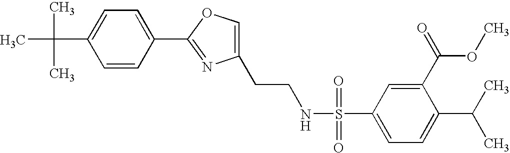 | 5-{2-[2-(4-tert-Butyl-phe- nyl)-oxazol-4-yl]-eth- ylsulfamoyl}-2-iso- propyl-benzoic acid methyl ester | 46% yield. MS: 485.1 (M + 1) |
| 17 | 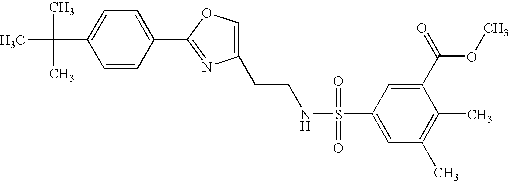 | 5-{2-[2-(4-tert-Butyl-phe- nyl)-oxazol-4-yl]-eth- ylsulfamoyl}-2,3-di- methyl-benzoic acid methyl ester | 53% yield. MS: 471.1 (M + 1) |
| 18 | 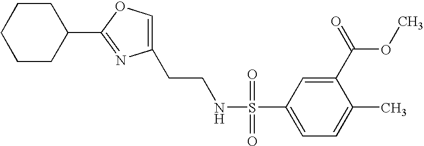 | 5-[2-(2-Cyclohexyl-oxa- zol-4-yl)-eth- ylsulfamoyl]-2-meth- yl-benzoic acid methyl ester | 22% yield. MS: 407.1 (M + 1) |
| 19 | 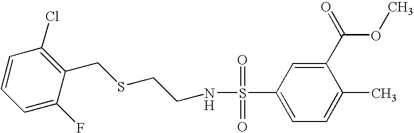 | 5-[2-(2-Chloro-6-fluor- o-benzylsulfanyl)-eth- ylsulfamoyl]-2-meth- yl-benzoic acid methyl ester | 54% yield. MS: 432.0 (M + 1) |
| 20 | 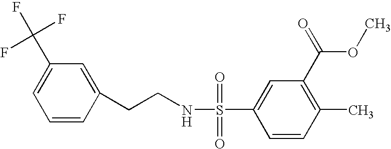 | 2-Methyl-5-[2-(3-tri- fluoromethyl-phe- nyl)-eth- ylsulfamoyl]-benzoic acid methyl ester | 76% yield. MS: 402.0 (M + 1) |
| 21 | 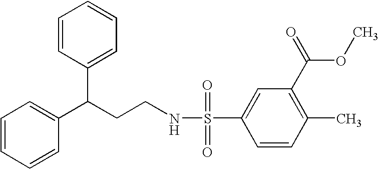 | 5-(3,3-Diphenyl-pro- pylsulfamoyl)-2-meth- yl-benzoic acid methyl ester | 71% yield. MS: 424.1 (M + 1) |
| 22 | 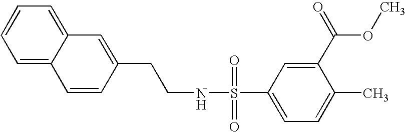 | 2-Methyl-5-(2-naph- thalen-2-yl-eth- ylsulfamoyl)-benzoic acid methyl ester | 42% yield. MS: 484.0 (M + 1) |
| 23 | 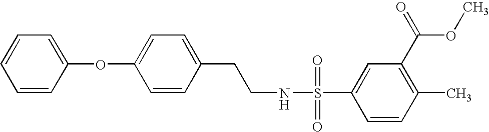 | 2-Methyl-5-[2-(4-phe- noxy-phenyl)-eth- ylsulfamoyl]-benzoic acid methyl ester | 64% yield. MS: 426.1 (M + 1) |
| 24 | 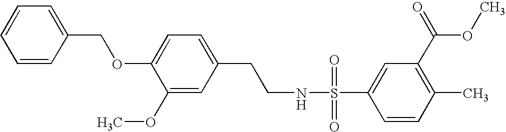 | 5-[2-(4-Benzyloxy-3-meth- oxy-phenyl)-eth- ylsulfamoyl]-2-meth- yl-benzoic acid methyl ester | 54% yield. MS: 468.0 (M + 1) |
| 25 | 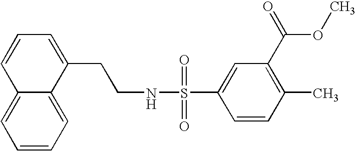 | 2-Methyl-5-(2-naph- thalen-1-yl-eth- ylsulfamoyl)-benzoic acid methyl ester | 53% yield. MS: 384.0 (M + 1) |
| 26 | 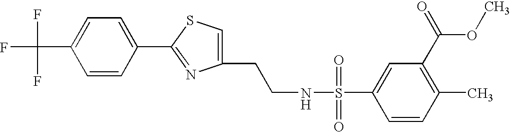 | 2-Methyl-5-{2-[2-(4-tri- fluoromethyl-phe- nyl)-thiazol-4-yl]-eth- ylsulfamoyl}-benzoic acid methyl ester | 48% yield. MS: 484.0 (M + 1) |
| 27 | 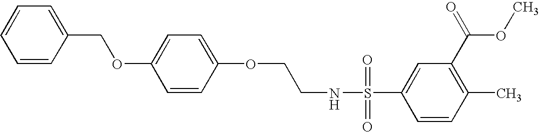 | 5-[2-(4-Benzyloxy-phe- noxy)-eth- ylsulfamoyl]-2-meth- yl-benzoic acid methyl ester | 38% yield. MS: 454.1 (M − 1) |
| 28 | 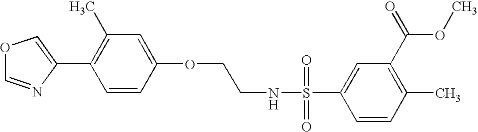 | 2-Methyl-5-[2-(3-meth- yl-4-oxazol-4-yl-phe- noxy)-eth- ylsulfamoyl]-benzoic acid methyl ester | 48% yield. MS: 431.1 (M + 1) |
| 29 | 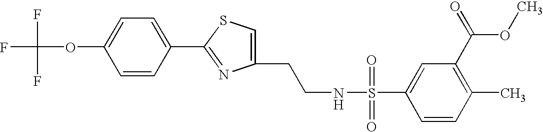 | 2-Methyl-5-{2-[2-(4-tri- fluoromethoxy-phe- nyl)-thiazol-4-yl]-eth- ylsulfamoyl}-benzoic acid methyl ester | 23% yield. MS: 501.0 (M + 1) |
| 30 | 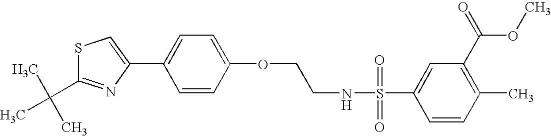 | 5-{2-[4-(2-tert-Butyl-thia- zol-4-yl)-phenoxy]-eth- ylsulfamoyl}-2-meth- yl-benzoic acid methyl ester | 41% yield. MS: 489.1 (M + 1) |

### Example 31

### 5-[2-(3,5-Dichloro-phenoxy)-ethylsulfamoyl]-2-methyl-benzoic acid methyl ester

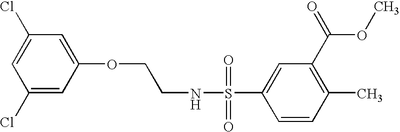

13% yield.

¹H NMR (400 MHz, CDCl₃): δ 2.67 (s, 3H), 3.4 (c, 2H), 3.92 (s+c, 5H), 6.65 (s, 2H), 6.96 (s, 1H), 7.39 (d, 1H), 7.8 (d, 1H), 8.4 (s, 1H).

### Example 32

### 5-{2-[2-(4-Chloro-phenyl)-thiazol-4-yl]-ethylsulfamoyl}-2-methyl-benzoic acid methyl ester

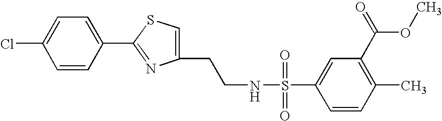

21% yield.

MS: 449.0 (M−1)

### Example 33

### 2-Methyl-5-{2-[4-(4-trifluoromethoxy-benzoylamino)-phenyl]-ethylsulfamoyl}-benzoic acid methyl ester

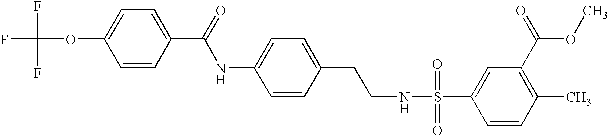

54% yield.

¹H NMR (400 MHz, CDCl₃): δ 2.67 (s, 3H), 2.77 (t, 2H), 3.24 (m, 2H), 3.92 (s, 3H), 7.08 (d, 2H), 7.34 (d, 1H), 7.38 (d, 1H), 7.49 (d, 2H), 7.80 (m, 2H), 7.94 (m, 2H), 8.3 (d, 1H).

### Example 34

### 2-Methyl-5-{2-[4-(4-trifluoromethyl-benzoylamino)-phenyl]-ethylsulfamoyl}-benzoic acid methyl ester

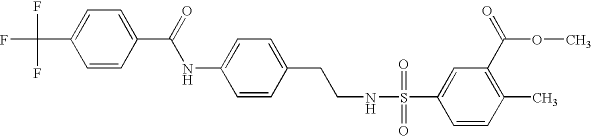

54% yield.

MS: 519.0 (M−1)

### Example 35

### 2-Methyl-5-(2-{4-[(naphthalene-2-carbonyl)-amino]-phenyl}-ethylsulfamoyl)-benzoic acid methyl ester

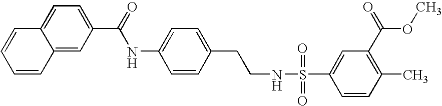

17% yield.

¹H NMR (400 MHz, CDCl₃): δ 2.68 (s, 3H), 2.78 (t, 2H), 3.25 (m, 2H), 3.92 (s, 3H), 7.1 (d, 2H), 7.39 (d, 1H), 7.59 (c, 4H), 7.81 (m, 1H), 7.94 (c, 4H), 8.32 (d, 1H), 8.40 (s, 1H).

### Example 36

### 2-Methyl-5-{2-[4-(3-trifluoromethyl-benzoylamino)-phenyl]-ethylsulfamoyl}-benzoic acid methyl ester

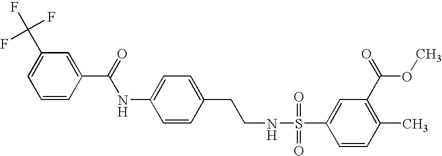

36% yield.

MS: 519.1 (M−1)

### Example 37

### 2-Methyl-5-[2-(5-methyl-2-naphthalen-2-yl-thiazol-4-yl)-ethylsulfamoyl]-benzoic acid methyl ester

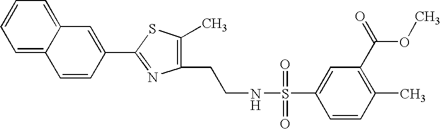

42% yield.

MS: 481.0 (M+1)

### Example 38

### 5-{2-[4-(4-Fluoro-benzenesulfonylamino)-phenyl]-ethylsulfamoyl}-2-methyl-benzoic acid methyl ester

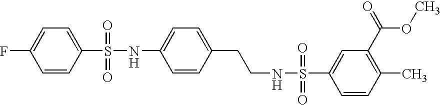

67% yield.

MS: 505.0 (M−1)

### Example 39

### 2-Methyl-5-(2-[4-(4-trifluoromethyl-benzenesulfonylamino)-phenyl]-ethylsulfamoyl-benzoic acid methyl ester

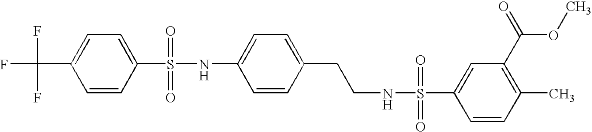

55% yield.

MS: 555.0 (M−1)

### Example 40

### 5-{2-[4-(4-tert-Butyl-benzenesulfonylamino)-phenyl]-ethylsulfamoyl}-2-methyl-benzoic acid methyl ester

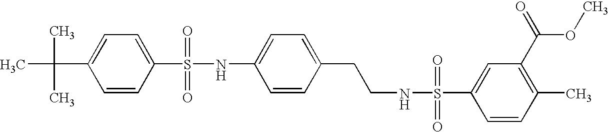

53% yield.

MS: 543.1 (M+1)

### Example 41

### 2-Methyl-5-(2-{4-[2-(4-trifluoromethoxy-phenyl)-acetylamino]-phenyl}-ethylsulfamoyl)-benzoic acid methyl ester

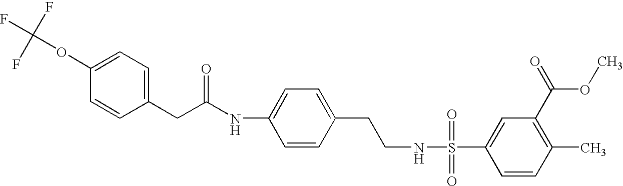

42% yield

MS: 551.0 (M+1)

### Example 42

### 5-(2-Benzooxazol-2-yl-ethylsulfamoyl)-2-methyl-benzoic acid methyl ester

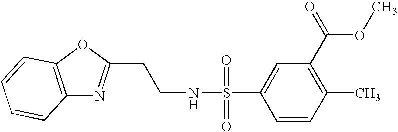

40% yield.

MS: 375.2 (M+1)

### Example 43

### 2-Methyl-5-[2-(5-methyl-benzooxazol-2-yl)-ethylsulfamoyl]-benzoic acid methyl ester

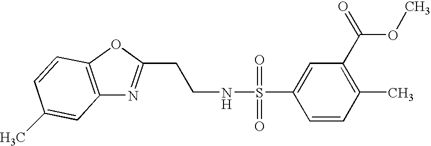

30% yield.

MS: 389.2 (M+1)

### Example 44

### 5-[2-(5-Chloro-benzooxazol-2-yl)-ethylsulfamoyl]-2-methyl-benzoic acid methyl ester

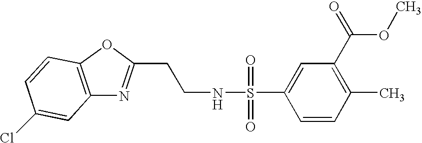

19% yield.

¹H NMR (400 MHz, CDCl₃): δ 2.66 (s, 3H), 3.10 (t, 2H), 3.53 (m, 2H), 3.9 (s, 3H), 7.29 (m, 1H), 7.36 (m, 2H), 7.61 (d, 1H), 7.86 (m, 1H), 8.39 (d, 1H).

### Example 45

### 5-(2-Benzothiazol-2-yl-ethylsulfamoyl)-2-methyl-benzoic acid methyl ester

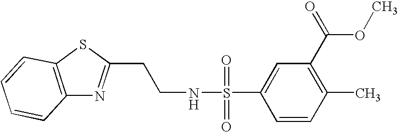

38% yield.

MS: 391.1 (M+1)

### Example 46

### 2-Methyl-5-[2-(5-trifluoromethyl-benzothiazol-2-yl)-ethylsulfamoyl-benzoic acid methyl ester

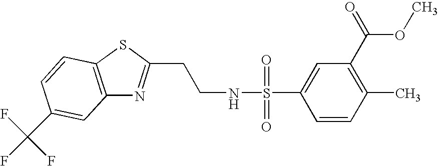

56% yield.

MS: 459.0 (M+1)

### Example 47

### 5-[2-(4-Cyclohexyl-phenoxy)-ethylsulfamoyl]-2-methyl-benzoic acid methyl ester

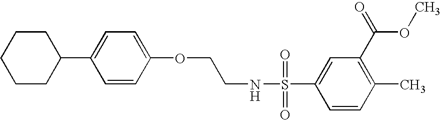

49% yield.

MS: 432.2 (M+1)

### Example 48

### 5-{2-[2-(4-tert-Butyl-phenyl)-5-methyl-thiazol-4-yl]-ethylsulfamoyl}-2-methyl-benzoic acid methyl ester

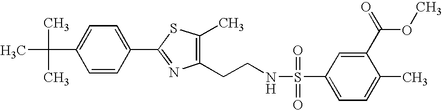

45% yield

MS: 487.1 (M+1)

### Example 49

### 5-{2-[2-(3-Chloro-4-fluoro-phenyl)-5-methyl-thiazol-4-yl]-ethylsulfamoyl}-2-methyl-benzoic acid methyl ester

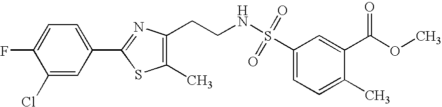

36% yield.

MS: 483.0 (M+1)

### Example 50

### 2-Methyl-5-{2-[5-methyl-2-(4-trifluoromethyl-phenyl)-thiazol-4-yl]-ethylsulfamoyl}-benzoic acid methyl ester

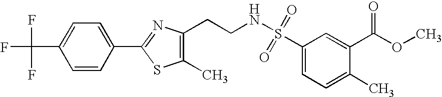

49% yield.

MS: 499.0 (M+1)

### Example 51

### 2-Ethyl-5-[2-(5-methyl-2-phenyl-oxazol-4-yl)-ethylsulfamoyl]-benzoic acid methyl ester

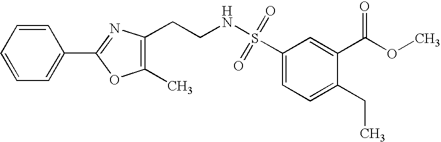

40% yield.

MS: 429.1 (M+1)

### Example 52

### 2-isopropyl-5-[2-(5-methyl-2-phenyl-oxazol-4-yl)-ethylsulfamoyl]-benzoic acid methyl ester

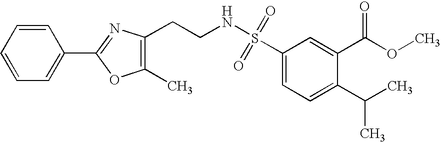

51% yield.

MS: 443.1 (M+1)

### Example 53

### 2,3-Dimethyl-5-[2-(5-methyl-2-phenyl-oxazol-4-yl)-ethylsulfamoyl]-benzoic acid methyl ester

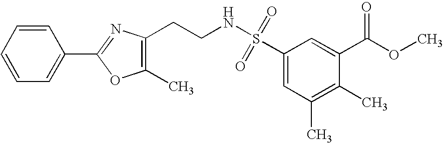

47% yield.

MS: 429.1 (M+1)

### Example 54

### 5-{2-[2-(4-tert-Butyl-phenyl)-5-methyl-thiazol-4-yl]-ethylsulfamoyl}-2-ethyl-benzoic acid methyl ester

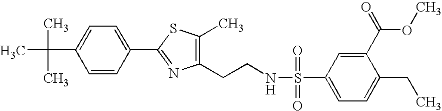

47% yield.

MS: 501.1 (M+1)

### Example 55

### 5-{2-[2-(4-tert-Butyl-phenyl)-5-methyl-thiazol-4-yl]-ethylsulfamoyl}-2,3-dimethyl-benzoic acid methyl ester

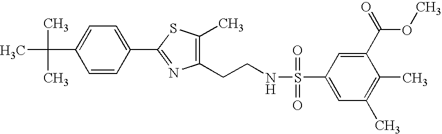

49% yield.

¹H NMR (400 MHz, CDCl₃): δ 1.34 (s, 9H), 2.21 (s, 3H), 2.31 (s, 3H), 2.45 (s, 3H), 2.82 (c, 2H), 3.32 (c, 2H), 3.8 (s, 3H), 7.45 (d, 2H), 7.68 (s, 1H), 7.75 (c, 2H), 8.09 (s, 1H).

### Example 56

### 5-{2-[2-(4-tert-Butyl-phenyl)-5-methyl-thiazol-4-yl]-ethylsulfamoyl}-2-isopropyl-benzoic acid methyl ester

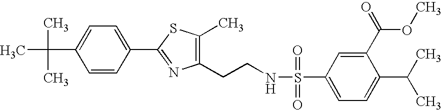

47% yield.

MS: 515.1 (M+1)

### Example 57

### 2-Ethyl-5-{2-[5-methyl-2-(4-trifluoromethyl-phenyl}-thiazol-4-yl]-15 ethylsulfamoyl]-benzoic acid methyl ester

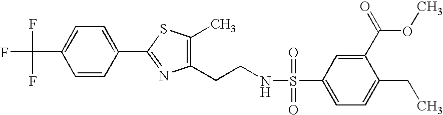

45% yield.

MS: 513.0 (M+1)

### Example 58

### 2,3-Dimethyl-5-{2-[5-methyl-2-(4-trifluoromethyl-phenyl)-thiazol-4-yl]-ethylsulfamoyl}-benzoic acid methyl ester

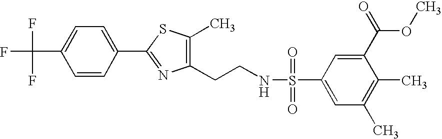

52% yield.

MS: 513.1 M+1)

### Example 59

### 5-{2-[2-(4-Chloro-phenyl)-5-methyl-thiazol-4-yl]-ethylsulfamoyl}-2-methyl-benzoic acid methyl ester

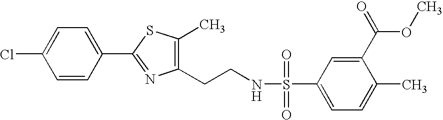

54% yield.

MS: 465.0 (M+1)

### Example 60

### 5-{2-[2-(4-Chloro-phenyl)-5-methyl-thiazol-4-yl]-ethylsulfamoyl}-2-ethyl-benzoic acid methyl ester

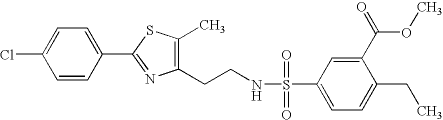

47% yield.

MS: 479.1 (M+1)

### Example 61

### 5-{2-[2-(4-Chloro-phenyl)-5-methyl-thiazol-4-yl]-ethylsulfamoyl}-2,3-dimethyl-benzoic acid methyl ester

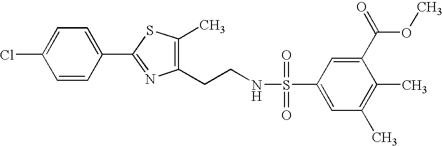

59% yield.

MS: 479.0 (M+1)

### Example 62

### 5-{2-[2-(3-Chloro-4-fluoro-phenyl)-5-methyl-thiazol-4-yl]-ethylsulfamoyl}-2-ethyl-benzoic acid methyl ester

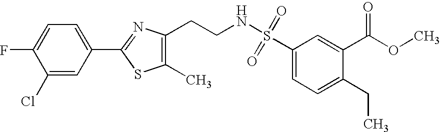

34% yield.

MS: 497.0 (M+1) dimethyl-benzoic acid methyl ester

### Example 63

### 5-{2-[2-(3-Chloro-4-fluoro-phenyl)-5-methyl-thiazol-4-yl]-ethylsulfamoyl}-2,3-dimethyl-benzoic acid methyl ester

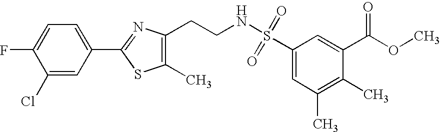

58% yield.

MS: 495.0 (M−1)

### Example 64

### 2-Methyl-5-[3-(5-methyl-benzooxazol-2-yl)-propylsulfamoyl]-benzoic acid methyl ester

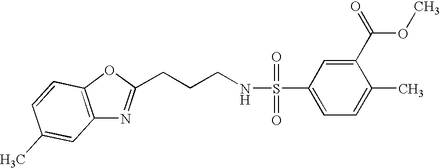

4% yield.

MS: 403.4 (M+1)

### Example 65

### 2-Ethyl-5-(2-hydroxy-ethylsulfamoyl)-benzoic acid methyl ester

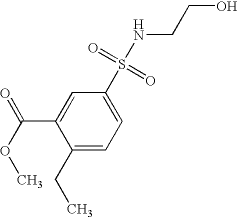

56% yield.

MS: 286.1 (M−1)

### Example 66

### 5-[2-(4-Ethyl-phenylsulfanyl)-ethylsulfamoyl]-2,3-dimethyl-benzoic acid methyl ester

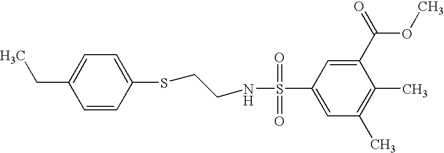

To a mixture of 2-(4-ethyl-phenylsulfanyl)-ethylamine (0.318 g, 1.76 mmol) and 5-chlorosulfonyl-2,3-dimethylbenzoic acid methyl ester (0.461 g, 1.76 mmol) in 5 ml tetrahydrofuran was added dropwise at room temperature, pyridine (0.426 ml, 5.23 mmol), followed by triethylamine (0.269 ml, 1.93 mmol). The resulting mixture was heated to 70° C. while dimethylformamide (−5 ml) was added to effect solution. The reaction mixture was heated at 70° C. for 2 hr, then cooled to room temperature and diluted with 100 ml ethyl acatate. The ethyl acetate solution was washed sequentially with 80 ml 1N aqueous hydrochloric acid solution, 80 ml water and 80 ml brine, dried (anhydrous sodium sulfate) and concentrated under reduced pressure. The residue (0.746 g) was purified by flash column chromatography (15 g silica gel), eluting with 85:15 hexane/ethyl acetate to yield the title compound as a yellowish oil (0.618 g, 86% yield). MS: 408.3 (M+1) The title compounds of EXAMPLES 67-141 were prepared using procedures analogous to that of EXAMPLE 66 from appropriate starting materials.

| Ex. | Compound | Compound Name | Data |
|---|---|---|---|
| 67 | 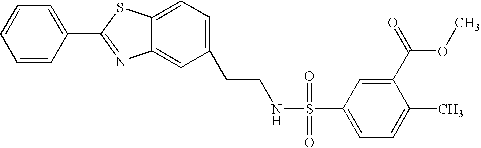 | 2-Methyl-5-[2-(2-phe- nyl-benzo- thiazol-5-yl)-eth- ylsulfamoyl]-benzoic acid methyl ester | 9% yield. MS: 467.0 (M + 1) |
| 68 | 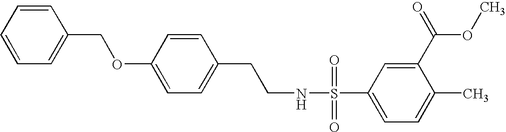 | 5-[2-(4-Benzyloxy-phe- nyl)-eth- ylsulfamoyl]-2-meth- yl-benzoic acid methyl ester | 76% yield. MS: 440.2 (M + 1) |
| 69 | 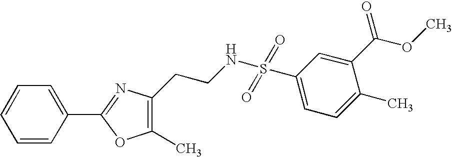 | 2-Methyl-5-[2-(5-meth- yl-2-phenyl-oxa- zol-4-yl)-eth- ylsulfamoyl]-benzoic acid methyl ester | 56% yield. MS: 415.1 (M + 1) |
| 70 | 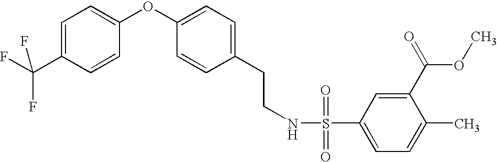 | 2-Methyl-5-{2-[4-(4-tri- fluoromethyl-phe- noxy)-phenyl]-eth- ylsulfamoyl}-benzoic acid methyl ester | 90% yield. MS: 494.0 (M + 1) |
| 71 | 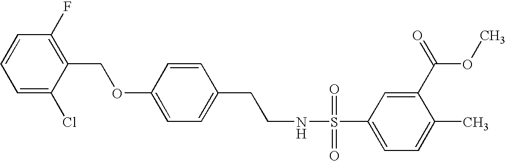 | 5-{2-[4-(2-Chloro-6-fluor- o-benzyloxy)-phe- nyl]-eth- ylsulfamoyl}-2-meth- yl-benzoic acid methyl ester | 19% yield. |
| 72 | 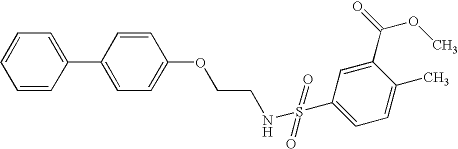 | 5-[2-(Biphenyl-4-yl- oxy)-ethyl- sulfamoyl]-2-meth- yl-benzoic acid methyl ester | 64% yield. |
| 73 | 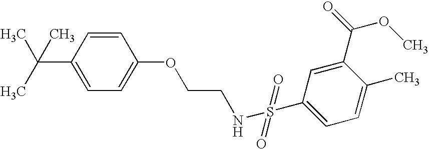 | 5-[2-(4-tert-Butyl-phe- noxy)-eth- ylsulfamoyl]-2-meth- yl-benzoic acid methyl ester | 100% yield. MS: 404.1 (M − 1) |
| 74 | 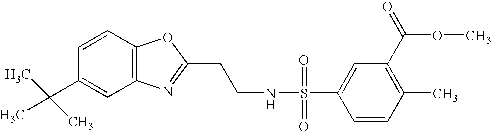 | 5-[2-(5-tert-Butyl-benzo- oxazol-2-yl)-eth- ylsulfamoyl]-2-meth- yl-benzoic acid methyl ester | 15% yield. MS: 431.1 (M + 1) |
| 75 | 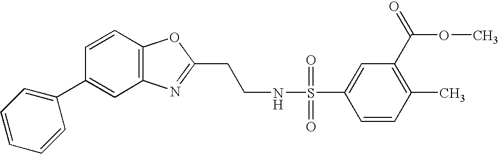 | 2-Methyl-5-[2-(5-phe- nyl-benzo- oxazol-2-yl)-eth- ylsulfamoyl]-benzoic acid methyl ester | 14% yield. MS: 451.0 (M + 1) |
| 76 | 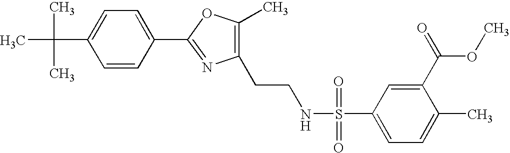 | 5-{2-[2-(4-tert-Butyl-phe- nyl)-5-methyl-oxa- zol-4-yl]-eth- ylsulfamoyl}-2-meth- yl-benzoic acid methyl ester | 41% yield. MS: 471.4 (M + 1) |
| 77 | 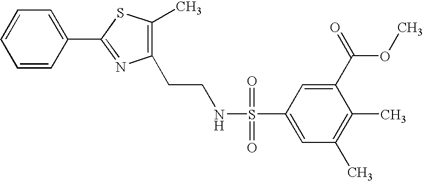 | 2,3-Dimethyl-5-[2-(5-meth- yl-2-phenyl-thia- zol-4-yl)-eth- ylsulfamoyl]-benzoic acid methyl ester | 67% yield. MS: 445.3 (M + 1) |
| 78 | 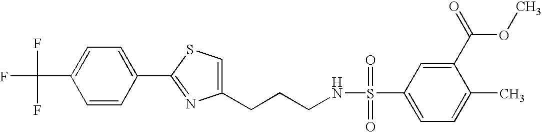 | 2-Methyl-5-{3-[2-(4-tri- fluoromethyl-phe- nyl)-thiazol-4-yl]-pro- pylsulfamoyl}-benzoic acid methyl ester | 22% yield. MS: 499.3 (M + 1) |
| 79 | 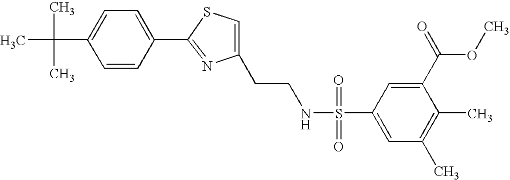 | 5-{2-[2-(4-tert-Butyl-phe- nyl)-thiazol-4-yl]-eth- ylsulfamoyl}-2,3-di- methyl-benzoic acid methyl ester | 50% yield. MS: 487.3 (M + 1) |
| 80 | 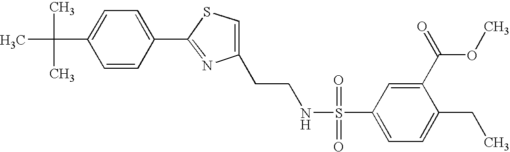 | 5-{2-[2-(4-tert-Butyl-phe- nyl)-thiazol-4-yl]-eth- ylsulfamoyl}-2-eth- yl-benzoic acid methyl ester | 41% yield. MS: 487.4 (M + 1) |
| 81 | 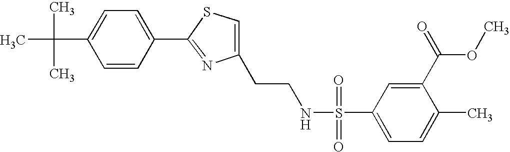 | 5-{2-[2-(4-tert-Butyl-phe- nyl)-thiazol-4-yl]-eth- ylsulfamoyl}-2-meth- yl-benzoic acid methyl ester | 56% yield. MS: 473.3 (M + 1) |
| 82 | 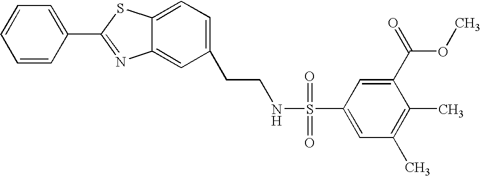 | 2,3-Dimethyl-5-[2-(2-phe- nyl-benzo- thiazol-5-yl)-eth- ylsulfamoyl]-benzoic acid methyl ester | 14% yield. MS: 481.3 (M + 1) |
| 83 | 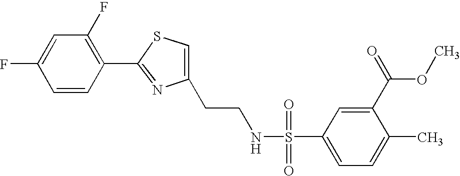 | 5-{2-[2-(2,4-Di- fluoro-phenyl)-thia- zol-4-yl]-eth- ylsulfamoyl}-2-meth- yl-benzoic acid methyl ester | 64% yield. MS: 453.3 (M + 1) |
| 84 | 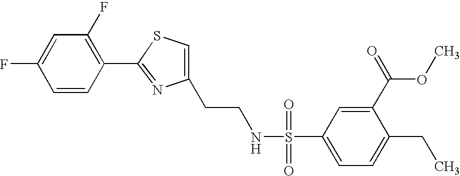 | 5-{2-[2-(2,4-Di- fluoro-phenyl)-thia- zol-4-yl]-eth- ylsulfamoyl}-2-eth- yl-benzoic acid methyl ester | 51% yield. MS: 467.3 (M + 1) |
| 85 | 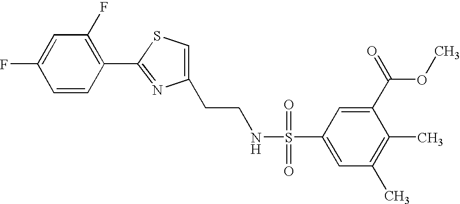 | 5-{[2-(2,4-Di- fluoro-phenyl)-thia- zol-4-yl]-eth- ylsulfamoyl}-2,3-di- methyl-benzoic acid methyl ester | 67% yield. MS: 467.3 (M + 1) |
| 86 | 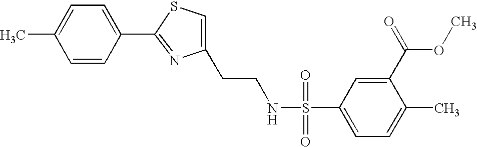 | 2-Methyl-5-[2-(2-p-to- lyl-thiazol-4-yl)-eth- ylsulfamoyl]-benzoic acid methyl ester | 57% yield. MS: 431.3 (M + 1) |
| 87 | 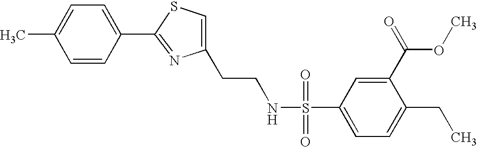 | 2-Ethyl-5-[2-(2-p-to- lyl-thiazol-4-yl)-eth- ylsulfamoyl]-benzoic acid methyl ester | 53% yield. MS: 445.4 (M + 1) |
| 88 | 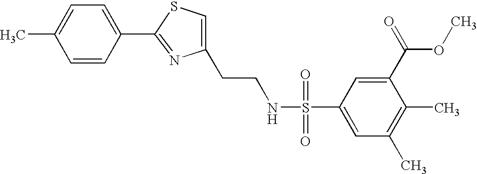 | 2,3-Dimethyl-5-[2-(2-p-to- lyl-thiazol-4-yl)-eth- ylsulfamoyl]-benzoic acid methyl ester | 67% yield. MS: 445.4 (M + 1) |
| 89 | 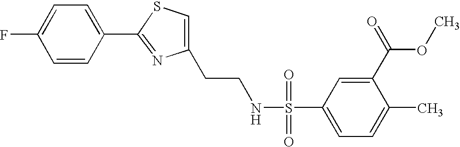 | 5-{2-[2-(4-Fluoro-phe- nyl)-thiazol-4-yl]-eth- ylsulfamoyl}-2-meth- yl-benzoic acid methyl ester | 55% yield. MS: 435.3 (M + 1) |
| 90 | 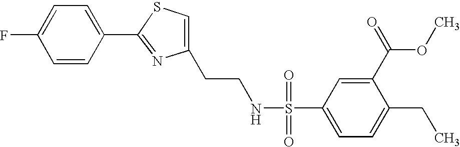 | 2-Ethyl-5-{2-[2-(4-fluor- o-phenyl)-thia- zol-4-yl]-eth- ylsulfamoyl}-benzoic acid methyl ester | 44% yield. MS: 449.3 (M + 1) |
| 91 | 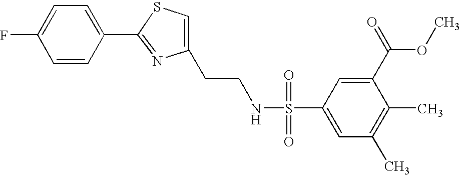 | 5-{2-[2-(4-Fluoro-phe- nyl)-thiazol-4-yl]-eth- ylsulfamoyl}-2,3-di- methyl-benzoic acid methyl ester | 68% yield. MS: 449.3 (M + 1) |
| 92 | 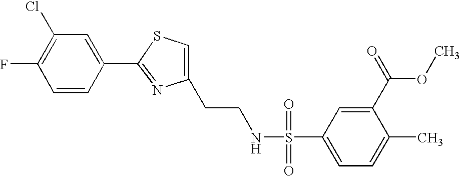 | 5-{2-[2-(3-Chloro-4-fluor- o-phenyl)-thia- zol-4-yl]-eth- ylsulfamoyl}-2-meth- yl-benzoic acid methyl ester | 31% yield. MS: 469.3 (M + 1) |
| 93 | 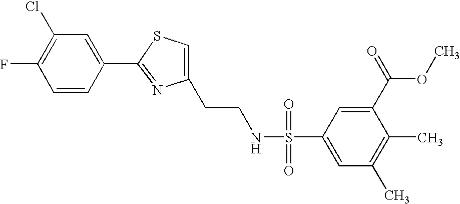 | 5-{2-[2-(3-Chloro-4-fluor- o-phenyl)-thia- zol-4-yl]-eth- ylsulfamoyl}-2,3-di- methyl-benzoic acid methyl ester | 59% yield. MS: 483.3 (M + 1) |
| 94 | 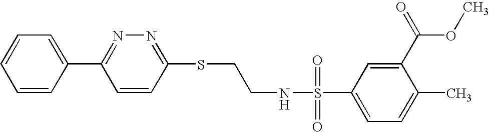 | 2-Methyl-5-[2-(6-phe- nyl-pyridazin-3-yl- sulfanyl)-eth- ylsulfamoyl]-benzoic acid methyl ester | 70% yield. MS: 444.3 (M + 1) |
| 95 | 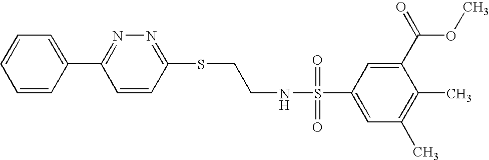 | 2,3-Dimethyl-5-[2-(6-phe- nyl-pyridazin-3-yl- sulfanyl)-eth- ylsulfamoyl]-benzoic acid methyl ester | 73% yield. MS: 458.3 (M + 1) |
| 96 | 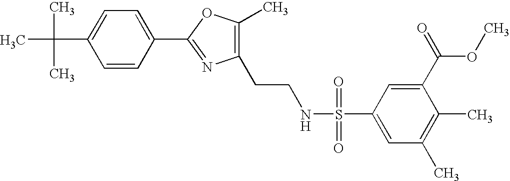 | 5-{2-[2-(4-tert-Bu- tyl-phenyl)-5-meth- yl-oxazol-4-yl]-eth- ylsulfamoyl}-2,3-di- methyl-benzoic acid methyl ester | 8% yield. MS: 485.4 (M + 1) |
| 97 | 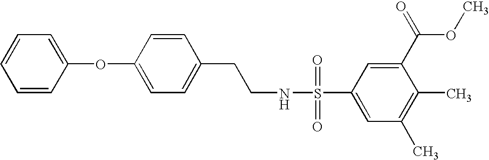 | 2,3-Dimethyl-5-[2-(4-phe- noxy-phenyl)-eth- ylsulfamoyl]-benzoic acid methyl ester | 89% yield. MS: 440.4 (M + 1) |
| 98 | 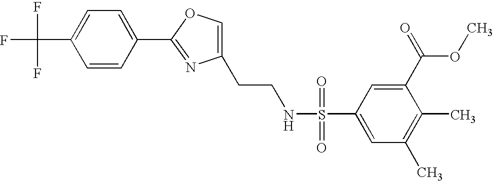 | 2,3-Dimethyl-5-{2-[2-(4-tri- fluoromethyl-phe- nyl)-oxazol-4-yl]-eth- ylsulfamoyl}-benzoic acid methyl ester | 21% yield. MS: 483.4 (M + 1) |
| 99 | 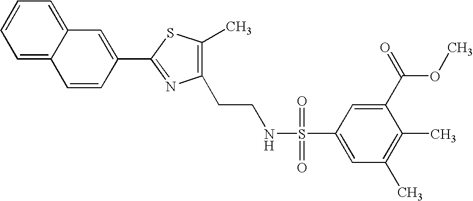 | 2,3-Dimethyl-5-[2-(5-meth- yl-2-naph- thalen-2-yl-thia- zol-4-yl)-eth- ylsulfamoyl]-benzoic acid methyl ester | 31% yield. MS: 495.4 (M + 1) |
| 100 | 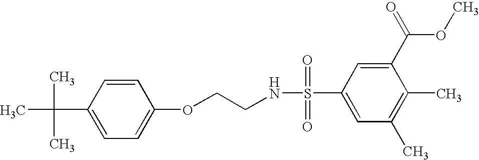 | 5-[2-(4-tert-Butyl-phe- noxy)-eth- ylsulfamoyl]-2,3-di- methyl-benzoic acid methyl ester | 26% yield. MS: 418.5 (M + 1) |
| 101 | 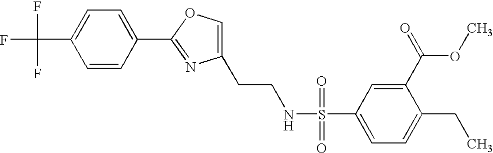 | 2-Ethyl-5-{2-[2-(4-tri- fluoromethyl-phe- nyl)-oxazol-4-yl]-eth- ylsulfamoyl}-benzoic acid methyl ester | 5% yield. MS: 483.4 (M + 1) |
| 102 | 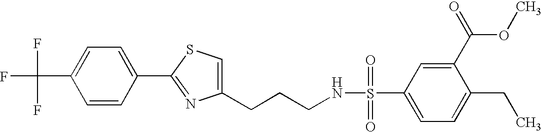 | 2-Ethyl-5-{3-[2-(4-tri- fluoromethyl-phe- nyl)-thiazol-4-yl]-pro- pylsulfamoyl}-benzoic acid methyl ester | 55% yield. MS: 513.3 (M + 1) |
| 103 | 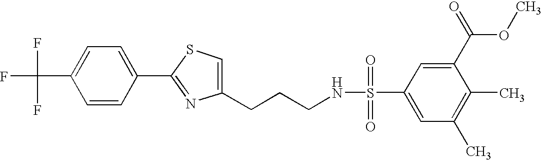 | 2,3-Dimethyl-5-{3-[2-(4-tri- fluoromethyl-phe- nyl)-thiazol-4-yl]-pro- pylsulfamoyl}-benzoic acid methyl ester | 69% yield. MS: 513.3 (M + 1) |
| 104 | 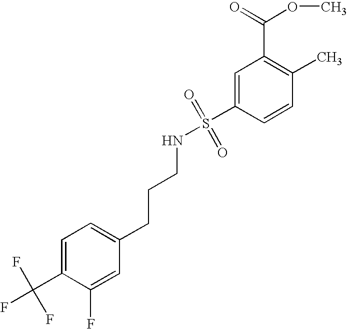 | 5-[3-(3-Fluoro-4-tri- fluoromethyl-phe- nyl)-propyl- sulfamoyl]-2-meth- yl-benzoic acid methyl ester | 18% yield. MS: 434.4 (M + 1) |
| 105 | 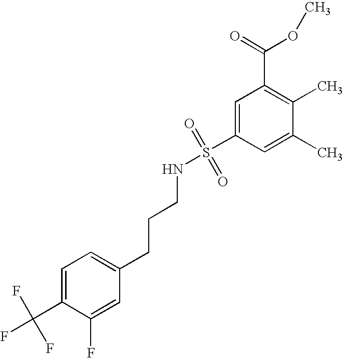 | 5-[3-(3-Fluoro-4-tri- fluoromethyl-phe- nyl)-propyl- sulfamoyl]-2,3-di- methyl-benzoic acid methyl ester | 20% yield. MS: 446.4 (M + 1) |
| 106 | 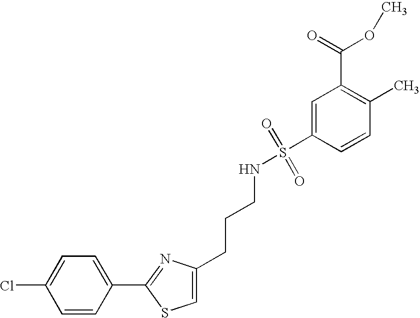 | 5-{3-[2-(4-Chloro-phe- nyl)-thiazol-4-yl]-pro- pylsulfamoyl}-2-meth- yl-benzoic acid methyl ester | 46% yield. MS: 465.2 (M + 1) |
| 107 | 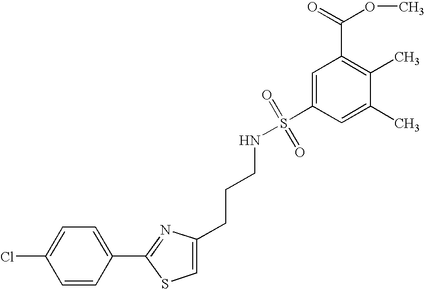 | 5-{3-[2-(4-Chloro-phe- nyl)-thiazol-4-yl]-pro- pylsulfamoyl}-2,3-di- methyl-benzoic acid methyl ester | 57% yield. MS: 479.2 (M) |
| 108 | 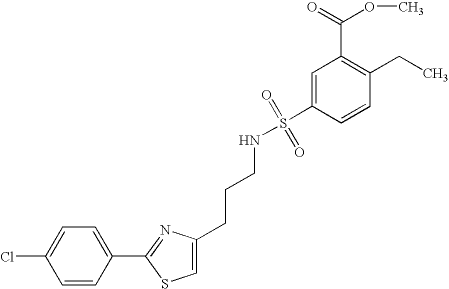 | 5-{3-[2-(4-Chloro-phe- nyl)-thiazol-4-yl]-pro- pylsulfamoyl}-2-eth- yl-benzoic acid methyl ester | 47% yield. MS: 479.2 (M + 1) |
| 109 | 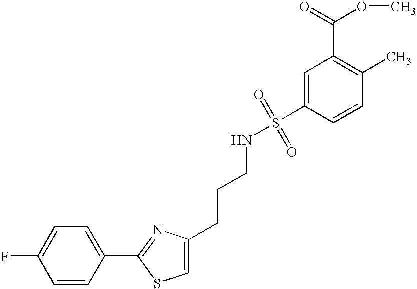 | 5-{3-[2-(4-Fluoro-phe- nyl)-thiazol-4-yl]-pro- pylsulfamoyl}-2-meth- yl-benzoic acid methyl ester | 17% yield. MS: 449.2 (M + 1) |
| 110 | 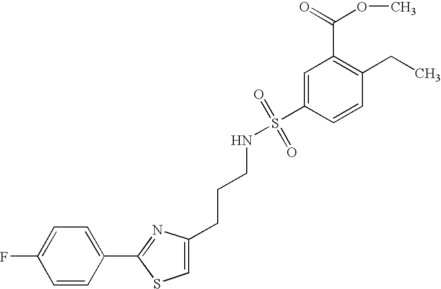 | 2-Ethyl-5-{3-[2-(4-fluor- o-phenyl)-thia- zol-4-yl]-propyl- sulfamoyl}-benzoic acid methyl ester | 13% yield. MS: 463.2 (M + 1) |
| 111 | 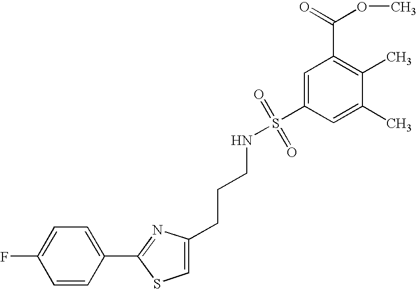 | 5-{3-[2-(4-Fluoro-phe- nyl)-thiazol-4-yl]-pro- pylsulfamoyl}-2,3-di- methyl-benzoic acid methyl ester | 11% yield. MS: 463.2 (M + 1) |
| 112 | 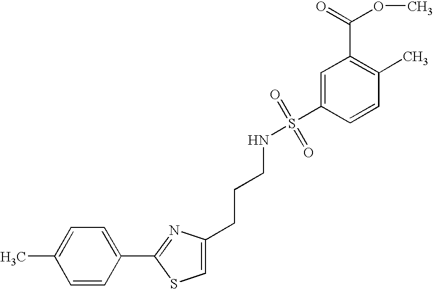 | 2-Methyl-5-[3-(2-p-to- lyl-thiazol-4-yl)-pro- pylsulfamoyl]-benzoic acid methyl ester | 30% yield. MS: 445.2 (M + 1) |
| 113 | 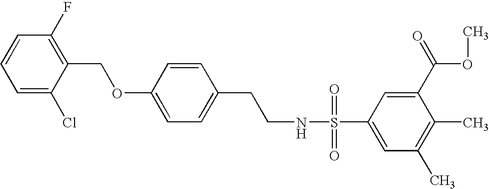 | 5-{2-[4-(2-Chloro-6-fluor- o-benzyloxy)-phe- nyl]-ethyl- sulfamoyl}-2,3-di- methyl-benzoic acid methyl ester | 6% yield. MS: 506.2 (M + 1) |
| 114 | 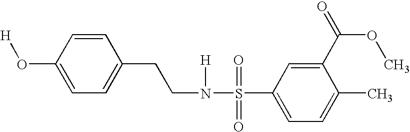 | 5-[2-(4-Hydroxy-phe- nyl)-eth- ylsulfamoyl]-2-meth- yl-benzoic acid methyl ester | 74% yield. MS: 348.2 (M − 1) |
| 115 | 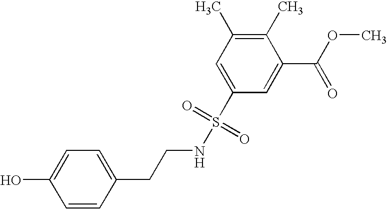 | 5-[2-(4-Hydroxy-phe- nyl)-eth- ylsulfamoyl]-2,3-di- methyl-benzoic acid methyl ester | 82% yield. MS: 364.2 (M + 1) |
| 116 | 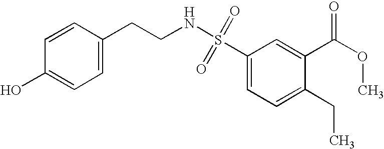 | 2-Ethyl-5-[2-(4-hy- droxy-phenyl)-eth- ylsulfamoyl]-benzoic acid methyl ester | 62% yield. MS: 362.0 (M − 1) |
| 117 | 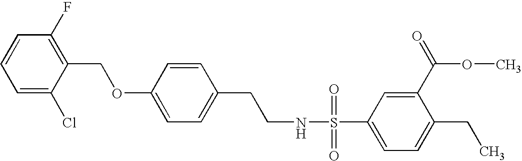 | 5-{2-[4-(2-Chloro-6-fluor- o-benzyloxy)-phe- nyl]-ethyl- sulfamoyl}-2-eth- yl-benzoic acid methyl ester | 20% yield. MS: 506.2 (M + 1) |
| 118 | 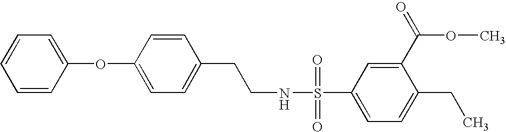 | 2-Ethyl-5-[2-(4-phe- noxy-phenyl)-eth- ylsulfamoyl]-benzoic acid methyl ester | 53% yield. MS: 440.2 (M + 1) |
| 119 | 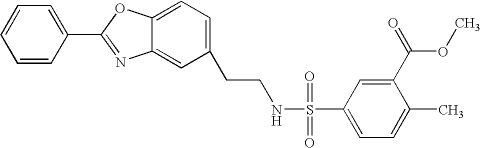 | 2-Methyl-5-[2-(2-phe- nyl-benzo- oxazol-5-yl)-eth- ylsulfamoyl]-benzoic acid methyl ester | 65% yield MS: 449.3 (M − 1) |
| 120 | 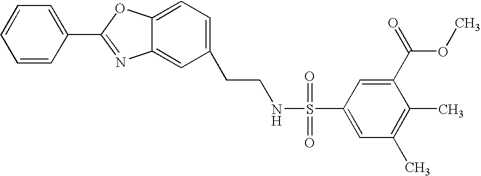 | 2,3-Dimethyl-5-[2-(2-phe- nyl-benzo- oxazol-5-yl)-eth- ylsulfamoyl]-benzoic acid methyl ester | 59% yield MS: 463.3 (M − 1) |
| 121 | 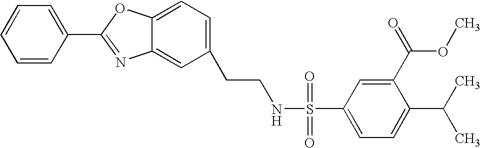 | 2-Isopropyl-5-[2-(2-phe- nyl-benzo- oxazol-5-yl)-eth- ylsulfamoyl]-benzoic acid methyl ester | 36% yield MS: 477.4 (M − 1) |
| 122 | 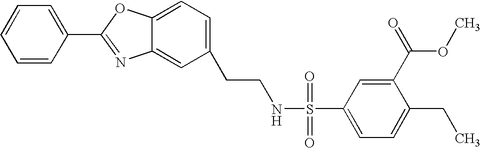 | 2-Ethyl-5-[2-(2-phe- nyl-benzo- oxazol-5-yl)-eth- ylsulfamoyl]-benzoic acid methyl ester | 53% yield MS: 465.4 (M − 1) |
| 123 | 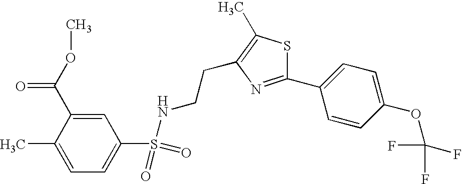 | 2-Methyl-5-(2-[5-meth- yl-2-(4-tri- fluoromethoxy-phe- nyl)-thiazol-4-yl]-eth- ylsulfamoyl}-benzoic acid methyl ester | 18% yield 515.3 (M + 1) |
| 124 | 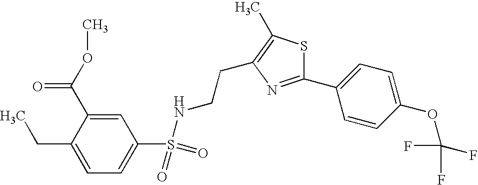 | 2-Ethyl-5-{2-[5-meth- yl-2-(4-tri- fluoromethoxy-phe- nyl)-thiazol-4-yl]-eth- ylsulfamoyl}-benzoic acid methyl ester | 23% yield MS: 527.3 (M − 1) |
| 125 | 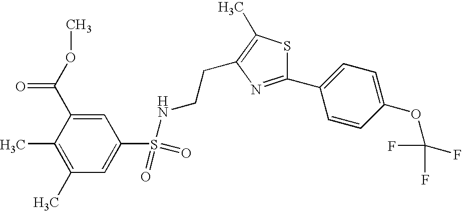 | 2,3-Dimethyl-5-{2-[5-meth- yl-2-(4-tri- fluoromethoxy-phe- nyl)-thiazol-4-yl]-eth- ylsulfamoyl}-benzoic acid methyl ester | 30% yield MS: 529.3 (M + 1) |
| 126 | 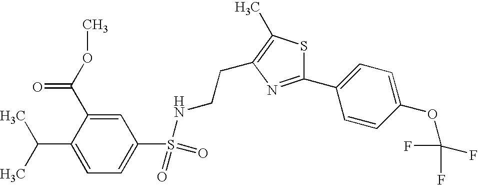 | 2-Isopropyl-5-{2-[5-meth- yl-2-(4-tri- fluoromethoxy-phe- nyl)-thiazol-4-yl]-eth- ylsulfamoyl}-benzoic acid methyl ester | 22% yield MS: 543.3 (M + 1) |
| 127 | 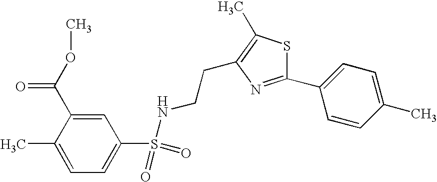 | 2-Methyl-5-[2-(5-meth- yl-2-p-tolyl-thia- zol-4-yl)-eth- ylsulfamoyl]-benzoic acid methyl ester | 33% yield 445.4 (M + 1) |
| 128 | 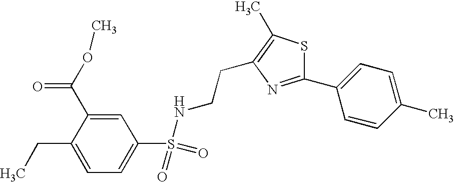 | 2-Ethyl-5-[2-(5-meth- yl-2-p-tolyl-thia- zol-4-yl)-eth- ylsulfamoyl]-benzoic acid methyl ester | 16% yield MS: 459.4 (M + 1) |
| 129 | 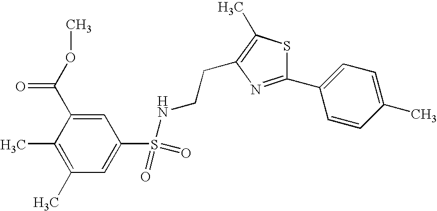 | 2,3-Dimethyl-5-[2-(5-meth- yl-2-p-tolyl-thia- zol-4-yl)-eth- ylsulfamoyl]-benzoic acid methyl ester | 27% yield MS: 459.4 (M + 1) |
| 130 | 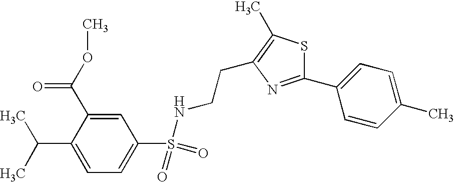 | 2-Isopropyl-5-[2-(5-meth- yl-2-p-tolyl-thia- zol-4-yl)-eth- ylsulfamoyl]-benzoic acid methyl ester | 20% yield MS: 473.4 (M + 1) |
| 131 | 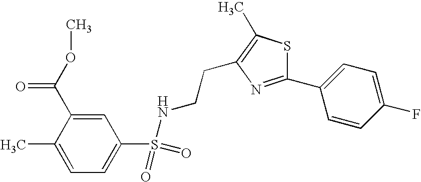 | 5-{2-[2-(4-Fluoro-phe- nyl)-5-methyl-thia- zol-4-yl]-eth- ylsulfamoyl}-2-meth- yl-benzoic acid methyl ester | 27% yield MS: 449.3 (M + 1) |
| 132 | 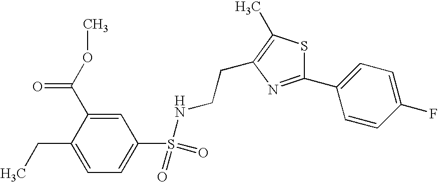 | 2-Ethyl-5-{2-[2-(4-Fluor- o-phenyl)-5-meth- yl-thiazol-4-yl]-eth- ylsulfamoyl}-benzoic acid methyl ester | 15% yield MS: 463.3 (M + 1) |
| 133 | 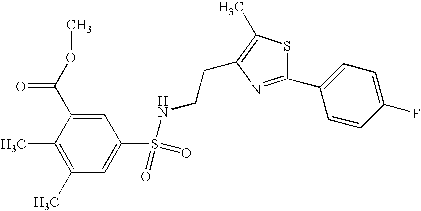 | 5-{2-[2-(4-Fluoro-phe- nyl)-5-methyl-thia- zol-4-yl]-eth- ylsulfamoyl}-2,3-di- methyl-benzoic acid methyl ester | 17% yield MS: 463.3 (M + 1) |
| 134 | 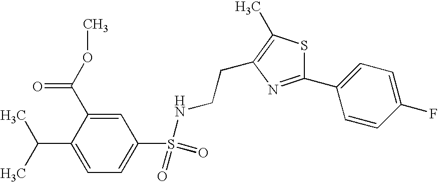 | 5-{2-[2-(4-Fluoro-phe- nyl)-5-methyl-thia- zol-4-yl]-ethyl- sulfamoyl}-2-iso- propyl-benzoic acid methyl ester | 11% yield MS: 477.4 (M + 1) |
| 135 | 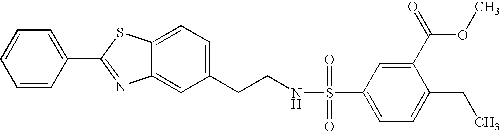 | 2-Ethyl-5-[2-(2-phenyl-benzo- thiazol-5-yl)-ethyl- sulfamoyl]-benzoic acid methyl ester | 88% yield MS: 481.3 (M + 1) |
| 136 | 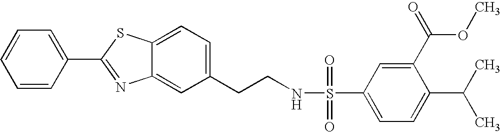 | 2-Isopropyl-5-[2-(2-phenyl-benzo- thiazol-5-yl)-ethyl- sulfamoyl]-benzoic acid methyl ester | 71% yield MS: 495.3 (M + 1) |
| 137 | 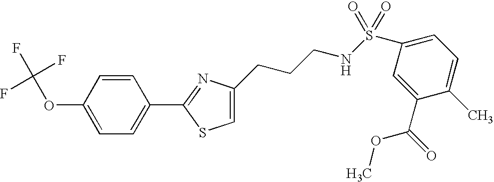 | 2-Methyl-5-{3-[2-(4-tri- fluoromethoxy-phe- nyl)-thiazol-4-yl]-pro- pylsulfamoyl}-benzoic acid methyl ester | 66% yield MS: 515.0 (M + 1) |
| 138 | 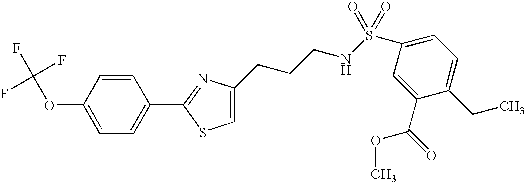 | 2-Ethyl-5-{3-[2-(4-tri- fluoromethoxy-phe- nyl)-thiazol-4-yl]-propyl- sulfamoyl}-benzoic acid methyl ester | 71% yield MS: 529.0 (M + 1) |
| 139 | 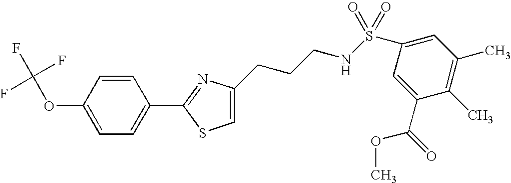 | 2,3-Dimethyl-5-{3-[2-(4-tri- fluoromethoxy-phe- nyl)-thiazol-4-yl]-propyl- sulfamoyl}-benzoic acid methyl ester | 62% yield MS: 529.0 (M + 1) |
| 140 | 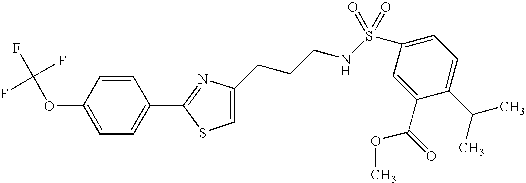 | 2-Isopropyl-5-{3-[2-(4-tri- fluoromethoxy-phe- nyl)-thiazol-4-yl]-pro- pylsulfamoyl}-benzoic acid methyl ester | 64% yield MS: 543.1 (M + 1) |
| 141 | 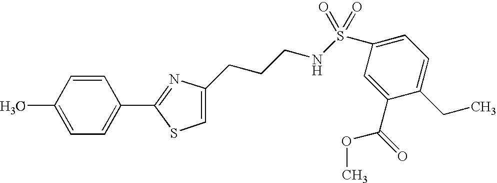 | 2-Ethyl-5-[3-(2-p-to- lyl-thiazol-4-yl)-propyl- sulfamoyl]-benzoic acid methyl ester | 87% yield MS: 459.1 (M + 1) |

### Example 142

### 5-[2-(4-Isopropyl-phenylsulfanyl)-ethylsulfamoyl]-2,3-dimethyl-benzoic acid methyl ester

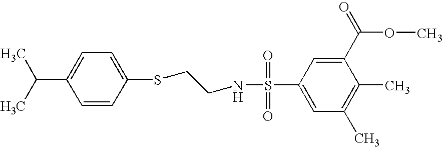

Sodium tert-butoxide (0.06 g, 0.628 mmol) was added slowly to a solution of 4-isopropylthiophenol (0.087 g, 0.571 mmol) in 10 ml anhydrous tetrahydrofuran cooled to 0° C. After stirring at room temperature for 5 min, 5-(2-bromo-ethylsulfamoyl)-2,3-dimethyl-benzoic acid methyl ester (0.20 g, 0.571 mmol) was added and the reaction mixture was stirred at room temperature overnight. The reaction mixture was then diluted with 100 ml ethyl acetate and the ethyl acetate solution was washed sequentially with 80 ml water and 80 ml brine, dried (anhydrous sodium sulfate) and concentrated under reduced pressure. The residual yellow oil (0.168 g) was purified by preparative thick layer chromatography (silica gel), eluting with 7:3 hexane/ethyl acetate to yield the title compound as a yellowish oil (0.0832 g, 35% yield).

MS: 420.3 (M−1)

The title compounds of EXAMPLES 143-171 were prepared using procedures analogous to that of EXAMPLE 142 from appropriate starting materials.

| Ex. | Compound | Compound Name | Data |
|---|---|---|---|
| 143 | 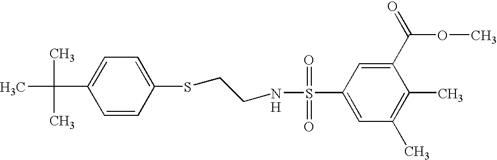 | 5-[2-(4-tert-Butyl-phenyl- sulfanyl)-eth- ylsulfamoyl]-2,3-di- methyl-benzoic acid methyl ester | 37% yield. MS: 436.3 (M + 1) |
| 144 | 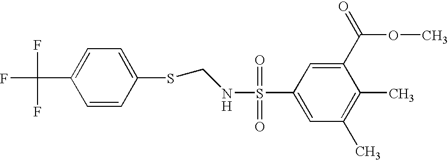 | 2,3-Dimethyl-5-[2-(4-tri- fluoromethyl-phenyl- sulfanyl)-eth- ylsulfamoyl]-benzoic acid methyl ester | 43% yield. MS: 446.2 (M − 1) |
| 145 | 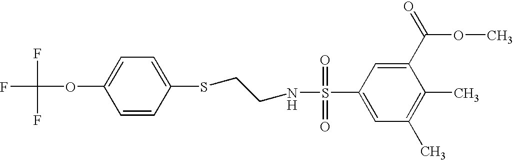 | 2,3-Dimethyl-5-[2-(4-tri- fluoromethoxy-phenyl- sulfanyl)-eth- ylsulfamoyl]-benzoic acid methyl ester | 52% yield. MS: 462.2 (M − 1) |
| 146 | 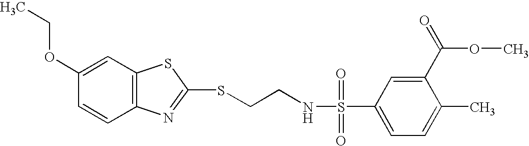 | 5-[2-(6-Ethoxy-benzo- thiazol-2-yl- sulfanyl)-ethyl- sulfamoyl]-2-meth- yl-benzoic acid methyl ester | 87% yield. MS: 467.2 (M + 1) |
| 147 | 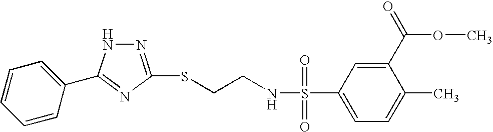 | 2-Methyl-5-[2-(5-phe- nyl-1H-[1,2,4]tri- azol-3-yl- sulfanyl)-ethyl- sulfamoyl]-benzoic acid methyl ester | 82% yield. MS: 433.3 (M + 1) |
| 148 | 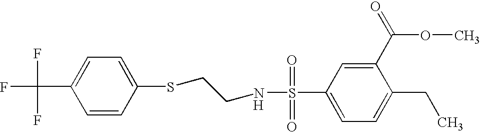 | 2-Ethyl-5-[2-(4-tri- fluoromethyl-phenyl- sulfanyl)-ethyl- sulfamoyl]-benzoic acid methyl ester | 63% yield. MS: 446.3 (M − 1) |
| 149 | 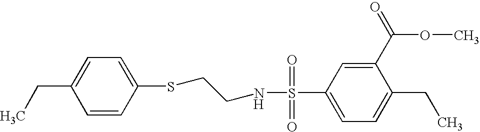 | 2-Ethyl-5-[2-(4-ethyl-phenyl- sulfanyl)-ethyl- sulfamoyl]-benzoic acid methyl ester | 49% yield. MS: 406.3 (M − 1) |
| 150 | 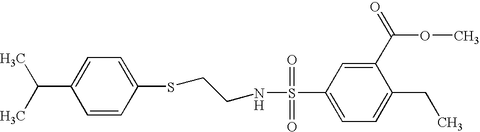 | 2-Ethyl-5-[2-(4-iso- propyl-phenyl- sulfanyl)-ethyl- sulfamoyl]-benzoic acid methyl ester | 41% yield. MS: 420.3 (M − 1) |
| 151 | 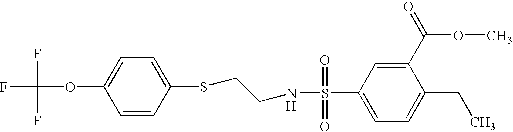 | 2-Ethyl-5-[2-(4-tri- fluoromethoxy-phenyl- sulfanyl)-ethyl- sulfamoyl]-benzoic acid methyl ester | 40% yield. MS: 462.3 (M − 1) |
| 152 | 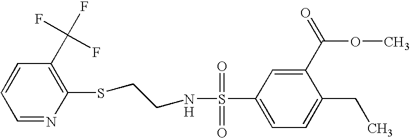 | 2-Ethyl-5-[2-(3-tri- fluoromethyl-pyri- din-2-yl- sulfanyl)-ethyl- sulfamoyl]-benzoic acid methyl ester | 79% yield. MS: 449.3 (M + 1) |
| 153 | 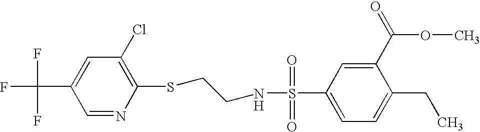 | 5-[2-(3-Chloro-5-tri- fluoromethyl-pyri- din-2-yl- sulfanyl)-ethyl- sulfamoyl]-2-eth- yl-benzoic acid methyl ester | 80% yield. MS: 483.3 (M + 1) |
| 154 | 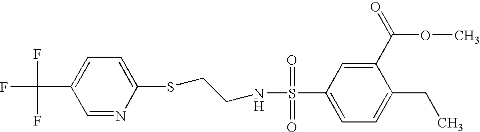 | 2-Ethyl-5-[2-(5-tri- fluoromethyl-pyri- din-2-yl- sulfanyl)-ethyl- sulfamoyl]-benzoic acid methyl ester | 68% yield. MS: 449.3 (M + 1) |
| 155 | 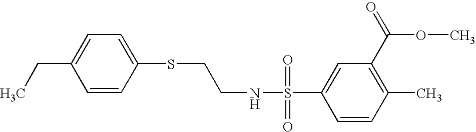 | 5-[2-(4-Ethyl-phenyl- sulfanyl)-ethyl- sulfamoyl]-2-meth- yl-benzoic acid methyl ester | 31% yield. MS: 394.2 (M + 1) |
| 156 | 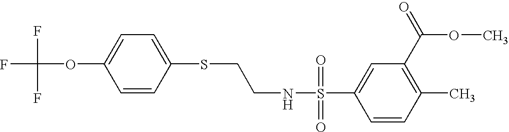 | 2-Methyl-5-[2-(4-tri- fluoromethoxy-phenyl- sulfanyl)-ethyl- sulfamoyl]-benzoic acid methyl ester | 25% yield. MS: 450.1 (M + 1) |
| 157 | 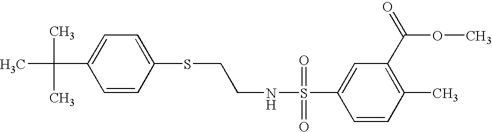 | 5-[2-(4-tert-Butyl-phenyl- sulfanyl)-ethyl- sulfamoyl]-2-meth- yl-benzoic acid methyl ester | 31% yield. MS: 422.2 (M + 1) |
| 158 | 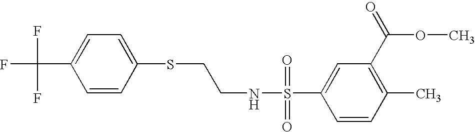 | 2-Methyl-5-[2-(4-tri- fluoromethyl-phenyl- sulfanyl)-ethyl- sulfamoyl]-benzoic acid methyl ester | 62% yield: MS: 434.1 (M + 1) |
| 159 | 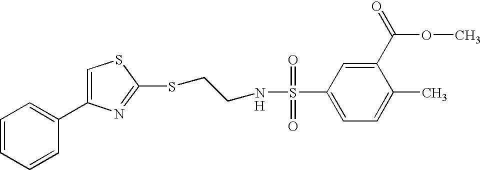 | 2-Methyl-5-[2-(4-phe- nyl-thiazol-2-yl- sulfanyl)-ethyl- sulfamoyl]-benzoic acid methyl ester | 96% yield. MS: 449.2 (M + 1) |
| 160 | 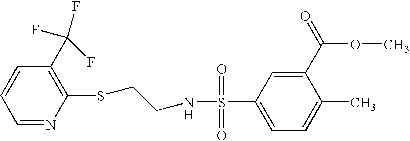 | 2-Methyl-5-[2-(3-tri- fluoromethyl-pyri- din-2-yl- sulfanyl)-ethyl- sulfamoyl]-benzoic acid methyl ester | 82% yield. MS: 435.2 (M + 1) |
| 161 | 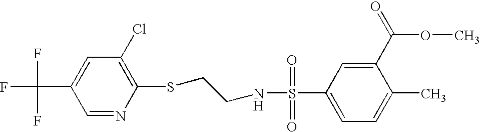 | 5-[2-(3-Chloro-5-tri- fluoromethyl-pyri- din-2-yl- sulfanyl)-ethyl- sulfamoyl]-2-meth- yl-benzoic acid methyl ester | 89% yield. MS: 469.2 (M + 1) |
| 162 | 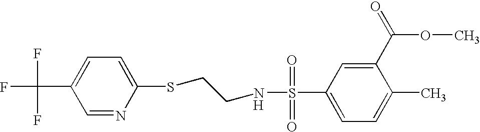 | 2-Methyl-5-[2-(5-tri- fluoromethyl-pyri- din-2-yl- sulfanyl)-ethyl- sulfamoyl]-benzoic acid methyl ester | 80% yield. MS: 435.3 (M + 1) |
| 163 | 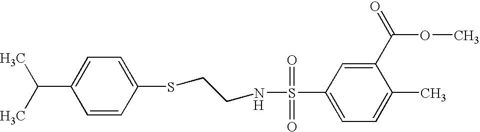 | 5-[2-(4-Isopropyl-phenyl- sulfanyl)-ethyl- sulfamoyl]-2-meth- yl-benzoic acid methyl ester | 44% yield. MS: 408.3 (M + 1) |
| 164 | 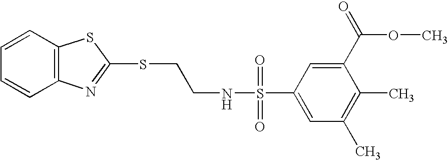 | 5-[2-(Benzo- thiazol-2-yl- sulfanyl)-ethyl- sulfamoyl]-2,3-di- methyl-benzoic acid methyl ester | 74% yield. MS: 437.3 (M + 1) |
| 165 | 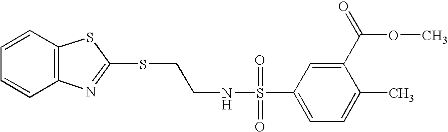 | 5-[2-(Benzo- thiazol-2-yl- sulfanyl)-ethyl- sulfamoyl]-2-meth- yl-benzoic acid methyl ester | 75% yield. MS: 423.2 (M + 1) |
| 166 | 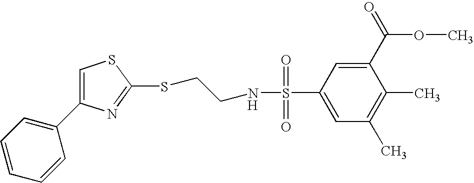 | 2,3-Dimethyl-5-[2-(4-phe- nyl-thiazol-2-yl- sulfanyl)-ethyl- sulfamoyl]-benzoic acid methyl ester | 91% yield. MS: 463.3 (M + 1) |
| 167 | 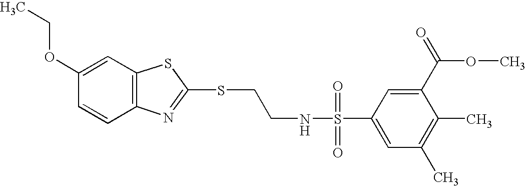 | 5-[2-(6-Ethoxy-benzo- thiazol-2-yl- sulfanyl)-ethyl- sulfamoyl]-2,3-di- methyl-benzoic acid methyl ester | 87% yield. MS: 481.3 (M + 1) |
| 168 | 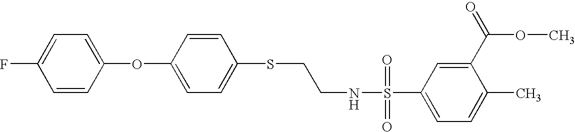 | 5-{2-[4-(4-Fluoro-phe- noxy)-phenyl- sulfanyl]-ethyl- sulfamoyl}-2-meth- yl-benzoic acid methyl ester | 32% yield. MS: 474.1 (M − 1) |
| 169 | 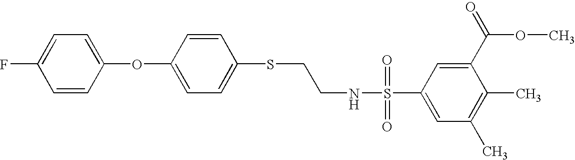 | 5-{2-[4-(4-Fluoro-phe- noxy)-phenyl- sulfanyl]-ethyl- sulfamoyl}-2,3-di- methyl-benzoic acid methyl ester | 16% yield. MS: 490.2 (M + 1) |
| 170 | 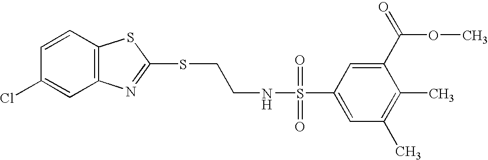 | 5-[2-(5-Chloro-benzo- thiazol-2-yl- sulfanyl)-ethyl- sulfamoyl]-2,3-di- methyl-benzoic acid methyl ester | 66% yield. MS: 471.1 (M + 1) |
| 171 | 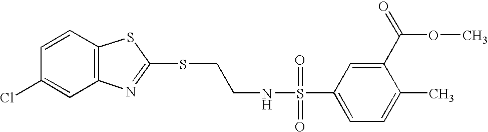 | 5-[2-(5-Chloro-benzo- thiazol-2-yl- sulfanyl)-ethyl- sulfamoyl]-2-meth- yl-benzoic methyl ester | 50% yield. MS: 457.1 (M + 1) |

### Example 172

### 2,3-Dimethyl-5-[2-(4-trifluoromethyl-phenoxy)-ethylsulfamoyl]-benzoic acid methyl ester

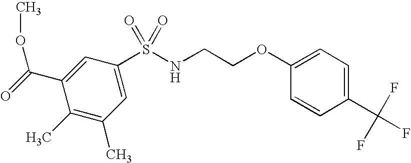

Sodium tert-butoxide (0.06 g, 0.627 mmol) was added to a solution of 4-trifluoromethylphenol (0.092 g, 0.57 mmol) in 4 ml dimethylformamide cooled to 0° C. The resulting solution was stirred at room temperature for 5 min. then a solution of 5-(2-bromo-ethylsulfamoyl)-2,3-dimethyl-benzoic acid methyl ester (0.20 g, 0.57 mmol) in 1 ml dimethylformamide was added.

The reaction mixture was stirred at 80° C. overnight, then cooled to room temperature and diluted with 80 ml ethyl acetate. The ethyl acetate solution was washed sequentially with 60 ml water and 60 ml brine, dried (anhydrous sodium sulfate) and concentrated under reduced pressure. The residual yellow oil (0.153 g) was purified by preparative thick layer chromatography (silica gel), eluting with 7:3 hexane/ethyl acetate to yield the title compound as a yellowish oil (0.0349 g, (14% yield). MS: 430.3 (M−1) acid methyl ester

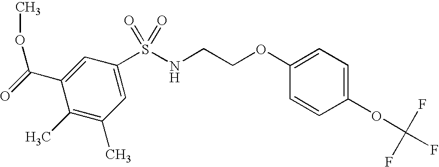

Cesium carbonate (0.372 g, 1.14 mmol) was added to a solution of 4-trifluoromethoxyphenol (0.102 g, 0.57 mmol) in 4 ml dimethylformamide. After stirring at room temperature for 15 min, a solution of 5-(2-bromo-ethylsulfamoyl)-2,3-dimethyl-benzoic acid methyl ester (0.2 g, 0.57 mmol) in 1 ml dimethylformamide was added and the reaction mixture was stirred at room temperature overnight. The reaction mixture was diluted with 80 ml ethyl acetate and the ethyl acetate solution was washed sequentially with 60 ml water and 60 ml brine, dried (anhydrous sodium sulfate) and concentrated under reduced pressure to a yellow oil (0.219 g). The crude product was purified by preparative thick layer chromatography (silica gel), eluting with 85:15 hexane/ethyl acetate to yield the title compound as a yellowish oil (0.043 g, 17% yield). MS: 448.3 (M+1)

### Example 174

### 2-Methyl-5-[2-(4′-trifluoromethoxy-biphenyl-4-yl)-ethylsulfamoyl]-benzoic acid methyl ester

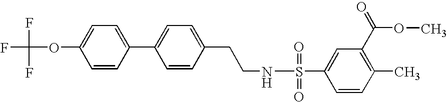

A solution of 5-[2-(4-bromo-phenyl)-ethylsulfamoyl]-2-methyl-benzoic acid methyl ester (0.20 g, 0.485 mmol), 4-trifluoromethoxybenzeneboronic acid (0.25 g, 1.21 mmol), potassium carbonate (0.485 ml of 2M aqueous solution, 0.971 mmol), 1,1′-bis(diphenylphosphino)ferrocene (0.013 g, 0.024 mmol), and 1,1′-bis(diphenylphosphino)ferrocenedichloropalladium(II) complex with dichloromethane (0.0198 g, 0.024 mmol) in 10 ml dioxane was degassed and backfilled with nitrogen 5 times. The reaction mixture was heated at reflux overnight, then cooled to room temperature and poured into 70 ml water. The aqueous solution was extracted with 2×70 ml ethyl acetate and the combined ethyl acetate extracts were washed with 100 ml brine, dried (anhydrous sodium sulfate) and concentrated under reduced pressure. The residual brownish oil (0.262 g) was purified by flash column chromatography (15 g silica gel), eluting with 85:15 hexane/ethyl acetate to give the title compound as an off-white solid (0.147 g, 62% yield). MS: 494.0 (M+1)

The title compounds of EXAMPLES 175-191 were prepared using procedures analogous to that of EXAMPLE 174 from appropriate starting materials.

| Ex. | Compound | Compound Name | Data |
|---|---|---|---|
| 175 | 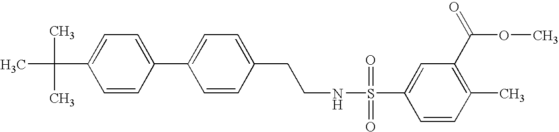 | 5-[2-(4′-tert-Butyl- biphenyl-4-yl)- ethylsulfamoyl]-2- methyl-benzoic acid methyl ester | 70% yield. MS: 466.1 (M + 1) |
| 176 | 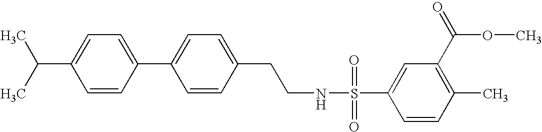 | 5-[2-(4′-Isopropyl- biphenyl-4-yl)- ethylsulfamoyl]-2- methyl-benzoic acid methyl ester | 83% yield. MS: 452.1 (M + 1) |
| 177 | 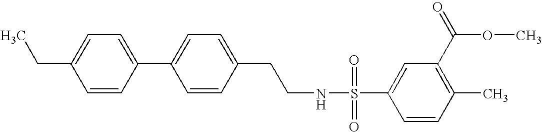 | 5-[2-(4′-Ethyl- biphenyl-4-yl)- ethylsulfamoyl]-2- methyl-benzoic acid methyl ester | 85% yield. MS: 438.1 (M + 1) |
| 178 | 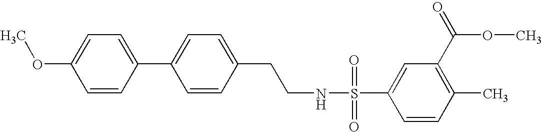 | 5-[2-(4′-Methoxy- biphenyl-4-yl)- ethylsulfamoyl]-2- methyl-benzoic acid methyl ester | 100% yield. MS: 438.1 (M − 1) |
| 179 | 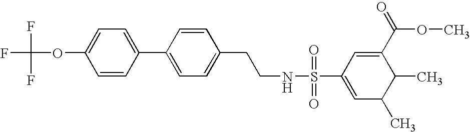 | 2,3-Dimethyl-5-[2- (4′-trifluoromethoxy- biphenyl-4-yl)- ethylsulfamoyl]- benzoic acid methyl ester | 48% yield. MS: 508.4 (M + 1) |
| 180 | 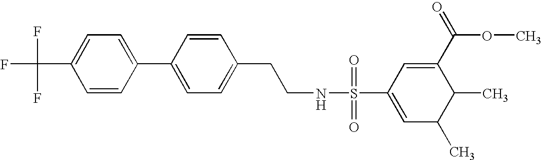 | 2,3-Dimethyl-5-[2- (4′-trifluoromethyl- biphenyl-4-yl)- ethylsulfamoyl]- benzoic acid methyl ester | 98% yield. MS: 490.4 (M + 1) |
| 181 | 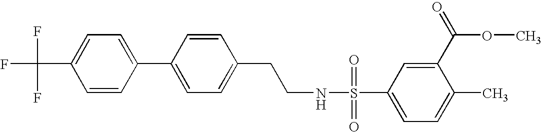 | 2-Methyl-5-[2-(4- trifluoromethyl- biphenyl-4-yl)- ethylsulfamoyl]- benzoic acid methyl ester | 68% yield. 478.4 (M + 1) |
| 182 | 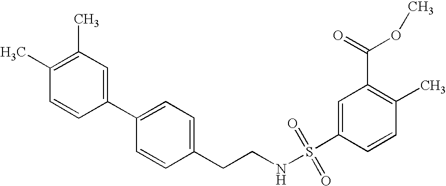 | 5-[2-(3′,4′-Dimethyl- biphenyl-4-yl)- ethylsulfamoyl]-2- methyl-benzoic acid methyl ester | 26% yield MS: 436.3 (M − 1) |
| 183 | 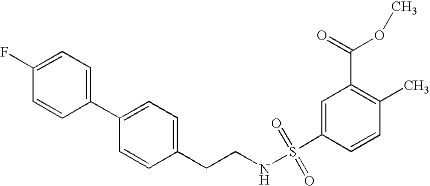 | 5-[2-(4′-Fluoro- biphenyl-4-yl)- ethylsulfamoyl]-2- methyl-benzoic acid methyl ester | 35% yield MS: 426.3 (M − 1) |
| 184 | 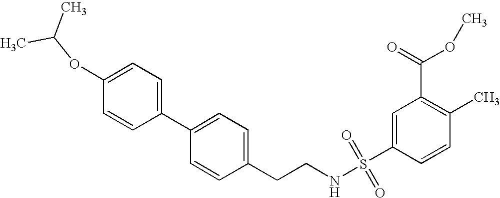 | 5-[2-(4′-Isopropoxy- biphenyl-4-yl)- ethylsulfamoyl]-2- methyl-benzoic acid methyl ester | 38% yield MS: 466.3 (M − 1) |
| 185 | 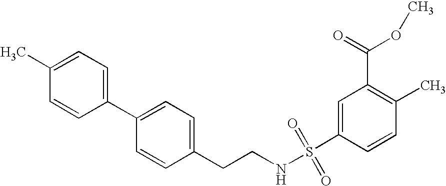 | 2-2-Methyl-5-[2-(4′- methyl-biphenyl-4- yl)-ethylsulfamoyl]- benzoic acid methyl ester | 38% yield MS: 422.3 (M − 1) |
| 186 | 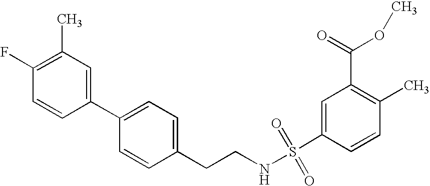 | 5-[2-(4′-Fluoro-3′- methyl-biphenyl-4- yl)-ethylsulfamoyl]- 2-methyl-benzoic acid methyl ester | 44% yield 440.3 (M − 1) |
| 187 | 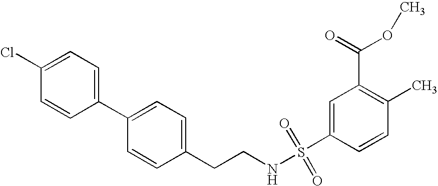 | 5-[2-(4′-Chloro- biphenyl-4-yl)- ethylsulfamoyl]-2- methyl-benzoic acid methyl ester | 50% yield MS: 442.2 (M − 1) |
| 188 | 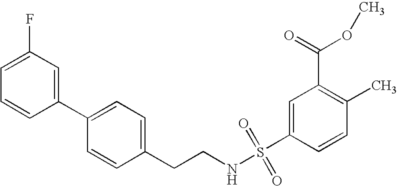 | 5-[2-(3′-Fluoro- biphenyl-4-yl)- ethylsulfamoyl]-2- methyl-benzoic acid methyl ester | 55% yield MS: 426.3 (M − 1) |
| 189 | 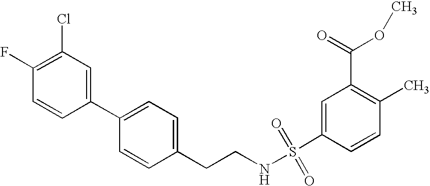 | 5-[2-(3′-Chloro-4′- fluoro-biphenyl-4- yl)-ethylsulfamoyl]- 2-methyl-benzoic acid methyl ester | 61% yield MS: 460.3 (M − 1) |
| 190 | 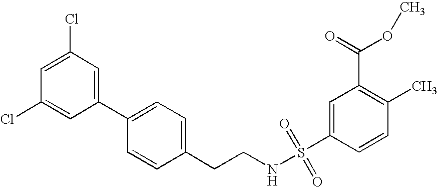 | 5-[2-(3′,5′-Dichloro- biphenyl-4-yl)- ethylsulfamoyl]-2- methyl-benzoic acid methyl ester | 56% yield MS: 477.3 (M − 1) |
| 191 | 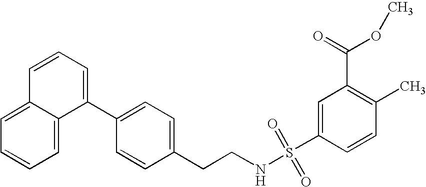 | 2-Methyl-5-[2-(4- naphthalen-1-yl- phenyl)-ethylsulfamoyl]- benzoic acid methyl ester | 53% yield MS: 458.3 (M − 1) |

### Example 192

### 5-{2-{4-(4-Chloro-phenoxy)-phenyl-ethylsulfamoyl}-2-methyl-benzoic acid methyl ester

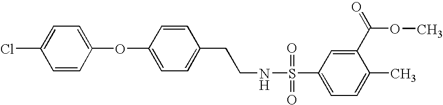

A mixture containing 5-[2-(4-hydroxy-phenyl)-ethylsulfamoyl]-2-methyl-benzoic acid methyl ester (0.167 g, 0.48 mmol), 4-chlorobenzeneboronic acid (0.15 g, 0.96 mmol), triethylamine (0.133 ml, 0.96 mmol) and cupric acetate (0.087 g, 0.48 mmol) in 5 ml methylene chloride was stirred at room temperature for 44 hr. The reaction mixture was then diluted with 35 ml methylene chloride and washed sequentially with 30 ml 1N aqueous hydrochloric acid solution, 30 ml saturated aqueous sodium bicarbonate solution, 30 ml water and 30 ml brine, dried (anhydrous sodium sulfate) and concentrated to dryness under reduced pressure. The crude product (0.186 g) was purified by flash column chromatography (15 g silica gel), eluting with 85:15 hexane/ethyl acetate to yield the title compound as a yellowish oil (0.105 g, 48% yield). MS: 460.1 (M+1)

The title compounds of EXAMPLES 193-234 were prepared using procedures analogous to that of EXAMPLE 192 from appropriate starting materials.

| Ex. | Compound | Compound Name | Data |
|---|---|---|---|
| 193 | 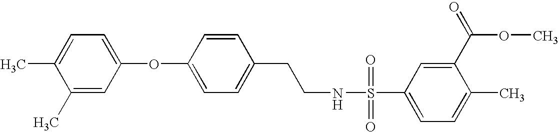 | 5-{2-[4-(3,4-Dimethyl- phenoxy)-phenyl]- ethylsulfamoyl}-2- methyl-benzoic acid methyl ester | 51% yield. MS: 454.2 (M + 1) |
| 194 | 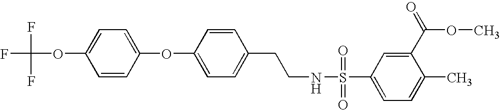 | 2-Methyl-5-{2-[4-(4- trifluoromethoxy- phenoxy)-phenyl]- ethylsulfamoyl}- benzoic acid methyl ester | 19% yield. MS: 509.1 (M + 1) |
| 195 | 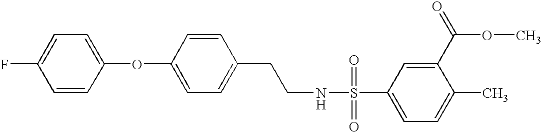 | 5-{2-[4-(4-Fluoro- phenoxy)-phenyl]- ethylsulfamoyl}-2- methyl-benzoic acid methyl ester | 39% yield. MS: 444.2 (M + 1) |
| 196 | 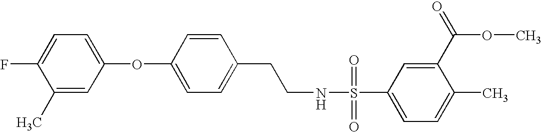 | 5-{2-[4-(4-Fluoro- 3-methyl- phenoxy)-phenyl]- ethylsulfamoyl}-2- methyl-benzoic acid methyl ester | 18% yield. MS: 458.2 (M + 1) |
| 197 | 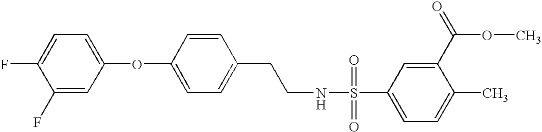 | 5-{2-[4-(3,4- Difluoro-phenoxy)-phenyl]- ethylsulfamoyl}-2- methyl-benzoic acid methyl ester | 26% yield. MS: 461.4 (M + 1) |
| 198 | 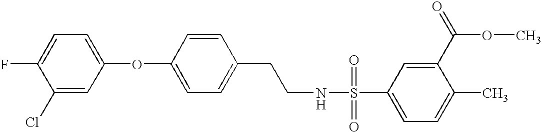 | 5-{2-[4-(3-Chloro- 4-fluoro-phenoxy)- phenyl]- ethylsulfamoyl}-2- methyl-benzoic acid methyl ester | 33% yield. MS: 478.1 (M + 1) |
| 199 | 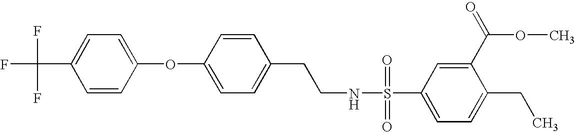 | 2-Ethyl-5-{2-[4-(4- trifluoromethyl- phenoxy)-phenyl]- ethylsulfamoyl}- benzoic acid methyl ester | 25% yield. MS: 508.2 (M + 1) |
| 200 | 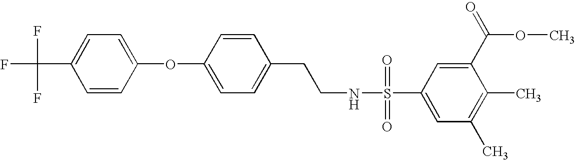 | 2,3-Dimethyl-5-{2-[4-(4- trifluoromethyl- phenoxy)-phenyl]- ethylsulfamoyl}- benzoic acid methyl ester | 28% yield. MS: 508.0 (M + 1) |
| 201 | 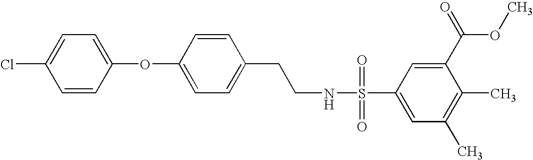 | 5-{2-[4-(4-Chloro- phenoxy)-phenyl]- ethylsulfamoyl}- 2,3-dimethyl- benzoic acid methyl ester | 11% yield MS: 472.2 (M − 1) |
| 202 | 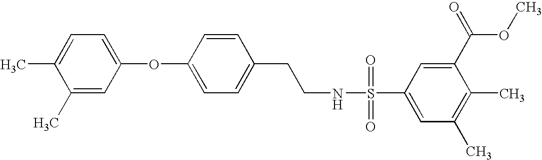 | 5-{2-[4-(3,4-Dimethyl- phenoxy)-phenyl]- ethylsulfamoyl}- 2,3-dimethyl- benzoic acid methyl ester | 46% yield MS: 466.3 (M − 1) |
| 203 | 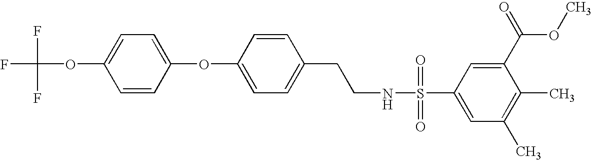 | 2,3-Dimethyl-5-{2-[4-(4- trifluoromethoxy- phenoxy)-phenyl]- ethylsulfamoyl}- benzoic acid methyl ester | 15% yield MS: 522.2 (M − 1) |
| 204 | 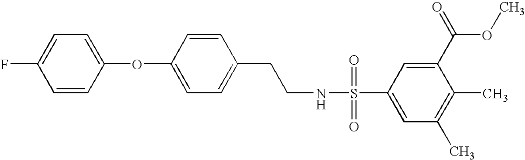 | 5-{2-[4-(4-Fluoro- phenoxy)-phenyl]- ethylsulfamoyl}- 2,3-dimethyl- benzoic acid methyl ester | 43% yield MS: 456.2 (M − 1) |
| 205 | 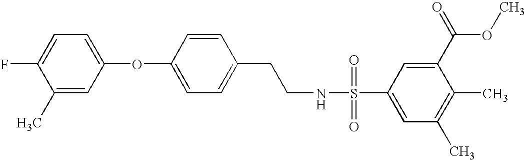 | 5-{2-[4-(4-Fluoro- 3-methyl- phenoxy)-phenyl]- ethylsulfamoyl}- 2,3-dimethyl- benzoic acid methyl ester | 14% yield MS: 470.3 (M − 1) |
| 206 | 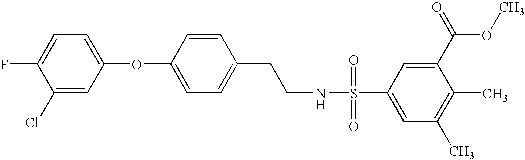 | 5-{2-[4-(3-Chloro-4-fluoro- phenoxy)-phenyl]- ethylsulfamoyl}- 2,3-dimethyl- benzoic acid methyl ester | 20% yield MS: 490.2 (M − 1) |
| 207 | 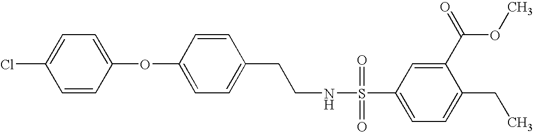 | 5-{2-[4-(4-Chloro- phenoxy)-phenyl]- ethylsulfamoyl}-2- ethyl-benzoic acid methyl ester | 18% yield MS: 472.3 (M − 1) |
| 208 | 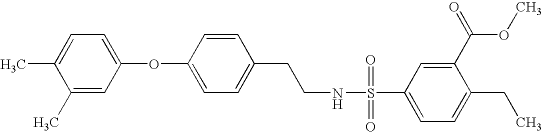 | 5-{2-[4-(3,4-Dimethyl- phenoxy)-phenyl]- ethylsulfamoyl}-2- ethyl-benzoic acid methyl ester | 66% yield MS: 466.3 (M − 1) |
| 209 | 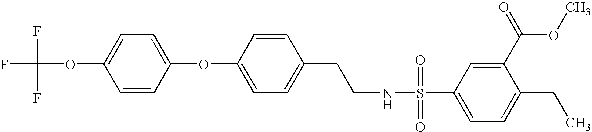 | 2-Ethyl-5-{2-[4-(4- trifluoromethoxy- phenoxy)-phenyl]- ethylsulfamoyl}- benzoic acid methyl ester | 32% yield MS: 522.3 (M − 1) |
| 210 | 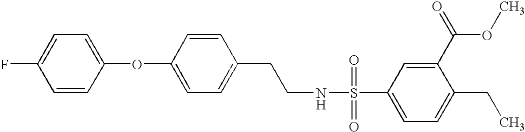 | 2-Ethyl-5-{2-[4-(4- fluoro-phenoxy)- phenyl]- ethylsulfamoyl}- benzoic acid methyl ester | 25% yield MS: 456.3 (M − 1) |
| 211 | 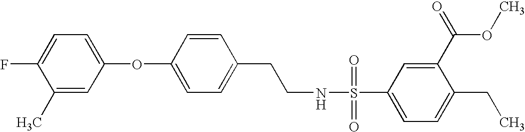 | 2-Ethyl-5-{2-[4-(4- fluoro-3-methyl- phenoxy)-phenyl]- ethylsulfamoyl}- benzoic acid methyl acid | 13% yield MS: 470.3 (M − 1) |
| 212 | 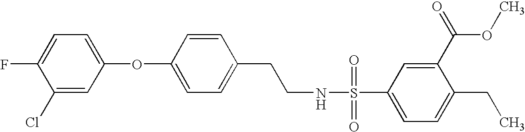 | 5-{2-[4-(3-Chloro-4-fluoro- phenoxy)-phenyl]- ethylsulfamoyl}-2- ethyl-benzoic acid methyl ester | 17% yield MS: 490.2 (M − 1) |
| 213 | 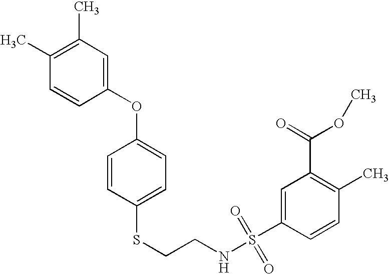 | 5-{2-[4-(3,4-Dimethyl- phenoxy)- phenylsulfanyl]- ethylsulfamoyl}-2- methyl-benzoic acid methyl ester | 29% yield MS: 486.2 (M + 1) |
| 214 | 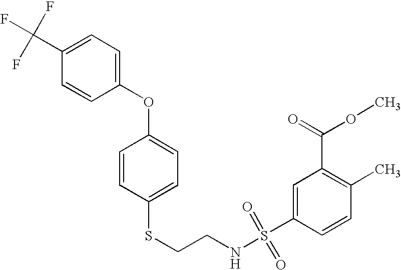 | 2-Methyl-5-{2-[4- (4-trifluoromethyl- phenoxy)- phenylsulfanyl]- ethylsulfamoyl}- benzoic acid methyl ester | 23% yield MS: 526.1 (M + 1) |
| 215 | 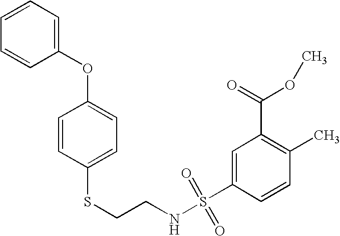 | 2-Methyl-5-[2-(4- phenoxy-phenylsulfanyl)- ethylsulfamoyl]- benzoic acid methyl ester | 27% yield MS: 458.2 (M + 1) |
| 216 | 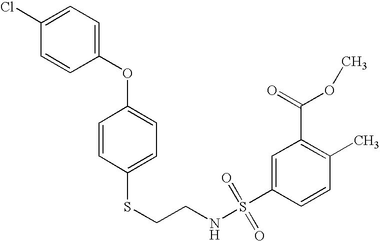 | 5-{2-[4-(4-Chloro- phenoxy)- phenylsulfanyl]- ethylsulfamoyl}-2- methyl-benzoic acid methyl ester | 14% yield MS: 492.1 (M) |

### Example 217

### 5-{2-[4-(4-Ethyl-phenoxy)-phenylsulfanyl]-ethylsulfamoyl}-2-methyl-benzoic acid ethyl ester

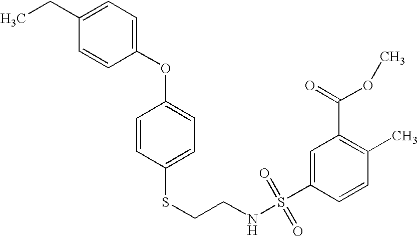

21% yield. ¹H NMR (400 MHz, CDCl₃): δ1.24 (t, 3H), 1.55 (s, 3H), 2.63 (m, 2H), 2.90 (t, 2H), 3.07 (m, 2H), 3.90 (s, 3H), 6.80 (m, 2H), 6.83 (m, 2H), 7.19 (m, 4H), 7.37 (d, 1H), 7.81 (m, 1H), 8.37 (d, 1H).

### Example 218

### 5-[2-{4-(4-Fluoro-3-methyl-phenoxy)-phenylsulfanyl]-ethylsulfamoyl}-2-methyl-benzoic acid methyl ester

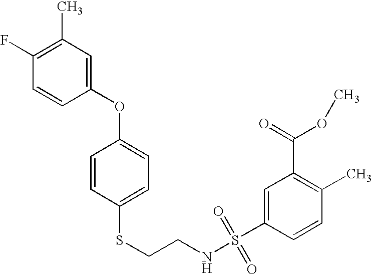

15% yield. ¹H NMR (400 MHz, CDCl₃): δ 2.25 (s, 3H), 2.66 (s, 3H), 2.90 (t, 2H), 3.08 (t, 2H), 3.91 (s, 3H), 6.80 (m, 4H), 6.98 (t, 1H), 7.20 (m, 2H), 7.37 (d, 1H), 7.82 (m, 1H), 8.37 (d, 1H).

### Example 219

### 2-Methyl-5-{2-[4-(4-trifluoromethoxy-phenoxy)-phenylsulfanyl]-ethylsulfamoyl}-benzoic acid methyl ester

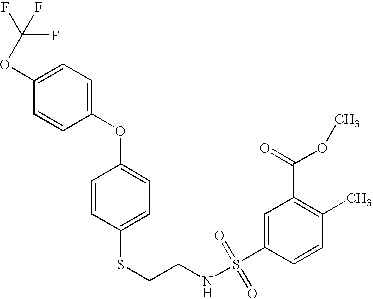

18% yield. ¹H NMR (400 MHz, CDCl₃): δ 2.65 (s, 3H), 2.93 (t, 2H), 3.10 (m, 2H), 3.91 (s, 3H), 6.86 (m, 2H), 6.99 (m, 2H), 7.22 (m, 4H), 7.39 (m, 1H), 7.82 (m, 1H), 8.37 (d, 1H).

### Example 220

### 5-{2-[4-(4-Methoxy-Phenoxy)-phenylsulfanyl]-ethylsulfamoyl}-2-methyl-benzoic acid methyl ester

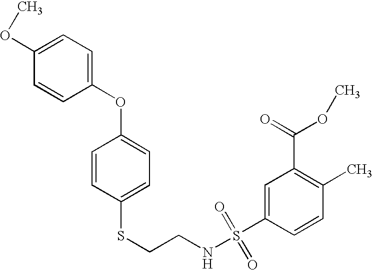

8% yield. MS: 488.3 (M+1)

### Example 221

### 2-Methyl-5-[2-(4-p-tolyloxy-phenylsulfanyl)-ethylsulfamoyl]-benzoic acid methyl ester

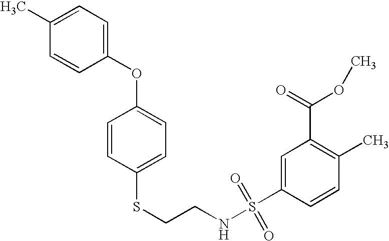

9% yield. MS: 472.3 (M+1)

### Example 222

### 5-{2-[4-(4-Isopropoxy-phenoxy)-phenylsulfanyl]-ethylsulfamoyl}-2-methyl-benzoic acid methyl ester

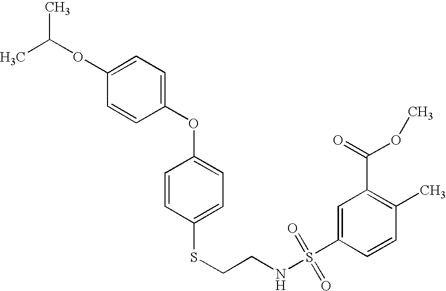

7% yield. MS: 514.2 (M+1)

### Example 223

### 2,3-Dimethyl-5-{2-[4-(4-trifluoromethyl-phenoxy)-phenylsulfanyl]-ethylsulfamoyl}-benzoic acid methyl ester

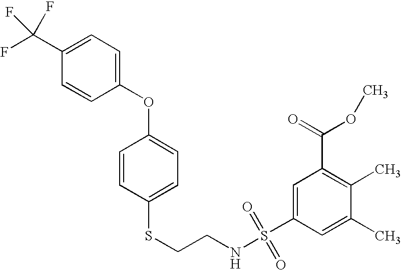

39% yield. MS: 538.3 (M−1)

### Example 224

### 2,3-Dimethyl-5-(2-[4-(4-trifluoromethoxy-phenoxy)-phenylsulfanyl]-ethylsulfamoyl}-benzoic acid methyl ester

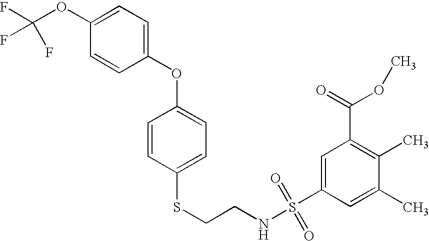

36% yield. MS: 554.3 (M−1)

### Example 225

### 2,3-Dimethyl-5-[2-(4-p-tolyloxy-phenylsulfanyl)-ethylsulfamoyl]-benzoic acid methyl ester

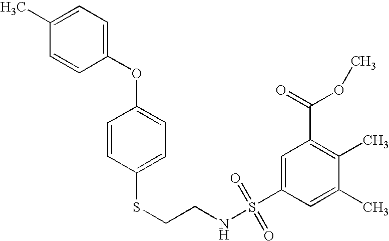

25% yield. MS: 484.3 (M−1)

### Example 226

### 5-{2-[4-(3,4-Dimethyl-phenoxy)-phenylsulfanyl]-ethylsulfamoyl}-2,3-dimethyl-benzoic acid methyl ester

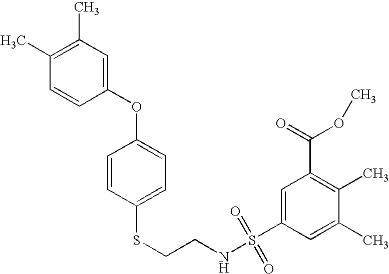

29% yield. MS: 498.4 (M−1)

### Example 227

### 5-{2-[4-(4-Methoxy-phenoxy)-phenylsulfanyl]-ethylsulfamoyl}-2,3-dimethyl-benzoic acid methyl ester

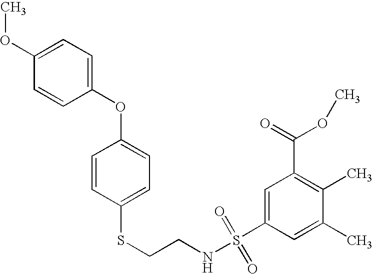

24% yield. MS: 500.4 (M−1)

### Example 228

### 5-{2-[4-(3,5-Dichloro-Phenoxy)-phenylsulfanyl]-ethylsulfamoyl}-2,3-dimethyl-benzoic acid methyl ester

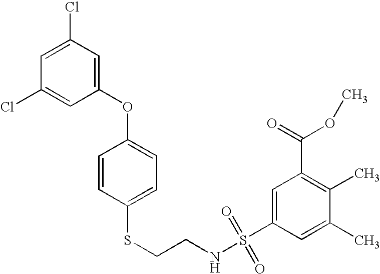

27% yield. ¹H NMR (400 MHz, CDCl₃): δ 2.37 (s, 3H), 2.51 (s, 3H), 2.98 (t, 2H), 3.12 (m, 2H), 3.90 (s, 3H), 6.85 (d, 1H), 6.9 (m, 3H), 7.1 (m, 1H), 7.27 (m, 3H), 7.72 (s, 1H), 8.10 (d, 1H).

### Example 229

### 5-{2-[4-(3-Fluoro-phenoxy)-phenylsulfanyl]-ethylsulfamoyl}-2,3-dimethyl-benzoic acid methyl ester

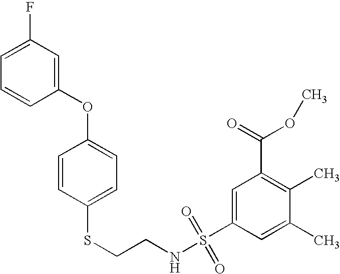

38% yield. MS: 490.4 (M+1)

### Example 230

### 2,3-Dimethyl-5-{2-[4-(naphthalen-2-yloxy)-phenylsulfanyl]-ethylsulfamoyl}-5 benzoic acid methyl ester

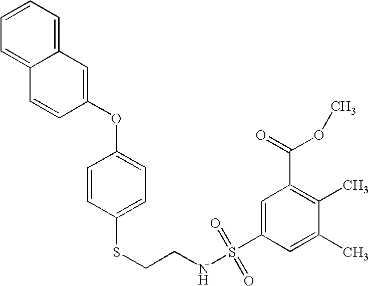

39% yield. MS: 522.4 (M+1)

### Example 231

### 5-{2-[4-(4-Ethyl-Phenoxy)-phenylsulfanyl]-ethylsulfamoyl}-2,3-dimethyl-benzoic acid methyl ester

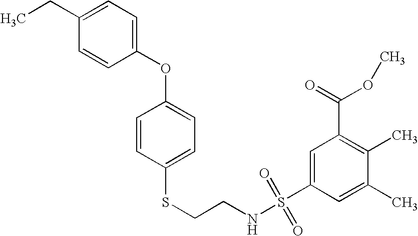

28% yield. MS: 500.4 (M+1)

### Example 232

### 5-{2-[4-(4-Fluoro-3-methyl-Phenoxy)-phenylsulfanyl]-ethylsulfamoyl}-2,3-dimethyl-benzoic acid methyl ester

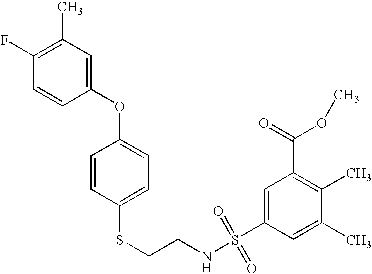

27% yield. MS: 504.4 (M+1)

### Example 233

### 5-{2-[4-(3-Chloro-4-fluoro-phenoxy)-phenylsulfanyl]-ethylsulfamoyl}-2,3-dimethyl-benzoic acid methyl ester

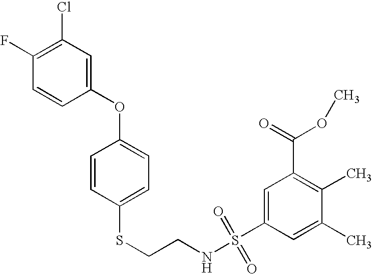

33% yield. MS: 524.5 (M)

### Example 234

### 2,3-Dimethyl-5-{2-[4-(naphthalen-1-yloxy)-phenylsulfanyl]-ethylsulfamoyl}-benzoic acid methyl ester

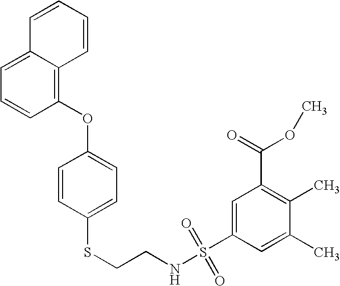

17% yield. MS: 520.3 (M−1)

The title compounds of EXAMPLES 235-240 were prepared using procedures analogous to that of EXAMPLE 192 from appropriate starting materials, in particular, using pyridine-3-boronic acid 1,3-propanediol cyclic ester and pyridine-4-boronic acid pinacol cyclic ester instead of the corresponding boronic acids.

### Example 235

### 2-Methyl-5-{2-[4-(pyridin-3-yloxy)-phenyl]-ethylsulfamoyl}-benzoic acid methyl ester

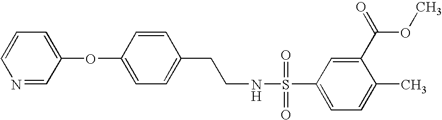

24% yield. MS: 427.2 (M+1)

### Example 236

### 2-Ethyl-5-{2-[4-(pyridin-3-yloxy)-phenyl]-ethylsulfamoyl}-benzoic acid methyl ester

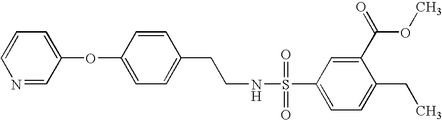

39% yield. MS: 441.2 (M+1)

### Example 237

### 2,3-Dimethyl-5-{2-[4-(Pyridin-3-yloxy)-phenyl]-ethylsulfamoyl}-benzoic acid methyl ester

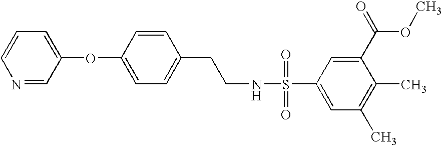

21% yield. MS: 441.2 (M+1)

### Example 238

### 2-Methyl-5-{2-[4-(pyridin-4-yloxy)-phenyl]-ethylsulfamoyl}-benzoic acid methyl ester

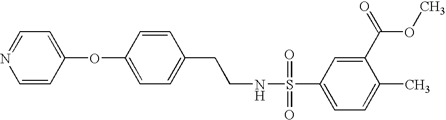

21% yield. MS: 427.2 (M+1)

### Example 239

### 2-Ethyl-5-{2-[4-(pyridin-4-yloxy)-phenyl]-ethylsulfamoyl}-benzoic acid methyl ester

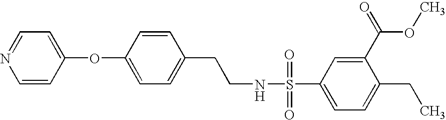

17% yield. MS: 441.2 (M+1)

### Example 240

### 2,3-Dimethyl-5-{2-[4-(pyridin-4-yloxy)-phenyl]-ethylsulfamoyl}-benzoic acid methyl ester

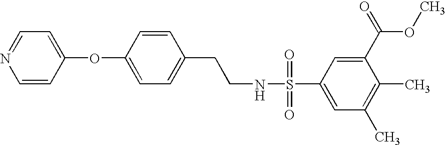

15% yield. MS: 441.2 (M+1)

### Examples 241 and 242

### 2-Methyl-5-((4-trifluoromethyl-benzyl)-{2-[4-(4-trifluoromethyl-benzyloxy)-phenyl]-ethyl}-sulfamoyl)-benzoic acid methyl ester methyl ester and 2-Methyl-5-{2-[4-(4-trifluoromethyl-benzyloxy)-phenyl]-ethylsulfamoyl}-benzoic acid methyl ester

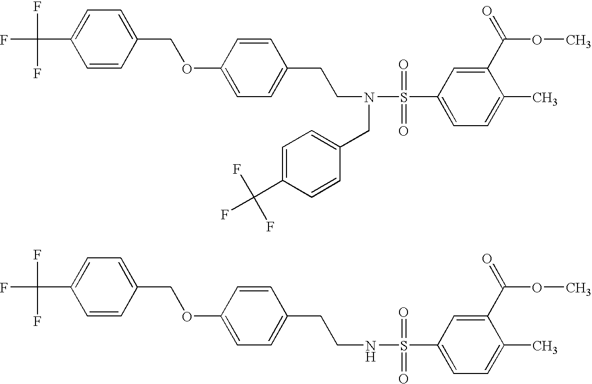

Diethyl azodicarboxylate (0.112 ml, 0.71 mmol) was added dropwise to a solution of 5-[2-(4-hydroxy-phenyl)-ethylsulfamoyl]-2-methyl-benzoic acid methyl ester (0.248 g, 0.71 mmol), 4-(trifluoromorome)benzyl alcohol (0.097 ml, 0.71 mmol) and triphenylphosphine (0.186 g, 0.71 mmol) in 5 ml anhydrous tetrahydrofuran and the resulting solution was stirred at room temperature overnight. 70 ml ethyl acetate was then added to the reaction mixture and the resulting solution was washed sequentially with 50 ml saturated aqueous sodium bicarbonate solution, 50 ml 1N aqueous hydrochloric acid solution, 50 ml water and 50 ml brine, dried (anhydrous sodium sulfate) and concentrated to dryness under reduced pressure. The crude product (0.27 g) was purified by flash column chromatography (15 g silica gel), eluting with 85:15 hexane/ethyl acetate to yield: 2-Methyl-5-((4-trifluoromethyl-benzyl)-{2-[4-(4-trifluoromethyl-benzyloxy)-phenyl]-ethyl}-sulfamoyl)-benzoic acid methyl ester, 7% yield, MS: 667.3 (M+1) and 2-Methyl-5-{2-[4-(4-trifluoromethyl-benzyloxy)-phenyl]-ethylsulfamoyl}-benzoic acid methyl ester, 35% yield, MS: 508.2 (M+1).

The title compounds of EXAMPLES 243-248 were prepared using procedures analogous to that of EXAMPLES 241 and 242 from appropriate starting materials.

### Example 243

### 5-((4-Chloro-benzyl)-{2-[4-(4-chloro-benzyloxy)-phenyl]-ethyl}-sulfamoyl)-2-methyl-benzoic acid methyl ester

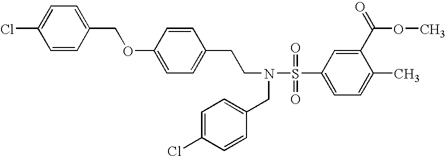

13% yield.

MS: 598.1 (M+1)

### Example 244

### 5-{2-[4-(4-Chloro-benzyloxy)-phenyl]-ethylsulfamoyl}-2-methyl-benzoic acid methyl ester

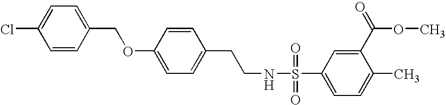

34% yield.

MS: 474.2 (M+1)

### Example 245

### 5-{Benzyl-[2-(4-benzyloxy-phenyl)-ethyl]-sulfamoyl}-2-methyl-benzoic acid methyl ester

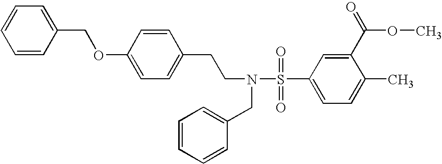

19% yield.

MS: 530.2 (M+1)

### Example 246

### 5-[2-(4-Benzyloxy-phenyl)-ethylsulfamoyl]-2-methyl-benzoic acid methyl ester

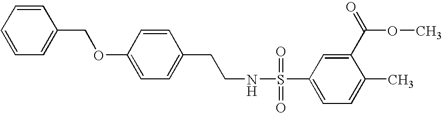

15% yield.

MS: 440.2 (M+1)

### Example 247

### 2-Methyl-5-((4-methyl-benzyl)-{2-[4-(4-methyl-benzyloxy)-phenyl]-ethyl}-sulfamoyl)-benzoic acid methyl ester

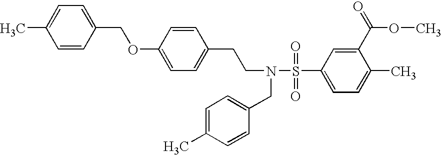

17% yield.

MS: 558.3 (M+1)

### Example 248

### 2-Methyl-5-{2-[4-(4-methyl-benzyloxy)-phenyl]-ethylsulfamoyl}-benzoic acid methyl ester

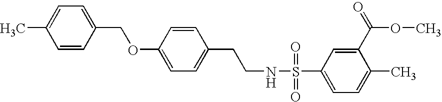

9% yield.

MS: 454.2 (M+1)

### Examples 249 and 250

### 5-((4-Fluoro-benzyl)-{2-[4-(4-fluoro-benzyloxy)-phenyl]-ethyl}-sulfamoyl)-2-methyl-benzoic acid methyl ester and 5-{2-[4-(4-Fluoro-benzyloxy)-phenyl]-ethylsulfamoyl}-2-methyl-benzoic acid methyl ester

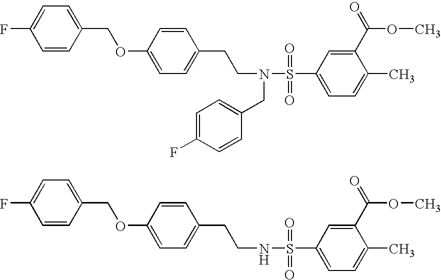

A solution of 5-[2-(4-hydroxy-phenyl)-ethylsulfamoyl]-2-methyl-benzoic acid methyl ester (0.3 g, 0.86 mmol), 4-fluorobenzyl alcohol (0.093 ml, 0.86 mmol), triphenylphosphine (0.225 g, 0.86 mmol) and diethyl azodicarboxylate (0.135 ml, 0.86 mmol) in 1 ml tetrahydrofuran was irradiated in a microwave oven (high power) at 120° C. for 5 min. The reaction mixture was cooled to room temperature and diluted with 30 ml ethyl acetate. The ethyl acetate solution was washed sequentially with 30 ml 1N aqueous hydrochloric acid, 30 ml water and 30 ml brine, dried (anhydrous sodium sulfate) and concentrated to dryness under reduced pressure. The residue (0.266 g) was purified by flash column chromatography (15 g silica gel), eluting with 85:15 hexane/ethyl acetate to yield 5-((4-Fluoro-benzyl)-{2-[4-(4-fluoro-benzyloxy)-phenyl]-ethyl}-sulfamoyl)-2-methyl-benzoic acid methyl ester, 12% yield; MS: 566.0 (M+1) and 5-{2-[4-(4-Fluoro-benzyloxy)-phenyl]-ethylsulfamoyl}-2-methyl-benzoic acid methyl ester, 40% yield; MS: 458.1 (M+1)

The title compounds of EXAMPLES 251-273 were prepared using procedures analogous to that of EXAMPLES 249 and 250 from appropriate starting materials.

### Example 251

### 5-((2,3-Difluoro-benzyl)-{2-[4-(2,3-difluoro-benzyloxy)-phenyl]-ethyl}-sulfamoyl)-2-methyl-benzoic acid methyl ester

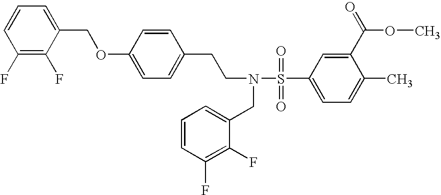

11% yield.

MS: 602.0 (M+1)

### Example 252

### 5-{2-[4-(2,3-Difluoro-benzyloxy)-phenyl]-ethylsulfamoyl}-2-methyl-benzoic acid methyl ester

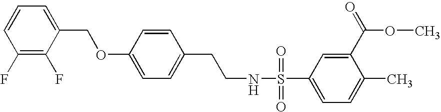

36% yield.

MS: 476.0 (M+1)

### Example 253

### 2-Methyl-5-{2-[4-(2,2,3,3-tetrafluoro-propoxy)-phenyl]-ethylsulfamoyl}-benzoic acid methyl ester

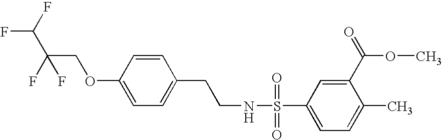

16% yield.

MS: 464.2 (M+1)

### Example 254

### 5-((3,4-Difluoro-benzyl)-{2-[4-(3,4-difluoro-benzyloxy)-phenyl]-ethyl}-sulfamoyl)-2-methyl-benzoic acid methyl ester

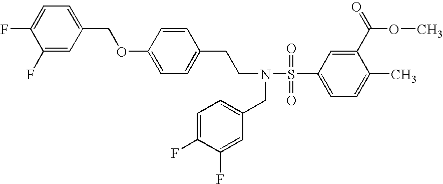

12% yield.

MS: 602.1 (M+1)

### Example 255

### 5-{2-[4-(3,4-Difluoro-benzyloxy)-phenyl]-ethylsulfamoyl}-2-methyl-benzoic acid methyl ester

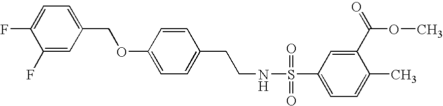

21% yield. 476.2 (M+1)

### Example 256

### 5-((3,5-Difluoro-benzyl)-{2-[4-(3,5-difluoro-benzyloxy)-phenyl]-ethyl}-sulfamoyl)-2-methyl-benzoic acid methyl ester

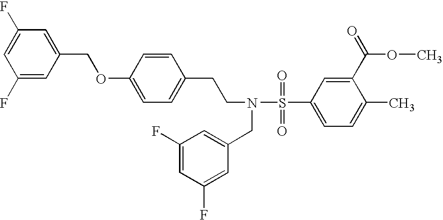

10% yield.

MS: 602.2 (M+1)

### Example 257

### 5-{2-[4-(3,5-Difluoro-benzyloxy)-phenyl]-ethylsulfamoyl}-2-methyl-benzoic acid methyl ester

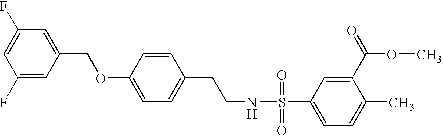

15% yield.

MS: 476.2 (M+1)

### Example 258

### 5-((3,5-Dimethyl-benzyl)-{2-[4-(3,5-dimethyl-benzyloxy)-phenyl]-ethyl}-sulfamoyl)-2-methyl-benzoic acid methyl ester

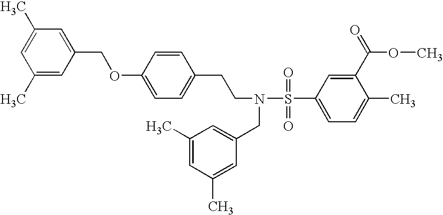

12% yield.

¹H NMR (400 MHz, CDCl₃): δ 2.25 (s, 3H), 2.31 (s, 6H), 2.59 (m, 2H), 2.67 (s, 3H), 3.28 (m, 2H), 3.90 (s, 3H), 4.27 (s, 2H), 4.92 (s, 2H), 6.84 (m, 7H), 6.95 (s, 1H), 7.02 (s, 2H), 7.36 (d, 1H), 7.78 (d, 1H), 8.34 (d, 1H).

### Example 259

### 5-{2-[4-(3,5-Dimethyl-benzyloxy)-phenyl]-ethylsulfamoyl}-2-methyl-benzoic acid methyl ester

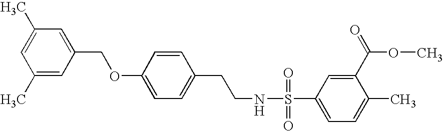

16% yield.

MS: 468.3 (M+1)

### Example 260

### 2,3-Dimethyl-5-{2-[4-(2,2,3,3-tetrafluoro-propoxyl]-phenyl-ethylsulfamoyl}-benzoic acid methyl ester

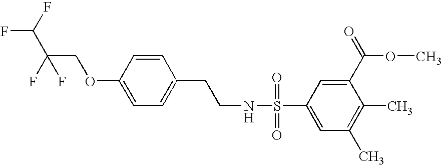

14% yield.

MS: 478.2 (M+1)

### Example 261

### 2-Ethyl-5-{2-[4-(4-fluoro-benzyloxy)-phenyl]-ethylsulfamoyl}-benzoic acid methyl ester

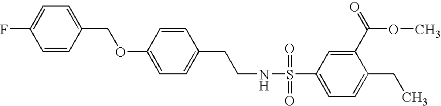

36% yield.

MS: 472.2 (M+1)

### Example 262

### 5-{2-[4-(4-Fluoro-benzyloxy)-phenyl]-ethylsulfamoyl}-2,3-dimethyl-benzoic acid methyl ester

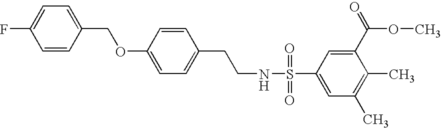

33% yield.

MS: 472.2 (M+1)

### Example 263

### 2-Ethyl-5-{2-[4-(4-trifluoromethyl-benzyloxy)-phenyl]-ethylsulfamoyl}-benzoic acid methyl ester

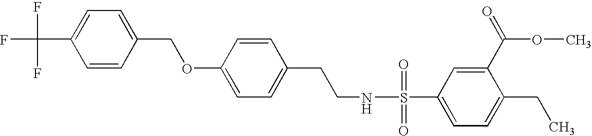

35% yield.

MS: 522.2 (M+1)

### Example 264

### 2,3-Dimethyl-5-{2-[4-(4-trifluoromethyl-benzyloxy)-phenyl]-ethylsulfamoyl}-benzoic acid methyl ester

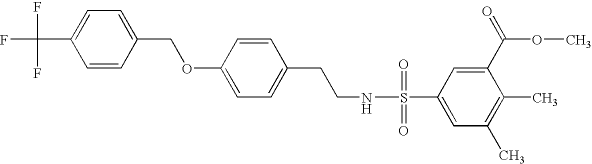

34% yield.

522.3 (M+1)

### Example 265

### 5-{2-[4-(2,3-Difluoro-benzyloxy)-phenyl]-ethylsulfamoyl}-2-ethyl-benzoic acid methyl ester

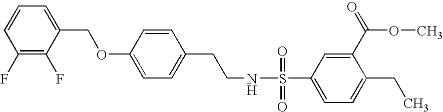

39% yield.

MS: 476.1 (M+1)

### Example 266

### 5-{2-[4-(2,3-Difluoro-benzyloxy)-phenyl]-ethylsulfamoyl}-2,3-dimethyl-benzoic acid methyl ester

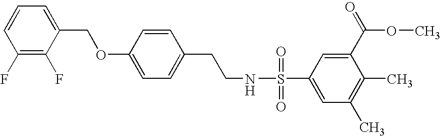

39% yield.

MS: 490.2 (M+1)

### Example 267

### 5-{2-[4-(3,4-Difluoro-benzyloxy)-phenyl]-ethylsulfamoyl}-2,3-dimethyl-benzoic acid methyl ester

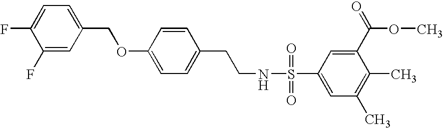

28% yield.

MS: 490.2 (M+1)

### Example 268

### 5-{2-[4-(3,4-Difluoro-benzyloxy)-phenyl]-ethylsulfamoyl}-2-ethyl-benzoic acid methyl ester

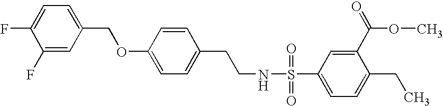

37% yield.

MS: 490.2 (M+1)

### Example 269

### 5-{2-[4-(3,5-Difluoro-benzyloxy)-phenyl]-ethylsulfamoyl}-2-ethyl-benzoic acid methyl ester

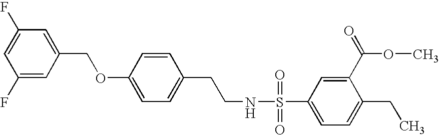

32% yield.

MS: 490.2 (M+1)

### Example 270

### 5-{2-[4-(3,5-Difluoro-benzyloxy)-phenyl]-ethylsulfamoyl}-2,3-dimethyl-benzoic acid methyl ester

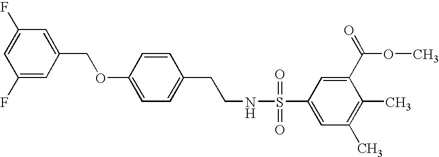

33% yield.

MS: 490.2 (M+1)

### Example 271

### 2-Ethyl-5-{2-[4-(2,2,3,3-tetrafluoro-propoxy)-phenyl]-ethylsulfamoyl}-benzoic acid methyl ester

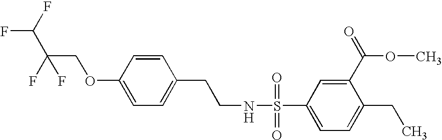

14% yield.

MS: 478.2 (M+1)

### Example 272

### 5-{2-[4-(2,3-Dimethyl-benzyloxy)-phenyl]-ethylsulfamoyl}-2-ethyl-benzoic acid methyl ester

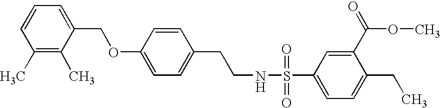

45% yield.

MS: 482.2 (M+1)

### Example 273

### 5-{2-[4-(2,3-Dimethyl-benzyloxy)-phenyl]-ethylsulfamoyl}-2,3-dimethyl-benzoic acid methyl ester

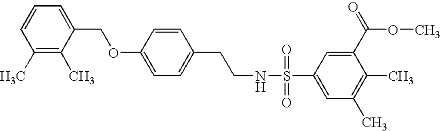

26% yield.

MS: 482.3 (M+1)

### Example 274

### 2-Methyl-5-{2-[4-(4-methyl-benzyloxy)-phenylsulfanyl]-ethylsulfamoyl}-benzoic acid methyl ester

The title compound was prepared using a procedure analogous to that of EXAMPLES 249 and 250 but using 5-[2-(4-hydroxy-phenylsulfanyl)-ethylsulfamoyl]-2-methyl benzoic acid methyl ester instead of 5-[2-(4-hydroxy-phenyl)-ethylsulfamoyl]-2-methyl-benzoic acid methyl ester.

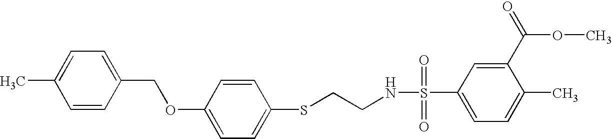

23% yield.

MS: 486.2 (M+1)

### Example 275

### 5-[2-(4-tert-Butyl-phenoxy)-ethylsulfamoyl]-2-ethyl-benzoic acid methyl ester

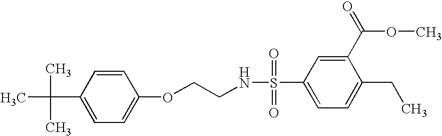

A solution of 2-ethyl-5-(2-hydroxy-ethylsulfamoyl)-benzoic acid methyl ester (0.2 g, 0.697 mmol), t-butylphenol (0.105 g, 0.697 mmol), triphenylphosphine (0.201 g, 0.697 mmol), and diethyl azodicarboxylate (0.135 ml, 0.697 mmol) in 1 ml tetrahydrofuran was irradiated in a microwave oven (high power) at 120° C. for 5 min. The reaction mixture was cooled to room temperature and diluted with 30 ml ethyl acetate. The ethyl acetate solution was washed sequentially with 30 ml 1N aqueous hydrochloric acid, 30 ml water and 30 ml brine, dried (anhydrous sodium sulfate) and concentrated to dryness under reduced pressure. The residue (0.161 g) was purified by flash column chromatography (40 g silica gel), eluting with 85:15 hexane/ethyl acetate to yield the title compound as a white solid (0.028 g, 10% yield).

MS: 418.2 (M−1)

### Example 276

### 5-{2-[2-(4-tert-Butyl-phenyl)-oxazol-4-yl]-ethylsulfamoyl}-2-methyl-benzoic acid

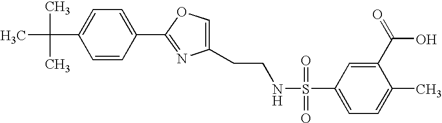

To a solution of 5-{2-[2-(4-tert-butyl-phenyl)-oxazol-4-yl]-ethylsulfamoyl}-2-methyl-benzoic acid methyl ester (0.1 g, 0.22 mmol) in 10 ml methanol was added 0.33 ml (0.33 mmol) of 1N aqueous sodium hydroxide solution. The reaction mixture was heated at 80° C. overnight, then cooled to room temperature and concentrated under reduced pressure. The solid residue was treated with 5 ml 1N aqueous hydrochloric acid solution, filtered, washed with 5 ml water and dried under suction to yield the title compound as a white solid (0.21 g, 53% yield).

MS: 443.1 (M+1)

The title compounds of EXAMPLES 277-550 were prepared using procedures analogous to that of EXAMPLE 276 from appropriate starting materials.

| Ex. | Compound | Compound Name | Data |
|---|---|---|---|
| 277 | 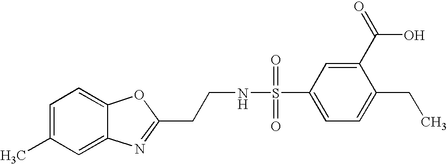 | 2-Ethyl-5-[2-(5-methyl- benzooxazol-2-yl)- ethylsulfamoyl]-benzoic acid | 77% yield. MS: 389.0 (M + 1) |
| 278 | 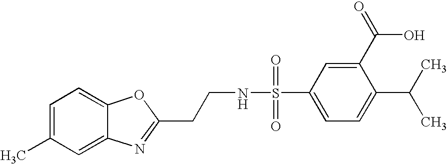 | 2-Isopropyl-5-[2-(5-methyl- benzooxazol-2-yl)- ethylsulfamoyl]-benzoic acid | 84% yield. MS: 403.0 (M + 1) |
| 279 | 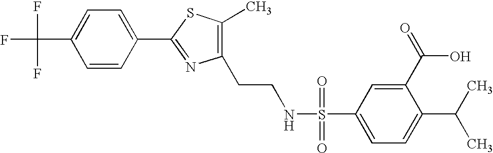 | 2-Isopropyl-5-{2-[5-methyl- 2-(4-trifluoromethyl- phenyl)-thiazol-4-yl]- ethylsulfamoyl}-benzoic acid | 61% yield. MS: 527.0 (M + 1) |
| 280 | 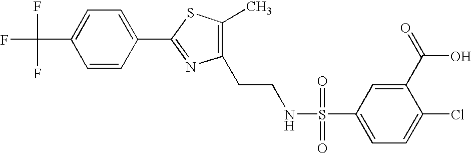 | 2-Chloro-5-{2-[5-methyl-2- (4-trifluoromethyl-phenyl)- thiazol-4-yl]- ethylsulfamoyl}-benzoic acid | 88% yield. MS: 504.9 (M + 1) |
| 281 | 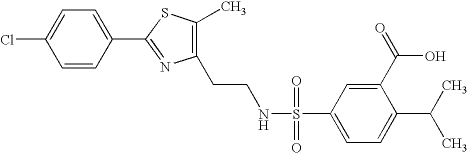 | 5-{2-[2-(4-Chloro-phenyl)- 5-methyl-thiazol-4-yl]- ethylsulfamoyl}-2-isopropyl- benzoic acid | 83% yield. MS: 479.0 (M + 1) |
| 282 | 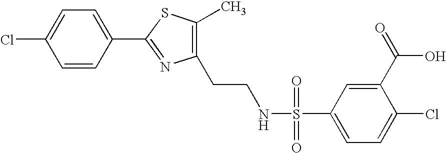 | 2-Chloro-5-{2-[2-(4-chloro- phenyl)-5-methyl-thiazol-4- yl]-ethylsulfamoyl}-benzoic acid | 42% yield. MS: 470.9 (M + 1) |
| 283 | 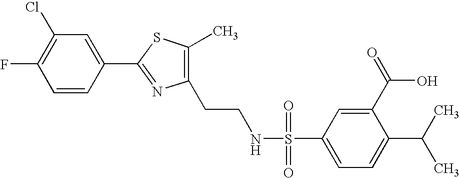 | 5-{2-[2-(3-Chloro-4-fluoro- phenyl)-5-methyl-thiazol-4- yl]-ethylsulfamoyl}-2- isopropyl-benzoic acid | 68% yield. MS: 497.0 (M + 1) |
| 284 | 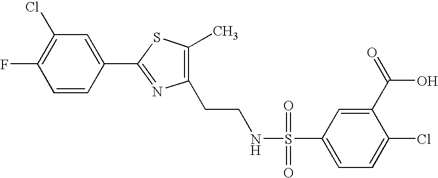 | 2-Chloro-5-{2-[2-(3-chloro- 4-fluoro-phenyl)-5-methyl- thiazol-4-yl]- ethylsulfamoyl}-benzoic acid | 70% yield. MS: 488.9 (M + 1) |
| 285 | 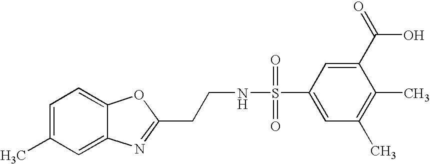 | 2,3-Dimethyl-5-[2-(5- methyl-benzooxazol-2-yl)- ethylsulfamoyl]-benzoic acid | 91% yield. MS: 389.1 (M + 1) |
| 286 | 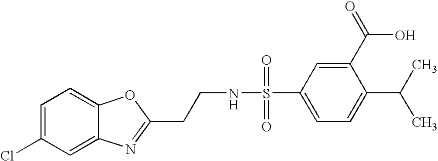 | 5-[2-(5-Chloro- benzooxazol-2-yl)- ethylsulfamoyl]-2-isopropyl- benzoic acid | 31% yield. MS: 423.0 (M + 1) |
| 287 | 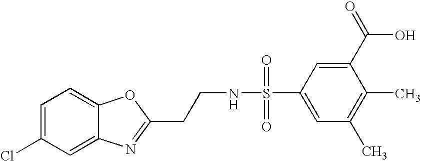 | 5-[2-(5-Chloro- benzooxazol-2-yl)- ethylsulfamoyl]-2,3- dimethyl-benzoic acid | 18% yield MS: 409.0 (M + 1) |
| 288 | 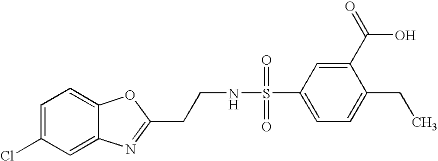 | 5-[2-(5-Chloro- benzooxazol-2-yl)- ethylsulfamoyl]-2-ethyl- benzoic acid | 13% yield. MS: 409.0 (M + 1) |
| 289 | 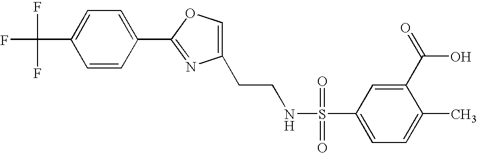 | 2-Methyl-5-{2-[2-(4- trifluoromethyl-phenyl)- oxazol-4-yl]- ethylsulfamoyl}-benzoic acid | 81% yield. MS: 455.0 (M + 1) |
| 290 | 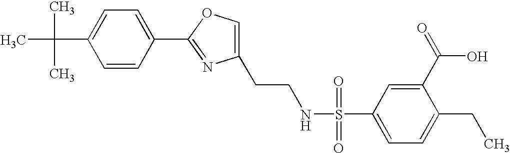 | 5-{2-[2-(4-tert-Butyl- phenyl)-oxazol-4-yl]- ethylsulfamoyl}-2-ethyl- benzoic acid | 78% yield. MS: 457.1 (M + 1) |
| 291 | 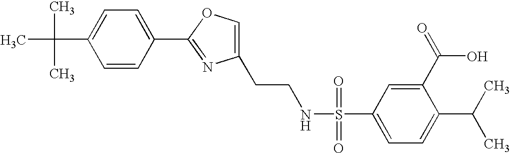 | 5-{2-[2-(4-tert-Butyl- phenyl)-oxazol-4-yl]- ethylsulfamoyl}-2- isopropyl-benzoic acid | 82% yield. MS: 471.1 (M + 1) |
| 292 | 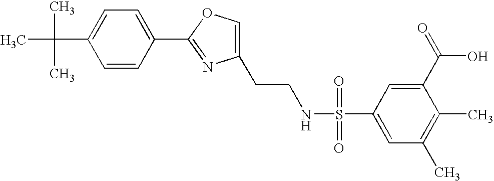 | 5-{2-[2-(4-tert-Butyl- phenyl)-oxazol-4-yl]- ethylsulfamoyl}-2,3- dimethyl-benzoic acid | 77% yield. MS: 457.1 (M + 1) |
| 293 | 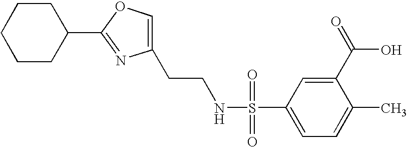 | 5-[2-(2-Cyclohexyl-oxazol- 4-yl)-ethylsulfamoyl]-2- methyl-benzoic acid | 87% yield. MS: 393.1 (M + 1) |
| 294 | 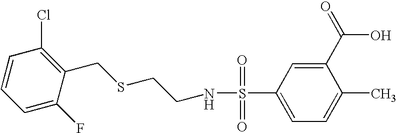 | 5-[2-(2-Chloro-6-fluoro- benzylsulfanyl)- ethylsulfamoyl]-2-methyl- benzoic acid | 92% yield. MS: 417.9 (M + 1) |
| 295 | 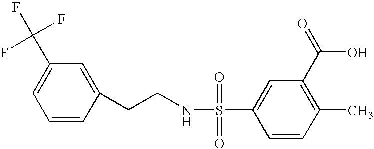 | 2-Methyl-5-[2-(3- trifluoromethyl-phenyl)- ethylsulfamoyl]-benzoic acid | 84% yield. MS: 386.0 (M + 1) |
| 296 | 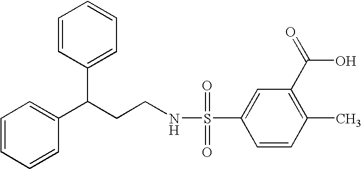 | 5-(3,3-Diphenyl- propylsulfamoyl)-2-methyl- benzoic acid | 90% yield. MS: 408.0 (M − 1) |
| 297 | 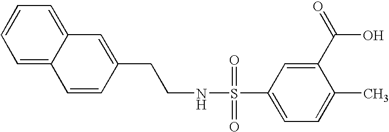 | 2-Methyl-5-(2-naphthalen- 2-yl-ethylsulfamoyl)- benzoic acid | 95% yield. MS: 368.0 (M − 1) |
| 298 | 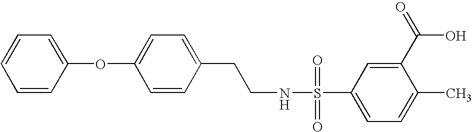 | 2-Methyl-5-[2-(4-phenoxy- phenyl)-ethylsulfamoyl]- benzoic acid | 86% yield. MS: 410.0 (M − 1) |
| 299 | 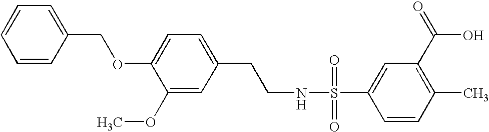 | 5-[2-(4-Benzyloxy-3- methoxy-phenyl)- ethylsulfamoyl]-2-methyl- benzoic acid | 87% yield. MS: 454.0 (M − 1) |
| 300 | 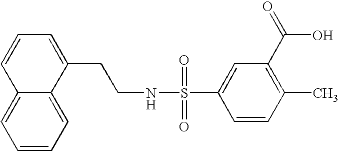 | 2-Methyl-5-(2-naphthalen- 1-yl-ethylsulfamoyl)- benzoic acid | 92% yield. MS: 368.0 (M − 1) |
| 301 | 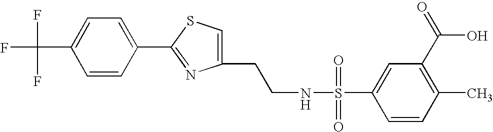 | 2-Methyl-5-{2-[2-(4- trifluoromethyl-phenyl)- thiazol-4-yl]- ethylsulfamoyl}-benzoic acid | 81% yield. MS: 471.0 (MS + 1) |
| 302 | 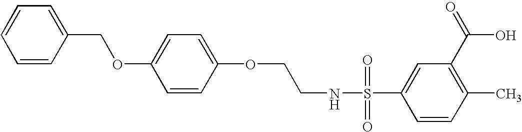 | 5-[2-(4-Benzyloxy- phenoxy)-ethylsulfamoyl]- 2-methyl-benzoic acid | 88% yield. MS: 440.1 (M − 1) |
| 303 | 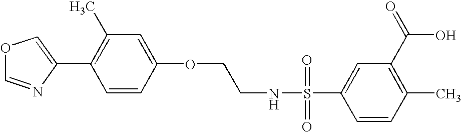 | 2-Methyl-5-[2-(3-methyl-4- oxazol-4-yl-phenoxy)- ethylsulfamoyl]-benzoic acid | 90% yield. MS: 417.1 (M + 1) |
| 304 | 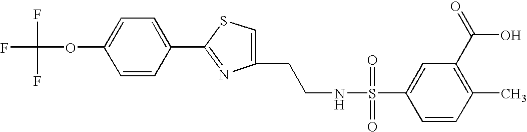 | 2-Methyl-5-{2-[2-(4- trifluoromethoxy-phenyl)- thiazol-4-yl]- ethylsulfamoyl}-benzoic acid | 85% yield. MS: 487.0 (M + 1) |
| 305 | 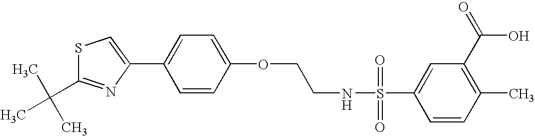 | 5-{2-[4-(2-tert-Butyl- thiazol-4-yl)-phenoxy]- ethylsulfamoyl}-2-methyl- benzoic acid | 76% yield. MS: 475.1 (M + 1) |
| 306 | 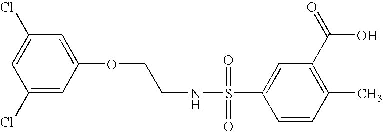 | 5-[2-(3,5-Dichloro- phenoxy)-ethylsulfamoyl]- 2-methyl-benzoic acid | 57% yield. MS: 405.0 (M + 1) |
| 307 | 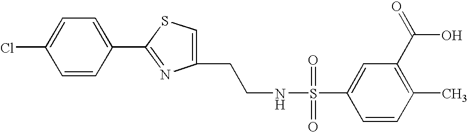 | 5-{2-[2-(4-Chloro-phenyl)- thiazol-4-yl]- ethylsulfamoyl}-2-methyl- benzoic acid | 78% yield. MS: 435.0 (M − 1) |
| 308 | 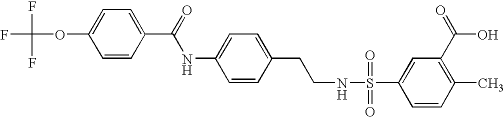 | 2-Methyl-5-{2-[4-(4- trifluoromethoxy- benzoylamino)-phenyl]- ethylsulfamoyl}-benzoic acid | 85% yield. MS: 523.0 (M + 1) |
| 309 | 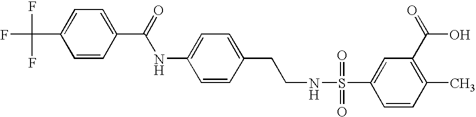 | 2-Methyl-5-{2-[4-(4- trifluoromethyl- benzoylamino)-phenyl]- ethylsulfamoyl}-benzoic acid | 96% yield. MS: 507.0 (M + 1) |
| 310 | 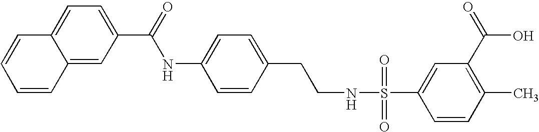 | 2-Methyl-5-(2-{4- [(naphthalene-2-carbonyl)- amino]-phenyl}- ethylsulfamoyl)-benzoic acid | 90% yield. MS: 489.0 (M + 1) |
| 311 | 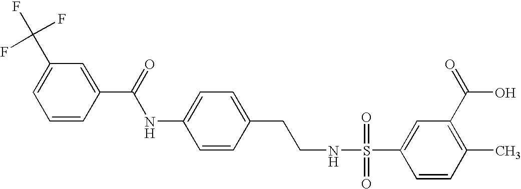 | 2-Methyl-5-{2-[4-(3- trifluoromethyl- benzoylamino)-phenyl]- ethylsulfamoyl}-benzoic acid | 85% yield. MS: 507.1 (M + 1) |
| 312 | 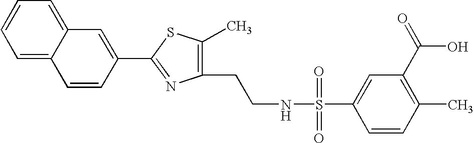 | 2-Methyl-5-[2-(5-methyl-2- naphthalen-2-yl-thiazol-4- yl)-ethylsulfamoyl]-benzoic acid | 88% yield. MS: 467.0 (M + 1) |
| 313 | 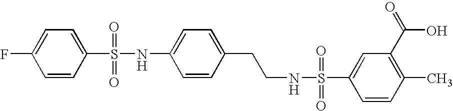 | 5-{2-[4-(4-Fluoro- benzenesulfonylamino)- phenyl]-ethylsulfamoyl}-2- methyl-benzoic acid | 92% yield. MS: 491.0 (M − 1) |
| 314 | 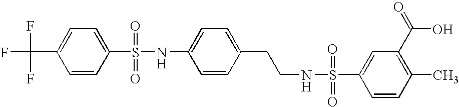 | 2-Methyl-5-{2-[4-(4- trifluoromethyl- benzenesulfonylamino)- phenyl]-ethylsulfamoyl}- benzoic acid | 90% yield. MS: 541.0 (M − 1) |
| 315 | 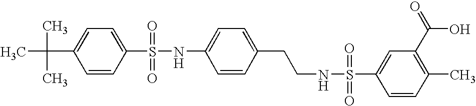 | 5-{2-[4-(4-tert-Butyl- benzenesulfonylamino)- phenyl]-ethylsulfamoyl}-2- methyl-benzoic acid | 93% yield. MS: 529.1 (M − 1) |
| 316 | 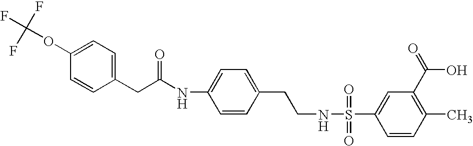 | 2-Methyl-5-(2-{4-[2-(4- trifluoromethoxy-phenyl)- acetylamino]-phenyl}- ethylsulfamoyl)-benzoic acid | 55% yield. MS: 537.0 (M + 1) |
| 317 | 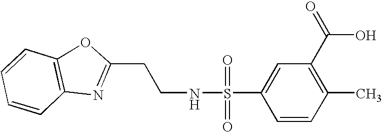 | 5-(2-Benzooxazol-2-yl- ethylsulfamoyl)-2-methyl- benzoic acid | 33% yield. MS: 361.2 (M + 1) |
| 318 | 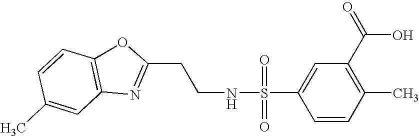 | 2-Methyl-5-[2-(5-methyl- benzooxazol-2-yl)- ethylsulfamoyl]-benzoic acid | 51% yield. MS: 375.2 (M + 1) |
| 319 | 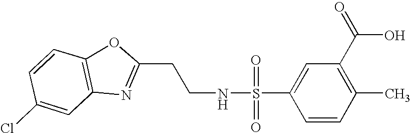 | 5-[2-(5-Chloro- benzooxazol-2-yl)- ethylsulfamoyl]-2-methyl- benzoic acid | 29% yield. MS: 395.1 (M + 1) |
| 320 | 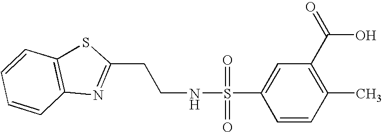 | 5-(2-Benzothiazol-2-yl- ethylsulfamoyl)-2-methyl- benzoic acid | 53% yield. MS: 377.1 (M + 1) |
| 321 | 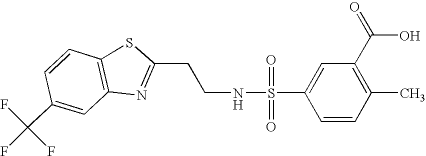 | 2-Methyl-5-[2-(5- trifluoromethyl- benzothiazol-2-yl)- ethylsulfamoyl]-benzoic acid | 47% yield. MS: 445.0 (M + 1) |
| 322 | 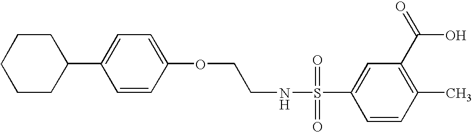 | 5-[2-(4-Cyclohexyl- phenoxy)-ethylsulfamoyl]- 2-methyl-benzoic acid | 86% yield. MS: 418.2 (M + 1) |
| 323 | 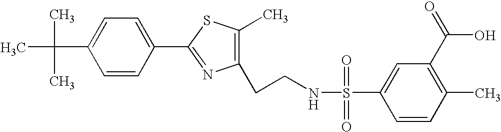 | 5-{2-[2-(4-tert-Butyl- phenyl)-5-methyl-thiazol-4- yl]-ethylsulfamoyl}-2- methyl-benzoic acid | 93% yield. MS: 473.1 (M + 1) |
| 324 | 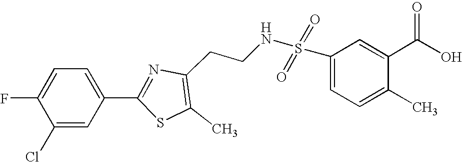 | 5-{2-[2-(3-Chloro-4-fluoro- phenyl)-5-methyl-thiazol-4- yl]-ethylsulfamoyl}-2- methyl-benzoic acid | 98% yield. MS: 469.0 (M + 1) |
| 325 | 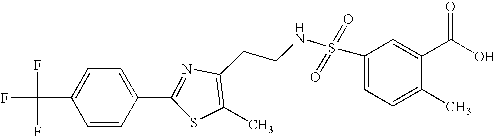 | 2-Methyl-5-{2-[5-methyl-2- (4-trifluoromethyl-phenyl)- thiazol-4-yl]- ethylsulfamoyl}-benzoic acid | 93% yield. MS: 485.0 (M + 1) |
| 326 | 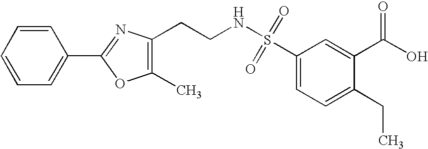 | 2-Ethyl-5-[2-(5-methyl-2- phenyl-oxazol-4-yl)- ethylsulfamoyl]-benzoic acid | 85% yield. MS: 415.1 (M + 1) |
| 327 | 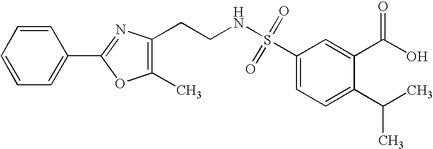 | 2-Isopropyl-5-[2-(5-methyl- 2-phenyl-oxazol-4-yl)- ethylsulfamoyl]-benzoic acid | 78% yield. MS: 429.1 (M + 1) |
| 328 | 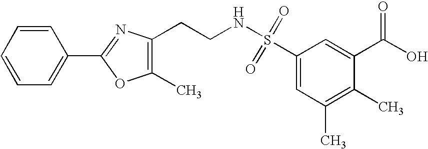 | 2,3-Dimethyl-5-[2-(5- methyl-2-phenyl-oxazol-4- yl)-ethylsulfamoyl]- benzoic acid | 52% yield. MS: 415.1 (M + 1) |
| 329 | 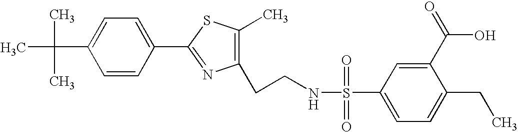 | 5-{2-[2-(4-tert-Butyl- phenyl)-5-methyl-thiazol-4- yl]-ethylsulfamoyl}-2-ethyl- benzoic acid | 80% yield MS: 486.9 (M + 1) |
| 330 | 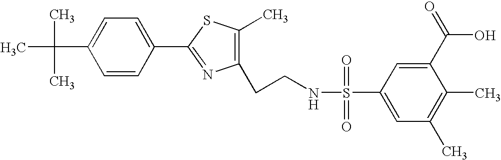 | 5-{2-[2-(4-tert-Butyl- phenyl)-5-methyl-thiazol-4- yl]-ethylsulfamoyl}-2,3- dimethyl-benzoic acid | 94% yield. MS: 488.1 (M + 1) |
| 331 | 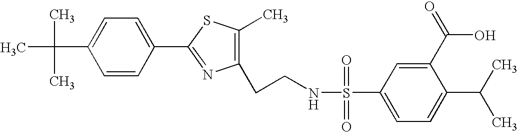 | 5-{2-[2-(4-tert-Butyl- phenyl)-5-methyl-thiazol-4- yl]-ethylsulfamoyl}-2- isopropyl-benzoic acid | 92% yield. MS: 500.9 (M + 1) |
| 332 | 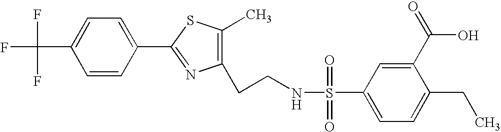 | 2-Ethyl-5-{2-[5-methyl-2-(4- trifluoromethyl-phenyl)- thiazol-4-yl]- ethylsulfamoyl}-benzoic acid | 91% yield. MS: 498.9 (M + 1) |
| 333 | 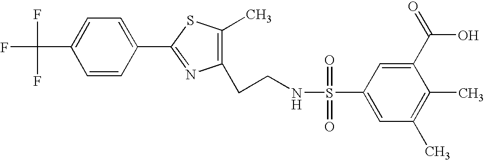 | 2,3-Dimethyl-5-{2-[5- methyl-2-(4-trifluoromethyl- methyl-2-(4-trifluoromethyl- phenyl)-thiazol-4-yl]- ethylsulfamoyl}-benzoic acid | 56% yield. MS: 498.9 (M + 1) |
| 334 | 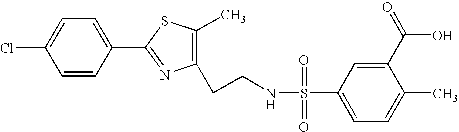 | 5-{2-[2-(4-Chloro-phenyl)- 5-methyl-thiazol-4-yl]- ethylsulfamoyl}-2-methyl- benzoic acid | 97% yield. MS: 450.9 (M + 1) |
| 335 | 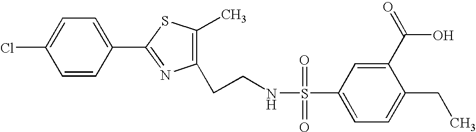 | 5-{2-[2-(4-Chloro-phenyl)- 5-methyl-thiazol-4-yl]- ethylsulfamoyl}-2-ethyl- benzoic acid | 91% yield. MS: 464.9 (M + 1) |
| 336 | 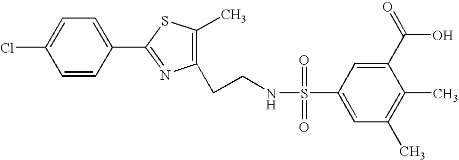 | 5-{2-[2-(4-Chloro-phenyl)- 5-methyl-thiazol-4-yl]- ethylsulfamoyl}-2,3- dimethyl-benzoic acid | 99% yield MS: 464.9 (M + 1) |
| 337 | 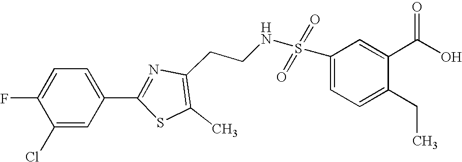 | 5-{2-[2-(3-Chloro-4-fluoro- phenyl)-5-methyl-thiazol-4- yl]-ethylsulfamoyl}-2-ethyl- benzoic acid | 72% yield. MS: 481.0 (M − 1) |
| 338 | 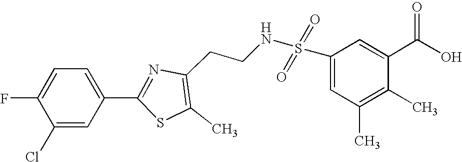 | 5-{2-[2-(3-Chloro-4-fluoro- phenyl)-5-methyl-thiazol-4- yl]-ethylsulfamoyl}-2,3- dimethyl-benzoic acid | 89% yield. MS: 481.0 (M − 1) |
| 339 | 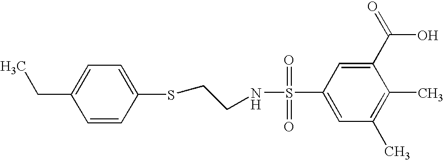 | 5-[2-(4-Ethyl- phenylsulfanyl)- ethylsulfamoyl]-2,3- dimethyl-benzoic acid | 59% yield. MS: 392.3 (M − 1) |
| 340 | 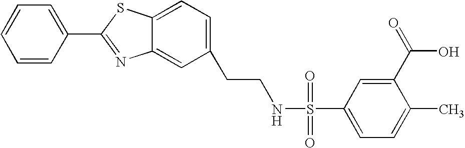 | 2-Methyl-5-[2-(2-phenyl- benzothiazol-5-yl)- ethylsulfamoyl]-benzoic acid | 83% yield. MS: 453.0 (M + 1) |
| 341 | 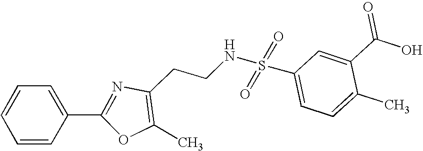 | 2-Methyl-5-(2-(5-methyl-2- phenyl-oxazol-4-yl)- ethylsulfamoyl]benzoic acid | 98% yield. MS: 401.3 (M + 1) |
| 342 | 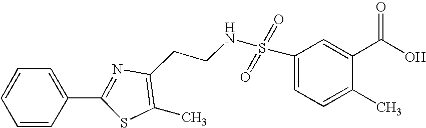 | 2-Methyl-5-[2-(5-methyl-2- phenyl-thiazol-4-yl)- ethylsulfamoyl]benzoic acid | 96% yield. MS: 417.0 (M + 1) |
| 343 | 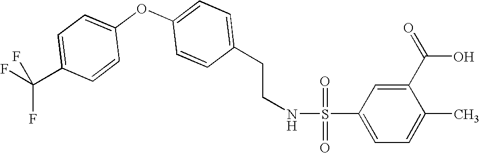 | 2-Methyl-5-{2-[4-(4- trifluoromethyl-phenoxy)- phenyl]-ethylsulfamoyl)- benzoic acid | 2% yield. MS: 478.0 (M − 1) |
| 344 | 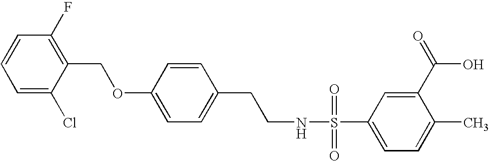 | 5-{2-[4-(2-Chloro-6-fluoro- benzyloxy)-phenyl]- ethylsulfamoyl}-2-methyl- benzoic acid | 52% yield. MS: 478.0 (M + 1) |
| 345 | 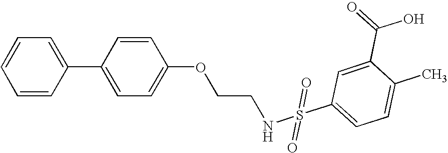 | 5-[2-(Biphenyl-4-yloxy)- ethylsulfamoyl]-2-methyl- benzoic acid | 25% yield. MS: 412.1 (M + 1) |
| 346 | 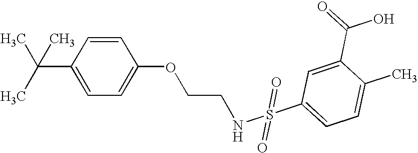 | 5-[2-(4-tert-Butyl-phenoxy)- ethylsulfamoyl]-2-methyl- benzoic acid | 28% yield. MS: 390.1 (M − 1) |
| 347 | 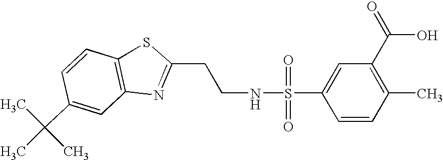 | 5-[2-(5-tert-Butyl- benzooxazol-2-yl)- ethylsulfamoyl]-2-methyl- benzoic acid | 73% yield. MS: 417.1 (M + 1) |
| 348 | 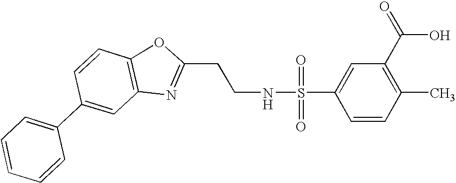 | 2-Methyl-5-[2-(5-phenyl- benzooxazol-2-yl)- ethylsulfamoyl]-benzoic acid | 59% yield. MS: 437.0 (M + 1) |
| 349 | 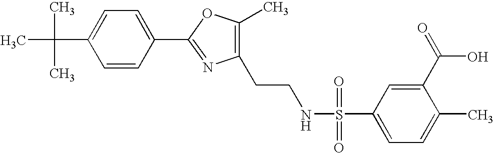 | 5-{2-[2-(4-tert-Butyl- phenyl)-5-methyl-oxazol-4- yl]-ethylsulfamoyl}-2- methyl-benzoic acid | 63% yield. MS: 457.5 (M + 1) |
| 350 | 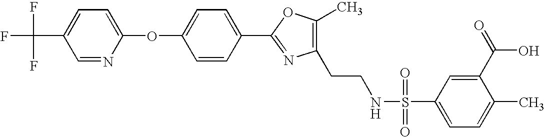 | 2-Methyl-5-(2-{5-methyl-2- [4-(5-trifluoromethyl-pyridin- 2-yloxy)-phenyl]-thiazol-4- yl}-ethylsulfamoyl)-benzoic acid | 53% yield. MS: 578.5 (M + 1) |
| 351 | 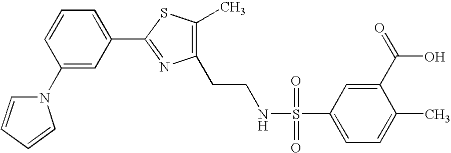 | 2-Methyl-5-{2-[5-methyl-2- (3-pyrrol-1-yl-phenyl)- thiazol-4-yl]- ethylsulfamoyl}-benzoic acid | 77% yield MS: 482.3 (M + 1) |
| 352 | 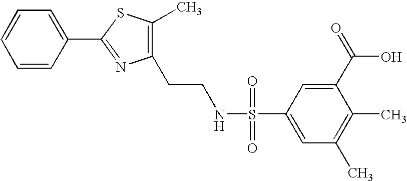 | 2,3-Dimethyl-5-[2-(5- methyl-2-phenyl-thiazol-4- yl)-ethylsulfamoyl]-benzoic acid | 89% yield. MS: 431.3 (M + 1) |
| 353 | 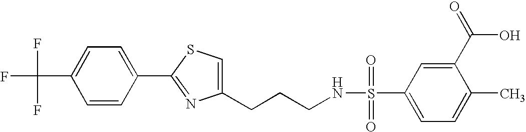 | 2-Methyl-5-{3-[2-(4- trifluoromethyl-phenyl)- thiazol-4-yl]- propylsulfamoyl}-benzoic acid | 44% yield. MS: 485.3 (M + 1) |
| 354 | 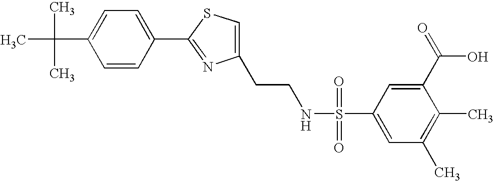 | 5-{2-[2-(4-tert-Butyl- phenyl)-thiazol-4-yl]- ethylsulfamoyl}-2,3- dimethyl-benzoic acid | 79% yield. MS: 473.4 (M + 1) |
| 355 | 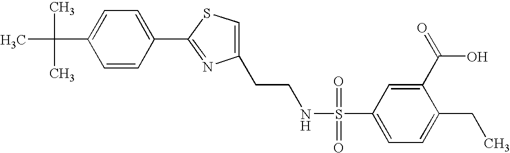 | 5-{2-[2-(4-tert-Butyl- phenyl)-thiazoi-4-yl]- ethylsulfamoyl}-2-ethyl- benzoic acid | 73% yield. MS: 473.4 (M + 1) |
| 356 | 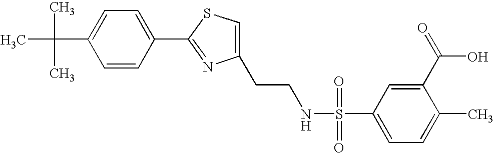 | 5-{2-[2-(4-tert-Butyl- phenyl)-thiazol-4-yl]- ethylsulfamoyl}-2-methyl- benzoic acid | 78% yield. MS: 459.4 (M + 1) |
| 357 | 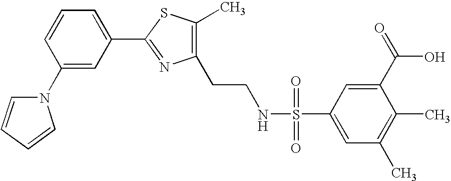 | 2,3-Dimethyl-5-{2-[5- methyl-2-(3-pyrrol-1-yl- phenyl)-thiazol-4-yl]- ethylsulfamoyl}-benzoic acid | 96% yield. MS: 496.4 (M + 1) |
| 358 | 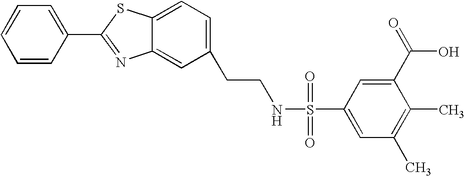 | 2,3-Dimethyl-5-[2-(2- phenyl-benzothiazol-5-yl)- ethylsulfamoyl]-benzoic acid | 47% yield. MS: 467.3 (M + 1) |
| 359 | 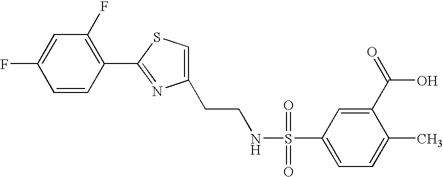 | 5-{2-[2-(2,4-Difluoro- phenyl)-thiazol-4-yl]- ethylsulfamoyl}-2-methyl- benzoic acid | 88% yield. MS: 439.3 (M + 1) |
| 360 | 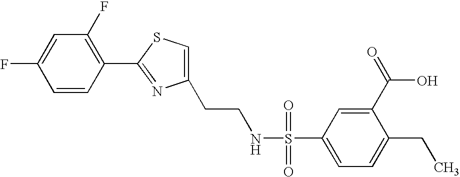 | 5-{2-[2-(2,4-Difluoro- phenyl)-thiazol-4-yl]- ethylsulfamoyl}-2-ethyl- benzoic acid | 94% yield. MS: 453.3 (M + 1) |
| 361 | 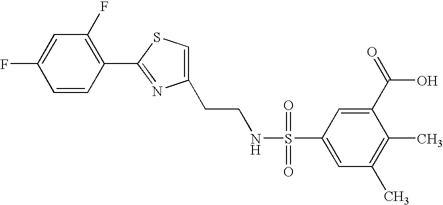 | 5-{2-[2-(2,4-Difluoro- phenyl)-thiazol-4-yl]- ethylsulfamoyl}-2,3- dimethyl-benzoic acid | 78% yield. MS: 453.3 (M + 1) |
| 362 | 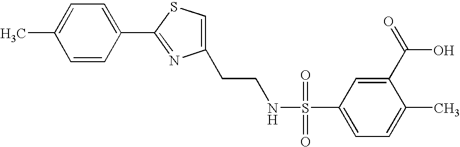 | 2-Methyl-5-[2-(2-p-tolyl- thiazol-4-yl)- ethylsulfamoyl]-benzoic acid | 93% yield. MS: 417.3 (M + 1) |
| 363 | 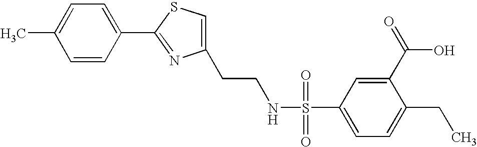 | 2-Ethyl-5-[2-(2-p-tolyl- thiazol-4-yl)- ethylsulfamoyl]-benzoic acid | 91% yield. MS: 431.3 (M + 1) |
| 364 | 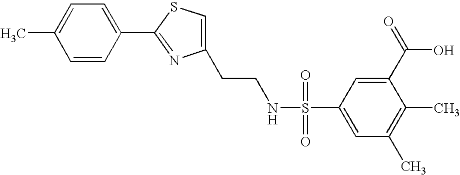 | 2,3-Dimethyl-5-[2-(2-p-tolyl- thiazol-4-yl)- ethylsulfamoyl]-benzoic acid | 100% yield. MS: 431.3 (M + 1) |
| 365 | 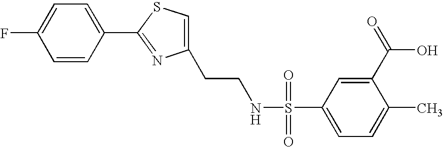 | 5-{2-[2-(4-Fluoro-phenyl)- thiazol-4-yl]- ethylsulfamoyl}-2- methyl-benzoic acid | 98% yield. MS: 421.3 (M + 1) |
| 366 | 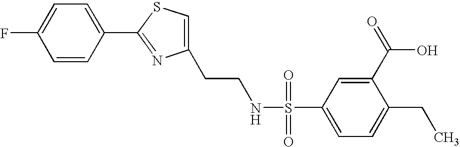 | 2-Ethyl-5-{2-[2-(4-fluoro- phenyl)-thiazol-4-yl]- ethylsulfamoyl}-benzoic acid | 96% yield. MS: 435.3 (M + 1) |
| 367 | 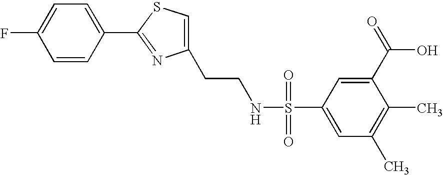 | 5-{2-[2-(4-Fluoro-phenyl)- thiazol-4-yl]- ethylsulfamoyl}-2,3- dimethyl-benzoic acid | 84% yield. MS: 435.3 (M + 1) |
| 368 | 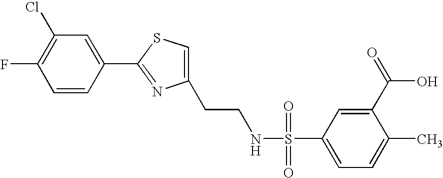 | 5-{2-[2-(3-Chloro-4-fluoro- phenyl)-thiazol-4-yl]- ethylsulfamoyl}-2-methyl- benzoic acid | 95% yield. MS: 455.2 (M + 1) |
| 369 | 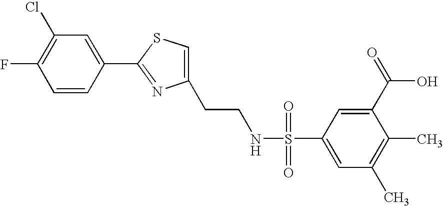 | 5-{2-[2-(3-Chloro-4-fluoro- phenyl)-thiazol-4-yl]- ethylsulfamoyl}-2,3- dimethyl-benzoic acid | 90% yield. MS: 469.3 (M + 1) |
| 370 | 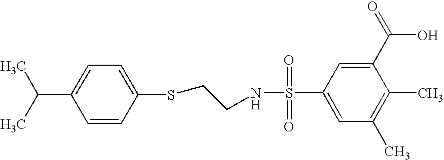 | 5-[2-(4-Isopropyl- phenylsulfanyl)- ethylsulfamoyl]-2,3- dimethyl-benzoic acid | 73% yield. MS: 406.3 (M − 1) |
| 371 | 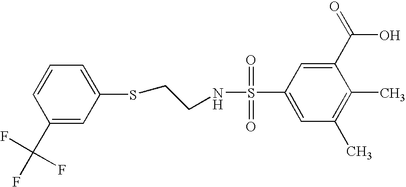 | 2,3-Dimethyl-5-[2-(3- trifluoromethyl- phenylsulfanyl)- ethylsulfamoyl]-benzoic acid | 85% yield. MS: 432.2 (M − 1) |
| 372 | 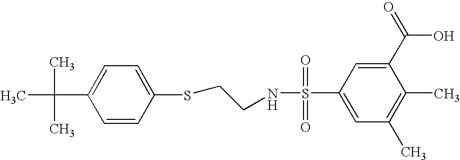 | 5-[2-(4-tert-Butyl- phenylsulfanyl)- ethylsulfamoyl]-2,3- dimethyl-benzoic acid | 62% yield. MS: 420.3 (M − 1) |
| 373 | 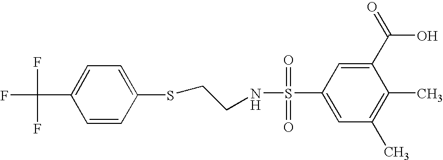 | 2,3-Dimethyl-5-[2-(4- trifluoromethyl- phenylsulfanyl)- ethylsulfamoyl]-benzoic acid | 98% yield. MS: 432.2 (M − 1) |
| 374 | 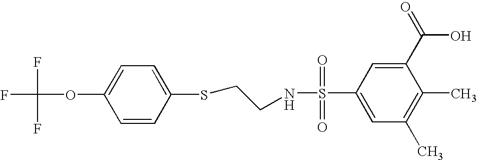 | 2,3-Dimethyl-5-[2-(4- trifluoromethoxy- phenylsulfanyl)- ethylsulfamoyl]-benzoic acid | 89% yield. MS: 448.2 (M − 1) |
| 375 | 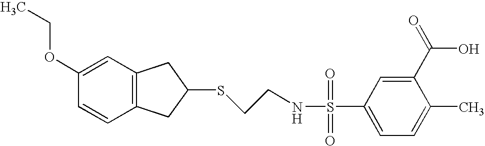 | 5-[2-(6-Ethoxy- benzothiazol-2-ylsulfanyl)- ethylsulfamoyl]-2-methyl- benzoic acid | 96% yield. MS: 453.2 (M + 1) |
| 376 | 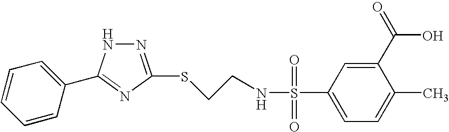 | 2-Methyl-5-[2-(5-phenyl- 1H-[1,2,4]triazol-3- ylsulfanyl)-ethylsulfamoyl]- benzoic acid | 96% yield. MS: 419.2 (M + 1) |
| 377 | 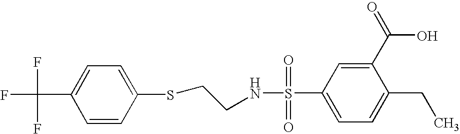 | 2-Ethyl-5-[2-(4- trifluoromethyl- phenylsulfanyl)- ethylsulfamoyl]-benzoic acid | 95% yield. MS: 432.3 (M − 1) |
| 378 | 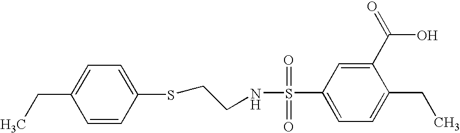 | 2-Ethyl-5-[2-(4-ethyl- phenylsulfanyl)- ethylsulfamoyl]-benzoic acid | 84% yield. MS: 392.3 (M − 1) |
| 379 | 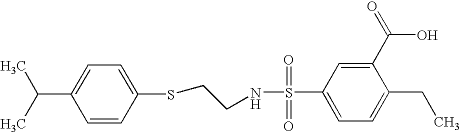 | 2-Ethyl-5-[2-(4-isopropyl- phenylsulfanyl)- ethylsulfamoyl]-benzoic acid | 63% yield. MS: 406.3 (M − 1) |
| 380 | 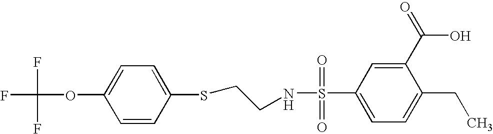 | 2-Ethyl-5-[2-(4- trifluoromethoxy- phenylsulfanyl)- ethylsulfamoyl]-benzoic acid | 93% yield. MS: 448.2 (M − 1) |
| 381 | 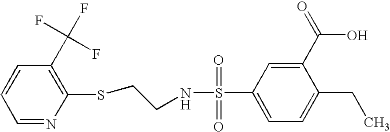 | 2-Ethyl-5-[2-(3- trifluoromethyl-pyridin-2- ylsulfanyl)-ethylsulfamoyl]- benzoic acid | 85% yield. MS: 435.3 (M + 1) |
| 382 | 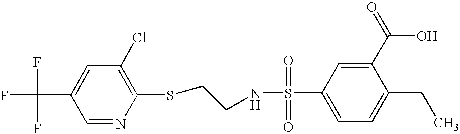 | 5-[2-(3-Chloro-5- trifluoromethyl-pyridin-2- ylsulfanyl)-ethylsulfamoyl]- 2-ethyl-benzoic acid | 92% yield. MS: 469.2 (M + 1) |
| 383 | 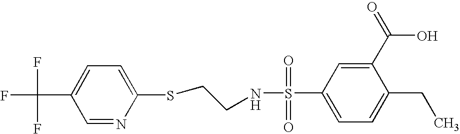 | 2-Ethyl-5-[2-(5-yield. trifluoromethyl-pyridin-2- ylsulfanyl)-ethylsulfamoyl]- benzoic acid | 87% yield. MS: 435.3 (M + 1) |
| 384 | 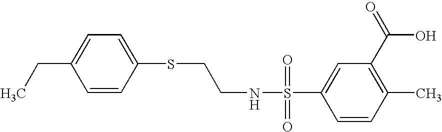 | 5-[2-(4-Ethyl- phenylsulfanyl)- ethylsulfamoyl]-2-methyl- benzoic acid | 89% yield. MS: 380.2 (M + 1) |
| 385 | 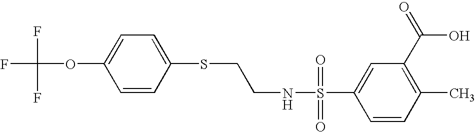 | 2-Methyl-5-[2-(4- trifluoromethoxy- phenylsulfanyl)- ethylsulfamoyl]- benzoic acid | 85% yield. MS: 436.1 (M + 1) |
| 386 | 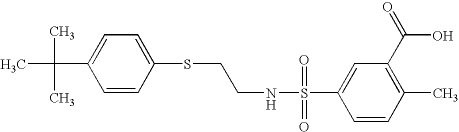 | 5-[2-(4-tert-Butyl- phenylsulfanyl)- ethylsulfamoyl]-2-methyl- benzoic acid | 89% yield. MS: 408.2 (M + 1) |
| 387 | 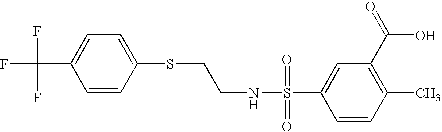 | 2-Methyl-5-[2-(4- trifluoromethyl- phenylsulfanyl)- ethylsulfamoyl]-benzoic acid | 80% yield: MS: 420.1 (M + 1) |
| 388 | 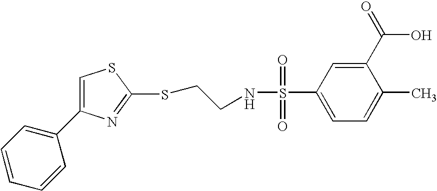 | 2-Methyl-5-[2-(4-phenyl- thiazol-2-ylsulfanyl)- ethylsulfamoyl]-benzoic acid | 87% yield. MS: 435.2 (M + 1) |
| 389 | 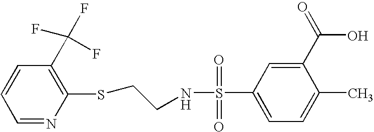 | 2-Methyl-5-[2-(3- trifluoromethyl-pyridin-2- ylsulfanyl)-ethylsulfamoyl]- benzoic acid | 95% yield. MS: 421.2 (M + 1) |
| 390 | 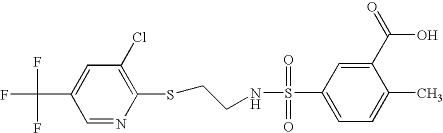 | 5-[2-(3-Chloro-5- trifluoromethyl-pyridin-2- ylsulfanyl)-ethylsulfamoyl]- 2-methyl-benzoic acid | 89% yield. MS: 455.2 (M + 1) |
| 391 | 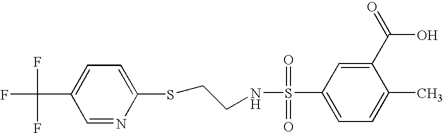 | 2-Methyl-5-[2-(5- trifluoromethyl-pyridin-2- ylsulfanyl)-ethylsulfamoyl]- benzoic acid | 68% yield. MS: 421.1 (M + 1) |
| 392 | 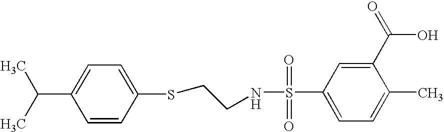 | 5-[2-(4-Isopropyl- phenylsulfanyl)- ethylsulfamoyl]-2-methyl- benzoic acid | 84% yield. MS: 394.3 (M + 1) |
| 393 | 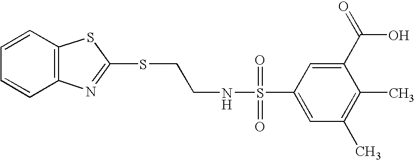 | 5-[2-(Benzothiazol-2- ylsulfanyl)-ethylsulfamoyl]- 2,3-dimethyl-benzoic acid | 55% yield. MS: 423.3 (M + 1) |
| 394 | 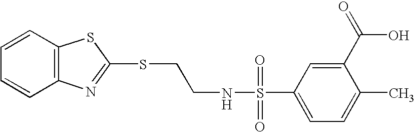 | 5-[2-(Benzothiazol-2- ylsulfanyl)-ethylsulfamoyl]- 2-methyl-benzoic acid | 59% yield. MS: 409.3 (M + 1) |
| 395 | 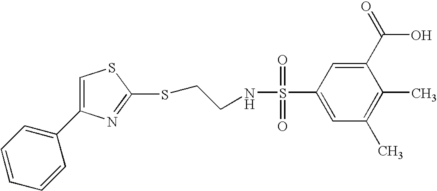 | 2,3-Dimethyl-5-[2-(4- phenyl-thiazol-2-ylsulfanyl)- ethylsulfamoyl]-benzoic acid | 99% yield. MS: 449.2 (M + 1) |
| 396 | 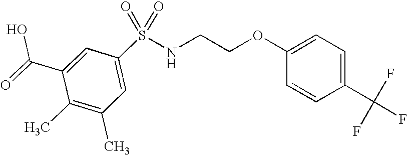 | 2,3-Dimethyl-5-[2-(4- trifluoromethyl-phenoxy)- ethylsulfamoyl]-benzoic acid | 53% yield. MS: 418.3 (M + 1) |
| 397 | 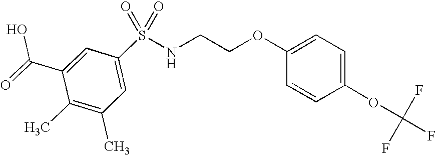 | 2,3-Dimethyl-5-[2-(4- trifluoromethoxy-phenoxy)- ethylsulfamoyl]-benzoic acid | 74% yield. MS: 434.1 (M + 1) |
| 398 | 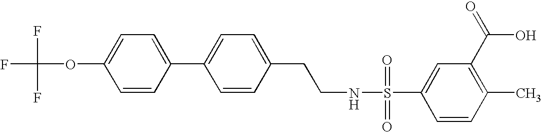 | 2-Methyl-5-[2-(4′- trifluoromethoxy-biphenyl- 4-yl)-ethylsulfamoyl]- benzoic acid | 85% yield. MS: 479.9 (M + 1) |
| 399 | 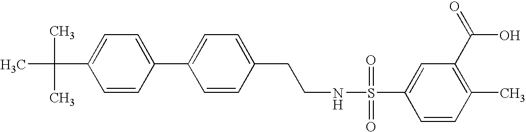 | 5-[2-(4-tert-Butyl-biphenyl- 4-yl)-ethylsulfamoyl]-2- methyl-benzoic acid | 87% yield. MS: 452.0 (M + 1) |
| 400 | 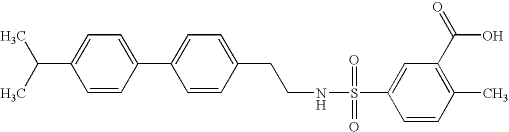 | 5-[2-(4′-Isopropyl-biphenyl- 4-yl)-ethylsulfamoyl]-2- methyl-benzoic acid | 80% yield. 438.0 (M + 1) |
| 401 | 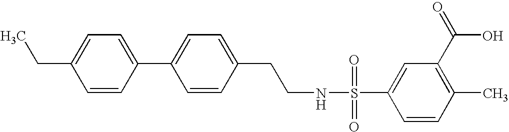 | 5-[2-(4′-Ethyl-biphenyl-4- yl)-ethylsulfamoyl]-2- methyl-benzoic acid | 70% yield. MS: 424.0 (M + 1) |
| 402 | 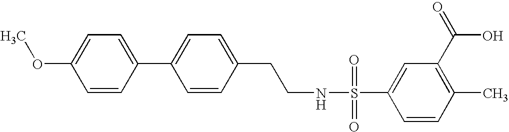 | 5-[2-(4′-Methoxy-biphenyl- 4-yl)-ethylsulfamoyl]-2- methyl-benzoic acid | 67% yield. MS: 426.0 (M + 1) |
| 403 | 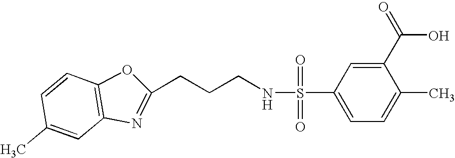 | 2-Methyl-5-[3-(5-methyl- benzooxazol-2-yl)- propylsulfamoyl]-benzoic acid | 85% yield. 389.4 (M + 1) |
| 404 | 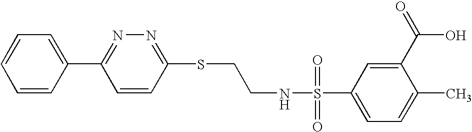 | 2-Methyl-5-[2-(6-phenyl- pyridazin-3-ylsulfanyl)- ethylsulfamoyl]-benzoic acid | 68% yield. MS: 430.1 (M + 1) |
| 405 | 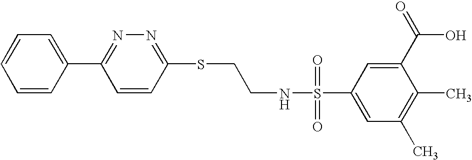 | 2,3-Dimethyl-5-[2-(6- phenyl-pyridazin-3- ylsulfanyl)-ethylsulfamoyl]- benzoic acid | 86% yield. MS: 444.2 (M + 1) |
| 406 | 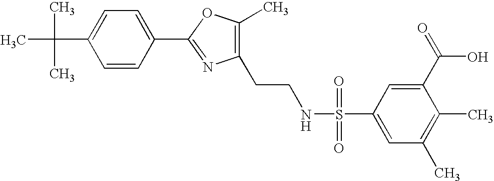 | 5-{2-[2-(4-tert-Butyl- phenyl)-5-methyl-oxazol-4- yl]-ethylsulfamoyl}-2,3- diethyl-benzoic acid | 96% yield. MS: 471.5 (M + 1) |
| 407 | 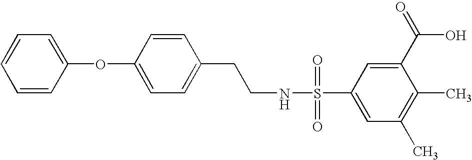 | 2,3-Dimethyl-5-[2-(4- phenoxy-phenyl)- ethylsulfamoyl]-benzoic acid | 86% yield. MS: 426.4 (M + 1) |
| 408 | 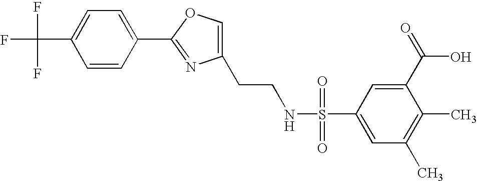 | 2,3-Dimethyl-5-{2-[2-(4- trifluoromethyl-phenyl)- oxazol-4-yl]- ethylsulfamoyl}-benzoic acid | 94% yield. MS: 469.4 (M + 1) |
| 409 | 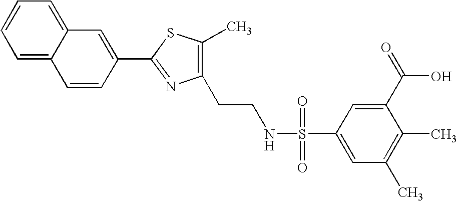 | 2,3-Dimethyl-5-[2-(5- methyl-2-naphthalen-2-yl- thiazol-4-yl)- ethylsulfamoyl]-benzoic acid | 55% yield. MS: 481.6 (M + 1) |
| 410 | 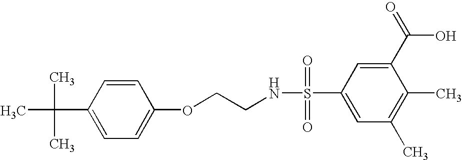 | 5-[2-(4-tert-Butyl- phenoxy)-ethylsulfamoyl]- 2,3-dimethyl-benzoic acid | 88% yield. MS: 406.4 (M + 1) |
| 411 | 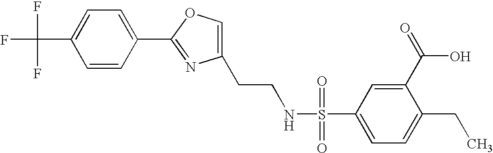 | 2-Ethyl-5-{2-[2-(4- trifluoromethyl-phenyl)- oxazol-4-yl]- ethylsulfamoyl}-benzoic | 61% yield. MS: 469.3 (M + 1) |
| 412 | 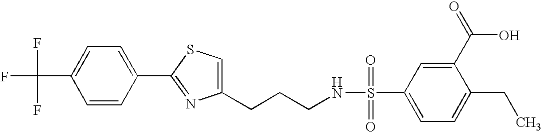 | 2-Ethyl-5-{3-[2-(4- trifluoromethyl-phenyl)- thiazol-4-yl]- propylsulfamoyl}- benzoic acid | 82% yield. MS: 499.2 (M + 1) |
| 413 | 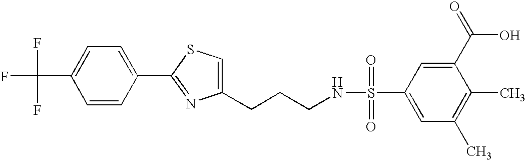 | 2,3-Dimethyl-5-{3-[2-(4- trifluoromethyl-phenyl)- thiazol-4-yl]- propylsulfamoyl}- benzoic acid | 19% yield. MS: 499.2 (M + 1) |
| 414 | 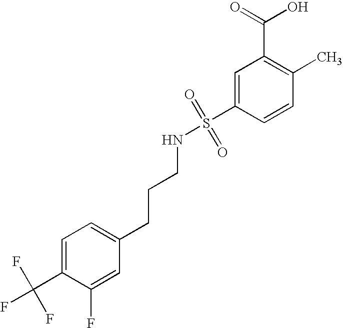 | 5-[3-(3-Fluoro-4- trifluoromethyl-phenyl)- propylsulfamoyl]-2-methyl- benzoic acid | 100% yield. MS: 418.3 (M + 1) |
| 415 | 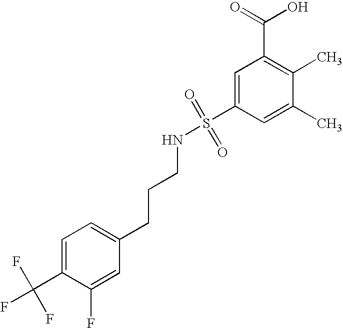 | 5-[3-(3-Fluoro-4- trifluoromethyl-phenyl)- propylsulfamoyl]-2,3- dimethyl-benzoic acid | 89% yield. MS: 434.2 (M + 1) |
| 416 | 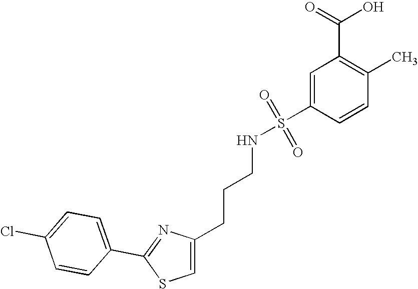 | 5-{3-[2-(4-Chloro-phenyl)- thiazol-4-yl]- propylsulfamoyl}-2-methyl- benzoic acid | 97% yield. MS: 451.0 (M + 1) |
| 417 | 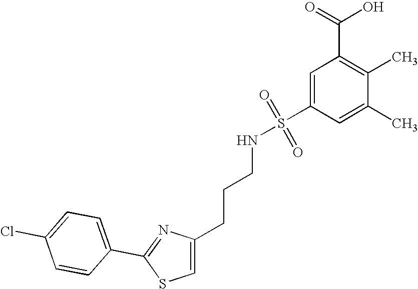 | 5-{3-[2-(4-Chloro-phenyl)- thiazol-4-yl]- propylsulfamoyl}-2,3- dimethyl-benzoic acid | 91% yield. MS: 463.0 (M − 1) |
| 418 | 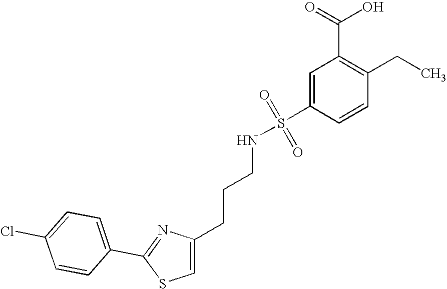 | 5-{3-[2-(4-Chloro-phenyl)- thiazol-4-yl]- propylsulfamoyl}-2-ethyl- benzoic acid | 90% yield. MS: 465.1 (M + 1) |
| 419 | 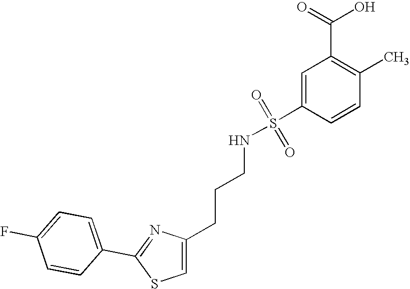 | 5-{3-[2-(4-Fluoro-phenyl)- thiazol-4-yl]- propylsulfamoyl}-2-methyl- benzoic acid | 87% yield. MS: 435.1 (M + 1) |
| 420 | 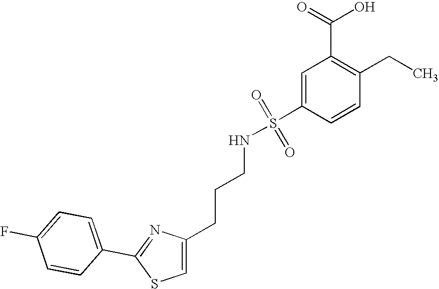 | 2-Ethyl-5-{3-[2-(4-fluoro- phenyl)-thiazol-4-yl]- propylsulfamoyl}-benzoic acid | 60% yield. MS: 449.1 (M + 1) |
| 421 | 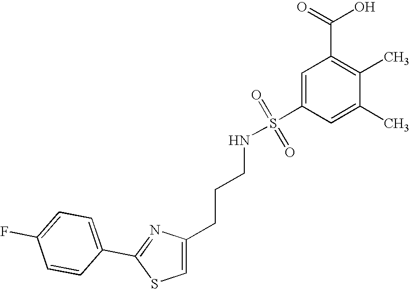 | 5-{3-[2-(4-Fluoro-phenyl)- thiazol-4-yl]- propylsulfamoyl}-2,3- dimethyl-benzoic acid | 96% yield. MS: 449.1 (M + 1) |
| 422 | 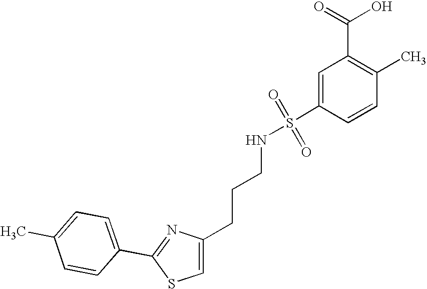 | 2-Methyl-5-[3-(2-p-tolyl- thiazol-4-yl)- propylsulfamoyl]-benzoic acid | 91% yield. MS: 431.1 (M + 1) |
| 423 | 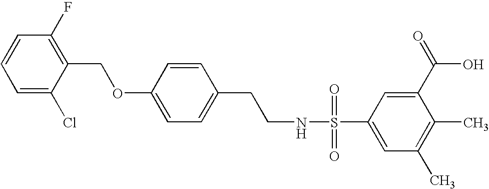 | 5-{2-[4-(2-Chloro-6-fluoro- benzyloxy)-phenyl]- ethylsulfamoyl}-2,3- dimethyl-benzoic acid | 97% yield. MS: 492.1 (M + 1) |
| 424 | 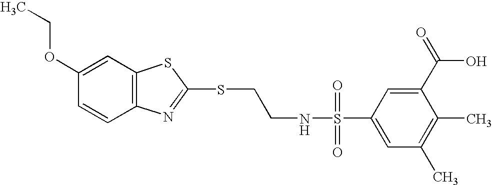 | 5-[2-(6-Ethoxy- benzothiazol-2-ylsulfanyl)- ethylsulfamoyl]-2,3- dimethyl-benzoic acid | 82% yield. MS: 467.3 (M + 1) |
| 425 | 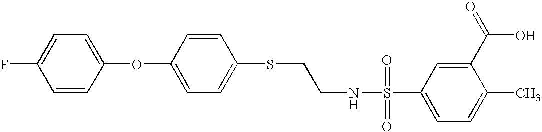 | 5-{2-[4-(4-Fluoro-phenoxy)- phenylsulfanyl]- ethylsulfamoyl}-2-methyl- benzoic acid | 75% yield. MS: 460.1 (M − 1) |
| 426 | 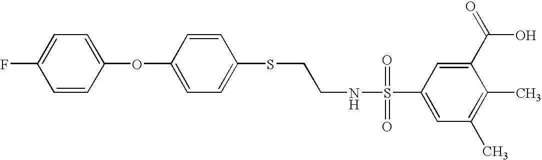 | 5-{2-[4-(4-Fluoro- phenoxy)-phenylsulfanyl]- ethylsulfamoyl}-2,3- dimethyl-benzoic acid | 96% yield. MS: 476.3 (M + 1) |
| 427 | 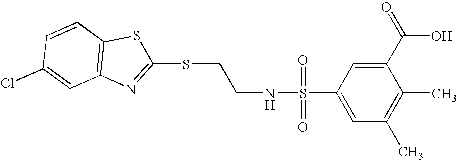 | 5-[2-(5-Chloro- benzothiazol-2-ylsulfanyl)- ethylsulfamoyl]-2,3- dimethyl-benzoic acid | 2% yield. MS: 457.1 (M + 1) |
| 428 | 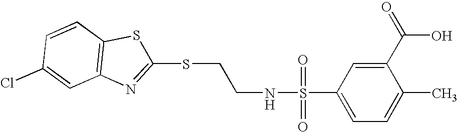 | 5-[2-(5-Chloro- benzothiazol-2-ylsulfanyl)- ethylsulfamoyl]-2-methyl- benzoic acid | 8% yield. MS: 443.1 (M + 1) |
| 429 | 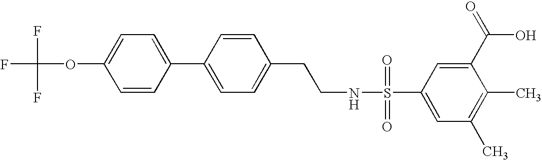 | 2,3-Dimethyl-5-[2-(4′- trifluoromethoxy-biphenyl- 4-yl)-ethylsulfamoyl]- benzoic acid | 84% yield. MS: 494.4 (M + 1) |
| 430 | 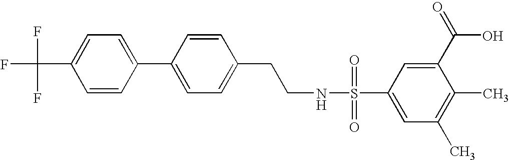 | 2,3-Dimethyl-5-[2-(4′- trifluoromethyl-biphenyl-4- yl)-ethylsulfamoyl]- benzoic acid | 80% yield. MS: 478.4 (M + 1) |
| 431 | 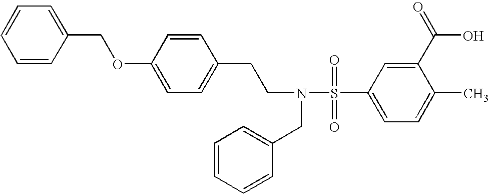 | 5-{Benzyl-[2-(4-benzyloxy- phenyl)-ethyl]-sulfamoyl}-2- methyl-benzoic acid | 75% yield. MS: 516.2 (M + 1) |
| 432 | 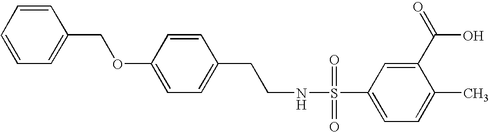 | 5-[2-(4-Benzyloxy-phenyl)- ethylsulfamoyl]-2-methyl- benzoic acid | 84% yield. MS: 426.2 (M + 1) |
| 433 | 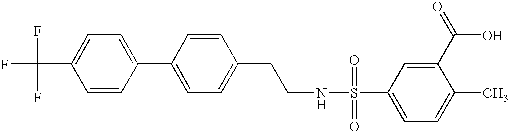 | 2-Methyl-5-[2-(4′- trifluoromethyl-biphenyl-4- yl)-ethylsulfamoyl]-benzoic acid | 90% yield. 464.4 (M + 1) |
| 434 | 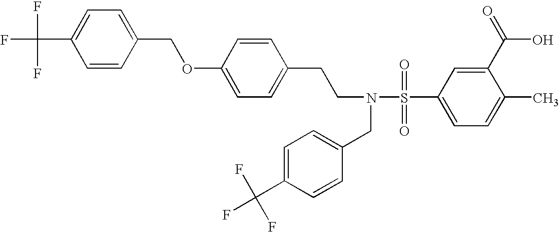 | 2-Methyl-5-((4- trifluoromethyl-benzyl)-{2- [4-(4-trifluoromethyl- benzyloxy)-phenyl]-ethyl}- sulfamoyl)-benzoic acid | 70% yield. MS: 652.1 (M + 1) |
| 435 | 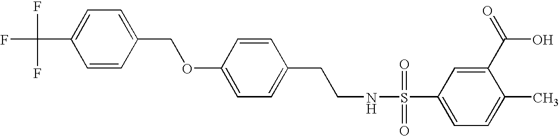 | 2-Methyl-5-{2-[4-(4- trifluoromethyl-benzyloxy)- phenyl]-ethylsulfamoyl}- benzoic acid | 85% yield. MS: 494.2 (M + 1) |
| 436 | 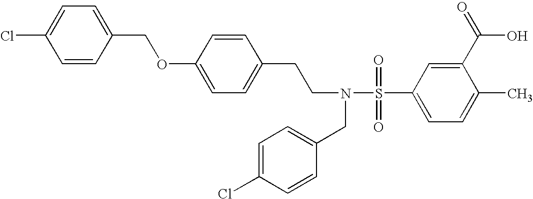 | 5-((4-Chloro-benzyl)-{2-[4- (4-chloro-benzyloxy)- phenyl]-ethyl}-sulfamoyl)-2- methyl-benzoic acid | 77% yield. MS: 584.1 (M) |
| 437 | 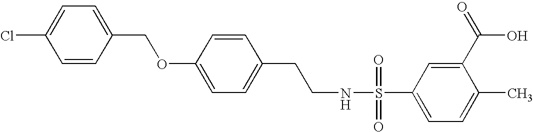 | 5-{2-[4-(4-Chloro- benzyloxy)-phenyl]- ethylsulfamoyl}-2-methyl- benzoic acid | 85% yield. MS: 460.2 (M + 1) |
| 438 | 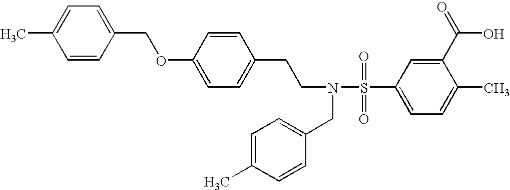 | 2-Methyl-5-((4-methyl- benzyl)-{2-[4-(4-methyl- benzyloxy)-phenyl]-ethyl}- sulfamoyl)-benzoic acid | 77% yield. MS: 544.3 (M + 1) |
| 439 | 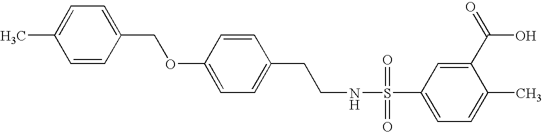 | 2-Methyl-5-{2-[4-(4-methyl- benzyloxy)-phenyl]- ethylsulfamoyl}-benzoic acid | 46% yield. MS: 440.2 (M + 1) |
| 440 | 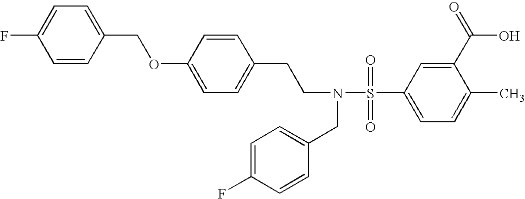 | 5-((4-Fluoro-benzyl)-{2-[4- (4-fluoro-benzyloxy)- phenyl]-ethyl}-sulfamoyl)-2- methyl-benzoic acid | 74% yield. MS: 552.2 (M + 1) |
| 441 | 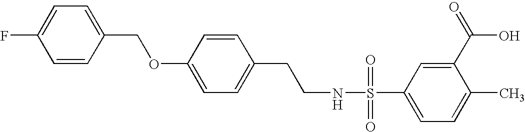 | 5-{2-[4-(4-Fluoro- benzyloxy)-phenyl]- ethylsulfamoyl}-2-methyl- benzoic acid | 78% yield. 444.2 (M + 1) |
| 442 | 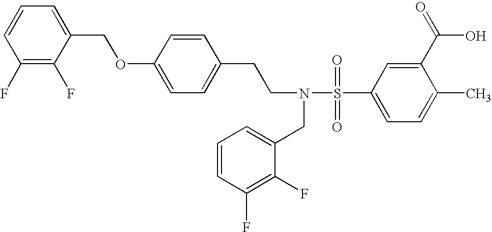 | 5-((2,3-Difluoro-benzyl)-{2- [4-(2,3-difluoro-benzyloxy)- phenyl]-ethyl}-sulfamoyl)-2- methyl-benzoic acid | 93% yield. MS: 588.2 (M + 1) |
| 443 | 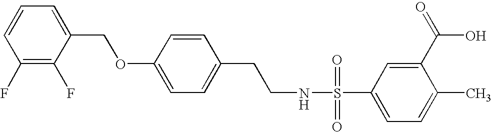 | 5-{2-[4-(2,3-Difluoro- benzyloxy)-phenyl]- ethylsulfamoyl}-2-methyl- benzoic acid | 75% yield. MS: 462.2 (M + 1) |
| 444 | 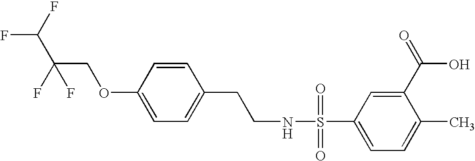 | 2-Methyl-5-{2-[4-(2,2,3,3- tetrafluoro-propoxy)- phenyl]-ethylsulfamoyl}- benzoic acid | 89% yield. MS: 450.1 (M + 1) |
| 445 | 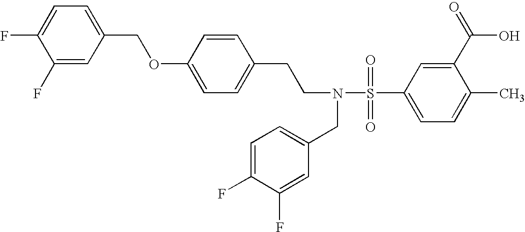 | 5-((3,4-Difluoro-benzyl)-{2- [4-(3,4-difluoro-benzyloxy)- phenyl]-ethyl}-sulfamoyl)- 2-methyl-benzoic acid | 80% yield. MS: 586.0 (M + 1) |
| 446 | 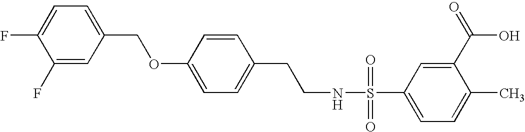 | 5-{2-[4-(3,4-Difiuoro- benzyloxy)-phenyl]- ethylsulfamoyl}-2-methyl- benzoic acid | 96% yield. 462.0 (M + 1) |
| 447 | 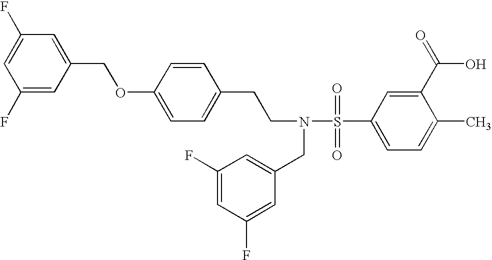 | 5-((3,5-Difluoro-benzyl)-{2- [4-(3,5-difluoro-benzyloxy)- phenyl]-ethyl}-sulfamoyl)-2- methyl-benzoic acid | 86% yield. MS: 586.0 (M + 1) |
| 448 | 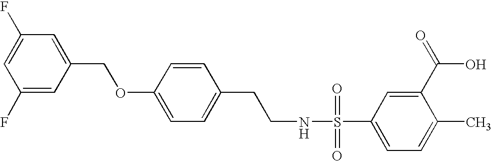 | 5-{2-[4-(3,5-Difluoro- benzyloxy)-phenyl]- ethylsulfamoyl}-2-methyl- benzoic acid | 92% yield. MS: 462.0 (M + 1) |
| 449 | 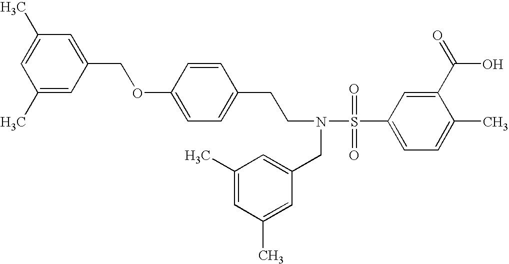 | 5-((3,5-Dimethyl-benzyl)-{2- [4-(3,5-dimethyl- benzyloxy)-phenyl]-ethyl}- sulfamoyl)-2-methyl- benzoic acid | 97% yield. MS: 572.2 (M + 1) |
| 450 | 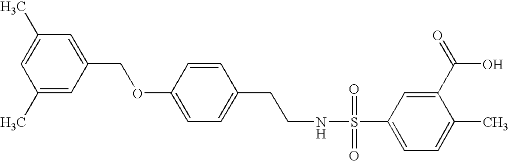 | 5-{2-[4-(3,5-Dimethyl- benzyloxy)-phenyl]- ethylsulfamoyl}-2-methyl- benzoic acid | 98% yield. MS: 454.2 (M + 1) |
| 451 | 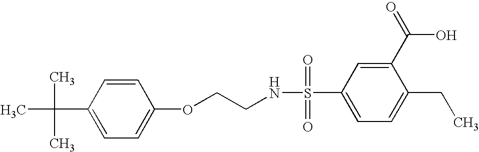 | 5-[2-(4-tert-Butyl-phenoxy)- ethylsulfamoyl]-2-ethyl- benzoic acid | 100% yield. MS: 404.3 (M + 1) |
| 452 | 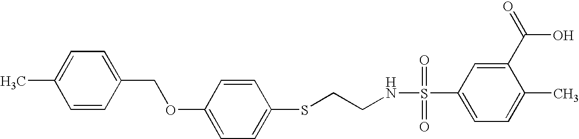 | 2-Methyl-5-{2-[4-(4-methyl- benzyloxy)-phenylsulfanyl]- ethylsulfamoyl}-benzoic acid | 85% yield. MS: 472.2 (M + 1) |
| 453 | 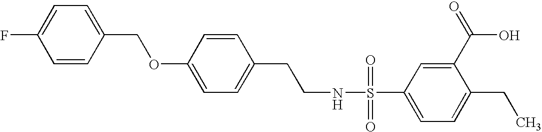 | 2-Ethyl-5-{2-[4-(4-fluoro- benzyloxy)-phenyl]- ethylsulfamoyl}-benzoic acid | 64% yield. MS: 456.3 (M + 1) |
| 454 | 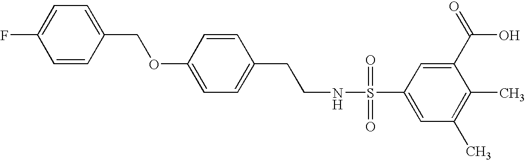 | 5-{2-[4-(4-Fluoro- benzyloxy)-phenyl]- ethylsulfamoyl}-2,3- dimethyl-benzoic acid | 77% yield. MS: 458.2 (M + 1) |
| 455 | 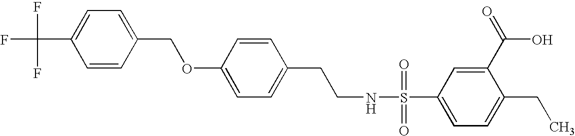 | 2-Ethyl-5-{2-[4-(4- trifluoromethyl-benzyloxy)- phenyl]-ethylsulfamoyl}- benzoic acid | 81% yield. MS: 508.2 (M + 1) |
| 456 | 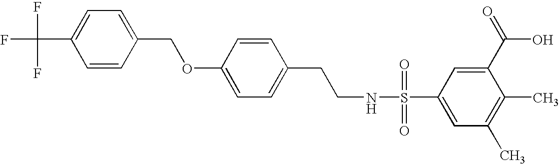 | 2,3-Dimethyl-5-{2-[4-(4- trifluoromethyl-benzyloxy)- phenyl]-ethylsulfamoyl}- benzoic acid | 85% yield. MS: 508.2 (M + 1) |
| 457 | 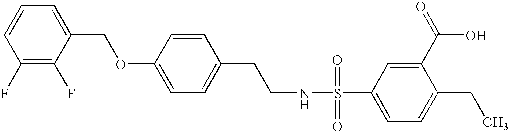 | 5-{2-[4-(2,3-Difluoro- benzyloxy)-phenyl]- ethylsulfamoyl}-2-ethyl- benzoic acid | 83% yield. MS: 490.2 (M + 1) |
| 458 | 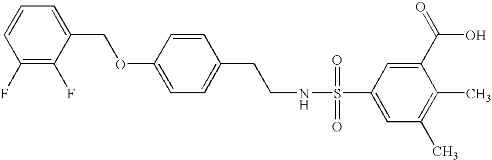 | 5-{2-[4-(2,3-Difluoro- benzyloxy)-phenyl]- ethylsulfamoyl}-2,3- dimethyl-benzoic acid | 90% yield. MS: 476.1 (M + 1) |
| 459 | 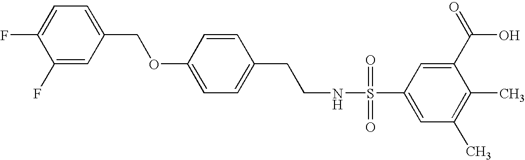 | 5-{2-[4-(3,4-Difluoro- benzyloxy)-phenyl]- ethylsulfamoyl}-2,3- dimethyl-benzoic acid | 100% yield. MS: 476.0 (M + 1) |
| 460 | 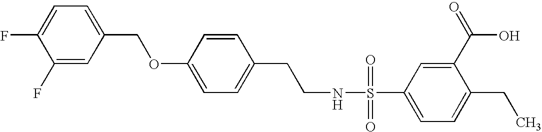 | 5-{2-[4-(3,4-Difluoro- benzyloxy)-phenyl]- ethylsulfamoyl}-2-ethyl- benzoic acid | 45% yield. MS: 476.0 (M + 1) |
| 461 | 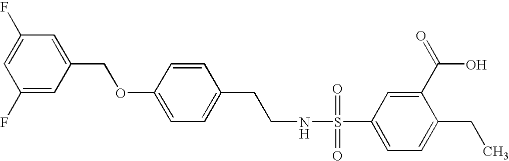 | 5-{2-[4-(3,5-Difluoro- benzyloxy)-phenyl]- ethylsulfamoyl}-2-ethyl- benzoic acid | 73% yield. MS: 476.1 (M + 1) |
| 462 | 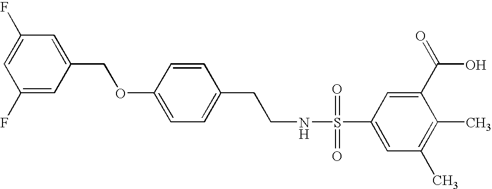 | 5-{2-[4-(3,5-Difluoro- benzyloxy)-phenyl]- ethylsulfamoyl}-2,3- dimethyl-benzoic acid | 86% yield. MS: 476.0 (M + 1) |
| 463 | 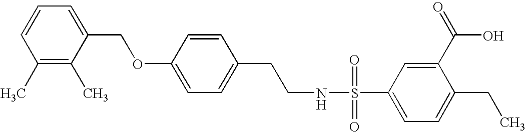 | 5-{2-[4-(2,3-Dimethyl- benzyloxy)-phenyl]- ethylsulfamoyl}-2-ethyl- benzoic acid | 70% yield. MS: 468.1 (M + 1) |
| 464 | 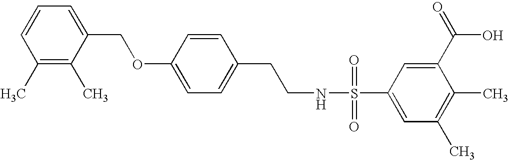 | 5-{2-[4-(2,3-Dimethyl- benzyloxy)-phenyl]- ethylsulfamoyl}-2,3- dimethyl-benzoic acid | 80% yield. MS: 468.1 (M + 1) |
| 465 | 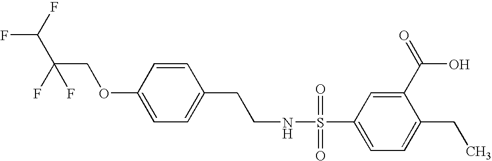 | 2-Ethyl-5-{2-[4-(2,2,3,3- tetrafluoro-propoxy)- phenyl]-ethylsulfamoyl}- benzoic acid | 74% yield. MS: 464.1 (M + 1) |
| 466 | 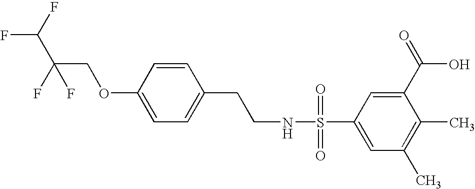 | 2,3-Dimethyl-5-{2-[4- (2,2,3,3-tetrafluoro- propoxy)-phenyl]- ethylsulfamoyl}-benzoic acid | 80% yield. MS: 464.0 (M + 1) |
| 467 | 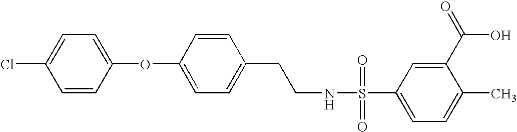 | 5-{2-[4-(4-Chloro- phenoxy)-phenyl]- ethylsulfamoyl}-2-methyl- benzoic acid | 86% yield. MS: 445.9 (M + 1) |
| 468 | 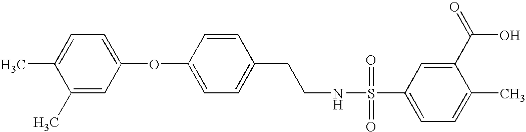 | 5-{2-[4-(3,4-Dimethyl- phenoxy)-phenyl]- ethylsulfamoyl}-2-methyl- benzoic acid | 82% yield. MS: 440.2 (M + 1) |
| 469 | 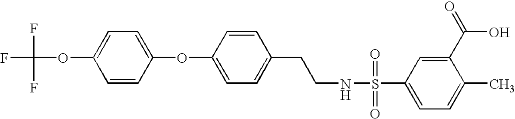 | 2-Methyl-5-{2-[4-(4- trifluoromethoxy-phenoxy)- phenyl]-ethylsulfamoyl}- benzoic acid | 91% yield. MS: 496.1 (M + 1) |
| 470 | 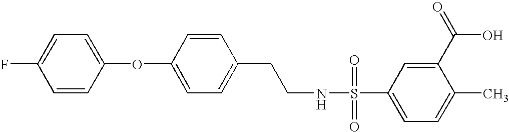 | 5-{2-[4-(4-Fluoro-phenoxy)- phenyl]-ethylsulfamoyl}-2- methyl-benzoic acid | 88% yield. MS: 430.2 (M + 1) |
| 471 | 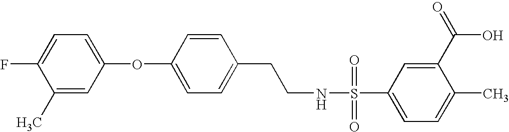 | 5-{2-[4-(4-Fluoro-3-methyl- phenoxy)-phenyl]- ethylsulfamoyl}-2-methyl- benzoic acid | 85% yield. MS: 444.2 (M + 1) |
| 472 | 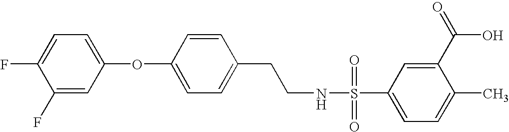 | 5-{2-[4-(3,4-Difluoro- phenoxy)-phenyl]- ethylsulfamoyl}-2-methyl- benzoic acid | 89% yield. MS: 448.2 (M + 1) |
| 473 | 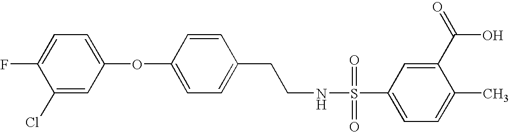 | 5-{2-[4-(3-Chloro-4-fluoro- phenoxy)-phenyl]- ethylsulfamoyl}-2-methyl- benzoic acid | 93% yield. MS: 462.1 (M + 1) |
| 474 | 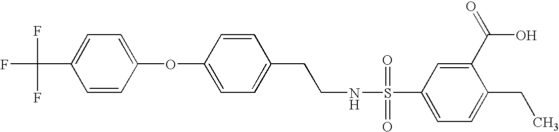 | 2-Ethyl-5-{2-[4-(4- trifluoromethyl-phenoxy)- phenyl]-ethylsulfamoyl}- benzoic acid | 67% yield. MS: 494.2 (M + 1) |
| 475 | 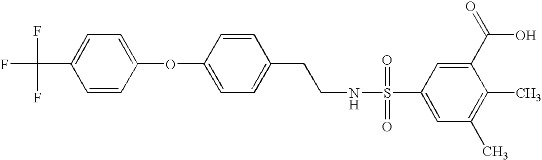 | 2,3-Dimethyl-5-{2-[4-(4- trifluoromethyl-phenoxy)- phenyl]-ethylsulfamoyl}- benzoic acid | 86% yield. MS: 494.2 (M + 1) |
| 476 | 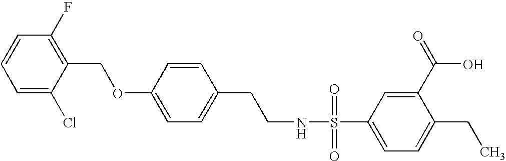 | 5-{2-[4-(2-Chloro-6-fluoro- benzyloxy)-phenyl]- ethylsulfamoyl}-2-ethyl- benzoic acid | 91% yield. MS: 492.2 (M + 1) |
| 477 | 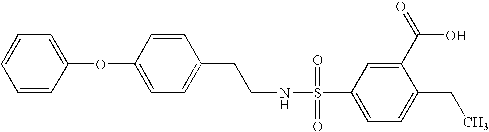 | 2-Ethyl-5-[2-(4-phenoxy- phenyl)-ethylsulfamoyl]- benzoic acid | 61% yield. MS: 426.2 (M + 1) |
| 478 | 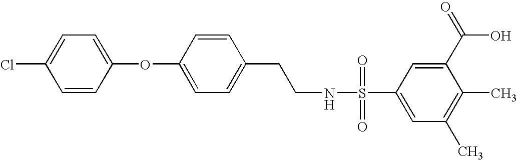 | 5-{2-[4-(4-Chloro-phenoxy)- phenyl]-ethylsulfamoyl}- 2,3-dimethyl-benzoic acid | 100% yield. MS: 458.2 (M − 1) |
| 479 | 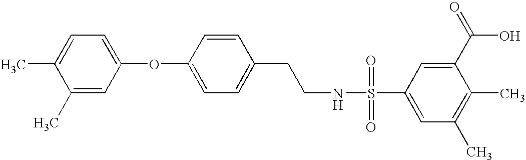 | 5-{2-[4-(3,4-Dimethyl- phenoxy)-phenyl]- ethylsulfamoyl}-2,3- dimethyl-benzoic acid | 99% yield MS: 452.3 (M − 1) |
| 480 | 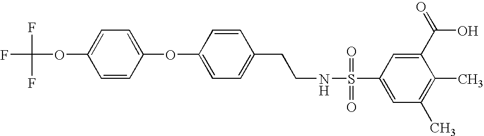 | 2,3-Dimethyl-5-{2-[4-(4- trifluoromethoxy-phenoxy)- phenyl]-ethylsulfamoyl}- benzoic acid | 100% yield MS: 508.2 (M − 1) |
| 481 | 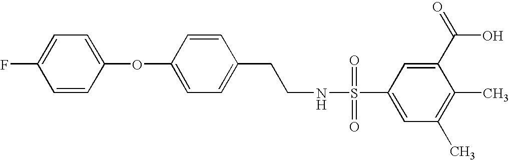 | 5-{2-[4-(4-Fluoro-phenoxy)- phenyl]-ethylsulfamoyl}- 2,3-dimethyl-benzoic acid | 67% yield. MS: 442.3 (M − 1) |
| 482 | 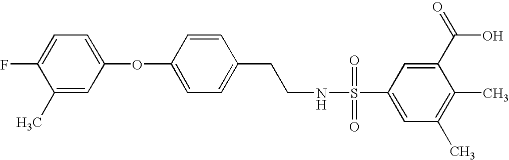 | 5-{2-[4-(4-Fluoro-3-methyl- phenoxy)-phenyl]- ethylsulfamoyl}-2,3- dimethyl-benzoic acid | 50% yield MS: 456.2 (M − 1) |
| 483 | 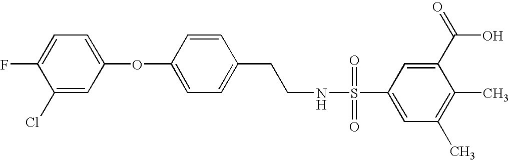 | 5-{2-[4-(3-Chloro-4-fluoro- phenoxy)-phenyl]- ethylsulfamoyl}-2,3- dimethyl-benzoic acid | 48% yield. MS: 472.3 (M − 1) |
| 484 | 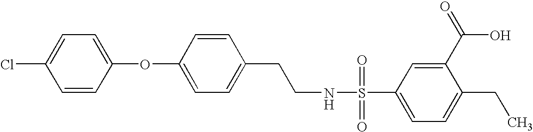 | 5-{2-[4-(4-Chloro-phenoxy)- phenyl]-ethylsulfamoyl}-2- ethyl-benzoic acid | 91% yield MS: 458.2 (M − 1) |
| 485 | 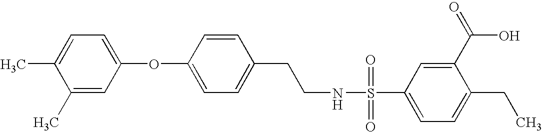 | 5-{2-[4-(3,4-Dimethyl- phenoxy)-phenyl]- ethylsulfamoyl}-2-ethyl- benzoic acid | 76% yield MS: 452.3 (M − 1) |
| 486 | 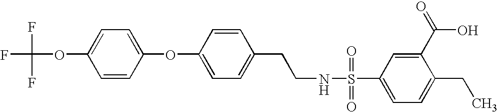 | 2-Ethyl-5-{2-[4-(4- trifluoromethoxy-phenoxy)- phenyl]-ethylsulfamoyl}- benzoic acid | 65% yield. MS: 502.3 (M − 1) |
| 487 | 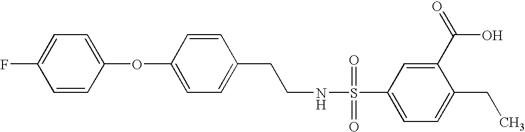 | 2-Ethyl-5-{2-[4-(4-fluoro- phenoxy)-phenyl]- ethylsulfamoyl}-benzoic acid | 62% yield MS: 442.3 (M − 1) |
| 488 | 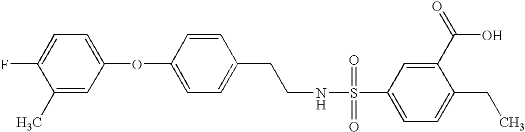 | 2-Ethyl-5-{2-[4-(4-fluoro-3- methyl-phenoxy)-phenyl]- ethylsulfamoyl}-benzoic acid | 57% yield MS: 456.3 (M − 1) |
| 489 | 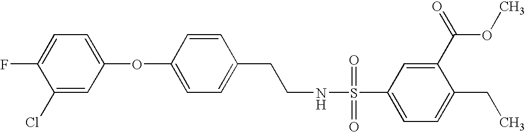 | 5-{2-[4-(3-Chloro-4-fluoro- phenoxy)-phenyl]- ethylsulfamoyl}-2-ethyl- benzoic acid | 89% yield MS: 476.2 (M − 1) |
| 490 | 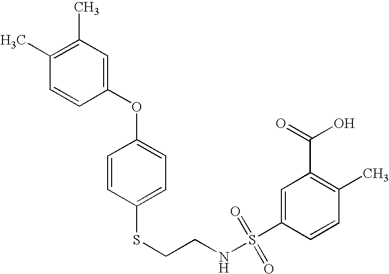 | 5-{2-[4-(3,4-Dimethyl- phenoxy)-phenylsulfanyl]- ethylsulfamoyl}-2-methyl- benzoic acid | 53% yield MS: 472.2 (M + 1) |
| 491 | 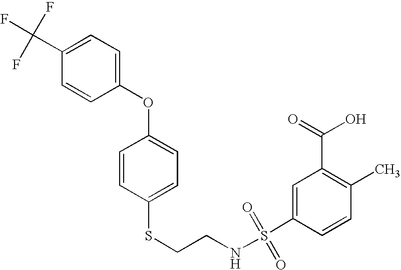 | 2-Methyl-5-{2-[4-(4- trifluoromethyl-phenoxy)- phenylsulfanyl]- ethylsulfamoyl}-benzoic acid | 74% yield MS: 510.1 (M − 1) |
| 492 | 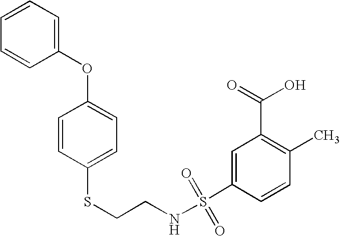 | 2-Methyl-5-[2-(4-phenoxy- phenylsulfanyl)- ethylsulfamoyl]-benzoic acid | 57% yield MS: 442.1 (M − 1) |
| 493 | 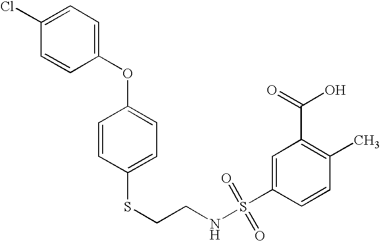 | 5-{2-[4-(4-Chloro-phenoxy)- phenylsulfanyl]- ethylsulfamol}-2-methyl- benzoic acid | 71% yield MS: 476.1 (M − 1) |
| 494 | 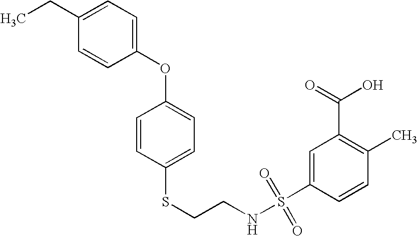 | 5-{2-[4-(4-Ethyl-phenoxy)- phenylsulfanyl]- ethylsulfamoyl}-2-methyl- benzoic acid | 82% yield MS: 472.4 (M + 1) |
| 495 | 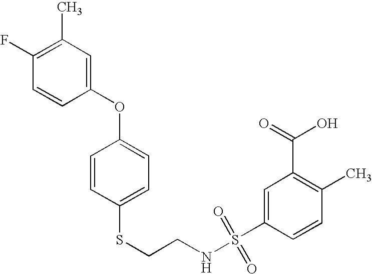 | 5-{2-[4-(4-Fluoro-3-methyl- phenoxy)-phenylsulfanyl]- ethylsulfamoyl}-2-methyl- benzoic acid | 57% yield MS: 474.2 (M − 1) |
| 496 | 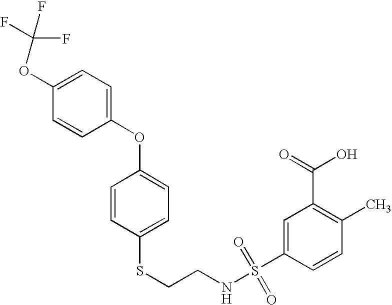 | 2-Methyl-5-{2-[4-(4- trifluoromethoxy-phenoxy)- phenylsulfanyl]- ethylsulfamoyl}-benzoic acid | 72% yield MS: 526.2 (M − 1) |
| 497 | 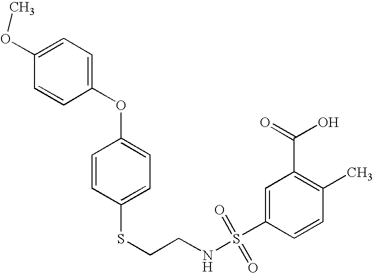 | 5-{2-[4-(4-Methoxy- phenoxy)-phenylsulfanyl]- ethylsulfamoyl}-2-methyl- benzoic acid | 72% yield MS: 472.2 (M − 1) |
| 498 | 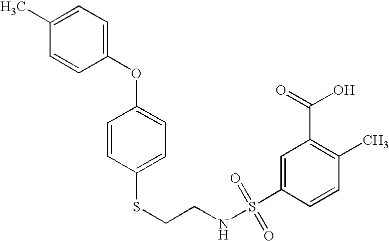 | 2-Methyl-5-[2-(4-p-tolyloxy- phenylsulfanyl)- ethylsulfamoyl]-benzoic acid | 97% yield MS: 456.2 (M − 1) |
| 499 | 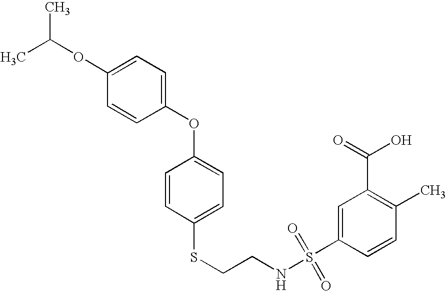 | 5-{2-[4-(4-Isopropoxy- phenoxy)-phenylsulfanyl]- ethylsulfamoyl}-2-methyl- benzoic acid | 58% yield MS: 500.2 (M − 1) |
| 500 | 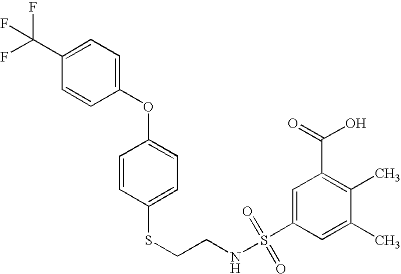 | 2,3-Dimethyl-5-{2-[4-(4- trifluoromethyl-phenoxy)- phenylsulfanyl]- ethylsulfamoyl}-benzoic acid | 92% yield ¹H NMR: See note 1 |
| 501 | 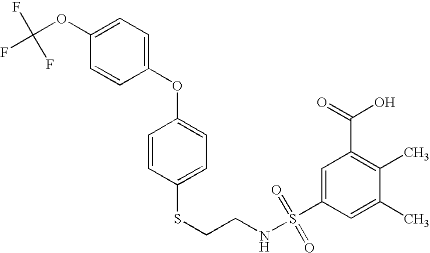 | 2,3-Dimethyl-5-{2-[4-(4- trifluoromethoxy-phenoxy)- phenylsulfanyl]- ethylsulfamoyl}-benzoic acid | 70% yield MS: 540.3 (M − 1) |
| 502 | 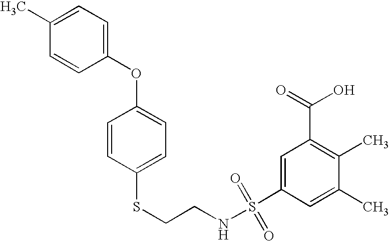 | 2,3-Dimethyl-5-[2-(4-p- tolyloxy-phenylsulfanyl)- ethylsulfamoyl]-benzoic acid | 38% yield MS: 470.3 (M − 1) |
| 503 | 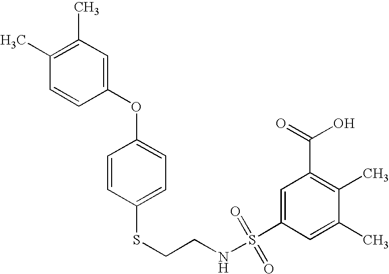 | 5-{2-[4-(3,4-Dimethyl- phenoxy)-phenylsulfanyl]- ethylsulfamoyl}-2,3- dimethyl-benzoic acid | 67% yield MS: 484.3 (M − 1) |
| 504 | 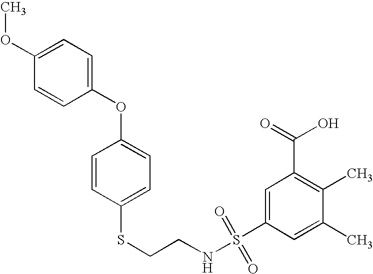 | 5-{2-[4-(4-Methoxy- phenoxy)-phenylsulfanyl]- ethylsulfamoyl}-2,3- dimethyl-benzoic acid | 75% yield MS: 486.3 (M − 1) |
| 505 | 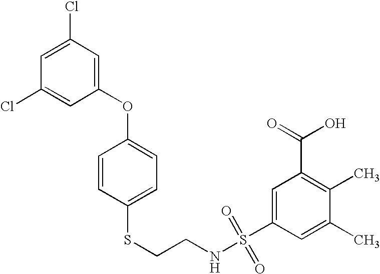 | 5-{2-[4-(3,5-Dichloro- phenoxy)-phenylsulfanyl]- ethylsulfamoyl}-2,3- dimethyl-benzoic acid | 50% yield MS: 525.2 (M − 1) |
| 506 | 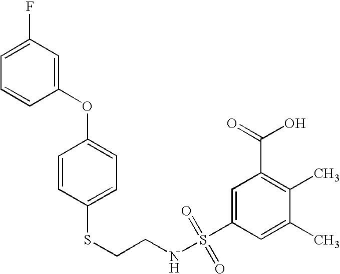 | 5-{2-[4-(3-Fluoro-phenoxy)- phenylsulfanyl]- ethylsulfamoyl}-2,3- dimethyl-benzoic acid | 81% yield MS: 474.3 (M − 1) |
| 507 | 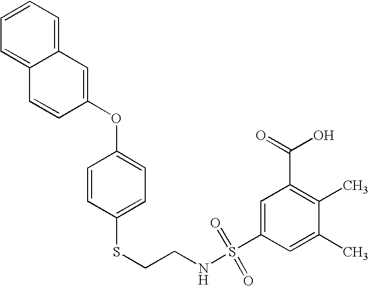 | 2,3-Dimethyl-5-{2-[4- (naphthalen-2-yloxy)- phenylsulfanyl]- ethylsulfamoyl}-benzoic acid | 80% yield MS: 506.3 (M − 1) |
| 508 | 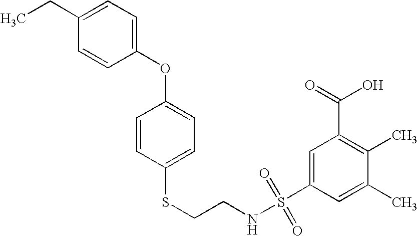 | 5-{2-[4-(4-Ethyl-phenoxy)- phenylsulfanyl]- ethylsulfamoyl}-2,3- dimethyl-benzoic acid | 69% yield MS: 484.3 (M − 1) |
| 509 | 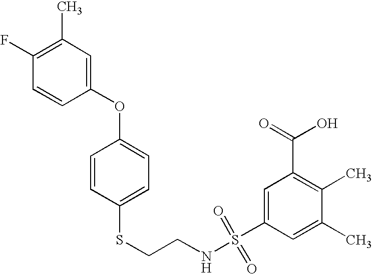 | 5-{2-[4-(4-Fluoro-3-methyl- phenoxy)-phenylsulfanyl]- ethylsulfamoyl}-2,3- dimethyl-benzoic acid | 76% yield MS: 488.3 (M − 1) |
| 510 | 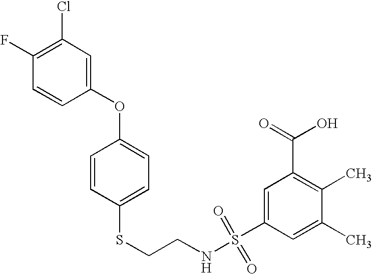 | 5-{2-[4-(3-Chloro-4-fluoro- phenoxy)-phenylsulfanyl]- ethylsulfamoyl}-2,3- dimethyl-benzoic acid | 66% yield MS: 508.2 (M − 1) |
| 511 | 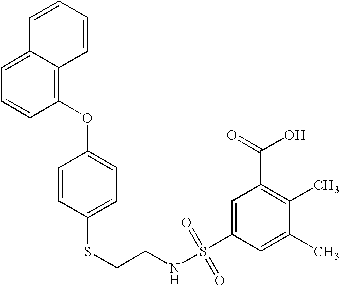 | 2,3-Dimethyl-5-{2-[4- (naphthalen-1-yloxy)- phenylsulfanyl]- ethylsulfamoyl}-benzoic acid | 59% yield MS: 506.3 (M − 1) |
| 512 | 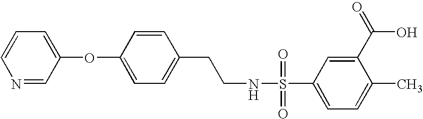 | 2-Methyl-5-{2-[4-(pyridin-3- yloxy)-phenyl]- ethylsulfamoyl}-benzoic acid | 39% yield MS: 413.2 (M + 1) |
| 513 | 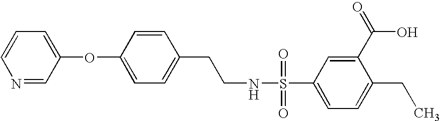 | 2-Ethyl-5-{2-[4-(pyridin-3- yloxy)-phenyl]- ethylsulfamoyl}-benzoic acid | 68% yield MS: 427.2 (M + 1) |
| 514 | 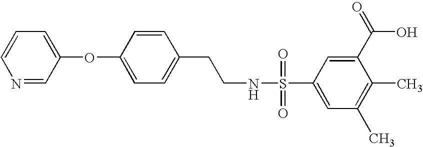 | 2,3-Dimethyl-5-{2-[4- (pyridin-3-yloxy)-phenyl]- ethylsulfamoyl}-benzoic acid | 59% yield MS: 427.2 (M + 1) |
| 515 | 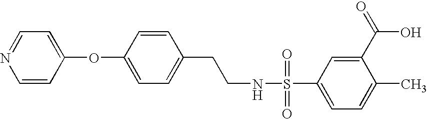 | 2-Methyl-5-{2-[4-(pyridin-4- yloxy)-phenyl]- ethylsulfamoyl}-benzoic acid | 40% yield MS: 413.2 (M + 1) |
| 516 | 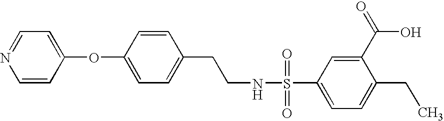 | 2-Ethyl-5-{2-[4-(pyridin-4- yloxy)-phenyl]- ethylsulfamoyl}-benzoic acid | 28% yield MS: 427.2 (M + 1) |
| 517 | 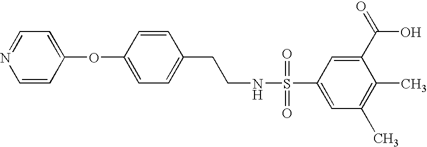 | 2,3-Dimethyl-5-{2-[4- (pyridin-4-yloxy)-phenyl]- ethylsulfamoyl}-benzoic acid | 21% yield MS: 427.2 (M + 1) |
| 518 | 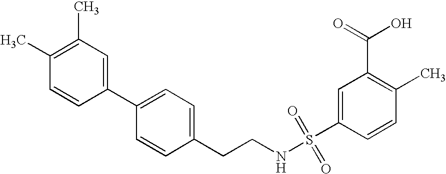 | 5-[2-(3′,4′-Dimethyl- biphenyl-4-yl)- ethylsulfamoyl]-2-methyl- benzoic acid | 88% yield MS: 422.3 (M − 1) |
| 519 | 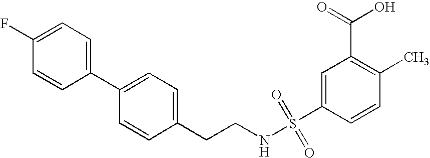 | 5-[2-(4′-Fluoro-biphenyl-4- yl)-ethylsulfamoyl]-2- methyl-benzoic acid | 95% yield MS: 412.3 (M − 1) |
| 520 | 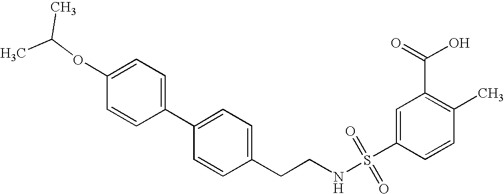 | 5-[2-(4′-Isopropoxy- biphenyl-4-yl)- ethylsulfamoyl]-2-methyl- benzoic acid | 78% yield MS: 452.3 (M − 1) |
| 521 | 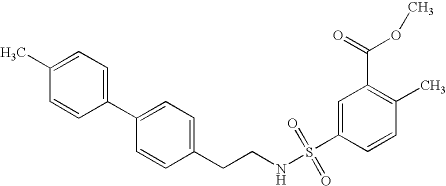 | 2-Methyl-5-[2-(4′-methyl- biphenyl-4-yl)- ethylsulfamoyl]-benzoic acid | 88% yield MS: 408.3 (M − 1) |
| 522 | 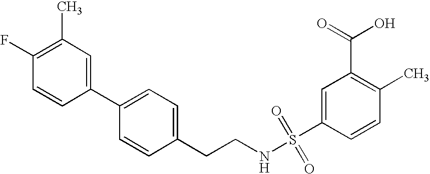 | 5-[2-(4′-Fluoro-3′-methyl- biphenyl-4-yl)- ethylsulfamoyl]-2-methyl- benzoic acid | 90% yield 426.3 (M − 1) |
| 523 | 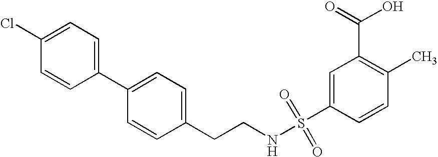 | 5-[2-(4′-Chloro-biphenyl-4- yl)-ethylsulfamoyl]-2- methyl-benzoic acid | 90% yield MS: 428.2 (M − 1) |
| 524 | 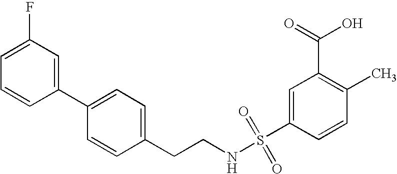 | 5-[2-(3′-Fluoro-biphenyl-4- yl)-ethylsulfamoyl]-2- methyl-benzoic acid | 96% yield MS: 412.3 (M − 1) |
| 525 | 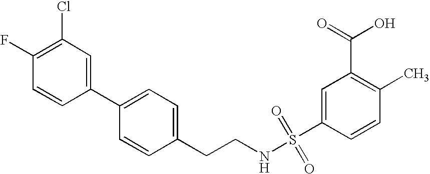 | 5-[2-(3′-Chloro-4′-fluoro- biphenyl-4-yl)- ethylsulfamoyl]-2-methyl- benzoic acid | 68% yield MS: 446.2 (M − 1) |
| 526 | 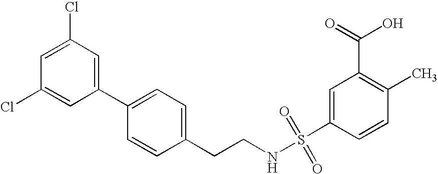 | 5-[2-(3′,5′-Dichloro- biphenyl-4-yl)- ethylsulfamoyl]-2-methyl- benzoic acid | 85% yield ¹H NMR: See Note 2 |
| 527 | 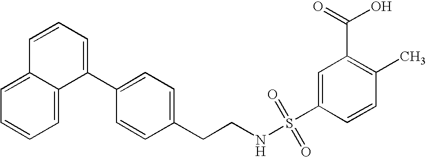 | 2-Methyl-5-[2-(4- naphthalen-1-yl-phenyl)- ethylsulfamoyl]-benzoic acid | 91% yield MS: 444.1 (M − 1) |
| 528 | 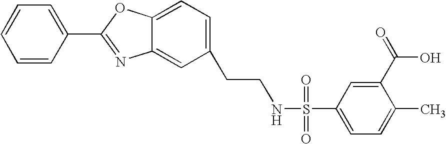 | 2-Methyl-5-[2-(2-phenyl- benzooxazol-5-yl)- ethylsulfamoyl]-benzoic acid | 93% yield MS: 435.2 (M − 1) |
| 529 | 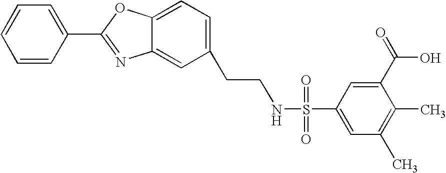 | 2,3-Dimethyl-5-[2-(2- phenyl-benzooxazol-5-yl)- ethylsulfamoyl]-benzoic acid | 99% yield MS: 449.3 (M − 1) |
| 530 | 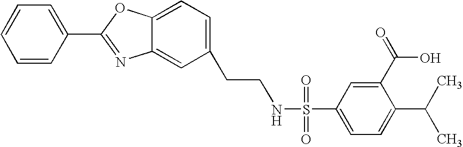 | 2-Isopropyl-5-[2-(2-phenyl- benzooxazol-5-yl)- ethylsulfamoyl]-benzoic acid | 97% yield MS: 463.3 (M − 1) |
| 531 | 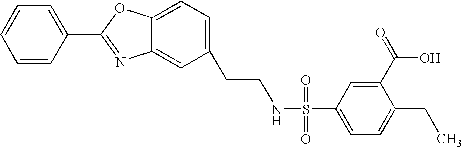 | 2-Ethyl-5-[2-(2-phenyl- benzooxazol-5-yl)- ethylsulfamoyl]-benzoic acid | 78% yield MS: 451.3 (M + 1) |
| 532 | 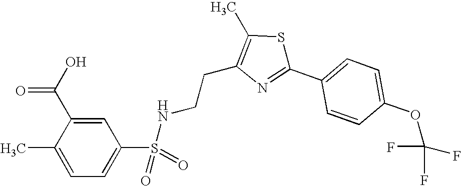 | 2-Methyl-5-{2-[5-methyl-2- (4-trifluoromethoxy- phenyl)-thiazol-4-yl]- ethylsulfamoyl}-benzoic acid | 96% yield MS: 501.3 (M + 1) |
| 533 | 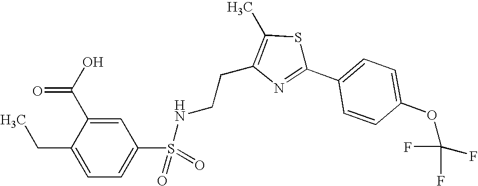 | 2-Ethyl-5-{2-[5-methyl-2-(4- trifluoromethoxy-phenyl)- thiazol-4-yl]- ethylsulfamoyl}-benzoic acid | 87% yield MS: 513.3 (M − 1) |
| 534 | 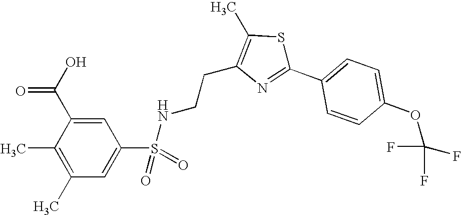 | 2,3-Dimethyl-5-{2-[5- methyl-2-(4- trifluoromethoxy-phenyl)- thiazol-4-yl]- ethylsulfamoyl}-benzoic acid | 97% yield MS: 513.3 (M − 1) |
| 535 | 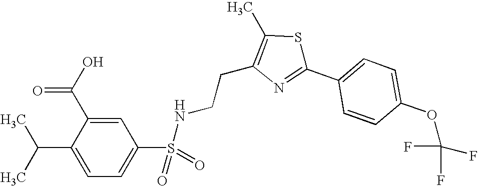 | 2-Isopropyl-5-{2-[5-methyl- 2-(4-trifluoromethoxy- phenyl)-thiazol-4-yl]- ethylsulfamoyl}-benzoic acid | 13% yield MS: 527.3 (M − 1) |
| 536 | 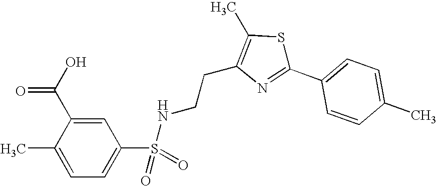 | 2-Methyl-5-[2-(5-methyl-2- p-tolyl-thiazol-4-yl)- ethylsulfamoyl]-benzoic acid | 89% yield 431.3 (M + 1) |
| 537 | 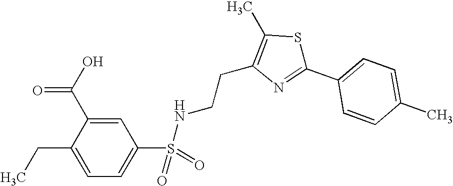 | 2-Ethyl-5-[2-(5-methyl-2-p- tolyl-thiazol-4-yl)- ethylsulfamoyl]-benzoic acid | 91% yield MS: 445.3 (M + 1) |
| 538 | 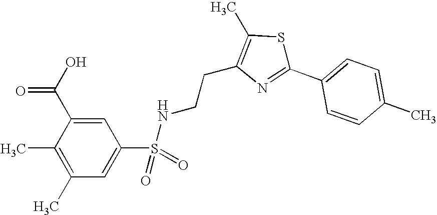 | 2,3-Dimethyl-5-[2-(5- methyl-2-p-tolyl-thiazol-4- yl)-ethylsulfamoyl]- benzoic acid | 82% yield MS: 445.3 (M + 1) |
| 539 | 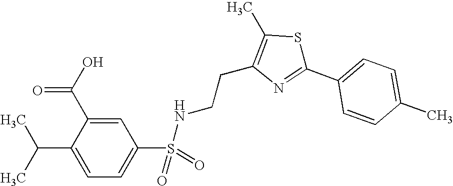 | 2-Isopropyl-5-[2-(5-methyl- 2-p-tolyl-thiazol-4-yl)- ethylsulfamoyl]-benzoic acid | 97% yield MS: 459.3 (M + 1) |
| 540 | 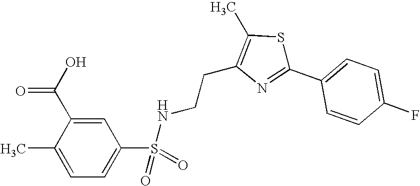 | 5-{2-[2-(4-Fluoro-phenyl)-5- methyl-thiazol-4-yl]- ethylsulfamoyl}-2-methyl- benzoic acid | 91% yield MS: 435.3 (M + 1) |
| 541 | 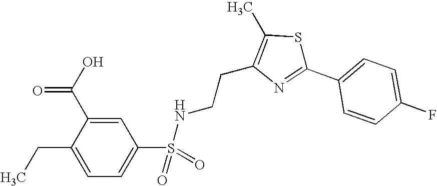 | 2-Ethyl-5-{2-[2-(4-Fluoro- phenyl)-5-methyl-thiazol-4- yl]-ethylsulfamoyl}- benzoic acid | 57% yield MS: 449.3 (M + 1) |
| 542 | 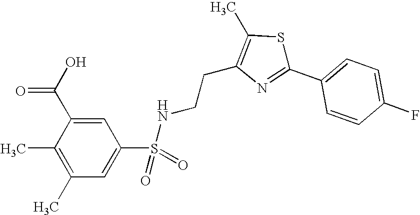 | 5-{2-[2-(4-Fluoro-phenyl)-5- methyl-thiazol-4-yl]- ethylsulfamoyl}-2,3- dimethyl-benzoic acid | 93% yield MS: 449.3 (M + 1) |
| 543 | 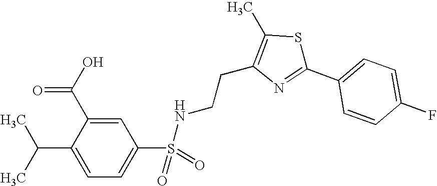 | 5-{2-[2-(4-Fluoro-phenyl)-5- methyl-thiazol-4-yl]- ethylsulfamoyl}-2- isopropyl-benzoic acid | 57% yield MS: 463.3 (M + 1) |
| 544 | 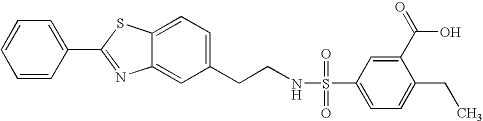 | 2-Ethyl-5-[2-(2-phenyl- benzothiazol-5-yl)- ethylsulfamoyl]-benzoic acid | 85% yield MS: 467.2 (M + 1) |
| 545 | 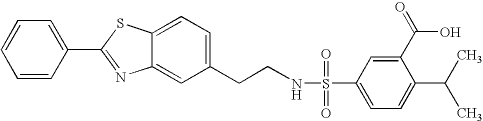 | 2-Isopropyl-5-[2-(2-phenyl- benzothiazol-5-yl)- ethylsulfamoyl]-benzoic acid | 99% yield MS: 481.2 (M + 1) |
| 546 | 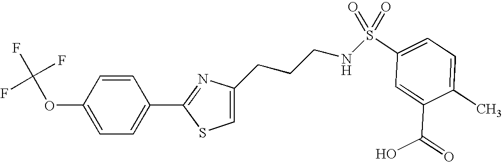 | 2-Methyl-5-{3-[2-(4- trifluoromethoxy-phenyl)- thiazol-4-yl]- propylsulfamoyl}-benzoic acid | 99% yield MS: 501.0 (M + 1) |
| 547 | 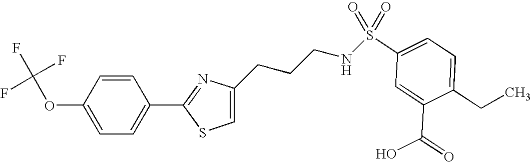 | 2-Ethyl-5-{3-[2-(4- trifluoromethoxy-phenyl)- thiazol-4-yl]- propylsulfamoyl}-benzoic acid | 58% yield MS: 515.0 (M + 1) |
| 548 | 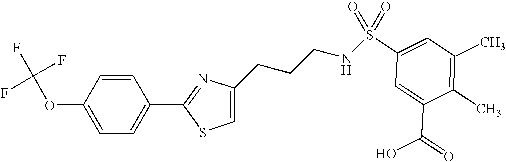 | 2,3-Dimethyl-5-{3-[2-(4- trifluoromethoxy-phenyl)- thiazol-4-yl]- propylsulfamoyl}-benzoic acid | 95% yield MS: 515.0 (M + 1) |
| 549 | 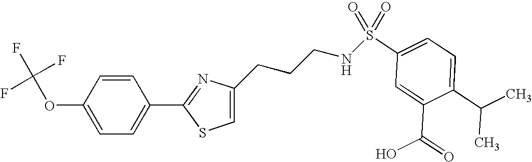 | 2-Isopropyl-5-{3-[2-(4- trifluoromethoxy-phenyl)- thiazol-4-yl]- propylsulfamoyl}-benzoic acid | 92% yield MS: 529.0 (M + 1) |
| 550 | 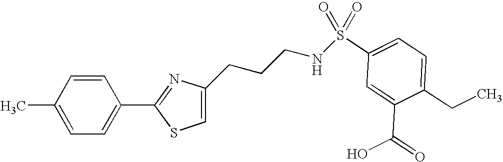 | 2-Ethyl-5-[3-(2-p-tolyl- thiazol-4-yl)- propylsulfamoyl]-benzoic acid | 62% yield MS: 445.0 (M + 1) |

### Note 1, Example 500

¹H NMR (400 MHz, CD₃OD): δ 2.38 (s, 3H), 2.50 (s, 3H), 2.93 (m, 2H), 3.00 (m, 2H), 6.97 m, 2H), 7.09 (d, 2H), 7.33 (m, 2H), 7.64 (d, 2H), 7.71 (d, 1H), 8.03 (d, 1H).

### Note 2. Example 526

¹H NMR (400 MHz, CD₃OD): δ 2.37 (s, 3H), 2.52 (s, 3H), 2.86 (t, 2H), 3.02 (m, 2H), 3.92 (s, 3H), 6.66 (m, 2H), 7.11 (m, 2H), 7.26 (m, 3H), 7.70 (d, 1H), 8.04 (d, 1H).

### Example 551

### 4-Methoxy-2-methyl-5-[2-(4-phenoxy-phenyl)-ethylsulfamoyl]-benzoic acid

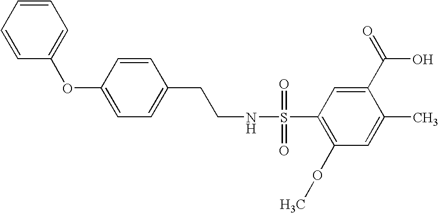

A mixture of 4-phenoxyphenethylamine (0.281 g, 1.32 mmol), 5-chlorosulfonyl-4-methoxy-2-methyl-benzoic acid 0.35 g, 1.32 mmol) and pyridine (0.321 ml, 3.96 mmol) in 20 ml anhydrous tetrahydrofuran and 4 ml dimethylformamide was heated at 60° C. for 3 hr. The reaction mixture was then cooled to room temperature and diluted with 120 ml ethyl acetate. The ethyl acetate solution was washed sequentially with 90 ml aqueous 1N hydrochloric acid solution, 90 ml water and 90 ml brine, dried (anhydrous sodium sulfate) and concentrated to dryness under reduced pressure. The residue was purified on a Shimadzu LCMS (reverse-phase column) using gradient elution with 0.1% formic acid in acetonitrile to yield the title compound (0.1 g, 17% yield).

¹H NMR (400 MHz, CDCl₃): δ 2.69 (s, 3H), 2.75 (m, 2H), 3.13 (m, 2H), 3.78 (s, 3H), 6.78 (s, 1H), 6.90 (m, 2H), 6.95 (m, 2H), 7.04 (m, 2H), 7.09 (m, 1H), 7.31 (m, 2H), 8.58 (s, 1H).

The title compound of EXAMPLE 552 was prepared using a procedure analogous to that of EXAMPLE 551 from appropriate starting materials.

### Example 552

### 5-[2-(4-Benzyloxy-3-methoxy-phenyl)-ethylsulfamoyl]-4-methoxy-2-meth benzoic acid

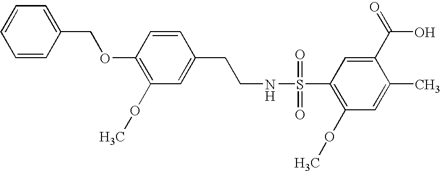

11% yield.

MS: 486.0 (M+1)

### Example 553

### 2-Methyl-5-[2-(4-p-tolylsulfanyl-phenyl)-ethylsulfamoyl]-benzoic acid

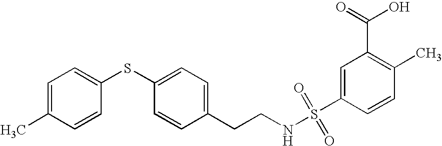

An oven-dried three-neck flask was charged with 4-methylbenzenethiol (32.5 mg, 0.26 mmol), cuprous iodide (8.3 mg, 0.043 mmol), potassium phosphate (115.5 mg, 0.544 mmol)) and N,N-dimethylglycine (4.5 mg, 0.043 mmol), evacuated and backfilled with nitrogen. A solution of 5-[2-(4-iodo-phenyl)-ethylsulfamoyl]-2-methyl-benzoic acid methyl ester (100 mg, 0.218 mmol) in 0.44 ml N,N-dimethylormamide was then added and the mixture was heated at 120° C. for 18 hr. The reaction mixture was cooled to room temperature and diluted with 50 ml ethyl acetate. The ethyl acetate solution was washed sequentially with 40 ml 1N aqueous hydrochloric acid solution and 40 ml brine, dried (anhydrous sodium sulfate) and concentrated to dryness under reduced pressure. The crude product was purified by flash column chromatography (8 g silica gel), eluting with 15% ethyl acetate in hexane followed by 2% methanol in chloroform, to yield the title compound as an off-white solid (10 mg, 10% yield). Under the reaction conditions the initially formed methyl ester hydrolyzed to the title compound.

MS: 440.3 (M−1)

The title compound of EXAMPLE 554 was prepared using a procedure analogous to that of EXAMPLE 553 from appropriate starting materials.

### Example 554

### 2-Methyl-5-{2-[4-(4-trifluoromethyl-phenylsulfanyl)-phenyl]-ethylsulfamoyl}-benzoic acid

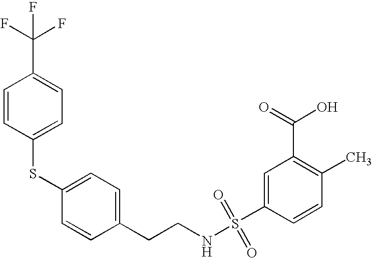

8% yield. MS: 494.2 (M−1)

### Example 555

### 5-(2-Bromo-ethylsulfamoyl)-2-methyl-benzoic acid methyl ester

Sodium bicarbonate (6.15 g, 73.2 mmol) was added to a solution of 2-bromoethylamine hydrobromide (5.0 g, 24.4 mmol) in a mixture of 12 ml water and 18 ml acetone cooled to 0° C., followed by addition of 5-chlorosulfonyl-2-methyl-benzoic acid methyl ester (6.05 g, 24.4 mmol). The reaction mixture was stirred at room temperature for 3 hr, then diluted with 150 ml water. The aqueous mixture was extracted with 150 ml ethyl acetate and the ethyl acetate solution was washed with 100 ml brine, dried (anhydrous sodium sulfate) and concentrated to dryness under reduced pressure. The crude product was purified by flash column chromatography (90 g silica gel), eluting with 4:1 hexane/ethyl acetate to yield the title compound as a colorless oil (6.14 g, 75% yield). MS: 336.9 (M+1)

The title compounds of EXAMPLES 556-557 were prepared using procedures analogous to that of EXAMPLE 555 from appropriate starting materials.

### Example 556

### 5-(2-Bromo-ethylsulfamoyl)-2,3-dimethyl-benzoic acid methyl ester 90% yield. MS: 351.2 (M+1)

### Example 557

### 5-(2-Bromo-ethylsulfamoyl)-2-ethyl-benzoic acid methyl ester 62% yield. MS: 350.3 (M)

### Example 558

### 5-[2-(4-Hydroxy-phenylsulfanyl)-ethylsulfamoyl]-2-methyl-benzoic acid methyl ester

A solution of 4-mercaptophenol (2.06 g, 16.4 mmol) in 5 ml methanol was added to a solution of sodium hydroxide (0.6 g, 14.9 mmol) in 5 ml methanol, followed by the addition of 5-(2-bromoethylsulfamoyl)-2-methyl-benzoic acid methyl ester (5.0 g, 14.9 mmol). The resulting solution was heated at reflux for 80 min, then concentrated to dryness under reduced pressure. The residue was dissolved in 100 ml ethyl acetate and the ethyl actate solution was washed sequentially with 90 ml water (acidified with 1 N aqueous hydrochloric acid solution) and 2×90 ml brine, dried (anhydrous sodium sulfate) and concentrated to dryness under reduced pressure. The crude product (5.92 g) was purified by column chromatography on silica gel (190 g), eluting with 1:2 ethyl acetate/hexane to yield the title compound as a white solid (4.31 g, 76% yield).

MS: 380.3 (M−1)

### Example 559

### 5-[2-(4-Bromo-phenyl)-ethylsulfamoyl]-2-methyl-benzoic acid methyl ester

A solution of 5-chlorosulfonyl-2-methyl-benzoic acid methyl ester (2.48 g, 10 mmol), 4-bromophenethylamine (2.0 g, 10 mmol) and pyridine (2.42 ml, 30 mmol) in a mixture of 40 ml tetrahydrofuran and 30 ml dimethylformamide was heated at 70° C. for 2 hr. The reaction mixture was then diluted with 350 ml ethyl acetate and the ethyl acetate solution was washed sequentially with 200 ml 1N aqueous sodium hydroxide solution, 200 ml water and 200 ml brine, dried (anhydrous sodium sulfate) and concentrated to dryness under reduced pressure. The crude product was purified by flash column chromatography (90 g silica gel), eluting with 85:15 hexane/ethyl acetate to yield the title compound as a colorless oil (1.52 g, 37% yield).

MS: 413.0 (M+1)

The title compound of EXAMPLE 560 was prepared using a procedure analogous to that of EXAMPLE 559 from appropriate starting materials.

### Example 560

### 5-[2-(4-Bromo-phenyl)-ethylsulfamoyl]-2,3-dimethyl-benzoic acid methyl ester

51% yield. MS: 427.3 (M+1)

### Example 561

### 5-[2-(4-Iodo-phenyl)-ethylsulfamoyl]-2-methyl-benzoic acid methyl ester

The title compound was prepared using a procedure analogous to that of EXAMPLE 66, using appropriate starting materials, in particular, using 2-(4-iodo-phenyl)-ethylamine and 5-chlorosulfonyl-2-methyl-benzoic acid methyl ester as reactants. 45% yield. MS: 460.3 (M+1)

### Example 562

### 5-[2-(4-Hydroxy-phenylsulfanyl)-ethylsulfamoyl]-2-methyl-benzoic acid methyl ester

A solution of 4-mercaptophenol (2.06 g, 16.4 mmol) in 5 ml methanol was added to a solution of sodium methoxide (0.6 g, 14.9 mmol) in 5 ml methanol. 5-(2-Bromo-ethylsulfamoyl)-2-methyl-benzoic acid methyl ester (5.00 g, 14.9 mmol) was then added and the resulting solution was heated at reflux for 80 min. The reaction mixture was then cooled to room temperature and concentrated to dryness under reduced pressure. The residue was dissolved in 100 ml ethyl acetate and the ethyl acetate solution was washed sequentially with 90 ml dilute aqueous hydrochloric acid solution and 2×90 ml brine, dried (anhydrous sodium sulfate) and concentrated to dryness under reduced pressure. The crude product (5.92 g) was purified by column chromatography (190 g silica gel), eluting with 2:1 hexane/ethyl acetate to yield the title compound (4.31 g, 76% yield). MS: 380.3 (M-1)

The title compound of EXAMPLE 563 was prepared using a procedure analogous to that of EXAMPLE 562 from appropriate starting materials.

### Example 563

### 5-[2-(4-Hydroxy-phenylsulfanyl)-ethylsulfamoyl]-2,3-dimethyl-benzoic acid methyl ester

80% yield. MS: 394.3 (M−1)

### Example 564

### 2-[2-(5-Chloro-benzooxazol-2-yl)-ethyl]-isoindole-1,3-dione

A mixture of N-phthaloyl-β-alanine (1.0 g, 4.56 mmol) and 5-chloro-2-hydroxyaniline (0.65 g, 4.56 mmol) in 20 ml polyphosphoric acid was heated to 190° C. for 6 hr. The reaction mixture was cooled to room temperature and 100 ml water was added to dissolve the polyphosphoric acid. The resulting mixture was filtered and the solid product was dissolved in 50 ml ethyl acetate. The ethyl acetate solution was washed sequentially with 2×40 ml saturated aqueous sodium bicarbonate solution, 40 ml water and 40 ml brine, dried (anhydrous sodium sulfate) and concentrated to dryness under reduced pressure to yield the title compound as a yellowish solid (1.13 g, 76% yield). MS: 327.1 (M+1)

The title compounds of EXAMPLES 565-566 were prepared using procedures analogous to that of EXAMPLE 564 from appropriate starting materials.

### Example 565

### 2-[2-(5-Methyl-benzooxazol-2-yl)-ethyl]-isoindole-1,3-dione

71% yield. MS: 307.0 (M+1)

### Example 566

### 2-[2-(5-Benzooxazol-2-yl)-ethyl]-isoindole-1,3-dione

76% yield. MS: 293.2 (M+1)

The title compounds of EXAMPLES 567-568 were prepared using procedures analogous to that of EXAMPLES 564 and 566 but using the appropriate thiophenol instead of the phenol.

### Example 567

### 2-[2-(5-Benzothiazol-2-yl)-ethyl]-isoindole-1,3-dione

87% yield. MS: 309.2 (M+1)

### Example 568

### 2-[2-(5-Trifluoromethyl-benzothiazol-2-yl)-ethyl]-isoindole-1,3-dione

66% yield. MS: 377.1 (M+1)

### Example 569

### 2-[2-(5-tert-Butyl-benzooxazol-2-yl)-ethyl]-isoindole-1,3-dione N-(5-tert-Butyl-2-hydroxy-phenyl)-3-(1,3-dioxo-1,3-dihydro-isoindol-2-yl)-propionamide

N-Phthaloyl β-alanine (1.0 g, 4.56 mmol) was added to 10 ml thionyl chloride and the reaction mixture was heated at reflux for 3 hr, cooled to room temperature and concentrated to dryness under reduced pressure to yield the corresponding the acid chloride (1.08 g, 100% yield). The acid chloride (0.35 g, 1.47 mmol) was dissolved in 10 ml methylene chloride, then 2-amino-4-tert-butylphenol (0.243 g, 1.47 mmol), and 4-dimethylaminopyridine (0.198 g, 1.62 mmol) were added to the resulting solution. After stirring overnight at room temperature, 40 ml methylene chloride was added to the reaction mixture and the methylene chloride solution was washed sequentially with 40 ml water and 40 ml brine, dried (anhydrous sodium sulfate) and concentrated to dryness under reduced pressure. The crude product (0.55 g) was purified by flash column chromatography (15 g silica gel) eluting with 7:3 hexane/ethyl acetate to yield the title compound as a yellowish solid (0.42 g, 78% yield). MS: 365.1 (M−1)

### 2-[2-(5-tert-Butyl-benzooxazol-2-yl)-ethyl]-isoindole-1,3-dione

Diethyl azodicarboxylate (0.20 ml, 1.27 mmol) was added dropwise with stirring to a solution of N-(5-tert-butyl-2-hydroxy-phenyl)-3-(1,3-dioxo-1,3-dihydro-isoindol-2-yl)-propionamide (0.423 g, 1.15 mmol) and triphenylphosphine (0.333 g, 1.27 mmol) in 5 ml tetrahydrofuran. The reaction mixture was stirred overnight at room temperature, then diluted with 50 ml ethyl acetate. The ethyl acetate solution was washed sequentially with 40 ml saturated aqueous sodium bicarbonate solution, 40 ml water and 40 ml brine, dried (anhydrous sodium sulfate) and concentrated to dryness under reduced pressure. The crude product was purified by flash column chromatography (15 g silica gel), eluting with 85:15 hexane/ethyl acetate to yield the title compund as a yellowish solid (0.287 g, 71% yield). MS: 349.1 (M+1)

The title compound of EXAMPLE 570 was prepared using a procedure analogous to that of EXAMPLE 569 from appropriate starting materials.

### Example 570

### 2-[2-(5-Phenyl-benzooxazol-2-yl)-ethyl]-isoindole-1,3-dione 3-(1,3-Dioxo-1,3-dihydro-isoindol-2-yl)-N-(4-hydroxy-biphenyl-3-yl)-propionamide

73% yield. MS: 387.1 (M+1)

### 2-[2-(5-Phenyl-benzooxazol-2-yl)-ethyl]-isoindole-1,3-dione

74% yield. MS: 369.1 (M+1)

### Example 571

### 2-(5-tert-Butyl-benzooxazol-2-yl)ethylamine

A solution of 2-[2-(5-tert-butylbenzooxazol-2-yl)-ethyl]-isoindole-1,3-dione (0.087 g, 0.249 mmol) and hydrazine monohydrate (0.013 ml, 0.274 mmol) in 3 ml ethanol in a 5 ml microwave vial was irradiated in a microwave oven (high power) at 160° C. for 20 min. The cooled reaction mixture was diluted with 2 ml ethanol and stirred at room temperature for 5 min. The precipitated solid was filtered and the filtrate was concentrated to dryness under reduced pressure. The crude product (0.072 g) was purified by flash column chromatography (15 g, silica gel), eluting with 9:1 chloroform/methanol to yield the title compund as a yellowish oil (0.043 g, 80% yield). MS: 219.1 (M+1)

The title compounds of EXAMPLES 572-579 were prepared using procedures analogous to that of EXAMPLE 571 from appropriate starting materials.

### Example 572

### 2-(5-Methyl-benzooxazol-2-yl)ethylamine

83% yield. MS: 177.1 (M+1)

### Example 573

### 2-(5-Chloro-benzooxazol-2-yl)ethylamine

92% yield. MS: 197.1 (M+1)

### Example 574

### 2-(5-Phenyl-benzooxazol-2-yl)ethylamine

20% yield. MS: 239.1 (M+1)

### Example 575

### 2-(Benzooxazol-2-yl)ethylamine

40% yield. ¹H NMR (400 MHz, CDCl₃): δ 3.1 (m, 2H), 3.28 (m, 2H), 7.29 (m, 2H), 7.47 (m, 1H), 7.64 (m, 1H).

### Example 576

### 2-(Benzothiazol-2-yl)ethylamine

27% yield. MS: 179.1 (M+1)

### Example 577

### 2-(5-Trifluoromethyl-benzothiazol-2-yl)ethylamine

66% yield. MS: 247.2 (M+1)

### Example 578

### 2-(4-Trifluoromethyl-phenylsulfanyl)-ethylamine

69% yield. MS: 222.2 (M+1)

### Example 579

### 2-(4-Cyclohexyl-phenoxy)-ethylamine

77% yield. MS: 220.3 (M+1)

### Example 580

### 2-[2-(4-tert-Butyl-phenoxy)-ethyl]-isoindole-1,3-dione

Diethyl azodicarboxylate (1.15 ml, 7.32 mmol) was added dropwise to a solution of 4-tert-butylphenol (1 g, 6.66 mmol), N-(2-hydroxyethyl)phthalimide (1.27 g, 6.66 mmol) and triphenylphosphine (1.92 g, 7.32 mmol) in 30 ml tetrahydrofuran and the reaction mixture was stirred at room temperature overnight. 120 ml ethyl acetate was then added and the ethyl acetate solution was washed sequentially with 100 ml saturated aqueous sodium bicarbonate solution, 100 ml water and 100 ml brine, dried (anhydrous sodium sulfate) and concentrated to dryness under reduced pressure. The crude product was purified by flash column chromatography (15 g silica gel), eluting with 7:3 hexane/ethyl acetate to yield the title compound (0.43 g, 20% yield)

¹H NMR (400 MHz, CDCl₃): 1.29 (s, 9H), 4.13 (m, 2H), 4.22 (m, 2H), 6.78 (m, 4H), 7.26 (m, 4H).

### Example 581

### 2-[2-(Biphenyl-4-yloxy)-ethyl]-isoindole-1,3-dione

The title compound was prepared using a procedure analogous to that of EXAMPLE 580 except that for the workup the reaction mixture was poured into 150 ml methanol and the title compound was obtained by filtering the mixture. 57% yield. ¹H NMR (400 MHz, CDCl₃): δ 4.13 (m, 2H), 4.27 (m, 2H), 6.94 (m, 2H), 7.27 (m, 1H), 7.39 (m, 2H), 7.44 (m, 4H), 7.72 (m, 2H), 7.86 (m, 2H).

### Example 582

### 2-(4-tert-Butyl-phenoxy)-ethylamine

A mixture of 2-[2-(4-tert-butyl-phenoxy)-ethyl]isoindole-1,3-dione (0.427 g, 1.32 mmol) in aqueous 4N sodium hydroxide solution (3 ml, 12 mmol) in a 5 ml microwave vial was irradiated in a microwave oven (high power) at 200° C. for 6 min. The cooled reaction mixture was diluted with 100 ml methanol and filtered. The filtrate was concentrated to dryness under reduced pressure and the residue was triturated with 50 ml ethyl acetate and filtered. The filtrate was concentrated to dryness to yield the title compound (0.08 g, 31% yield). MS: 194.1 (M+1)

The title compound of EXAMPLE 583 and was obtained using a procedure analogous to that of EXAMPLE 582 from appropriate starting materials.

### Example 583

### 2-(Biphenyl-4-yloxy)-ethylamine

89% yield. MS: 214.1 (M+1)

### Example 584

### [5-Methyl-2-(4-trifluoromethyl-phenyl)-thiazol-4-yl]-acetic acid ethyl ester

A solution of 4-bromo-3-oxo-pentanoic acid ethyl ester (1.0 g, 4.48 mmol) and 4-(trifluoromethyl)thiobenzamide (0.919 g, 4.48 mmol) in 20 ml ethanol was heated at 80° C. for 2 hr. The cooled reaction mixture was poured into 100 ml water and the aqueous mixture was extracted with 130 ml ethyl acetate. The ethyl acetate solution was washed with 80 ml brine, dried (anhydrous sodium sulfate) and concentrated to dryness under reduced pressure. The crude product (1.42 g) was purified by flash column chromatography (40 g silica gel), eluting with 93:7 hexane/ethyl acetate to yield the title compound as a yellowish solid (0.9 g, 61% yield).

The title compounds of EXAMPLES 585-589 were prepared using procedures analogous to that of EXAMPLE 584 from appropriate starting materials.

### Example 585

### [2-(4-Chloro-phenyl)-5-methyl-thiazol-4-yl]-acetic acid ethyl ester

69% yield. MS: 296.1 (M+1)

### Example 586

### [2-(3-Chloro-4-fluoro-phenyl)-5-methyl-thiazol-4-yl]-acetic acid ethyl ester

55% yield. MS: 314.1 (M+1)

### Example 587

### [2-(4-tert-Butyl-phenyl)-5-methyl-thiazol-4-yl]-acetic acid ethyl ester

79% yield. MS: 318.2 (M+1)

### Example 588

### [5-Methyl-2-[4-(5-trifluoromethyl-pyridin-2-yloxy)-phenyl]-thiazol-4-yl]-acetic acid ethyl ester

64% yield. MS: 423.3 (M+1)

### Example 589

### [5-Methyl-2-(3-pyrrol-1-yl-phenyl)-thiazol-4-yl]-acetic acid ethyl ester

70% yield. MS: 327.3 (M+1)

The title compounds of EXAMPLES 590-595 were prepared using procedures analogous to that of EXAMPLES 584 and 589 but using ethyl 4-chloroacetoacetate instead of 4-bromo-3-oxo-pentanoic acid ethyl ester.

### Example 590

### [2-(4-tert-Butyl-phenyl)-thiazol-4-yl]-acetic acid ethyl ester

82% yield. MS: 304.3 (M+1)

### Example 591

### [2-(2,4-Difluoro-phenyl)-thiazol-4-yl]-acetic acid ethyl ester

96% yield. MS: 284.3 (M+1)

### Example 592

### (2-p-Tolyl-thiazol-4-yl)-acetic acid ethyl ester

100% yield. MS: 262.3 (M+1)

### Example 593

### [2-(4-Fluoro-phenyl)-thiazol-4-yl]-acetic acid ethyl ester

90% yield. MS: 266.3 (M+1)

### Example 594

### [2-(3-Chloro-4-fluoro-phenyl)-thiazol-4-yl]-acetic acid ethyl ester

76% yield. MS: 300.2 (M+1)

### Example 595

### [2-(4-Trifluoromethyl-phenyl)-thiazol-4-yl]-acetic acid ethyl ester

80% yield. MS: 316.1 (M+1)

### Example 596

### 3-[2-(4-Fluorophenyl)-thiazol-4-yl]-propionic acid ethyl ester

A solution of 5-bromo-4-oxo-pentanoic acid methyl ester (1.01 g, 4.83 mmol) and 4-fluorothiobenzamide (0.5 g, 3.22 mmol) in 20 ml ethanol was heated at 80° C. for 4 hr. The reaction mixture was then cooled to room temperature, poured into 100 ml water and the aqueous mixture was extracted with 130 ml ethyl acetate. The ethyl acetate solution was washed with 80 ml brine, dried (anhydrous sodium sulfate) and concentrated to dryness under reduced pressure. The crude product (1.24 g) was purified by flash column chromatography (15 g silica gel), eluting with 93:7 hexane/ethyl acetate to yield the title compound as a yellowish oil (0.92 g, 100% yield). During the reaction transesterification of the original product occurred, to yield the ethyl ester as product. MS: 280.3 (M+1)

The title compound of EXAMPLES 597-598 were prepared using a procedure analogous to that of EXAMPLE 596 from appropriate starting materials.

### Example 597

### 3-[2-(4-Trifluoromethyl-phenyl)-thiazol-4-yl]-propionic acid ethyl ester

70% yield. MS: 330.4 (M+1)

### Example 598

### 3-[2-(4-Fluorophenyl)-thiazol-4-yl]-propionic acid ethyl ester

45% yield. MS: 280.3 (M+1)

### Example 599

### [2-(4-Trifluoromethyl-phenyl)-oxazol-4-yl]-acetic acid ethyl ester

A mixture of 4-tert-butylbenzamide (1.0 g, 5.64 mmol), ethyl 4-chloroacetoacetate (1.16 g, 7.05 mmol), and p-toluenesulphonic acid (0.194 g, 1.13 mmol) in 2 ml ethanol was irradiated in a microwave oven (high power) at 170° C. for 20 min. The reaction mixture was cooled to room temperature and diluted with 40 ml ethyl acetate. The ethyl acetate solution was washed sequentially with 30 ml 1N aqueous hydrochloric acid solution, 30 ml water and 30 ml brine, dried (anhydrous sodium sulfate) and concentrated to dryness under reduced pressure to yield the title compound as a brownish oil (1.53 g, 93% yield). MS: 300.1 (M+1)

The title compounds of EXAMPLES 600-601 were prepared using procedures analogous to that of EXAMPLE 599 from appropriate starting materials.

### Example 600

### [2-(4-tert-Butyl-phenyl)-oxazol-4-yl]-acetic acid ethyl ester

95% yield. MS: 288.2 (M+1)

### Example 601

### (2-Cyclohexyl-oxazol-4-yl)-acetic acid ethyl ester

64% yield. MS: 238.2 (M+1)

### Example 602

### [2-(4-tert-Butyl-phenyl)-5-methyl-oxazol-4-yl]-acetic acid ethyl ester

A mixture of 4-tert-butylbenzamide (1.0 g, 5.64 mmol), 4-bromo-3-oxo-pentanoic acid ethyl ester 1.26 g, (5.64 mmol) and p-toluenesulphonic acid (0.194 g, (1.13 mmol) in 5 ml ethanol was heated at reflux for 65 hr. The reaction mixture was cooled to room temperature and diluted with 60 ml ethyl acetate. The ethyl acetate solution was washed sequentially with 40 ml 1N aqueous hydrochloric acid solution, 40 ml water and 40 ml brine, dried (anhydrous sulfate) and concentrated to dryness under reduced pressure. The crude product was purified by flash column chromatography (40 g silica gel), eluting with 97:3 hexane/ethyl acetate to yield the title compound (0.232 g, 14% yield). MS: 302.4 (M+1)

### Example 603

### 2-[5-Methyl-2-(4-trifluoromethyl-phenyl)-thiazol-4-yl]-ethanol

A solution of [5-methyl-2-(4-trifluoromethyl-phenyl)-thiazol-4-yl]-acetic acid ethyl ester (0.814 g, 2.47 mmol) in 1 ml tetrahydrofuran was added to a solution of lithium aluminum hydride (1.24 ml of 1M solution in tetrahydrofuran, 1.24 mmol) in 4 ml tetrahydrofuran cooled to 0° C. The reaction mixture was stirred at 0° C. for 2 hr, then was quenched at that temperature by the sequential addition of 6.5 ml diethyl ether, 0.09 ml water, 0.09 ml aqueous 1N sodium hydroxide solution and 0.231 ml water. The resulting mixture was stirred at room temperature for 15 min, then filtered. The filtrate was concentrated to dryness under reduced pressure to yield the title compound as a white solid (0.674 g, 95% yield). MS: 288.1 (M+1)

The title compounds of EXAMPLES 604-615 were prepared using procedures analogous to that of EXAMPLE 603 from appropriate starting materials.

### Example 604

### 2-[2-(4-Chloro-phenyl)-5-methyl-thiazol-4-yl]-ethanol

100% yield. MS: 254.1 (M+1)

### Example 605

### 2-[2-(3-Chloro-4-fluoro-phenyl)-5-methyl-thiazol-4-yl]-ethanol

100% yield. MS: 272.1 (M+1)

### Example 606

### 2-(5-Methyl-2-phenyl-thiazol-4-yl)-ethanol

100% yield. MS: 220.1 (M+1)

### Example 607

### 2-[2-(4-tert-Butyl-phenyl)-5-methyl-thiazol-4-yl]-ethanol

92% yield. MS: 276.2 (M+1)

### Example 608

### 2-{5-Methyl-2-[4-(5-trifluoromethyl-pyridin-2-yloxy)-phenyl]-thiazol-4-yl}-ethanol

100% yield. MS: 381.3 (M+1)

### Example 609

### 2-[5-Methyl-2-(3-pyrrol-1-yl-phenyl)-thiazol-4-yl]-ethanol

91% yield. MS: 285.3 (M+1)

### Example 610

### 2-[2-(2,4-Difluoro-phenyl)-thiazol-4-yl]-ethanol

87% yield. MS: 242.2 (M+1)

### Example 611

### 2-(2-p-Tolyl-thiazol-4-yl)-ethanol

90% yield. MS: 220.3 (M+1)

### Example 612

### 2-[2-(4-Fluoro-phenyl)-thiazol-4-yl]-ethanol

100% yield. MS: 224.2 (M+1)

### Example 613

### 2-[2-(3-Chloro-4-fluoro-phenyl)-thiazol-4-yl]-ethanol

50% yield. MS: 258.2 (M+1)

### Example 614

### 2-(2-Cyclohexyl-oxazol-4-yl)-ethanol

69% yield. MS: 196.1 (M+1)

### Example 615

### 2-[2-(4-tert-Butyl-phenyl)-5-methyl-oxazol-4-yl]-ethanol

86% yield. MS: 260.4 (M+1)

The title compounds of EXAMPLES 616-621 were prepared using procedures analogous to that of EXAMPLE 602 from appropriate starting materials except that the crude products were purified by flash column chromatography on silica gel, eluting with 7:3 hexane/ethyl acetate.

### Example 616

### 2-(5-Methyl-2-naphthalen-2-yl-thiazol-4-yl)-ethanol

87% yield. MS: 270.1 (M+1)

### Example 617

### 2-[2-(4-Trifluoromethyl-phenyl)-thiazol-4-yl]-ethanol

74% yield. MS: 274.2 (M+1)

### Example 618

### 2-[2-(4-tert-Butyl-phenyl)-thiazol-4-yl]-ethanol

89% yield. MS: 262.3 (M+1)

### Example 619

### 3-[2-(4-Trifluoromethyl-phenyl)-thiazol-4-yl]-propan-1-ol

60% yield. MS: 288.3 (M+1)

### Example 620

### 2-[2-(4-Trifluoromethyl-phenyl)-oxazol-4-yl]-ethanol

9% yield. MS: 258.1 (M+1)

### Example 621

### 2-[2-(4-tert-Butyl-phenyl)-oxazol-4-yl]-ethanol

32% yield. MS: 246.2 (M+1)

### Example 622

### 4-(2-Azido-ethyl)-5-methyl-2-(4-trifluoromethyl-phenyl)-thiazole

Methanesulfonyl chloride (0.20 ml, 2.58 mmol) was added dropwise to a solution of 2-[5-methyl-2-(4-trifluoromethyl-phenyl)-thiazol-4-yl]-ethanol (0.674 g, 2.35-mmol) and triethylamine (0.49 ml, 3.52 mmol) in 10 ml methylene chloride cooled to 0° C. The reaction mixture was stirred overnight at room temperature, then diluted with 30 ml methylene chloride. The methylene chloride solution was washed sequentially with 40 ml aqueous 1N hydrochloride solution, 40 ml water and 40 ml brine, dried (anhydrous sodium sulfate) and concentrated to dryness under reduced pressure to yield the corresponding methanesulfonate. The methanesulfonate was dissolved in 10 ml dimethylformamide, sodium azide (0.165 g, 2.30 mmol) was added and the reaction mixture was heated at 80° C. overnight. The reaction mixture was cooled to room temperature and diluted with 80 ml ethyl acetate. The ethyl acetate solution was washed sequentially with 70 ml aqueous 1N hydrochloric acid solution, 70 ml water and 70 ml brine, dried (anhydrous sodium sulfate) and concentrated to dryness under reduced pressure to yield the title compound as a yellowish solid (0.668 g, 91% yield).

MS: 313.1 (M+1).

The title compounds of EXAMPLES 623-640 were prepared using procedures analogous to that of EXAMPLE 622 from appropriate starting materials.

### Example 623

### 4-(2-Azido-ethyl)-2-(4-chloro-phenyl)-5-methyl-thiazole

86% yield. MS: 279.1 (M+1)

### Example 624

### 4-(2-Azido-ethyl)-2-(3-chloro-4-fluoro-phenyl)-5-methyl-thiazole

64% yield. MS: 297.1 (M+1)

### Example 625

### 4-(2-Azido-ethyl)-5-methyl-2-naphthalen-2-yl-thiazole

100% yield. ¹H NMR (400 MHz, CDCl₃): δ 2.49 (s, 3H), 3.04 (m, 2H), 3.75 (m, 2H), 7.51 (m, 2H), 7.85 (m, 2H), 7.92 (m, 1H), 8.02 (m, 1H), 8.39 (s, 1H).

### Example 626

### 4-(2-Azido-ethyl)-5-methyl-2-phenyl-thiazole

96% yield. MS: 245.1 (M+1)

### Example 627

### 4-(2-Azido-ethyl)-2-(4-tert-butyl-phenyl)-5-methyl-thiazole

95% yield. MS: 301.2 (M+1)

### Example 628

### 2-{4-[4-(2-Azido-ethyl)-5-methyl-thiazol-2-yl]-phenoxy}-5-trifluoromethyl-pyridine

80% yield. MS: 406.3 (M+1)

### Example 629

### 4-(2-Azido-ethyl)-5-methyl-2-(3-pyrrol-1-yl-phenyl)-thiazole

91% yield. MS: 310.3 (M+1)

### Example 630

### 4-(2-Azido-ethyl)-2-(4-trifluoromethyl-phenyl)-thiazole

100% yield. MS: 299.1 (M+1)

### Example 631

### 4-(2-Azido-ethyl)-2-(4-tert-butyl-phenyl)-thiazole

78% yield. MS: 287.3 (M+1)

### Example 632

### 4-(2-Azido-ethyl)-2-(2,4-difluoro-phenyl)-thiazole

93% yield. MS: 267.3 (M+1)

### Example 633

### 4-(2-Azido-ethyl)-2-p-tolyl-thiazole

75% yield. MS: 220.3 (M+1)

### Example 634

### 4-(2-Azido-ethyl)-2-(4-fluoro-phenyl)-thiazole

76% yield. MS: 249.3 (M+1)

### Example 635

### 4-(2-Azido-ethyl)-2-(3-chloro-4-fluoro-phenyl)-thiazole

100% yield. MS: 283.2 (M+1)

### Example 636

### 4-(3-Azido-propyl)-2-(4-trifluoromethyl-phenyl)-thiazole

42% yield. MS: 313.3 (M+1)

### Example 637

### 4-(2-Azido-ethyl)-2-(4-trifluoromethyl-phenyl)-oxazole

67% yield. MS: 283.1 (M+1)

### Example 638

### 4-(2-Azido-ethyl)-2-(4-tert-butyl-phenyl)-oxazole

92% yield. MS: 271.3 (M+1)

### Example 639

### 4-(2-Azido-ethyl)-2-cyclohexyl-oxazole

55% yield. MS: 221.2 (M+1)

### Example 640

### 4-(2-Azido-ethyl)-2-(4-tert-butyl-phenyl)-5-methyl-oxazole

94% yield. MS: 285.4 (M+1)

### Example 641

### 2-[5-Methyl-2-(4-trifluoromethyl-phenyl)-thiazol-4-yl]-ethylamine

A mixture containing 4-(2-azido-ethyl)-5-methyl-2-(4-trifluoromethyl-phenyl)-thiazole (0.668 g, 2.14 mmol) and 0.668 g 10% palladium-on-celite in 30 ml methanol was hydrogenated at 50 psi overnight. The reaction mixture was then filtered and the filtrate was concentrated to dryness under reduced pressure to yield the title compound as a yellowish solid (0.579 g, 94% yield). MS: 287.2 (M+1)

The title compounds of EXAMPLES 642-658 were prepared using procedures analogous to that of EXAMPLE 641 from appropriate starting materials.

### Example 642

### 2-(5-Methyl-2-naphthalen-2-yl-thiazol-4-yl)-ethylamine

66% yield. MS: 269.1 (M+1)

### Example 643

### 2-(5-Methyl-2-phenyl-thiazol-4-yl)-ethylamine

75% yield. MS: 219.1 (M+1)

### Example 644

### 2-[2-(4-tert-Butyl-phenyl)-5-methyl-thiazol-4-yl]-ethylamine

95% yield. MS: 275.2 (M+1)

### Example 645

### 2-{5-Methyl-2-[4-(5-trifluoromethyl-Pyridin-2-yloxy)-phenyl]-thiazol-4-yl}-ethylamine

97% yield. MS: 380.3 (M+1)

### Example 646

### 2-[5-Methyl-2-(3-pyrrol-1-yl-phenyl)-thiazol-4-yl]-ethylamine

100% yield. MS: 284.3 (M+1)

### Example 647

### 3-[2-(4-Trifluoromethyl-phenyl)-thiazol-4-yl]-propylamine

72% yield. MS: 287.3 (M+1)

### Example 648

### 2-[2-(4-Trifluoromethyl-phenyl)-thiazol-4-yl]-ethylamine

76% yield. MS: 273.1 (M+1)

### Example 649

### 2-[2-(4-tert-Butyl-phenyl)-thiazol-4-yl]-ethylamine

75% yield. MS: 261.3 (M+1)

### Example 650

### 2-[2-(2,4-Difluoro-phenyl)-thiazol-4-yl]-ethylamine

99% yield. MS: 241.3 (M+1)

### Example 651

### 2-(2-p-Tolyl-thiazol-4-yl)-ethylamine

100% yield. MS: 219.3 (M+1)

### Example 652

### 2-[2-(4-Fluoro-phenyl)-thiazol-4-yl]-ethylamine 100% yield. MS: 223.2 (M+1)

### Example 653

### 2-[2-(3-Chloro-4-fluoro-phenyl)-thiazol-4-yl]-ethylamine

65% yield. MS: 257.0 (M+1) 506

### Example 654

### 2-[2-(4-Trifluoromethyl-phenyl)-oxazol-4-yl]-ethylamine

40% yield. MS: 257.1 (M+1)

### Example 655

### 2-[2-(4-tert-Butyl-phenyl)-oxazol-4-yl]-ethylamine

100% yield. MS: 245.2 (M+1)

### Example 656

### 2-(2-Cyclohexyl-oxazol-4-yl)-ethylamine

22% yield. MS: 195.2 (M+1)

### Example 657

### 2-(5-Methyl-2-phenyl-oxazol-4-yl)-ethylamine

75% yield. MS: 203.1 (M+1)

### Example 658

### 2-[2-(4-tert-Butyl-phenyl)-5-methyl-oxazol-4-yl]-ethylamine

86% yield. MS: 259.4 (M+1)

The title compounds of EXAMPLES 659-660 were prepared using procedures analogous to that of EXAMPLE 642 from appropriate starting materials except that Lindlar's catalyst was used instead of 10% palladium-on-celite.

### Example 659

### 2-[2-(4-Chloro-phenyl)-5-methyl-thiazol-4-yl]-ethylamine

93% yield. MS: 253.1 (M+1)

### Example 660

### 2-[2-(3-Chloro-4-fluoro-phenyl)-5-methyl-thiazol-4-yl]-ethylamine

97% yield. MS: 271.1 (M+1)

### Example 661

### [2-(4-Chloro-phenyl)-thiazol-4-yl]-acetonitrile

A solution of 4-chlorothiobenzamide (1.0 g, 5.82 mmol) and 1,3-dichloroacetone (0.88 g, 7.0 mmol) in 10 ml ethanol was heated at 80° C. for 2 hr. The reaction mixture was cooled to room temperature and poured into 50 ml water. The aqueous mixture was extracted with 50 ml ethyl acetate and the ethyl acetate solution was washed with 40 ml brine, dried (anhydrous sodium sulfate) and concentrated to dryness under reduced pressure. The residue was dissolved in 5 ml dimethylformamide, sodium cyanide (0.35 g, 7.14 mmol) was added and the reaction mixture was stirred at room temperature overnight. The reaction mixture was then poured into 50 ml water and the aqueous mixture was extracted with 60 ml ethyl acetate. The ethyl acetate solution was washed with 50 ml brine, dried (anhydrous sodium sulfate) and concentrated to dryness under reduced pressure to yield the title compound as a brownish solid (1.53 g, 100% yield)

### Example 662

### [2-(4-Trifluoromethoxy-phenyl)-thiazol-4-yl]-acetonitrile

The title compound was prepared using a procedure analogous to that of EXAMPLE 661. 100% yield. MS: 285.1 (M+1)

### Example 663

### 2-[2-(4-Chloro-phenyl)-thiazol-4-yl]-ethylamine

A solution of trifluoroacetic acid (0.502 ml, 6.5 mmol) in 5 ml tetrahydrofuran was added dropwise to a suspension of sodium borohydride (0.246 g, 6.5 mmol) in 30 ml tetrahydrofuran, followed by a solution of [2-(4-chloro-phenyl)-thiazol-4-yl-acetonitrile (1.53 g, 6.5 mmol) in 5 ml tetrahydrofuran. The reaction mixture was stirred at room temperature overnight, then poured into 150 ml water. The aqueous mixture was extracted with 200 ml ethyl acetate and the ethyl acetate solution was washed sequentially with 2×100 ml water and 100 ml brine, dried (anhydrous sulfate) and concentrated to dryness under reduced pressure. The crude product (1.68 g) was purified by flash column chromatography (40 g silica gel), eluting with 4:1 chloroform/methanol to yield the title compound as a yellowish oil (0.065 g, 4% yield).

MS: 239.1 (M+1)

### Example 664

### 2-[2-(4-Trifluoromethoxy-phenyl)-thiazol-4-yl]-ethylamine

The title compound was prepared using a procedure analogous to that of EXAMPLE 663 from appropriate starting materials. 13% yield. MS: 289.12 (M+1)

### Example 665

### 2-(4-Phenoxy-phenyl)-ethylamine

Ammonia (2.08 g) was bubbled into a mixture of 4-phenoxy]phenylacetonitrile (1.0 g, 4.78 mmol) in 70 ml methanol. Raney nickel (0.69 g) was then added and the mixture was hydrogenated overnight at 50 psi. The mixture was then filtered and the filtrate was concentrated to dryness under reduced pressure. The residue was dissolved in 40 ml ethyl acetate and the ethyl acetate solution was washed sequentially with 30 ml water and 30 ml brine, dried (anhydrous sodium sulfate) and concentrated to dryness under reduced pressure to yield the title compound as a yellowish oil (1.0 g, 100% yield). MS: 214.1 (M+1)

The title compounds of EXAMPLES 666-667 were prepared using procedures analogous to that of EXAMPLE 665 from appropriate starting materials.

### Example 666

### 2-(4-Benzyl-phenyl)-ethylamine

70% yield. MS: 212.1 (M+1)

### Example 667

### 2-Naphthalen-2-yl-ethylamine

71% yield. MS: 172.0 (M+1)

### Example 668

### Trans-4-(2-chloro-6-fluorobenzyloxy)-β-nitrostyrene

A mixture of 4-(2-chloro-6-fluorobenzyloxy)benzaldehyde (1 g, 3.82 mmol) and ammonium acetate (0.294 g, 3.82 mmol) in 10 ml nitromethane was heated at 110° C. for 15 min. The reaction mixture was then cooled to room temperature and concentrated under reduced pressure. The residue was partitioned between 150 ml water and 150 ml ethyl acetate. The aqueous layer was extracted with 100 ml ethyl acetate and the combined ethyl acetate extracts were washed with 150 ml brine. The ethyl acetate solution was dried (anhydrous sodium sulfate) and concentrated to dryness under reduced pressure to yield the title compound (0.922 g, 78% yield).

MS: 308.0 (M+1)

### Example 669

### Trans-4-(4-trifluoromethylphenoxy)-β-nitrostyrene

The title compound was prepared using a procedure analogous to that of EXAMPLE 668 from appropriate starting materials. 100% yield. ¹H NMR (400 MHz, CDCl₃): δ 7.17 (m, 3H), 7.26 (c, 2H), 7.3-7.55 (c, 4H), 7.96 (d, 1H).

### Example 670

### 2-(4-Benzyloxy-3-methoxy-phenyl)-ethylamine

A solution of trans-4-benzyloxy-3-methoxy-p-nitrostyrene (2.0 g, 7.01 mmol) in 20 tetrahydrofuran was added dropwise to a solution of lithium aluminum hydride (22.4 ml of a 1M solution, 22.4 mmol) in tetrahydrofuran. The reaction mixture was stirred at room temperature overnight, then quenched by the sequential addition, dropwise, of 1 ml aqueous 1N sodium hydroxide solution and 3 ml water. The resulting precipitate was filtered and the filtrate concentrated to dryness under reduced pressure to yield the title compound as a yellowish oil (1.53 g, 85% yield). MS: 258.1 (M+1)

The title compounds of EXAMPLES 671-673 were prepared using procedures analogous to that of EXAMPLE 670 from appropriate starting materials.

### Example 671

### 2-Naphthalen-1-yl-ethylamine

100% yield. MS: 170.0 (M−1)

### Example 672

### 2-[4-(4-Trifluoromethyl-phenoxy)-phenyl]-ethylamine

100% yield. MS: 282.1 (M+1)

### Example 673

### 2-[4-(2-Chloro-6-fluoro-benzyloxy)-phenyl]-ethylamine

100% yield. MS: 280.0 (M+1)

### Example 674

### 2-(6-Phenyl-pyridazin-3-ylsulfanyl)-ethylamine

Sodium t-butoxide (1.69 g, 17.6 mmol) was added to a solution of 2-aminoethanethiol hydrochloride (1.0 g, 8.8 mmol) in 20 ml anhydrous tetrahydrofuran cooled in an ice bath. The ice bath was removed and the solution was stirred at room temperature for 10 min. A solution of 3-chloro-6-phenylpyridazine (1.0 g, 5.2 mmol) in 3 ml tetrahydrofuran was added and the reaction mixture was stirred at room temperature overnight. 150 ml ethyl acetate was then added to the reaction mixture and the resulting solution was washed with 80 ml water and 80 ml brine, dried (anhydrous sodium sulfate) and concentrated to dryness under reduced pressure to yield the title compound as a yellowish solid (1.2 g, 98% yield). MS: 232.3 (M+1)

### Examples 675 and 676

### 2-Isopropylbenzoic Acid and Methyl Ester 2-Isopropylbenzonitrile

o-Isopropyl iodobenzene (8 g, 32.5 mmol), Pd₂(dba)₃ [tris(dibenzylidene-acetone)dipalladium] (1.19 g, 1.3 mmol), DPPF ((diphenylphosphinoferrocene)) (2.88 g, 5.2 mmol), tetraethylammonium cyanide (5.2 g, 32.5 mmol), and copper(1) cyanide (11.6 g, 130 mmol) were dissolved in 100 ml anhydrous tetrahydrofuran. The reaction was heated at reflux for 1.5 hr, then cooled to room temperature. The solution was concentrated to half volume under reduced pressure, diluted with 250 ml ethyl acetate and the resulting solution was filtered through a pad of diatomaceous earth (Celite). The filtrate was washed with 150 ml saturated aqueous sodium bicarbonate solution, dried over anhydrous sodium sulfate and concentrated to dryness under reduced pressure. The crude product was purified by flash column chromatography on silica gel, eluting with 99:1 hexane/ethyl acetate to yield the product (4.9 g, 100% yield). MS: 146.0 (M+1)

### 2-isopropylbenzoic Acid

A solution of 2-isopropylbenzonitrile (2.5 g, 17.5 mmol) and potassium hydroxide (3.24 g, 57.7 mmol) in 15 ml ethylene glycol was heated at 170° C. for 3.5 hr, then cooled to room temperature. The reaction mixture was poured into 120 ml water and 120 ml ethyl acetate and shaken. The ethyl acetate layer was discarded and the water layer was acidified to pH 1 with 6N aqueous hydrochloric acid solution and extracted with 3×90 ml ethyl acetate. The combined ethyl acetate extracts were sequentially washed with 150 ml water, and 150 ml brine, dried (anhydrous sodium sulfate) and concentrated to dryness under reduced pressure to a brownish oil that solidified (2.5 g, 87% yield). MS: 164.1 (M+1)

### 2-Isopropylbenzoic Acid Methyl Ester

2-Isopropylbenzoic acid (2.49 gm, 15.2 mmol) was added to 4.8 ml thionyl chloride, followed by 2 drops dimethylformamide. The reaction mixture was heated at reflux for 3 hr, cooled and concentrated to dryness under reduced pressure. 10 ml methylene chloride was added to the residue and the resulting solution was concentrated to dryness under reduced pressure. The procedure was repeated twice to remove the last traces of thionyl chloride. 25 ml anhydrous methanol was added to the residue, followed by 1.29 ml (15.9 mmol) pyridine. The resulting solution was heated at reflux overnight, then cooled to room temperature and concentrated to dryness under reduced pressure. The residue was dissolved in 130 ml ethyl acetate and the ethyl acetate solution was washed sequentially with 90 ml water, 90 ml 1N aqueous hydrochloric acid solution, 90 ml water and 90 ml brine, dried (anhydrous sodium sulfate) and concentrated to dryness under reduced pressure to a brownish oil (2.3 gm, 85% yield). ¹H NMR (400 MHz, CDCl₃) δ 1.26 (d, 6H), 3.70 (m, 1H), 3.89 (s, 3H), 7.21 (m, 2H), 7.44 (m, 2H), 7.71 (m, 1H).

### Example 677

### 5-Chlorosulfonyl-2-methyl-benzoic acid

A mixture of o-toluic acid (15 g, 0.11 mol) and chlorosulfonic acid (30 ml) was heated at 100° C. under nitrogen for 2.5 h. The reaction mixture was then poured onto ice (500 ml) and the resulting precipitate was filtered, yielding the title compound as an off-white solid (20 g, 78% yield). MP 151-155° C. MS: 233.4 (M−1)

The title compounds of EXAMPLES 678-680 were prepared using a procedure analogous to that of EXAMPLE 677 from appropriate starting materials.

### Example 678

### 3-Chlorosulfonyl-2,6-dimethyl-benzoic acid

28% yield. ¹H NMR (400 MHz, CD₃OD) δ 2.44 (s, 3H), 2.72 (s, 3H), 7.41 (d, 1H), 8.02 (d, 1H),

### Example 679

### 5-Chlorosulfonyl-2,3-dimethyl-benzoic acid

77% yield. ¹H NMR (400 MHz, CDCl₃) δ 2.49 (s, 3H), 2.66 (s, 3H), 7.98 (s, 1H), 8.47 (s, 1H).

### Example 680

### 5-Chlorosulfonyl-2-ethyl-benzoic acid

76% yield. MS: 247.0 (M−1)

### Example 681

### 5-Chlorosulfonyl-2-methyl-benzoic acid methyl ester

Chlorosulfonic acid (106.2 ml) was carefully added over 1 min with stirring under nitrogen to 2-methyl-benzoic acid methyl ester (55.9 ml, 0.4 mol). The reaction mixture was placed in an oil bath preheated to 100° C. for 15 min, then poured onto ice (1000 ml). The resulting precipitate was filtered and dissolved in ethyl acetate (400 ml). The ethyl acetate solution was washed sequentially with 10×300 ml saturated aqueous sodium bicarbonate, 300 ml water and 300 ml brine, dried (anhydrous sodium sulfate) and concentrated under reduced pressure to yield the title compound as a yellowish oil (37.3 g, 37% yield). ¹H NMR (400 MHz, CDCl₃) 8, 2.74 (s, 3H), 3.96 (s, 3H), 7.52 (d, 1H), 8.04 (m, 1H), 8.58 (d, 1H).

The title compounds of EXAMPLES 682-686 were prepared using procedures analogous to that of EXAMPLE 681 from appropriate starting materials.

### Example 682

### 5-Chlorosulfonyl-2-ethyl-benzoic acid methyl ester

42% yield. ¹H NMR (400 MHz, CDCl₃) δ 1.29 (t, 3H), 3.11 (q, 2H), 3.96 (s, 3H), 7.54 (d, 1H), 8.06 (m, 1H), 8.53 (d, 1H). MS: 249.5 (M+1)

### Example 683

### 5-Chlorosulfonyl-2-isopropyl-benzoic acid methyl ester

47% yield. ¹H NMR (400 MHz, CDCl₃) δ 1.3 (d, 6H), 3.87 (m, 1H), 3.96 (s, 3H), 7.67 (d, 1H), 8.08 (m, 1H), 8.41 (d, 1H).

### Example 684

### 5-Chlorosulfonyl-2,3-dimethyl-benzoic acid methyl ester

41% yield. ¹H NMR (400 MHz, CDCl₃) δ 2.45 (s, 3H), 2.58 (s, 3H), 3.95 (s, 3H), 7.92 (d, 1H), 8.31 (d, 1H),

### Example 685

### 5-Chlorosulfonyl-2-ethoxy-benzoic acid ethyl ester

10% yield. ¹H NMR (400 MHz, CDCl₃) δ1.43 (t, 3H), 1.52 (t, 3H), 4.24 (q, 2H), 4.40 (q, 2H), 7.10 (d, 1H), 8.09 (m, 1H), 8.43 (d, 1H).

### Example 686

### 5-Chlorosulfonyl-2-methylsulfanyl-benzoic acid methyl ester

58% yield. ¹H NMR (400 MHz, CDCl₃) δ 2.55 (s, 3H), 3.98 (s, 3H), 7.47 (d, 1H), 8.05 (m, 1H), 8.64 (d, 1H).

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a Formula I 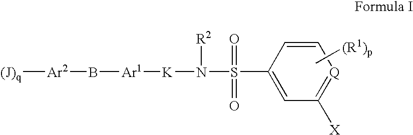
or a pharmaceutically acceptable salt of said compound, wherein
Q is carbon;
each R¹ is independently hydrogen, halo, (C₁-C₅)alkyl optionally substituted with one to eleven halo or with (C₁-C₃)alkoxy, (C₁-C₅)alkoxy optionally substituted with one to eleven halo, (C₁-C₅)alkylthio optionally substituted with one or more halo, or R¹ in conjunction with the two adjacent carbon atoms forms a C₅-C₆ fused fully saturated, partially unsaturated or fully unsaturated five or six membered carbocyclic ring wherein each carbon in the carbon chain may optionally be replaced with one heteroatom selected from oxygen and sulfur;
R² is hydrogen, (C₁-C₅)alkyl optionally substituted with C₁-C₃ alkoxy, or benzyl optionally substituted with one to three substituents selected from the group consisting of halo, (C₁-C₄)alkyl optionally substituted with one to nine halo, (C₁-C₄)alkoxy optionally substituted with one to nine halo, and (C₁-C₄)alkylthio optionally substituted with one to nine halo;
K is —O—(CZ₂)ₜ—, —S—(CZ₂)ₜ—, —(CZ₂)ᵤ—, or K and R² together form a fully saturated or partially unsaturated four to six membered cyclic carbon chain and wherein each Z is independently hydrogen or (C₁-C₃)alkyl, t is 2, 3 or 4, and u is 1, 2, 3 or 4;
X is —COOR⁴, —O—(CR³₂)—COOR⁴, —S—(CR³₂)—COOR⁴, —CH₂—(CR⁵₂), —COOR⁴, 1H-tetrazol-5-yl-E- or thiazolidinedione-5-yl-G-; wherein w is 0, 1 or 2; E is (CH₂)ᵣ and r is 0, 1, 2 or 3, and G is (CH₂), or methylidene and s is 0 or 1;
each R³ is independently hydrogen, (C₁-C₄)alkyl optionally substituted with one to nine halo, or (C₁-C₃)alkoxy optionally substituted with one or more halo, or R³ and the carbon to which it is attached form a 3, 4, 5, or 6 membered carbocyclic ring;
R⁴ is H, (C₁-C₄)alkyl, benzyl or p-nitrobenzyl;
each R⁵ is independently hydrogen, (C₁-C₄)alkyl optionally substituted with one to nine halo or with (C₁-C₃)alkoxy, (C₁-C₄)alkoxy optionally substituted with one to nine halo, (C₁-C₄)alkylthio optionally substituted with one to nine halo or with (C₁-C₃)alkoxy, or R⁵ and the carbon to which it is attached form a 3, 4, 5, or 6 membered carbocyclic ring wherein any carbon of the 5- or 6-membered ring may be replaced by an oxygen atom;
Ar¹ is thiazolyl, oxazolyl, pyridinyl, triazolyl, pyridazyl, or phenyl, wherein phenyl is optionally fused to a member selected from thiazolyl, furanyl, oxazolyl, pyridine, pyrimidine, phenyl, or thienyl wherein Ar¹ is optionally mono-, di- or tri-substituted with Z, wherein each Z is independently: hydrogen, halo, (C₁-C₃)alkyl optionally substituted with one to seven halo, (C₁-C₃)alkoxy optionally substituted with one to seven halo or (C₁-C₃)alkylthio optionally substituted with one to seven halo;
B is a bond, CO, (CY₂)ₙ, CYOH, CY═CY, -L-(CY₂)ₙ—, —(CY₂)ₙ-L-, -L-(CY₂)₂-L-, NY—OC—, —CONY—, —SO₂NY—, —NY—SO₂— wherein each L is independently O, S, SO, or SO₂, each Y is independently hydrogen or (C₁-C₃) alkyl, and n is 0, 1, 2 or 3;
Ar² is a bond, phenyl, phenoxybenzyl, phenoxyphenyl, benzyloxyphenyl, benzyloxybenzyl, pyrimidinyl, pyridinyl, pyrazolyl, imidazolyl, thiazolyl, thiadiazolyl, oxazolyl, oxadiazolyl or phenyl fused to a ring selected from the group consisting of: phenyl, pyrimidinyl, thienyl, furanyl, pyrrolyl, thiazolyl, oxazolyl, pyrazolyl, and imidazolyl;
each J is independently hydrogen, hydroxy, halo, (C₁-C₈)alkyl optionally substituted with one to seventeen halo, (C₁-C₈)alkoxy optionally substituted with one to seventeen halo, (C₁-C₈)alkylthio optionally substituted with one to seventeen halo, (C₃-C₇)cycloalkyl, (C₃-C₇)cycloalkyloxy, (C₃-C₇)cycloalkylthio, or phenyl optionally substituted with one to four substituents from the group consisting of: halo, (C₁-C₃)alkyl optionally substituted with one to seven halo, (C₁-C₃)alkoxy optionally substituted with one to seven halo, and (C₁-C₃)alkylthio optionally substituted with one to seven halo; and
p and q are each independently 0, 1, 2 or 3; and
with the provisos:
a) if Ar¹ is phenyl, B is a bond, Ar² is a bond or phenyl, K is (CH₂)ₜ and X is —COOH then q is other than 0 and J is other than hydrogen; and
b) if Ar¹ is phenyl, B is not a bond, Ar² is phenyl, K is —(CH₂)ₜ— and X is —COOR⁴ then B is attached to Ar¹ para to K.

2. A compound according to claim 1, wherein Ar¹ is: 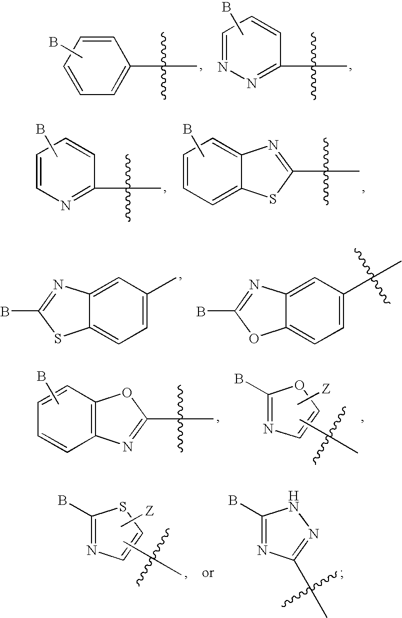
wherein Z is hydrogen or (C₁-C₃)alkyl optionally substituted with one to seven halo.

3. A compound according to claim 1 or 2, wherein Ar² is

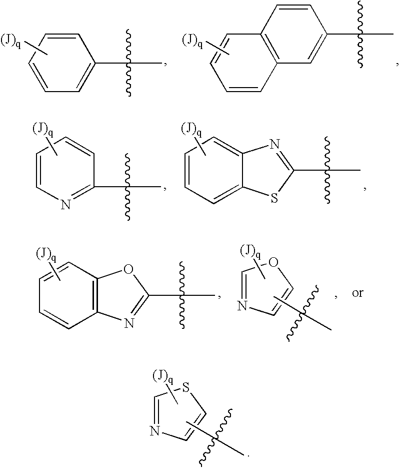

4. A compound according to claim 1, wherein,
B is a bond or -L-(CY₂)ₙ— or —(CY₂)ₙ-L-;
L is O or S;
K is —(CH₂)ᵤ— and u is 1, 2, or 3;
n is 0, 1 or 2;
p is 1, 2, or 3 and at least one R¹ is attached at Q;
Ar¹ is oxazolyl, thiazolyl, phenyl or phenyl fused to oxazolyl or thiazolyl wherein Ar¹ is optionally mono-, di- or tri-substituted with Z; and
Ar² is phenyl or a bond.

5. A compound according to claim 1, wherein
X is —COOR⁴;
K is —O—(CH₂)ₜ—, —S—(CH₂)ₜ—, or —(CH₂)ᵤ— wherein t is 2 or 3 and u is 1, 2 or 3;
B is a bond;
p is 1, 2, or 3 and at least one R¹ is attached at Q;
Ar¹ is oxazolyl, thiazolyl, phenyl or phenyl fused to oxazolyl or thiazolyl wherein Ar¹ is optionally mono-, di- or tri-substituted with Z; and
Ar² is a bond or is phenyl.

6. A compound according to claim 5, wherein K is —(CH₂)ᵤ— and u is 1, 2, or 3;
p is 1 or 2;
R⁴ is H or (C₁-C₃)alkyl; and
Ar¹ is: 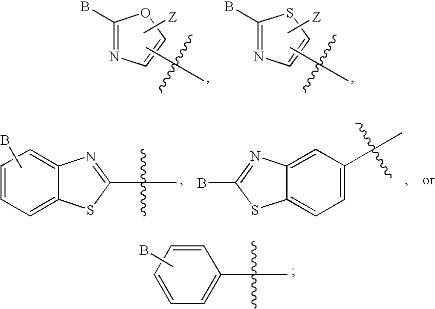
wherein Z is hydrogen or (C₁-C₃)alkyl optionally substituted with one to seven halo.

7. A compound according to claim 1, wherein X is —COOR⁴;
K is —O—(CH₂)ₜ—, —S—(CH₂)ₜ—, or —(CH₂)ᵤ— wherein t is 2 or 3 and u is 1, 2 or 3;
B is -L-(CY₂)ₙ— or —(CY₂)ₙ-L-, and L is O or S, and n is 0, 1 or 2;
p is 1, 2, or 3 and at least one R¹ is attached at Q;
Ar¹ is oxazolyl, thiazolyl, phenyl, or phenyl fused to oxazolyl or thiazolyl wherein Ar¹ is optionally mono-, di- or tri-substituted with Z; and
Ar² is a bond or is phenyl.

8. A compound according to claim 7, wherein
K is —(CH₂)ᵤ—;
L is O and n is 0 or 1;
p is 1 or 2 and R⁴ is H or (C₁-C₃)alkyl;
Ar¹ is phenyl; and
Ar² is phenyl.

9. A compound selected from the group consisting of:
5-{2-[2-(4-Chloro-phenyl)-5-methyl-thiazol-4-yl]-ethylsulfamoyl}-2-isopropyl-benzoic acid;
5-{3-[2-(4-Chloro-phenyl)-thiazol-4-yl]-propylsulfamoyl}-2-methyl-benzoic acid;
2-Isopropyl-5-[2-(5-methyl-2-phenyl-oxazol-4-yl)-ethylsulfamoyl]-benzoic acid;
5-{2-[4-(3,4-Difluoro-phenoxy)-phenyl]-ethylsulfamoyl}-2-methyl-benzoic acid;
5-{2-[4-(4-Fluoro-phenoxy)-phenyl]-ethylsulfamoyl}-2-methyl-benzoic acid;
5-{2-[4-(4-Fluoro-phenoxy)-phenyl]-ethylsulfamoyl}-2,3-dimethyl-benzoic acid;
5-{2-[4-(4-Fluoro-3-methyl-phenoxy)-phenyl]-ethylsulfamoyl}-2-methyl-benzoic acid;
2-[4-(3-Chloro-4-fluoro-phenoxy)-phenyl]-ethylsulfamoyl)-2-ethyl-benzoic acid;
2-Isopropyl-5-[2-(2-phenyl-benzooxazol-5-yl)-ethylsulfamoyl]-benzoic acid;
2-Methyl-5-(2-[2-(4-trifluoromethyl-phenyl)-oxazol-4-yl]-ethylsulfamoyl)-benzoic acid;
2-Isopropyl-5-[2-(2-phenyl-benzothiazol-5-yl)-ethylsulfamoyl]-benzoic acid;
2-Isopropyl-5-{2-[5-methyl-2-(4-trifluoromethyl-phenyl)-thiazol-4-yl]-ethylsulfamoyl}-benzoic acid;
2-Ethyl-5-[2-(2-phenyl-benzothiazol-5-yl)-ethylsulfamoyl]-benzoic acid;
2-Ethyl-5-{2-[5-methyl-2-(4-trifluoromethyl-phenyl)-thiazol-4-yl]-ethylsulfamoyl}-benzoic acid;
2-Methyl-5-{2-[5-methyl-2-(4-trifluoromethoxy-phenyl)-thiazol-4-yl]-ethylsulfamoyl}-benzoic acid;
2-Methyl-5-{3-[2-(4-trifluoromethyl-phenyl)-thiazol-4-yl]-propylsulfamoyl}-benzoic acid;
2-Ethyl-5-{3-[2-(4-trifluoromethyl-phenyl)-thiazol-4-yl]-propylsulfamoyl}-benzoic acid;
2-Ethyl-5-[2-(4-phenoxy-phenyl)-ethylsulfamoyl]-benzoic acid;
5-{2-[4-(4-Fluoro-phenoxy)-phenylsulfanyl]-ethylsulfamoyl}-2,3-dimethyl-benzoic acid; and
2-Ethyl-5-{2-[4-(4-trifluoromethyl-phenoxy)-phenyl]-ethylsulfamoyl}-benzoic acid;
or a pharmaceutically acceptable salt of said compound.

10. A method for treating dyslipidemia, obesity, overweight condition, hypertriglyceridemia, hyperlipidemia, hypoalphalipoproteinemia, metabolic syndrome, diabetes mellitus (Type I and/or Type II), hyperinsulinemia, impaired glucose tolerance, insulin resistance, diabetic complications, atherosclerosis, hypertension, coronary heart disease, coronary artery disease hypercholesterolemia, inflammation, osteoporosis, thrombosis, peripheral vascular disease, cognitive dysfunction, or congestive heart failure in a mammal by administering to a mammal in need of such treatment a therapeutically effective amount of a compound of claim 1 or 9, or a pharmaceutically acceptable salt of said compound.

11. A pharmaceutical composition which comprises a therapeutically effective amount of a compound of claim 1 or 9, or a pharmaceutically acceptable salt of said compound and a pharmaceutically acceptable carrier, vehicle or diluent.

12. A pharmaceutical combination composition comprising: a therapeutically effective amount of a composition comprising
a first compound, said first compound being a compound of claim 1 or 9, or a pharmaceutically acceptable salt of said compound;
a second compound, said second compound being a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, a combination of niacin and lovastatin, an ion-exchange resin, an antioxidant, an ACAT inhibitor, a bile acid sequestrant, or a pharmaceutically acceptable salt of said compound; and
a pharmaceutically acceptable carrier, vehicle or diluent.

13. A pharmaceutical combination composition of claim 12 wherein the second compound is rosuvastatin, rivastatin, pitavastatin, lovastatin, simvastatin, pravastatin, fluvastatin, atorvastatin or cerivastatin or a pharmaceutically acceptable salt of said compound.

14. A method for treating atherosclerosis in a mammal comprising administering to a mammal in need of treatment thereof;
a first compound, said first compound being a compound of claim 1 or 9, or a pharmaceutically acceptable salt of said compound; and
a second compound, said second compound being a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, a combination of niacin and lovastatin, an ion-exchange resin, an antioxidant, an ACAT inhibitor or a bile acid sequestrant
wherein the amounts of first and second compounds result in a therapeutic effect.

15. A method for treating atherosclerosis of claim 14 wherein the second compound is rosuvastatin, pitavastatin, lovastatin, simvastatin, pravastatin, fluvastatin, atorvastatin or cerivastatin or a pharmaceutically acceptable salt of said compound.
